SEQUENCE LISTING (196 sequences)

SEQ ID NO: 1 - RNA, 57 nt, Synthetic construct
  source 1..57: mol_type = other RNA
gggaaataagagagaaaagaagagtaagaagaaatataagaccccggcgccgccacc

SEQ ID NO: 2 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 3 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICNFRGDVTHITGSRNYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 4 - RNA, 119 nt, Synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - RNA, 1590 nt, Synthetic construct
  source 1..1590: mol_type = other RNA
atgatgatggccagcaaggacgccaccagcagcgtggacggcgcttctggggccggacagctggtgcccgaggtgaacgccagcgacccactggccatggaccccgtggccggaagcagcaccgccgtggccaccgcaggacaggtgaaccccatcgacccttggatcatcaacaacttcgtgcaggcaccccagggcgagttcaccatcagccccaacaacactcccggcgacgtgctgttcgacctgtccctgggcccacacctgaacccctttctgctgcacctgagccagatgtacaacggctgggtgggcaacatgcgggtgcggatcatgctggccggcaacgcctttaccgccggcaagatcatcgtgagctgcatcccacccggctttggcagccacaacctgaccatcgcccaggccaccctgttcccacacgtgatcgccgacgtccggaccctggatcccatcgaggtgcccctggaggacgtgcggaacgtgcttttccacaacaacgaccggaaccagcagaccatgcgcctggtgtgcatgctgtacactccacttcggacaggcggcggaaccggcgacagcttcgtggtggccggccgggtgatgacctgcccttccccggacttcaacttcctgttcctggtgccacccaccgtggagcagaagacccggcccttcaccctgcccaacctgcccctgagcagcctgagcaacagccgggcacccctgcctatcagcagcatgggcatctcacccgacaacgtgcagagcgtgcagttccagaacggcaggtgcaccctcgacggccggctggtgggcaccacgcccgtgagcctgtctcacgtggccaagatccgcggcaccagtaacggcaccgtgattaatctgaccgagctggacggcactcccttccaccctttcgagggccccgcgccaatcggattccccgacttaggtggctgcgactggcacatcaacatgacccagttcggccacagcagccagacccagtacgacgtggacaccacccctgacaccttcgtgccccacctgggcagcatccaggccaacggcatcggcagcggcaactacgtgggcgtgctgagctggatcagccctcccagccaccccagcggcagccaagtggacctgtggaagatccccaactacgggagcagcatcaccgaagctacccacctcgctcccagcgtgtaccctcctggcttcggcgaggtgctggtgttcttcatgagcaagatgcctggccctggcgcctacaatctgccctgcctgctgccccaggagtacatcagtcacctggccagcgagcaggctccaacagtgggcgaggccgccttactgcactacgtcgaccccgacaccggccggaacctgggagagttcaaggcctaccccgacggcttcctgacctgcgtgcccaacggcgcaagcagcggccctcagcagctgcccatcaacggcgtgttcgtgtttgtgagctgggtgagccggttctaccagctgaagcccgtaggcaccgccagctccgctcggggaagactgggcctgcggcgg

SEQ ID NO: 6 - protein, 530 aa, Synthetic construct
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 7 - RNA, 1623 nt, Synthetic construct
  source 1..1623: mol_type = other RNA
atgcggatgagcgacggcgccgcccctaaggccaacggcagtgaggccagcggccaggacttggtgcccaccgccgtggagcaggcagtgccaatccaacccgtggctggcgccgctttagccgccccagcagctggccagatcaaccagatagacccctggatcttccagaactttgtgcagtgtcccctgggcgagttcagcatcagccctcggaacacgcccggcgagatcctgttcgatctggcgctgggacccggcctgaacccctacctggcccacctgagcgccatgtacaccggctgggtgggcaacatggaagtgcagctggtgctggccggtaacgcctttaccgccggcaaggtggtggtggccctggtgccaccctacttccccaagggcagcctgaccaccgcccagatcacctgcttccctcacgtgatgtgcgacgtgagaaccctggagcccatccagctgcccctgctggacgtgcggcgggtgctgtggcacgccacccaggaccaggaggagagcatgcggctggtgtgcatgctgtacaccccactgcggaccaacagccccggcgacgagagcttcgtggtgagcggccggctgctcagcaagcccgccgccgacttcaacttcgtgtacctgacccctccaatcgagcggaccatctaccggatggtggacctgcccgtgctgcaaccccggctgtgcacccacgccaggtggcctgcgccaatctacggcctgctggtggatcccagcctgcccagcaacccacagtggcagaacggccgggtgcacgtggacggcaccctgcttggcaccacccctgtgagcgggagctgggtgagctgcttcgccgcagaggccgcctacgagttccagagcggcatcggcgaggtcgccaccttcaccctgatcgagcaggacggcagcgcctacgtgcctggagacagggccgcgcctctgggctaccccgactttagcggacagctggagatcgaggtgcagaccgagaccaccaagaagggcgagaagctgaaggtgaccaccttcgagatgatcctgggccccacgaccaacgtggaccaggtgccctaccagggacgggtgtacgcaagcctgactgccgccgccagcctggacctggtggacggacgagtaagagccgtgccccggagcgtgtacggcttccaggacgtggtgcccgagtacaacgacggcctgttagtgcccctggccccacccatcggccccttcttaccaggcgaagtcctgctgcggttccggacctacatgcggcagatcgacagcaccgacgctgctgccgaggccatcgactgcgccctgccccaggagttcgtgagctggttcgccagcaacgccttcaccgtgcagagcgaggccctgctgctgagataccggaacacccttaccggtcaactgctgttcgagtgcaagctgtatagcgagggctacatcgccctgagctacagcggcagcggccctctgaccttccccaccgacggcttcttcgaggtggtgtcttgggtgccacggctgtaccagttggccagcgtgggctctctggccaccggccggaccctgaagcag

SEQ ID NO: 8 - protein, 541 aa, Synthetic construct
MRMSDGAAPKANGSEASGQDLVPTAVEQAVPIQPVAGAALAAPAAGQINQIDPWIFQNFVQCPLGEFSISPRNTPGEILFDLALGPGLNPYLAHLSAMYTGWVGNMEVQLVLAGNAFTAGKVVVALVPPYFPKGSLTTAQITCFPHVMCDVRTLEPIQLPLLDVRRVLWHATQDQEESMRLVCMLYTPLRTNSPGDESFVVSGRLLSKPAADFNFVYLTPPIERTIYRMVDLPVLQPRLCTHARWPAPIYGLLVDPSLPSNPQWQNGRVHVDGTLLGTTPVSGSWVSCFAAEAAYEFQSGIGEVATFTLIEQDGSAYVPGDRAAPLGYPDFSGQLEIEVQTETTKKGEKLKVTTFEMILGPTTNVDQVPYQGRVYASLTAAASLDLVDGRVRAVPRSVYGFQDVVPEYNDGLLVPLAPPIGPFLPGEVLLRFRTYMRQIDSTDAAAEAIDCALPQEFVSWFASNAFTVQSEALLLRYRNTLTGQLLFECKLYSEGYIALSYSGSGPLTFPTDGFFEVVSWVPRLYQLASVGSLATGRTLKQ

SEQ ID NO: 9 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 10 - protein, 541 aa, Synthetic construct; identical to SEQ ID NO: 8

SEQ ID NO: 11 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcgccgacacccacaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgctaccgacaccgctgctgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 12 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICNFRGDVTHIADTHNYTMNLASQNWNNYDPTEEIPAPLGTPDFVGKIQGMLTQTTRTDGSTRGHKATVYTGSADFAPKLGRVQFATDTAADFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 13 - RNA, 1590 nt, Synthetic construct
  source 1..1590: mol_type = other RNA
atgatgatggccagcaaggacgccaccagcagcgtggacggcgcttctggggccggacagctggtgcccgaggtgaacgccagcgacccactggccatggaccccgtggccggaagcagcaccgccgtggccaccgcaggacaggtgaaccccatcgacccttggatcatcaacaacttcgtgcaggcaccccagggcgagttcaccatcagccccaacaacactcccggcgacgtgctgttcgacctgtccctgggcccacacctgaacccctttctgctgcacctgagccagatgtacaacggctgggtgggcaacatgcgggtgcggatcatgctggccggctgcgcctttaccgccggcaagatcatcgtgagctgcatcccacccggctttggcagccacaacctgaccatcgcccaggccaccctgttcccacacgtgatcgccgacgtccggaccctggatcccatcgaggtgcccctggaggacgtgcggaacgtgcttttccacaacaacgaccggaaccagcagaccatgcgcctggtgtgcatgctgtacactccacttcggacatgcggcggaaccggcgacagcttcgtggtggccggccgggtgatgacctgcccttccccggacttcaacttcctgttcctggtgccacccaccgtggagcagaagacccggcccttcaccctgcccaacctgcccctgagcagcctgagcaacagccgggcacccctgcctatcagcagcatgggcatctcacccgacaacgtgcagagcgtgcagttccagaacggcaggtgcaccctcgacggccggctggtgggcaccacgcccgtgagcctgtctcacgtggccaagatccgcggcaccagtaacggcaccgtgattaatctgaccgagctggacggcactcccttccaccctttcgagggccccgcgccaatcggattccccgacttaggtggctgcgactggcacatcaacatgacccagttcggccacagcagccagacccagtacgacgtggacaccacccctgacaccttcgtgccccacctgggcagcatccaggccaacggcatcggcagcggcaactacgtgggcgtgctgagctggatcagccctcccagccaccccagcggcagccaagtggacctgtggaagatccccaactacgggagcagcatcaccgaagctacccacctcgctcccagcgtgtaccctcctggcttcggcgaggtgctggtgttcttcatgagcaagatgcctggccctggcgcctacaatctgccctgcctgctgccccaggagtacatcagtcacctggccagcgagcaggctccaacagtgggcgaggccgccttactgcactacgtcgaccccgacaccggccggaacctgggagagttcaaggcctaccccgacggcttcctgacctgcgtgcccaacggcgcaagcagcggccctcagcagctgcccatcaacggcgtgttcgtgtttgtgagctgggtgagccggttctaccagctgaagcccgtaggcaccgccagctccgctcggggaagactgggcctgcggcgg

SEQ ID NO: 14 - protein, 530 aa, Synthetic construct
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGCAFTAGKIIVSCIPPGFGSHNLTIAQATLFPHVIADVRTLDPIEVPLEDVRNVLFHNNDRNQQTMRLVCMLYTPLRTCGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 15 - RNA, 1641 nt, Synthetic construct
  source 1..1641: mol_type = other RNA
atgaagatggccagcaacgacgccgccccaagcaacgacggcgccgccaacctggtgcccgaggccaccaacgaggtgatggccctggagccagtggtgggcgcctccatcgctgcccctgtggtgggccagcagaacatcatcgacccctggatccgggagaacttcgtgcaggcccctcagggcgagttcaccgtgtctccccggaacagccccggcgagatgctgctgaacctggagctgggccccgagctgaacccctacctgagccacctgagccggatgtacaacggctacgccggcggcatgcaggtgcaggtggtgctggccggcaacgccttcaccgccggcaagatcatcttcgccgccgtgccaccccacttccccgtggagaacatcagcgccgcccagatcaccatgtgtccccacgtgatcgtggacgtgcggcagctggagcccgtgctgctgcccctgcccgacatccggaaccggttcttccactacaaccaggagaacacaccccggatgcggctggtggccatgctgtatactcccctgcgggccaatagcggcgaggacgtgttcaccgtgagctgtcgggtgctgaccaggcccgcacccgacttcgagttcaccttcctggtgccgcccaccgtggagagcaagaccaagcccttcaccctgcccatcctgaccctgggcgagctgagcaacagccggttccccgcccctatcgacatgctgtacaccgaccccaacgaggccatcgtggtgcagccccagaacggccggtgcaccttggacggcaccctgcagggcaccacccagctggtgcccacccagatctgcagcttccggggcaccctgatcagccagaccagccggagcgccgacagcaccgacagcgcaccccgggtgcggaaccatcccctccacgtgcagctgaagaacctggacggcactccctacgaccccaccgacgaagtgcccgcagtgctgggcgccatcgacttcaagggcaccgtgttcggcgtggccagccagcggaacaccaccggcaactctataggcgccaccagagcccacgaggtgcacatcgacaccaccaatccccggtacacccctaagctgggcagcgtgctgatgtacagcgagagcaccgacttcgacgacggccagcccacccggttcactcccatcggcatgggcgccgacgactggcaccagtgggagctgcccgagtacagcggccacctgaccctgaacatgaacttagccccagccgtggctccagcattccccggcgagcggatcctgttcttccggagcgtggtgccctcagccggcggttacggcagcggccacatcgactgcctgattccccaggagtgggtgcagcacttctaccaggaggccgcccctagccagagcgccgtggccctgatccggtacgtgaaccccgacaccggccggaacatcttcgaggccaagctgcaccgggagggcttcatcaccgtcgccaacagcggcaacaaccccatcgtggttcctccgaacggctacttccggttcgaggcctgggtgaaccagttctacaccctgaccccaatgggcacaggccagggccgacggagggtgcag

SEQ ID NO: 16 - protein, 547 aa, Synthetic construct
MKMASNDAAPSNDGAANLVPEATNEVMALEPVVGASIAAPVVGQQNIIDPWIRENFVQAPQGEFTVSPRNSPGEMLLNLELGPELNPYLSHLSRMYNGYAGGMQVQVVLAGNAFTAGKIIFAAVPPHFPVENISAAQITMCPHVIVDVRQLEPVLLPLPDIRNRFFHYNQENTPRMRLVAMLYTPLRANSGEDVFTVSCRVLTRPAPDFEFTFLVPPTVESKTKPFTLPILTLGELSNSRFPAPIDMLYTDPNEAIVVQPQNGRCTLDGTLQGTTQLVPTQICSFRGTLISQTSRSADSTDSAPRVRNHPLHVQLKNLDGTPYDPTDEVPAVLGAIDFKGTVFGVASQRNTTGNSIGATRAHEVHIDTTNPRYTPKLGSVLMYSESTDFDDGQPTRFTPIGMGADDWHQWELPEYSGHLTLNMNLAPAVAPAFPGERILFFRSVVPSAGGYGSGHIDCLIPQEWVQHFYQEAAPSQSAVALIRYVNPDTGRNIFEAKLHREGFITVANSGNNPIVVPPNGYFRFEAWVNQFYTLTPMGTGQGRRRVQ

SEQ ID NO: 17 - RNA, 1626 nt, Synthetic construct
  source 1..1626: mol_type = other RNA
atgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggcgagttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccacccaccgtggagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgacagctgggtgaaccagttctacagcctggccccaatgggcaccggtaacggacgccggcgggtgcag

SEQ ID NO: 18 - protein, 542 aa, Synthetic construct
MKMASNDAAPSTDGAAGLVPESNNEVMALEPVAGAALAAPVTGQTNIIDPWIRANFVQAPNGEFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTVESKTKPFTLPILTLGELSNSRFPVSIDQMYTSPNEIISVQCQNGRCTLDGELQGTTQLQVSGICAFKGEVTAHLHDNDHLYNVTITNLNGSPFDPSEDIPAPLGVPDFQGRVFGIISQRDKHNSPGHNEPANRGHDAVVPTYTAQYTPKLGQIQIGTWQTDDLTVNQPVKFTPVGLNDTEHFNQWVVPRYAGALNLNTNLAPSVAPVFPGERLLFFRSYIPLKGGYGNPAIDCLLPQEWVQHFYQEAAPSMSEVALVRYINPDTGRALFEAKLHRAGFMTVSSNTSAPVVVPANGYFRFDSWVNQFYSLAPMGTGNGRRRVQ

SEQ ID NO: 19 - RNA, 1620 nt, Synthetic construct
  source 1..1620: mol_type = other RNA
atgaagatggccagcaacgacgccgccccaagcaacgacggagccgctggcctggtgcccgagggcaacaacgagaccctgcccctggaacccgtggccggcgcagccattgccgccccagtgaccggccagaacaacatcatcgacccctggatccggaccaacttcgtgcaggctcccaacggcgagttcaccgtgtctccccggaacagccccggcgagatcctgctgaacctggagctgggccccgacctgaacccctacctggcccacctgagccggatgtacaacggctacgccggcggcgttgaggtgcaggtgctgctggccggcaacgccttcaccgccggcaagatcctgttcgccgccgtgccacccaacttccccgtggagttcctgagccccgcccagatcaccatgctgccccacctgatcgtggacgtgcggaccctggagcccatcatgattcccctgcccgacgtgcggaacaccttcttccactacagcaaccagcccaacagccggatgcggctggtggccatgctgtacactcccctgcggagcaacggcagcggcgacgacgtgttcaccgtgagctgccgggtgctgacacggcccactcccgacttcgagttcacctacctggtgcctcccagcgtggagagcaagaccaagcccttcagcctgcccatcctgaccctgagcgagctgaccaacagccggttccccgtgcccatcgacagcctgttcaccgcccagaacaacgtgctgcaggtgcagtgccagaacggccggtgcaccctggacggcgagctgcagggcaccacccagctgctgcccagcggcatctgtgcctttcggggccgagtgaccgcccagatcaaccagcgggaccggtggcacatgcagctgcagaacctgaacggcaccacctacgaccccaccgacgacgtgcccgcacccctgggaacccctgacttcaagggcgtggtgttcggcatggtgagccagcggaacgtgggcaacgacgctcccggcagcaccagagcccagcaggcctgggtgagcacctacagcccacagttcgtgcccaagctgggcagcgtgaacctgcggatcagcgacaacgacgacttccagttccagcccaccaagttcactcccgtgggcgtgaacgacgacgacgacggccaccccttccggcagtgggagctgcccaactacagcggcgagctgaccctgaacatgaacctggcacctcctgtggcccctaacttccccggcgagcagctgctgttcttccggagcttcgtgccctgcagcggcggctacaaccagggcatcatcgactgcctgataccccaggagtggatccagcacttctaccaggagagcgcccctagccagagcgacgtggccctgatccggtacgtgaaccccgacacaggccggaccctgttcgaggccaagctgcaccggagcggctacatcaccgtggcccacagcggcgactatcccctggtggtgcccgccaacggccacttccggttcgacagctgggtgaaccagttctacagcctggccccaatggggacaggcaacggccggagacgggcccag

SEQ ID NO: 20 - protein, 540 aa, Synthetic construct
MKMASNDAAPSNDGAAGLVPEGNNETLPLEPVAGAAIAAPVTGQNNIIDPWIRTNFVQAPNGEFTVSPRNSPGEILLNLELGPDLNPYLAHLSRMYNGYAGGVEVQVLLAGNAFTAGKILFAAVPPNFPVEFLSPAQITMLPHLIVDVRTLEPIMIPLPDVRNTFFHYSNQPNSRMRLVAMLYTPLRSNGSGDDVFTVSCRVLTRPTPDFEFTYLVPPSVESKTKPFSLPILTLSELTNSRFPVPIDSLFTAQNNVLQVQCQNGRCTLDGELQGTTQLLPSGICAFRGRVTAQINQRDRWHMQLQNLNGTTYDPTDDVPAPLGTPDFKGVVFGMVSQRNVGNDAPGSTRAQQAWVSTYSPQFVPKLGSVNLRISDNDDFQFQPTKFTPVGVNDDDDGHPFRQWELPNYSGELTLNMNLAPPVAPNFPGEQLLFFRSFVPCSGGYNQGIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

SEQ ID NO: 21 - RNA, 1644 nt, Synthetic construct
  source 1..1644: mol_type = other RNA
atgaagatggccagcaacgacgccgccccaagcagcgacggagctgccggcctggtgcccgagagcaacaacgaggtgatggcccttgagcctgtggctggcgcctctcttgccgcccctgtgaccggccagaccaacatcatcgacccctggatccggatgaacttcgtgcaggcccctaacggcgagttcaccgtgtcccctcggaacagccccggcgaggtgctgctgaacctggagctgggccccgagctgaacccctacctggcccacctgagccggatgtacaacggctacgccggcggcatggaggtgcagatcatgctggccggcaacgccttcaccgccggcaagctgatcttcgccgccgtgcctccccacttccccatcgagaacctgtctcctcctcagatcaccatgtttccccacgtgatcatcgacgtgcggaccctggagcccgtgctgctgcccatgcccgacatccggaacagcttcttccacttcatccagcgggacgagcccaagatgcggctggtggccatgctgtacacacccctgcggagcaacggcagcggcgacgacgtgttcaccgtgagctgccgggtgctgacccggccaacacccgacttcgacttcacctacctggtgccaccaaccgtggagagcaagagcaagcccttcaccctgcccatcctgaccctgggcgagctgaccaacagccggtttcccctgcccattgacgtgctgtacaccaaccccaacgagagcgccatcgtgcagtgccagaacggccggtgcaccctggacggcgagctgcagggcaccacccagctgctgcctaccggcatctgcgccttccggggcaaggtgacccagcaggtgcaggacgagcaccggggcacccactggaacatgaccgtgaccaacctgaacggcaccccattcgaccccaccgaggacgtgcccgccccactgggcactcccgacttcagcggccagatctacggcgtgatcagccagcggaacaccaacactgtacccggcgagggagacctgcccgccaatcgggcccacgaggccgtgatcgccacctacagccccaagttcaccccgaagctgggcaacatccagttcagcacctgggagacccaggacgtgagcagcggccagcccaccaagttcactcccgtgggcctggccagcgtggacgccaacagccacttcgaccagtggaccctgcccagctacagcggcgccctgaccctgaacatgaacctggcacccagcgtggcccctgtgttccccggcgagtgcctgctgttcttccggagcttcatacccctgaagggcggctacggcaaccccgccatcgactgcctgatgccccaggagtgggtgcagcacctgtaccaggagagcgctcccagcctgagcgacgtggccctggtgcggtacgtgaaccccgagactggccggaccctgttcgaggccaagctgcaccggaacggcttcctgaccgtggccaggaacagcgccggaccagtggtggccccaaccaacggctacttccggttcgacagctgggtgaaccagttctacaccctggctcccatgggcaacggctctggacggcggcggatgcag

SEQ ID NO: 22 - protein, 548 aa, Synthetic construct
MKMASNDAAPSSDGAAGLVPESNNEVMALEPVAGASLAAPVTGQTNIIDPWIRMNFVQAPNGEFTVSPRNSPGEVLLNLELGPELNPYLAHLSRMYNGYAGGMEVQIMLAGNAFTAGKLIFAAVPPHFPIENLSPPQITMFPHVIIDVRTLEPVLLPMPDIRNSFFHFIQRDEPKMRLVAMLYTPLRSNGSGDDVFTVSCRVLTRPTPDFDFTYLVPPTVESKSKPFTLPILTLGELTNSRFPLPIDVLYTNPNESAIVQCQNGRCTLDGELQGTTQLLPTGICAFRGKVTQQVQDEHRGTHWNMTVTNLNGTPFDPTEDVPAPLGTPDFSGQIYGVISQRNTNTVPGEGDLPANRAHEAVIATYSPKFTPKLGNIQFSTWETQDVSSGQPTKFTPVGLASVDANSHFDQWTLPSYSGALTLNMNLAPSVAPVFPGECLLFFRSFIPLKGGYGNPAIDCLMPQEWVQHLYQESAPSLSDVALVRYVNPETGRTLFEAKLHRNGFLTVARNSAGPVVAPTNGYFRFDSWVNQFYTLAPMGNGSGRRRMQ

SEQ ID NO: 23 - RNA, 1635 nt, Synthetic construct
  source 1..1635: mol_type = other RNA
atgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggcaacgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagc

SEQ ID NO: 24 - protein, 545 aa, Synthetic construct
MMMASKDAPTNMDGTSGAGQLVPEANTAEPISMEPVAGAATAAATAGQVNMIDPWIMNNYVQAPQGEFTISPNNTPGDILFDLQLGPHLNPFLSHLAQMYNGWVGNMKVKVLLAGNAFTAGKIIISCIPPGFVAQNVSIAQATMFPHVIADVRVLEPIEVPLEDVRNVLFHNNDNTPTMRLVCMLYTPLRASGSSSGTDPFVIAGRVLTCPSPDFSFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 25 - RNA, 636 nt, Synthetic construct
  source 1..636: mol_type = other RNA
atggcccaggccatcatcggcgccatcgccgctagcaccgccggcagcgcacttggcgccggcatccaggtgggcggagaagccgccctgcagagccagcggtaccagcagaacctccagctgcaggagaacagcttcaagcacgaccgggagatgatcggctaccaggtggaggccagcaaccagctgctggccaagaacctggccacccggtactctctgctgagggccggcggtctgacctctgccgacgctgcccgcagcgtggctggagccccagtgacccggatcgtggactggaacggagtgcgggtgtccgccccagagagcagcgccaccaccctgagaagcggcggcttcatgagcgtgcccatccccttcgccagcaagcagaagcaggtgcagagcagcggcatcagcaaccccaactacagccccagcagcatcagccggaccaccagctgggtggagagccagaacagcagccggttcggcaacctgagcccctaccacgccgaggccctgaacaccgtgtggctgacacctcccggcagcaccgccagcagcaccctgagcagcgtgccccggggctacttcaacaccgaccggctgcccctgttcgccaacaaccggcgg

SEQ ID NO: 26 - protein, 212 aa, Synthetic construct
MAQAIIGAIAASTAGSALGAGIQVGGEAALQSQRYQQNLQLQENSFKHDREMIGYQVEASNQLLAKNLATRYSLLRAGGLTSADAARSVAGAPVTRIVDWNGVRVSAPESSATTLRSGGFMSVPIPFASKQKQVQSSGISNPNYSPSSISRTTSWVESQNSSRFGNLSPYHAEALNTVWLTPPGSTASSTLSSVPRGYFNTDRLPLFANNRR

SEQ ID NO: 27 - RNA, 804 nt, Synthetic construct
  source 1..804: mol_type = other RNA
atggccggagccttcttcgccggactggccagcgacgtgttgggctctggcctgggcagcctgattaacgccggggccggcgccattaaccagaaggtggagttcgagaacaaccggaaactgcagcaagccagctttcagttcagcagcaacctgcagcaggccagcttccagcacgacaaggagatgctgcaggcccagatcgaggccaccaagaagctgcagcaggagatgatgaaggtgaagcaggccatgctgctggagggcggcttcagcgagaccgacgccgccagaggcgctatcaacgctcccatgaccaaggccctggactggagcggcacccggtactgggccccagacgcccggacaaccacctacaacgccggccggttcagcacccctcaacccagcggcgctctgccaggaagagccaatctgcgtgacgccgtgccagcccacggttccagcagcaagagcagcaacttcagcaccgccaccagcgtgtacagcaaccagaccaccagcacacggctgggaagcaccgccggcagcggaaccagcgtgagcagcctgcccagcaccgcacggacccggagctgggtggaggaccagagccggaacctgagccccttcatgcggggcgcccacaacatcagcttcgtgacccctcccagcagccggagcagcagccagggcaccgtgagcaccgtgcccaaggaggtgctggacagctggaccggcgccttcaacacccggcggcagcccctgttcgcccacatccggaagcggggcgagagccgggtg

SEQ ID NO: 28 - protein, 268 aa, Synthetic construct
MAGAFFAGLASDVLGSGLGSLINAGAGAINQKVEFENNRKLQQASFQFSSNLQQASFQHDKEMLQAQIEATKKLQQEMMKVKQAMLLEGGFSETDAARGAINAPMTKALDWSGTRYWAPDARTTTYNAGRFSTPQPSGALPGRANLRDAVPAHGSSSKSSNFSTATSVYSNQTTSTRLGSTAGSGTSVSSLPSTARTRSWVEDQSRNLSPFMRGAHNISFVTPPSSRSSSQGTVSTVPKEVLDSWTGAFNTRRQPLFAHIRKRGESRV

SEQ ID NO: 29 - RNA, 621 nt, Synthetic construct
  source 1..621: mol_type = other RNA
atggcccaggccatcatcggcgccatcgccgctagcaccgccggcagcgcacttggcgccggcatccaggtgggcggagaagccgccctgcagagccagcggtaccagcagaacctccagctgcaggagaacagcttcaagcacgaccgggagatgatcggctaccaggtggaggccagcaaccagctgctggccaagaacctggccacccggtactctctgctgagggccggcggtctgacctctgccgacgctgcccgcagcgtggctggagccccagtgacccggatcgtggactggaacggagtgcgggtgtccgccccagagagcagcgccaccaccctgagaagcggcggcttcatgagcgtgcccatccccttcgccagcaagcagaagcaggtgcagagcagcggcatcagcaaccccaactacagccccagcagcatcagccggaccaccagctgggtggagagccagaacagcagccggttcggcaacctgagcccctaccacgccgaggccctgaacaccgtgtggctgacacctcccggcagcaccgccagcagcaccctgagcagcgtgccccggggctacttcaacaccgaccggctgcccctgttc

SEQ ID NO: 30 - protein, 207 aa, Synthetic construct
MAQAIIGAIAASTAGSALGAGIQVGGEAALQSQRYQQNLQLQENSFKHDREMIGYQVEASNQLLAKNLATRYSLLRAGGLTSADAARSVAGAPVTRIVDWNGVRVSAPESSATTLRSGGFMSVPIPFASKQKQVQSSGISNPNYSPSSISRTTSWVESQNSSRFGNLSPYHAEALNTVWLTPPGSTASSTLSSVPRGYFNTDRLPLF

SEQ ID NO: 31 - RNA, 762 nt, Synthetic construct
  source 1..762: mol_type = other RNA
atggccggagccttcttcgccggactggccagcgacgtgttgggctctggcctgggcagcctgattaacgccggggccggcgccattaaccagaaggtggagttcgagaacaaccggaaactgcagcaagccagctttcagttcagcagcaacctgcagcaggccagcttccagcacgacaaggagatgctgcaggcccagatcgaggccaccaagaagctgcagcaggagatgatgaaggtgaagcaggccatgctgctggagggcggcttcagcgagaccgacgccgccagaggcgctatcaacgctcccatgaccaaggccctggactggagcggcacccggtactgggccccagacgcccggacaaccacctacaacgccggccggttcagcacccctcaacccagcggcgctctgccaggaagagccaatctgcgtgacgccgtgccagcccacggttccagcagcaagagcagcaacttcagcaccgccaccagcgtgtacagcaaccagaccaccagcacacggctgggaagcaccgccggcagcggaaccagcgtgagcagcctgcccagcaccgcacggacccggagctgggtggaggaccagagccggaacctgagccccttcatgcggggcgcccacaacatcagcttcgtgacccctcccagcagccggagcagcagccagggcaccgtgagcaccgtgcccaaggaggtgctggacagctggaccggcgccttcaacacccggcggcag

SEQ ID NO: 32 - protein, 254 aa, Synthetic construct
MAGAFFAGLASDVLGSGLGSLINAGAGAINQKVEFENNRKLQQASFQFSSNLQQASFQHDKEMLQAQIEATKKLQQEMMKVKQAMLLEGGFSETDAARGAINAPMTKALDWSGTRYWAPDARTTTYNAGRFSTPQPSGALPGRANLRDAVPAHGSSSKSSNFSTATSVYSNQTTSTRLGSTAGSGTSVSSLPSTARTRSWVEDQSRNLSPFMRGAHNISFVTPPSSRSSSQGTVSTVPKEVLDSWTGAFNTRRQ

SEQ ID NO: 33 - RNA, 546 nt, Synthetic construct
  source 1..546: mol_type = other RNA
atggccccacccaccctgtggagccgggtgaccaagttcggcagcggctggggcttctgggtgagccccaccgtgttcatcaccaccacccacgtggtgcccaccggcgtgaaggagttcttcggcgagcccctgagcagcatcgccatccaccaggccggcgagttcacccagttccggttcagcaagaagatgcggcccgacctgaccggcatggtgctggaggagggctgtcccgagggcaccgtgtgcagcgtgctgatcaagcgggacagcggcgagctgctgcccctggccgtgcggatgggcgccatcgccagcatgcggatccagggcagactggtgcacggccagagcggcatgctgctgaccggcgccaacgccaagggcatggacctgggcaccatccccggagactgcggcgctccctacgtgcacaagcggggcaacgactgggtggtgtgtggagtccacgccgccgccaccaagagcggcaacaccgtggtgtgcgccgtgcaggccggagagggcgagaccgccctggag

SEQ ID NO: 34 - protein, 182 aa, Synthetic construct
MAPPTLWSRVTKFGSGWGFWVSPTVFITTTHVVPTGVKEFFGEPLSSIAIHQAGEFTQFRFSKKMRPDLTGMVLEEGCPEGTVCSVLIKRDSGELLPLAVRMGAIASMRIQGRLVHGQSGMLLTGANAKGMDLGTIPGDCGAPYVHKRGNDWVVCGVHAAATKSGNTVVCAVQAGEGETALE

SEQ ID NO: 35 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 36 - protein, 540 aa, Synthetic construct
MKMASSDANPSDGSAANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICNFRGDVTHITGSRNYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 37 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcctgcccatcctgaccctgagcgagctgaccaacagccggttccccgtgcccatcgacagcctgttcaccgcccagaacaacgtgctgcaggtgcagtgccagaacggccggtgcaccctggacggcgagctgcagggcaccacccagctgctgcccagcggcatctgtgcctttcggggccgagtgaccgcccagatcaaccagcgggaccggtggcacatgcagctgcagaacctgaacggcaccacctacgaccccaccgacgacgtgcccgcacccctgggaacccctgacttcaagggcgtggtgttcggcatggtgagccagcggaacgtgggcaacgacgctcccggcagcaccagagcccagcaggcctgggtgagcacctacagcccacagttcgtgcccaagctgggcagcatcaacctgcggatcagcgacaacgacgacttccagttccagcccaccaagttcactcccgtgggcgtgaacgacgacgacgacggccaccccttccggcagtgggagctgcccaactacagcggcgagctgaccctgaacatgaacctggcacctcctgtggcccctaacttccccggcgagcagctgctgttcttccggagcttcgtgccctgcagcggcggctacaaccagggcatcatcgactgcctgataccccaggagtggatccagcacttctaccaggagagcgcccctagccagagcgacgtggccctgatccggtacgtgaaccccgacacaggccggaccctgttcgaggccaagctgcaccggagcggctacatcaccgtggcccacagcggcgactatcccctggtggtgcccgccaacggccacttccggttcgacagctgggtgaaccagttctacagcctggccccaatggggacaggcaacggccggagacgggcccag

SEQ ID NO: 38 - protein, 540 aa, Synthetic construct
MKMASSDANPSDGSAANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSLPILTLSELTNSRFPVPIDSLFTAQNNVLQVQCQNGRCTLDGELQGTTQLLPSGICAFRGRVTAQINQRDRWHMQLQNLNGTTYDPTDDVPAPLGTPDFKGVVFGMVSQRNVGNDAPGSTRAQQAWVSTYSPQFVPKLGSINLRISDNDDFQFQPTKFTPVGVNDDDDGHPFRQWELPNYSGELTLNMNLAPPVAPNFPGEQLLFFRSFVPCSGGYNQGIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

SEQ ID NO: 39 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcctgcccatcctgaccctgagcgagctgaccaacagccggttccccgtgcccatcgacagcctgttcaccgcccagaacaacgtgctgcaggtgcagtgccagaacggccggtgcaccctggacggcgagctgcagggcaccacccagctgctgcccagcggcatctgtgcctttcggggccgagtgaccgcccagatcaaccagcgggaccggtggcacatgcagctgcagaacctgaacggcaccacctacgaccccaccgacgacgtgcccgcacccctgggaacccctgacttcaagggcgtggtgttcggcatggtgagccagcggaacgtgggcaacgacgctcccggcagcaccagagcccagcaggcctgggtgagcacctacagcccacagttcgtgcccaagctgggcagcatcaacctgcggatcagcgacaacgacgacttccagttccagcccaccaagttcactcccgtgggcgtgaacgacgacgacgacggccaccccttccggcagtgggagctgcccaactacagcggcgagctgaccctgaacatgaacctggcacctcctgtggcccctaacttccccggcgagcagctgctgttcttccggagcttcgtgccctgcagcggcggctacaaccagggcatcatcgactgcctgataccccaggagtggatccagcacttctaccaggagagcgcccctagccagagcgacgtggccctgatccggtacgtgaaccccgacacaggccggaccctgttcgaggccaagctgcaccggagcggctacatcaccgtggcccacagcggcgactatcccctggtggtgcccgccaacggccacttccggttcgacagctgggtgaaccagttctacagcctggccccaatggggacaggcaacggccggagacgggcccag

SEQ ID NO: 40 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSLPILTLSELTNSRFPVPIDSLFTAQNNVLQVQCQNGRCTLDGELQGTTQLLPSGICAFRGRVTAQINQRDRWHMQLQNLNGTTYDPTDDVPAPLGTPDFKGVVFGMVSQRNVGNDAPGSTRAQQAWVSTYSPQFVPKLGSINLRISDNDDFQFQPTKFTPVGVNDDDDGHPFRQWELPNYSGELTLNMNLAPPVAPNFPGEQLLFFRSFVPCSGGYNQGIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

SEQ ID NO: 41 - RNA, 960 nt, Synthetic construct
  source 1..960: mol_type = other RNA
atgcctggcagcaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 42 - protein, 320 aa, Synthetic construct
MPGSKPFSVPVLTVEEMTNSRFPIPLEKLFTGPSSAFVVQPQNGRCTTDGVLLGTTQLSPVNICNFRGDVTHITGSRNYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 43 - RNA, 1062 nt, Synthetic construct
  source 1..1062: mol_type = other RNA
atgaacccaaaccagaagatcatcaccatcggcagtatctgcatgaccatcggcaccgccaacctgatcctgcagatcggcaacatcatcagcatctgggtgagccctggcagcaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccgggactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcgacggcaccggacgtagacgggtggtg

SEQ ID NO: 44 - protein, 354 aa, Synthetic construct
MNPNQKIITIGSICMTIGTANLILQIGNIISIWVSPGSKPFSVPVLTVEEMTNSRFPIPLEKLFTGPSSAFVVQPQNGRCTTDGVLLGTTQLSPVNICNFRGDVTHITGSRDYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGDGTGRRRVV

SEQ ID NO: 45 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 46 - protein, 540 aa, Synthetic construct
MKMASSDANPSDGSAANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSHDYTMNLASQNWNNYDPTEEIPAPLGTPDFVGKIQGMLTQTTRTDGSTRGHKATVYTGSVHFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 47 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaacttttgctgcgcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagcccctgccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 48 - protein, 540 aa, Synthetic construct
MKMASSDANPSDGSAANLVPEVNNEVMALEPVVGAAIAAPVAGQQNVIDPWIRNNFCCAPGGEFTVSPRNAPGEILWSAPLGPDLNPYLSHLARMYNGYAGGFEVQVILAGNAFTAGKIIFAAVPPNFPTEGLSPCQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSHDYTMNLASQNWNNYDPTEEIPAPLGTPDFVGKIQGMLTQTTRTDGSTRGHKATVYTGSVHFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 49 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaacttttgctgcgcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagcccctgccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 50 - protein, 540 aa, Synthetic construct
MKMASSDANPSDGSAANLVPEVNNEVMALEPVVGAAIAAPVAGQQNVIDPWIRNNFCCAPGGEFTVSPANAPGEILWSAPLGPDLNPYLSHLARMYNGYAGGFEVQVILAGNAFTAGKIIFAAVPPNFPTEGLSPCQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSHDYTMNLASQNWNNYDPTEEIPAPLGTPDFVGKIQGMLTQTTRTDGSTRGHKATVYTGSVHFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 51 - RNA, 1620 nt, Synthetic construct; identical to SEQ ID NO: 49
  source 1..1620: mol_type = other RNA

SEQ ID NO: 52 - protein, 540 aa, Synthetic construct
MKMASSDANPSDGSAANLVPEVNNEVMALEPVVGAAIAAPVAGQQNVIDPWIRNNFCCAPGGEFTVSPANAPGEILWSAPLGPDLNPYLSHLARMYNGYAGGFEVQVILAGNAFTAGKIIFAAVPPNFPTEGLSPCQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICNFRGDVTHITGSRNYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 53 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccaaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcgccggacgtagacgggccctg

SEQ ID NO: 54 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSHNYTMNLASQNWNNYDPTEEIPAPLGTPDFVGKIQGMLTQTTRTDGSTRGHKATVYTGSADFAPKLGRVQFETDTNHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGAGRRRAL

SEQ ID NO: 55 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 56 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 57 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgtgcgcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagcccctgccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 58 - protein, 540 aa, Synthetic construct
MKMASSDANPSDGSAANLVPEVNNEVMALEPVVGAAIAAPVAGQQNVIDPWIRNNFVCAPGGEFTVSPRNAPGEILWSAPLGPDLNPYLSHLARMYNGYAGGFEVQVILAGNAFTAGKIIFAAVPPNFPTEGLSPCQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 59 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgtgcgcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgtgcatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 60 - protein, 540 aa, Synthetic construct
MKMASSDANPSDGSAANLVPEVNNEVMALEPVVGAAIAAPVAGQQNVIDPWIRNNFVCAPGGEFTVSPRNAPGEILWSAPLGPDLNPYLSHLARMYNGYAGGFEVQVILAGNAFTAGKIIFAAVPPNFPTEGLSPSQVCMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 61 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggctgcgccttcacctgcggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 62 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGCAFTCGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 63 - RNA, 1644 nt, Synthetic construct
  source 1..1644: mol_type = other RNA
atgaagatggccagcaacgacgccgccccaagcaacgacggtgctgctgggctggtgcccgagatcaacaacgaggccatggcactcgaaccagtagcaggggctgctattgctgctccactgaccggccagcagaacatcatcgacccctggatcatgaacaacttcgtgcaggcaccaggcggcgagttcaccgtgtctccccggaacagccccggcgaagtccttctgaacctggagctgggccccgagatcaacccctacctggcccacctggcccggatgtacaacgggtacgcaggaggattcgaggtgcaagtcgtgctcgccggcaacgccttcaccgccggcaagatcatcttcgccgccattcctcctaactttccaatcgacaacctgagcgccgcccagatcaccatgtgtcctcacgtcatcgtggacgtcaggcagctggagcccgtgaacctgcccatgccagacgtaaggaacaacttcttccactacaaccagggcagcgacagccggctgcggctcatcgctatgctgtacactccactgcgggccaacaacagcggcgacgacgtgttcaccgtgagctgtagggttctaacccggcccagccccgacttcagcttcaacttcctggtgcctcctaccgtggagagcaagaccaagcccttcagcctgcccatcctgaccatcagcgagatgagcaacagccggttccccgtgcccatcgacagcctgcacaccagccccaccgagaacatcgtggtgcagtgccagaacgggcgcgtgaccctagacggggagctgatgggcaccacccagctgctgcccagccagatctgcgccttccggggcgtgctgatccggagcaccagtagggcaagcgatcaagcggacaccgccactcctcggctgttcaactactactggcacatccagctggacaacctgaacggcactccttacgaccccgccgaggacatccccgggcctctgggcactccagacttccggggcaaggtgttcggcgtggccagccagcggaaccctgacacaacaactcgggcacacgaagccaagatcgacaccacctcaggccggttcactccaaagctgggcagcctggagatcagcaccgagagcgacgacttcgaccagaacaagcctacccggttcacacctgtgggcatcggcgtggaccacgaggccgacttccagcagtggaccctgcccgactacgccggccagttcacccacaacatgaacctggctccagccgtggctcctaacttccccggcgagcagctgctgttcttccggagccaattaccatcctctggaggtcgcagcaacggcatcctggactgcctggtgccccaggagtgggtgcagcacttctaccaggagagcgctccaagccagagccaggtggccctggtgcggtacatcaaccccgacaccggacgagttctgttcgaggccaagctgcacaagctgggcttcatcaccatcgccaagaacggcgacagccccatcaccgtgccccctaacggctacttccggttcgagagctgggtgaaccccttctacaccctggcgccaatgggcaccggcaacggacgtcgccggatccag

SEQ ID NO: 64 - protein, 548 aa, Synthetic construct
MKMASNDAAPSNDGAAGLVPEINNEAMALEPVAGAAIAAPLTGQQNIIDPWIMNNFVQAPGGEFTVSPRNSPGEVLLNLELGPEINPYLAHLARMYNGYAGGFEVQVVLAGNAFTAGKIIFAAIPPNFPIDNLSAAQITMCPHVIVDVRQLEPVNLPMPDVRNNFFHYNQGSDSRLRLIAMLYTPLRANNSGDDVFTVSCRVLTRPSPDFSFNFLVPPTVESKTKPFSLPILTISEMSNSRFPVPIDSLHTSPTENIVVQCQNGRVTLDGELMGTTQLLPSQICAFRGVLIRSTSRASDQADTATPRLFNYYWHIQLDNLNGTPYDPAEDIPGPLGTPDFRGKVFGVASQRNPDTTTRAHEAKIDTTSGRFTPKLGSLEISTESDDFDQNKPTRFTPVGIGVDHEADFQQWTLPDYAGQFTHNMNLAPAVAPNFPGEQLLFFRSQLPSSGGRSNGILDCLVPQEWVQHFYQESAPSQSQVALVRYINPDTGRVLFEAKLHKLGFITIAKNGDSPITVPPNGYFRFESWVNPFYTLAPMGTGNGRRRIQ

SEQ ID NO: 65 - RNA, 1605 nt, Synthetic construct
  source 1..1605: mol_type = other RNA
atgaagatggccagcaacgacgccgccccaagcaacgacggtgctgctgggctggtgcccgaggtgaacaacgagaccatggcactcgaaccagtagcaggggcttctattgctgctccactgaccggccagaacaacgtgatcgacccctggatccggctgaacttcgtgcaggcaccaaacggcgagttcaccgtgtctccccggaacagccccggcgaagtccttctgaacctggagctgggccccgagctgaacccctacctggcccacctgagccggatgtacaacgggtacgcaggaggagtcgaggtgcaagtcttgctcgccggcaacgccttcaccgccggcaagctggtgttcgccgccgtgcctcctcactttccactggagaacatcagccccggccagatcaccatgttccctcacgtcatcattgacgtcaggaccctggagcccgtgctgctgcccctgccagacgtaaggaacagcttcttccactacaaccagcagaacgagccccggatgcggctcgtcgctatgctgtacactccactgcggagcaacggcagcggcgacgacgtgttcaccgtgagctgtagggttctaacccggcccagccccgacttcgacttcaactacctggtgcctcctaccgtggagagcaagaccaagcccttcaccctgcccgtgctgaccatcggcgagctgaccaacagccggttccccgtgcccatcgacgagctgtacaccagccccaacgagagcctggtggtgcagccccagaacgggcgctgtgcactagacggggagctgcagggcaccacccagctgctgcccaccgccatctgcagcttccggggccggatcaaccagaaggtgagcggcgagaaccacgtttggaacatgcaggtgaccaacatcaacggcactcctttcgaccccaccgaggacgtgcccgctcctctgggcactccagacttcagcggcaagctgttcggcgtgctgagccagcgggaccacgacaacgcctgccggagccacgacgccgtgatcgccaccgactcaacgaagttcactccaaagctgggcgccgtgcagatcggcacctgggagcaggacgacgtgcacatcaaccagcctaccaagttcacacctgtgggcctgttcgagaacgagggcttcaaccagtggaccctgcccaactacagcggcgccctgaccctgaacatgggcctggctccacccgtggctcctaccttccccggcgagcagatcctgttcttccggagccacattccactgaaaggaggtgtagctgatccagtgatcgactgcctgctgccccaggagtggatccagcacctgtaccaggagagcgctccaagccagaccgacgtggccctgatccggttcaccaaccccgacaccggacgagttctgttcgaggccaagctgcaccggagcggctacatcaccgtggccaacaccggcagccggcccatcgtggtgcccgccaacggctacttccggttcgacagctgggtgaaccagttctacagcctggcgccaatgggcaccggcaacggacgtcgccggatgcag

SEQ ID NO: 66 - protein, 535 aa, Synthetic construct
MKMASNDAAPSNDGAAGLVPEVNNETMALEPVAGASIAAPLTGQNNVIDPWIRLNFVQAPNGEFTVSPRNSPGEVLLNLELGPELNPYLAHLSRMYNGYAGGVEVQVLLAGNAFTAGKLVFAAVPPHFPLENISPGQITMFPHVIIDVRTLEPVLLPLPDVRNSFFHYNQQNEPRMRLVAMLYTPLRSNGSGDDVFTVSCRVLTRPSPDFDFNYLVPPTVESKTKPFTLPVLTIGELTNSRFPVPIDELYTSPNESLVVQPQNGRCALDGELQGTTQLLPTAICSFRGRINQKVSGENHVWNMQVTNINGTPFDPTEDVPAPLGTPDFSGKLFGVLSQRDHDNACRSHDAVIATDSTKFTPKLGAVQIGTWEQDDVHINQPTKFTPVGLFENEGFNQWTLPNYSGALTLNMGLAPPVAPTFPGEQILFFRSHIPLKGGVADPVIDCLLPQEWIQHLYQESAPSQTDVALIRFTNPDTGRVLFEAKLHRSGYITVANTGSRPIVVPANGYFRFDSWVNQFYSLAPMGTGNGRRRMQ

SEQ ID NO: 67 - RNA, 1635 nt, Synthetic construct
  source 1..1635: mol_type = other RNA
atgatgatggccagcaaggacgcccctaccaacatggacggcactagcggcgcaggccagctggtgcccgaggccaacaccgccgagcccatcagcatggaacccgtgtgcggcgctgcaaccgcagcctgtaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggcaacgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagc

SEQ ID NO: 68 - protein, 545 aa, Synthetic construct
MMMASKDAPTNMDGTSGAGQLVPEANTAEPISMEPVCGAATAACTAGQVNMIDPWIMNNYVQAPQGEFTISPNNTPGDILFDLQLGPHLNPFLSHLAQMYNGWVGNMKVKVLLAGNAFTAGKIIISCIPPGFVAQNVSIAQATMFPHVIADVRVLEPIEVPLEDVRNVLFHNNDNTPTMRLVCMLYTPLRASGSSSGTDPFVIAGRVLTCPSPDFSFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 69 - RNA, 1635 nt, Synthetic construct
  source 1..1635: mol_type = other RNA
atgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgtgcgcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggcaacgccttcaccgccggcaagatcatcatcagctgcatcccacccggcttcgtggcccagaacgtgagcatctgccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagc

SEQ ID NO: 70 - protein, 545 aa, Synthetic construct
MMMASKDAPTNMDGTSGAGQLVPEANTAEPISMEPVAGAATAAATAGQVNMIDPWIMNNYVCAPQGEFTISPNNTPGDILFDLQLGPHLNPFLSHLAQMYNGWVGNMKVKVLLAGNAFTAGKIIISCIPPGFVAQNVSICQATMFPHVIADVRVLEPIEVPLEDVRNVLFHNNDNTPTMRLVCMLYTPLRASGSSSGTDPFVIAGRVLTCPSPDFSFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 71 - RNA, 1635 nt, Synthetic construct
  source 1..1635: mol_type = other RNA
atgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggctgcgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgaggtgcagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagc

SEQ ID NO: 72 - protein, 545 aa, Synthetic construct
MMMASKDAPTNMDGTSGAGQLVPEANTAEPISMEPVAGAATAAATAGQVNMIDPWIMNNYVQAPQGEFTISPNNTPGDILFDLQLGPHLNPFLSHLAQMYNGWVGNMKVKVLLAGCAFTAGKIIISCIPPGFVAQNVSIAQATMFPHVIADVRVLEPIEVPLEDVRNVLFHNNDNTPTMRLVCMLYTPLRCSGSSSGTDPFVIAGRVLTCPSPDFSFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 73 - RNA, 1635 nt, Synthetic construct
  source 1..1635: mol_type = other RNA
atgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggctgcgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggcctgcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagc

SEQ ID NO: 74 - protein, 545 aa, Synthetic construct
MMMASKDAPTNMDGTSGAGQLVPEANTAEPISMEPVAGAATAAATAGQVNMIDPWIMNNYVQAPQGEFTISPNNTPGDILFDLQLGPHLNPFLSHLAQMYNGWVGNMKVKVLLAGCAFTAGKIIISCIPPGFVAQNVSIAQATMFPHVIADVRVLEPIEVPLEDVRNVLFHNNDNTPTMRLVCMLYTPLRACGSSSGTDPFVIAGRVLTCPSPDFSFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 75 - RNA, 1635 nt, Synthetic construct
  source 1..1635: mol_type = other RNA
atgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggctgcgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagctgcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagc

SEQ ID NO: 76 - protein, 545 aa, Synthetic construct
MMMASKDAPTNMDGTSGAGQLVPEANTAEPISMEPVAGAATAAATAGQVNMIDPWIMNNYVQAPQGEFTISPNNTPGDILFDLQLGPHLNPFLSHLAQMYNGWVGNMKVKVLLAGCAFTAGKIIISCIPPGFVAQNVSIAQATMFPHVIADVRVLEPIEVPLEDVRNVLFHNNDNTPTMRLVCMLYTPLRASCSSSGTDPFVIAGRVLTCPSPDFSFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 77 - RNA, 1632 nt, Synthetic construct
  source 1..1632: mol_type = other RNA
atgatgatggccagcaaggacgccaccagcagcgtggacggcgcaagcggcgcaggccagctggtgcccgaggtgaacgccagcgaccctctggccatggatcccgtggcaggctccagcaccgcagtggcaaccgcagggcaggtgaaccccatcgacccgtggatcatcaacaacttcgtgcaagcccctcagggcgagttcactatcagccccaacaacacgcccggcgacgttctgttcgacctgagcctgggtccccacctgaaccccttccttctgcatctgagccagatgtacaacggttgggtgggcaacatgcgggtgcggatcatgctggccggctgcgccttcaccgccggcaagatcatcgtgagctgcatcccacccggctttggcagccacaacctgaccatcgcacaggccaccctgttcccacacgtgattgccgacgttcggaccctggatcccatcgaggtgcccctggaggacgtgcggaacgtgctgttccacaacaacgaccggaaccagcagaccatgcggctggtgtgcatgctgtacacccctctgaggacctgcggcggcacaggcgacagcttcgtggtggccggacgggtgatgacgtgtcccagccccgacttcaacttcctgttcctggtgccacctaacgtggagcagaagaccaagcccttcagcgtgcccaacctgcccctgaacaccctgagcaacagccgggttcccagcctgatcaagagcatgatggtgagccgggaccacggccagatggtacagttccagaacggccgggtcaccctggacggtcagctgcaaggcaccacacctaccagcgccagccagctgtgcaagatccggggcagcgtgttccacgccaacggcggcaacggctacaaccttaccgagctggacggcagcccctaccacgccttcgagagccccgccccaatcggcttccccgacctgggcgagtgcgactggcacatggaggccagccccaccacccagttcaacaccggggacgtgatcaagcagatcaacgtgaagcaggagagcgccttcgccccacacctgggcaccatccaggccgacggcctcagcgacgtgagcgtgaacaccaacatgatcgccaagctgggctgggttagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggccccaacccggggtgaagccgccctgctgcactacctggaccccgacacccaccggaatctgggtgagttcaagctgtaccccgacggcttcatgacctgtgtgcccaatagctctggcaccggtccccagaccctgcccatcaacggcgtgttcgtgtttgtgagctgggtgtcacggttctaccagctgaagcccgtgggcaccgctggacccgccagacggctgggcatccggcggagc

SEQ ID NO: 78 - protein, 544 aa, Synthetic construct
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGCAFTAGKIIVSCIPPGFGSHNLTIAQATLFPHVIADVRTLDPIEVPLEDVRNVLFHNNDRNQQTMRLVCMLYTPLRTCGGTGDSFVVAGRVMTCPSPDFNFLFLVPPNVEQKTKPFSVPNLPLNTLSNSRVPSLIKSMMVSRDHGQMVQFQNGRVTLDGQLQGTTPTSASQLCKIRGSVFHANGGNGYNLTELDGSPYHAFESPAPIGFPDLGECDWHMEASPTTQFNTGDVIKQINVKQESAFAPHLGTIQADGLSDVSVNTNMIAKLGWVSPVSDGHKGDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 79 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccaccggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtg

SEQ ID NO: 80 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTTGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICNFRGDVTHITGSRNYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 81 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccaccgccacctgagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 82 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPPPESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 83 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcagattcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgattggtgggtgaaccagttctacaccctggcgcctatgggcaacggcaccggacgtagacgggccgtg

SEQ ID NO: 84 - protein, 540 aa, Synthetic construct
MKMASSDANPSDGSAANLVPEVNNEVMALEPVVGAAIAAPVAGQQNVIDPWIRNNFVQAPGGRFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDWWVNQFYTLAPMGNGTGRRRAV

SEQ ID NO: 85 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcagattcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccaccaccacctgagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgattggtgggtgaaccagttctacaccctggcgcctatgggcaacggcaccggacgtagacgggccgtg

SEQ ID NO: 86 - protein, 540 aa, Synthetic construct
MKMASSDANPSDGSAANLVPEVNNEVMALEPVVGAAIAAPVAGQQNVIDPWIRNNFVQAPGGRFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPPPESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDWWVNQFYTLAPMGNGTGRRRAV

SEQ ID NO: 87 - RNA, 1626 nt, Synthetic construct
  source 1..1626: mol_type = other RNA
atgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggcgagttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccacctcctcccgagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgacagctgggtgaaccagttctacagcctggccccaatgggaaccggtaacggacgccggcgggtgcag

SEQ ID NO: 88 - protein, 542 aa, Synthetic construct
MKMASNDAAPSTDGAAGLVPESNNEVMALEPVAGAALAAPVTGQTNIIDPWIRANFVQAPNGEFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPPPESKTKPFTLPILTLGELSNSRFPVSIDQMYTSPNEIISVQCQNGRCTLDGELQGTTQLQVSGICAFKGEVTAHLHDNDHLYNVTITNLNGSPFDPSEDIPAPLGVPDFQGRVFGIISQRDKHNSPGHNEPANRGHDAVVPTYTAQYTPKLGQIQIGTWQTDDLTVNQPVKFTPVGLNDTEHFNQWVVPRYAGALNLNTNLAPSVAPVFPGERLLFFRSYIPLKGGYGNPAIDCLLPQEWVQHFYQEAAPSMSEVALVRYINPDTGRALFEAKLHRAGFMTVSSNTSAPVVVPANGYFRFDSWVNQFYSLAPMGTGNGRRRVQ

SEQ ID NO: 89 - RNA, 1626 nt, Synthetic construct
  source 1..1626: mol_type = other RNA
atgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggccggttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccacccaccgtggagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgactggtgggtgaaccagttctacagcctggccccaatgggaaccggtaacggacgccggcgggtgcag

SEQ ID NO: 90 - protein, 542 aa, Synthetic construct
MKMASNDAAPSTDGAAGLVPESNNEVMALEPVAGAALAAPVTGQTNIIDPWIRANFVQAPNGRFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTVESKTKPFTLPILTLGELSNSRFPVSIDQMYTSPNEIISVQCQNGRCTLDGELQGTTQLQVSGICAFKGEVTAHLHDNDHLYNVTITNLNGSPFDPSEDIPAPLGVPDFQGRVFGIISQRDKHNSPGHNEPANRGHDAVVPTYTAQYTPKLGQIQIGTWQTDDLTVNQPVKFTPVGLNDTEHFNQWVVPRYAGALNLNTNLAPSVAPVFPGERLLFFRSYIPLKGGYGNPAIDCLLPQEWVQHFYQEAAPSMSEVALVRYINPDTGRALFEAKLHRAGFMTVSSNTSAPVVVPANGYFRFDWWVNQFYSLAPMGTGNGRRRVQ

SEQ ID NO: 91 - RNA, 1626 nt, Synthetic construct
  source 1..1626: mol_type = other RNA
atgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggccggttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccaccacctcccgagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgactggtgggtgaaccagttctacagcctggccccaatgggaaccggtaacggacgccggcgggtgcag

SEQ ID NO: 92 - protein, 542 aa, Synthetic construct
MKMASNDAAPSTDGAAGLVPESNNEVMALEPVAGAALAAPVTGQTNIIDPWIRANFVQAPNGRFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPPPESKTKPFTLPILTLGELSNSRFPVSIDQMYTSPNEIISVQCQNGRCTLDGELQGTTQLQVSGICAFKGEVTAHLHDNDHLYNVTITNLNGSPFDPSEDIPAPLGVPDFQGRVFGIISQRDKHNSPGHNEPANRGHDAVVPTYTAQYTPKLGQIQIGTWQTDDLTVNQPVKFTPVGLNDTEHFNQWVVPRYAGALNLNTNLAPSVAPVFPGERLLFFRSYIPLKGGYGNPAIDCLLPQEWVQHFYQEAAPSMSEVALVRYINPDTGRALFEAKLHRAGFMTVSSNTSAPVVVPANGYFRFDWWVNQFYSLAPMGTGNGRRRVQ

SEQ ID NO: 93 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtactgcccactgcgggccaacaacgccggcgacgacgtgtttaccgtgtgctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 94 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYCPLRANNAGDDVFTVCCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 95 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgtgcttccctaccgagggcctgagccccagccaggtgaccatgttccctcacatcatcgtggacgtgcgacagctggaacccgtgctgatacccctgcccgacgtgcggaacaatttctaccactgcaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 96 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPCFPTEGLSPSQVTMFPHIIVDVRQLEPVLIPLPDVRNNFYHCNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 97 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgacctgcttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgtgccccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 98 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTCFPHIIVDVRQLEPVLIPLPDVRNNFYHYNQSNDSTIKLIAMLYTPLRANNAGDDVFTVSCRVLTRPSPDFDFIFLVCPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 99 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgtgcttccctaccgagggcctgagccccagccaggtgaccatgttccctcacatcatcgtggacgtgcgacagctggaacccgtgctgatacccctgcccgacgtgcggaacaatttctaccactgcaaccagagcaacgacagcaccatcaagctgatcgccatgctgtactgcccactgcgggccaacaacgccggcgacgacgtgtttaccgtgtgctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 100 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPCFPTEGLSPSQVTMFPHIIVDVRQLEPVLIPLPDVRNNFYHCNQSNDSTIKLIAMLYCPLRANNAGDDVFTVCCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 101 - RNA, 1620 nt, Synthetic construct
  source 1..1620: mol_type = other RNA
atgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgacctgcttccctcacatcatcgtggacgtgcgacagctggaacccgtgctgatacccctgcccgacgtgcggaacaatttctaccactacaaccagagcaacgacagcaccatcaagctgatcgccatgctgtactgcccactgcgggccaacaacgccggcgacgacgtgtttaccgtgtgctgccgggttctgacccggcccagcccagacttcgacttcatctttctggtgtgccccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtg

SEQ ID NO: 102 - protein, 540 aa, Synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTCFPHIIVDVRQLEPVLIPLPDVRNNFYHYNQSNDSTIKLIAMLYCPLRANNAGDDVFTVCCRVLTRPSPDFDFIFLVCPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGVLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 103 - RNA, 47 nt, Synthetic construct
  source 1..47: mol_type = other RNA
gggaaataagagagaaaagaagagtaagaagaaatataagagccacc

SEQ ID NO: 104 - RNA, 57 nt, Synthetic construct; identical to SEQ ID NO: 1
  source 1..57: mol_type = other RNA

SEQ ID NO: 105 - RNA, 119 nt, Synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 106 - RNA, 119 nt, Synthetic construct; identical to SEQ ID NO: 4
  source 1..119: mol_type = other RNA

SEQ ID NO: 107 - RNA, 75 nt, Synthetic construct
  source 1..75: mol_type = other RNA
ggaaatcgcaaaatttgctcttcgcgttagatttcttttagttttctcgcaactagcaagctttttgttctcgcc

SEQ ID NO: 108 - RNA, 203 nt, Synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 109 - RNA, 76 nt, Synthetic construct
  source 1..76: mol_type = other RNA
ggaaataagagagaaaagaagagtaagaagaaatataagatctccctgagcttcagggagccccggcgccgccacc

SEQ ID NO: 110 - RNA, 89 nt, Synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 111 - RNA, 59 nt, Synthetic construct
  source 1..59: mol_type = other RNA
ggagaacttccgcttccgttggcgcaagcgctttcattttttctgctaccgtgactaag

SEQ ID NO: 112 - RNA, 46 nt, Synthetic construct
  source 1..46: mol_type = other RNA
ggaaataagagagaaaagaagagtaagaagaaatataagagccacc

SEQ ID NO: 113 - RNA, 56 nt, Synthetic construct
  source 1..56: mol_type = other RNA
ggaaataagagagaaaagaagagtaagaagaaatataagaccccggcgccgccacc

SEQ ID NO: 114 - RNA, 81 nt, Synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 115 - RNA, 81 nt, Synthetic construct
  source 1..81: mol_type = other RNA
ggaaatcgcaaaattttcttttcgcgttagatttcttttagttttctttcaactagcaagctttttgttctcgccgccgcc

SEQ ID NO: 116 - RNA, 83 nt, Synthetic construct
  source 1..83: mol_type = other RNA
  variation 14..18: note = May be absent
  variation 19: note = May be absent
  variation 22: note = May be absent
  variation 23..27: note = May be absent
  variation 53: note = May be uracil or cytosine
  variation 54: note = May be uracil or guanine
  variation 81: note = May be adenine or guanine
  variation 81: note = May be absent
ggaaatcgcaaaatttttgctctttttcgcgttagatttcttttagttttctnncaactagcaagctttttgttctcgccncc

SEQ ID NO: 117 - RNA, 79 nt, Synthetic construct
  source 1..79: mol_type = other RNA
ggaaaattttagcctggaacgttagataactgtcctgttgtctttatatacttggtccccaagtagtttgtcttccaaa

SEQ ID NO: 118 - RNA, 79 nt, Synthetic construct
  source 1..79: mol_type = other RNA
ggaaactttatttagtgttactttattttctgtttatttgtgtttcttcagtgggtttgttctaatttccttggccgcc

SEQ ID NO: 119 - RNA, 79 nt, Synthetic construct
  source 1..79: mol_type = other RNA
ggaaaatctgtattaggttggcgtgttctttggtcggttgttagtattgttgttgattcgtttgtggtcggttgccgcc

SEQ ID NO: 120 - RNA, 79 nt, Synthetic construct
  source 1..79: mol_type = other RNA
ggaaaattattaacatcttggtattctcgataaccattcgttggattttattgtattcgtagtttgggttcctgccgcc

SEQ ID NO: 121 - RNA, 79 nt, Synthetic construct
  source 1..79: mol_type = other RNA
ggaaattattattatttctagctacaatttatcattgtattattttagctattcatcattatttacttggtgatcaaca

SEQ ID NO: 122 - RNA, 79 nt, Synthetic construct
  source 1..79: mol_type = other RNA
ggaaataggttgttaaccaagttcaagcctaataagcttggattctggtgacttgcttcaccgttggcgggcaccgatc

SEQ ID NO: 123 - RNA, 74 nt, Synthetic construct
  source 1..74: mol_type = other RNA
ggaaatcgtagagagtcgtacttagtacatatcgactatcggtggacaccatcaagattataaaccaggccaga

SEQ ID NO: 124 - RNA, 79 nt, Synthetic construct
  source 1..79: mol_type = other RNA
ggaaacccgcccaagcgaccccaacatatcagcagttgcccaatcccaactcccaacacaatccccaagcaacgccgcc

SEQ ID NO: 125 - RNA, 79 nt, Synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 126 - RNA, 79 nt, Synthetic construct
  source 1..79: mol_type = other RNA
ggaaactaatcgaaataaaagagccccgtactcttttatttctattaggttaggagccttagcatttgtatcttaggta

SEQ ID NO: 127 - RNA, 79 nt, Synthetic construct
  source 1..79: mol_type = other RNA
ggaaatgtgatttccagcaacttcttttgaatatattgaattcctaattcaaagcgaacaaatctacaagccatatacc

SEQ ID NO: 128 - RNA, 74 nt, Synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 129 - RNA, 74 nt, Synthetic construct
  source 1..74: mol_type = other RNA
ggaaatcgtagagagtcgtacttagaataaacagagtcgggtcgacttgtctctgatactacgacgtcacaatc

SEQ ID NO: 130 - RNA, 79 nt, Synthetic construct
  source 1..79: mol_type = other RNA
ggaaaatttgccttcggagttgcgtatcctgaactgcccagcctcctgatatacaactgttccgcttattcgggccgcc

SEQ ID NO: 131 - RNA, 79 nt, Synthetic construct
  source 1..79: mol_type = other RNA
ggaaatctgagcaggaatcctttgtgcattgaagactttagattcctctctgcggtagacgtgcacttataagtatttg

SEQ ID NO: 132 - RNA, 79 nt, Synthetic construct
  source 1..79: mol_type = other RNA
ggaaagcgattgaaggcgtcttttcaactactcgattaaggttgggtatcgtcgtgggacttggaaatttgttgccacc

SEQ ID NO: 133 - RNA, 104 nt, Synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 134 - RNA, 57 nt, Synthetic construct
  source 1..57: mol_type = other RNA
ggaaataagagagaaaagaagagtaagaagaaatataagagccaaaaaaaaaaaacc

SEQ ID NO: 135 - RNA, 76 nt, Synthetic construct
  source 1..76: mol_type = other RNA
ggaaatctccctgagcttcagggagtaagagagaaaagaagagtaagaagaaatataagaccccggcgccgccacc

SEQ ID NO: 136 - skipped

SEQ ID NO: 137 - RNA, 36 nt, Synthetic construct
  source 1..36: mol_type = other RNA
ggactcactatttgttttcgcgcccagttgcaaaaa

SEQ ID NO: 138 - RNA, 119 nt, Synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 139 - RNA, 119 nt, Synthetic construct
  source 1..119: mol_type = other RNA
taagtctaagctggagcctcctgagagacctgtgtgaactattgagaagatcggaacagctccttactctgaggaagttggtacccccgtggtctttgaataaagtctgagtgggcggc

SEQ ID NO: 140 - RNA, 185 nt, Synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 141 - RNA, 170 nt, Synthetic construct
  source 1..170: mol_type = other RNA
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 142 - RNA, 141 nt, Synthetic construct
  source 1..141: mol_type = other RNA
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 143 - RNA, 185 nt, Synthetic construct
  source 1..185: mol_type = other RNA
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 144 - RNA, 187 nt, Synthetic construct
  source 1..187: mol_type = other RNA
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 145 - RNA, 170 nt, Synthetic construct
  source 1..170: mol_type = other RNA
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 146 - RNA, 141 nt, Synthetic construct
  source 1..141: mol_type = other RNA
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 147 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 148 - RNA, 1766 nt, Synthetic construct
  source 1..1766: mol_type = other RNA
gggaaataagagagaaaagaagagtaagaagaaatataagaccccggcgccgccaccatgatgatggccagcaaggacgccaccagcagcgtggacggcgcttctggggccggacagctggtgcccgaggtgaacgccagcgacccactggccatggaccccgtggccggaagcagcaccgccgtggccaccgcaggacaggtgaaccccatcgacccttggatcatcaacaacttcgtgcaggcaccccagggcgagttcaccatcagccccaacaacactcccggcgacgtgctgttcgacctgtccctgggcccacacctgaacccctttctgctgcacctgagccagatgtacaacggctgggtgggcaacatgcgggtgcggatcatgctggccggcaacgcctttaccgccggcaagatcatcgtgagctgcatcccacccggctttggcagccacaacctgaccatcgcccaggccaccctgttcccacacgtgatcgccgacgtccggaccctggatcccatcgaggtgcccctggaggacgtgcggaacgtgcttttccacaacaacgaccggaaccagcagaccatgcgcctggtgtgcatgctgtacactccacttcggacaggcggcggaaccggcgacagcttcgtggtggccggccgggtgatgacctgcccttccccggacttcaacttcctgttcctggtgccacccaccgtggagcagaagacccggcccttcaccctgcccaacctgcccctgagcagcctgagcaacagccgggcacccctgcctatcagcagcatgggcatctcacccgacaacgtgcagagcgtgcagttccagaacggcaggtgcaccctcgacggccggctggtgggcaccacgcccgtgagcctgtctcacgtggccaagatccgcggcaccagtaacggcaccgtgattaatctgaccgagctggacggcactcccttccaccctttcgagggccccgcgccaatcggattccccgacttaggtggctgcgactggcacatcaacatgacccagttcggccacagcagccagacccagtacgacgtggacaccacccctgacaccttcgtgccccacctgggcagcatccaggccaacggcatcggcagcggcaactacgtgggcgtgctgagctggatcagccctcccagccaccccagcggcagccaagtggacctgtggaagatccccaactacgggagcagcatcaccgaagctacccacctcgctcccagcgtgtaccctcctggcttcggcgaggtgctggtgttcttcatgagcaagatgcctggccctggcgcctacaatctgccctgcctgctgccccaggagtacatcagtcacctggccagcgagcaggctccaacagtgggcgaggccgccttactgcactacgtcgaccccgacaccggccggaacctgggagagttcaaggcctaccccgacggcttcctgacctgcgtgcccaacggcgcaagcagcggccctcagcagctgcccatcaacggcgtgttcgtgtttgtgagctgggtgagccggttctaccagctgaagcccgtaggcaccgccagctccgctcggggaagactgggcctgcggcggtgataataggctggagcctcggtggcctagcttcttgccccttgggcctccccccagcccctcctccccttcctgcacccgtacccccgtggtctttgaataaagtctgagtgggcggc

SEQ ID NO: 149 - RNA, 1799 nt, Synthetic construct
  source 1..1799: mol_type = other RNA
gggaaataagagagaaaagaagagtaagaagaaatataagaccccggcgccgccaccatgcggatgagcgacggcgccgcccctaaggccaacggcagtgaggccagcggccaggacttggtgcccaccgccgtggagcaggcagtgccaatccaacccgtggctggcgccgctttagccgccccagcagctggccagatcaaccagatagacccctggatcttccagaactttgtgcagtgtcccctgggcgagttcagcatcagccctcggaacacgcccggcgagatcctgttcgatctggcgctgggacccggcctgaacccctacctggcccacctgagcgccatgtacaccggctgggtgggcaacatggaagtgcagctggtgctggccggtaacgcctttaccgccggcaaggtggtggtggccctggtgccaccctacttccccaagggcagcctgaccaccgcccagatcacctgcttccctcacgtgatgtgcgacgtgagaaccctggagcccatccagctgcccctgctggacgtgcggcgggtgctgtggcacgccacccaggaccaggaggagagcatgcggctggtgtgcatgctgtacaccccactgcggaccaacagccccggcgacgagagcttcgtggtgagcggccggctgctcagcaagcccgccgccgacttcaacttcgtgtacctgacccctccaatcgagcggaccatctaccggatggtggacctgcccgtgctgcaaccccggctgtgcacccacgccaggtggcctgcgccaatctacggcctgctggtggatcccagcctgcccagcaacccacagtggcagaacggccgggtgcacgtggacggcaccctgcttggcaccacccctgtgagcgggagctgggtgagctgcttcgccgcagaggccgcctacgagttccagagcggcatcggcgaggtcgccaccttcaccctgatcgagcaggacggcagcgcctacgtgcctggagacagggccgcgcctctgggctaccccgactttagcggacagctggagatcgaggtgcagaccgagaccaccaagaagggcgagaagctgaaggtgaccaccttcgagatgatcctgggccccacgaccaacgtggaccaggtgccctaccagggacgggtgtacgcaagcctgactgccgccgccagcctggacctggtggacggacgagtaagagccgtgccccggagcgtgtacggcttccaggacgtggtgcccgagtacaacgacggcctgttagtgcccctggccccacccatcggccccttcttaccaggcgaagtcctgctgcggttccggacctacatgcggcagatcgacagcaccgacgctgctgccgaggccatcgactgcgccctgccccaggagttcgtgagctggttcgccagcaacgccttcaccgtgcagagcgaggccctgctgctgagataccggaacacccttaccggtcaactgctgttcgagtgcaagctgtatagcgagggctacatcgccctgagctacagcggcagcggccctctgaccttccccaccgacggcttcttcgaggtggtgtcttgggtgccacggctgtaccagttggccagcgtgggctctctggccaccggccggaccctgaagcagtgataataggctggagcctcggtggcctagcttcttgccccttgggcctccccccagcccctcctccccttcctgcacccgtacccccgtggtctttgaataaagtctgagtgggcggc

SEQ ID NO: 150 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 151 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcgccgacacccacaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgctaccgacaccgctgctgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 152 - RNA, 1766 nt, Synthetic construct
  source 1..1766: mol_type = other RNA
gggaaataagagagaaaagaagagtaagaagaaatataagaccccggcgccgccaccatgatgatggccagcaaggacgccaccagcagcgtggacggcgcttctggggccggacagctggtgcccgaggtgaacgccagcgacccactggccatggaccccgtggccggaagcagcaccgccgtggccaccgcaggacaggtgaaccccatcgacccttggatcatcaacaacttcgtgcaggcaccccagggcgagttcaccatcagccccaacaacactcccggcgacgtgctgttcgacctgtccctgggcccacacctgaacccctttctgctgcacctgagccagatgtacaacggctgggtgggcaacatgcgggtgcggatcatgctggccggctgcgcctttaccgccggcaagatcatcgtgagctgcatcccacccggctttggcagccacaacctgaccatcgcccaggccaccctgttcccacacgtgatcgccgacgtccggaccctggatcccatcgaggtgcccctggaggacgtgcggaacgtgcttttccacaacaacgaccggaaccagcagaccatgcgcctggtgtgcatgctgtacactccacttcggacatgcggcggaaccggcgacagcttcgtggtggccggccgggtgatgacctgcccttccccggacttcaacttcctgttcctggtgccacccaccgtggagcagaagacccggcccttcaccctgcccaacctgcccctgagcagcctgagcaacagccgggcacccctgcctatcagcagcatgggcatctcacccgacaacgtgcagagcgtgcagttccagaacggcaggtgcaccctcgacggccggctggtgggcaccacgcccgtgagcctgtctcacgtggccaagatccgcggcaccagtaacggcaccgtgattaatctgaccgagctggacggcactcccttccaccctttcgagggccccgcgccaatcggattccccgacttaggtggctgcgactggcacatcaacatgacccagttcggccacagcagccagacccagtacgacgtggacaccacccctgacaccttcgtgccccacctgggcagcatccaggccaacggcatcggcagcggcaactacgtgggcgtgctgagctggatcagccctcccagccaccccagcggcagccaagtggacctgtggaagatccccaactacgggagcagcatcaccgaagctacccacctcgctcccagcgtgtaccctcctggcttcggcgaggtgctggtgttcttcatgagcaagatgcctggccctggcgcctacaatctgccctgcctgctgccccaggagtacatcagtcacctggccagcgagcaggctccaacagtgggcgaggccgccttactgcactacgtcgaccccgacaccggccggaacctgggagagttcaaggcctaccccgacggcttcctgacctgcgtgcccaacggcgcaagcagcggccctcagcagctgcccatcaacggcgtgttcgtgtttgtgagctgggtgagccggttctaccagctgaagcccgtaggcaccgccagctccgctcggggaagactgggcctgcggcggtgataataggctggagcctcggtggcctagcttcttgccccttgggcctccccccagcccctcctccccttcctgcacccgtacccccgtggtctttgaataaagtctgagtgggcggc

SEQ ID NO: 153 - RNA, 1817 nt, Synthetic construct
  source 1..1817: mol_type = other RNA
gggaaataagagagaaaagaagagtaagaagaaatataagaccccggcgccgccaccatgaagatggccagcaacgacgccgccccaagcaacgacggcgccgccaacctggtgcccgaggccaccaacgaggtgatggccctggagccagtggtgggcgcctccatcgctgcccctgtggtgggccagcagaacatcatcgacccctggatccgggagaacttcgtgcaggcccctcagggcgagttcaccgtgtctccccggaacagccccggcgagatgctgctgaacctggagctgggccccgagctgaacccctacctgagccacctgagccggatgtacaacggctacgccggcggcatgcaggtgcaggtggtgctggccggcaacgccttcaccgccggcaagatcatcttcgccgccgtgccaccccacttccccgtggagaacatcagcgccgcccagatcaccatgtgtccccacgtgatcgtggacgtgcggcagctggagcccgtgctgctgcccctgcccgacatccggaaccggttcttccactacaaccaggagaacacaccccggatgcggctggtggccatgctgtatactcccctgcgggccaatagcggcgaggacgtgttcaccgtgagctgtcgggtgctgaccaggcccgcacccgacttcgagttcaccttcctggtgccgcccaccgtggagagcaagaccaagcccttcaccctgcccatcctgaccctgggcgagctgagcaacagccggttccccgcccctatcgacatgctgtacaccgaccccaacgaggccatcgtggtgcagccccagaacggccggtgcaccttggacggcaccctgcagggcaccacccagctggtgcccacccagatctgcagcttccggggcaccctgatcagccagaccagccggagcgccgacagcaccgacagcgcaccccgggtgcggaaccatcccctccacgtgcagctgaagaacctggacggcactccctacgaccccaccgacgaagtgcccgcagtgctgggcgccatcgacttcaagggcaccgtgttcggcgtggccagccagcggaacaccaccggcaactctataggcgccaccagagcccacgaggtgcacatcgacaccaccaatccccggtacacccctaagctgggcagcgtgctgatgtacagcgagagcaccgacttcgacgacggccagcccacccggttcactcccatcggcatgggcgccgacgactggcaccagtgggagctgcccgagtacagcggccacctgaccctgaacatgaacttagccccagccgtggctccagcattccccggcgagcggatcctgttcttccggagcgtggtgccctcagccggcggttacggcagcggccacatcgactgcctgattccccaggagtgggtgcagcacttctaccaggaggccgcccctagccagagcgccgtggccctgatccggtacgtgaaccccgacaccggccggaacatcttcgaggccaagctgcaccgggagggcttcatcaccgtcgccaacagcggcaacaaccccatcgtggttcctccgaacggctacttccggttcgaggcctgggtgaaccagttctacaccctgaccccaatgggcacaggccagggccgacggagggtgcagtgataataggctggagcctcggtggcctagcttcttgccccttgggcctccccccagcccctcctccccttcctgcacccgtacccccgtggtctttgaataaagtctgagtgggcggc

SEQ ID NO: 154 - RNA, 1802 nt, Synthetic construct
  source 1..1802: mol_type = other RNA
gggaaataagagagaaaagaagagtaagaagaaatataagaccccggcgccgccaccatgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggcgagttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccacccaccgtggagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgacagctgggtgaaccagttctacagcctggccccaatgggcaccggtaacggacgccggcgggtgcagtgataataggctggagcctcggtggcctagcttcttgccccttgggcctccccccagcccctcctccccttcctgcacccgtacccccgtggtctttgaataaagtctgagtgggcggc

SEQ ID NO: 155 - RNA, 1796 nt, Synthetic construct
  source 1..1796: mol_type = other RNA
gggaaataagagagaaaagaagagtaagaagaaatataagaccccggcgccgccaccatgaagatggccagcaacgacgccgccccaagcaacgacggagccgctggcctggtgcccgagggcaacaacgagaccctgcccctggaacccgtggccggcgcagccattgccgccccagtgaccggccagaacaacatcatcgacccctggatccggaccaacttcgtgcaggctcccaacggcgagttcaccgtgtctccccggaacagccccggcgagatcctgctgaacctggagctgggccccgacctgaacccctacctggcccacctgagccggatgtacaacggctacgccggcggcgttgaggtgcaggtgctgctggccggcaacgccttcaccgccggcaagatcctgttcgccgccgtgccacccaacttccccgtggagttcctgagccccgcccagatcaccatgctgccccacctgatcgtggacgtgcggaccctggagcccatcatgattcccctgcccgacgtgcggaacaccttcttccactacagcaaccagcccaacagccggatgcggctggtggccatgctgtacactcccctgcggagcaacggcagcggcgacgacgtgttcaccgtgagctgccgggtgctgacacggcccactcccgacttcgagttcacctacctggtgcctcccagcgtggagagcaagaccaagcccttcagcctgcccatcctgaccctgagcgagctgaccaacagccggttccccgtgcccatcgacagcctgttcaccgcccagaacaacgtgctgcaggtgcagtgccagaacggccggtgcaccctggacggcgagctgcagggcaccacccagctgctgcccagcggcatctgtgcctttcggggccgagtgaccgcccagatcaaccagcgggaccggtggcacatgcagctgcagaacctgaacggcaccacctacgaccccaccgacgacgtgcccgcacccctgggaacccctgacttcaagggcgtggtgttcggcatggtgagccagcggaacgtgggcaacgacgctcccggcagcaccagagcccagcaggcctgggtgagcacctacagcccacagttcgtgcccaagctgggcagcgtgaacctgcggatcagcgacaacgacgacttccagttccagcccaccaagttcactcccgtgggcgtgaacgacgacgacgacggccaccccttccggcagtgggagctgcccaactacagcggcgagctgaccctgaacatgaacctggcacctcctgtggcccctaacttccccggcgagcagctgctgttcttccggagcttcgtgccctgcagcggcggctacaaccagggcatcatcgactgcctgataccccaggagtggatccagcacttctaccaggagagcgcccctagccagagcgacgtggccctgatccggtacgtgaaccccgacacaggccggaccctgttcgaggccaagctgcaccggagcggctacatcaccgtggcccacagcggcgactatcccctggtggtgcccgccaacggccacttccggttcgacagctgggtgaaccagttctacagcctggccccaatggggacaggcaacggccggagacgggcccagtgataataggctggagcctcggtggcctagcttcttgccccttgggcctccccccagcccctcctccccttcctgcacccgtacccccgtggtctttgaataaagtctgagtgggcggc

SEQ ID NO: 156 - RNA, 1820 nt, Synthetic construct
  source 1..1820: mol_type = other RNA
gggaaataagagagaaaagaagagtaagaagaaatataagaccccggcgccgccaccatgaagatggccagcaacgacgccgccccaagcagcgacggagctgccggcctggtgcccgagagcaacaacgaggtgatggcccttgagcctgtggctggcgcctctcttgccgcccctgtgaccggccagaccaacatcatcgacccctggatccggatgaacttcgtgcaggcccctaacggcgagttcaccgtgtcccctcggaacagccccggcgaggtgctgctgaacctggagctgggccccgagctgaacccctacctggcccacctgagccggatgtacaacggctacgccggcggcatggaggtgcagatcatgctggccggcaacgccttcaccgccggcaagctgatcttcgccgccgtgcctccccacttccccatcgagaacctgtctcctcctcagatcaccatgtttccccacgtgatcatcgacgtgcggaccctggagcccgtgctgctgcccatgcccgacatccggaacagcttcttccacttcatccagcgggacgagcccaagatgcggctggtggccatgctgtacacacccctgcggagcaacggcagcggcgacgacgtgttcaccgtgagctgccgggtgctgacccggccaacacccgacttcgacttcacctacctggtgccaccaaccgtggagagcaagagcaagcccttcaccctgcccatcctgaccctgggcgagctgaccaacagccggtttcccctgcccattgacgtgctgtacaccaaccccaacgagagcgccatcgtgcagtgccagaacggccggtgcaccctggacggcgagctgcagggcaccacccagctgctgcctaccggcatctgcgccttccggggcaaggtgacccagcaggtgcaggacgagcaccggggcacccactggaacatgaccgtgaccaacctgaacggcaccccattcgaccccaccgaggacgtgcccgccccactgggcactcccgacttcagcggccagatctacggcgtgatcagccagcggaacaccaacactgtacccggcgagggagacctgcccgccaatcgggcccacgaggccgtgatcgccacctacagccccaagttcaccccgaagctgggcaacatccagttcagcacctgggagacccaggacgtgagcagcggccagcccaccaagttcactcccgtgggcctggccagcgtggacgccaacagccacttcgaccagtggaccctgcccagctacagcggcgccctgaccctgaacatgaacctggcacccagcgtggcccctgtgttccccggcgagtgcctgctgttcttccggagcttcatacccctgaagggcggctacggcaaccccgccatcgactgcctgatgccccaggagtgggtgcagcacctgtaccaggagagcgctcccagcctgagcgacgtggccctggtgcggtacgtgaaccccgagactggccggaccctgttcgaggccaagctgcaccggaacggcttcctgaccgtggccaggaacagcgccggaccagtggtggccccaaccaacggctacttccggttcgacagctgggtgaaccagttctacaccctggctcccatgggcaacggctctggacggcggcggatgcagtgataataggctggagcctcggtggcctagcttcttgccccttgggcctccccccagcccctcctccccttcctgcacccgtacccccgtggtctttgaataaagtctgagtgggcggc

SEQ ID NO: 157 - RNA, 1811 nt, Synthetic construct
  source 1..1811: mol_type = other RNA
gggaaataagagagaaaagaagagtaagaagaaatataagaccccggcgccgccaccatgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggcaacgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 158 - RNA, 812 nt, Synthetic construct
  source 1..812: mol_type = other RNA
gggaaataagagagaaaagaagagtaagaagaaatataagaccccggcgccgccaccatggcccaggccatcatcggcgccatcgccgctagcaccgccggcagcgcacttggcgccggcatccaggtgggcggagaagccgccctgcagagccagcggtaccagcagaacctccagctgcaggagaacagcttcaagcacgaccgggagatgatcggctaccaggtggaggccagcaaccagctgctggccaagaacctggccacccggtactctctgctgagggccggcggtctgacctctgccgacgctgcccgcagcgtggctggagccccagtgacccggatcgtggactggaacggagtgcgggtgtccgccccagagagcagcgccaccaccctgagaagcggcggcttcatgagcgtgcccatccccttcgccagcaagcagaagcaggtgcagagcagcggcatcagcaaccccaactacagccccagcagcatcagccggaccaccagctgggtggagagccagaacagcagccggttcggcaacctgagcccctaccacgccgaggccctgaacaccgtgtggctgacacctcccggcagcaccgccagcagcaccctgagcagcgtgccccggggctacttcaacaccgaccggctgcccctgttcgccaacaaccggcggtgataataggctggagcctcggtggcctagcttcttgccccttgggcctccccccagcccctcctccccttcctgcacccgtacccccgtggtctttgaataaagtctgagtgggcggc

SEQ ID NO: 159 - RNA, 980 nt, Synthetic construct
  source 1..980: mol_type = other RNA
gggaaataagagagaaaagaagagtaagaagaaatataagaccccggcgccgccaccatggccggagccttcttcgccggactggccagcgacgtgttgggctctggcctgggcagcctgattaacgccggggccggcgccattaaccagaaggtggagttcgagaacaaccggaaactgcagcaagccagctttcagttcagcagcaacctgcagcaggccagcttccagcacgacaaggagatgctgcaggcccagatcgaggccaccaagaagctgcagcaggagatgatgaaggtgaagcaggccatgctgctggagggcggcttcagcgagaccgacgccgccagaggcgctatcaacgctcccatgaccaaggccctggactggagcggcacccggtactgggccccagacgcccggacaaccacctacaacgccggccggttcagcacccctcaacccagcggcgctctgccaggaagagccaatctgcgtgacgccgtgccagcccacggttccagcagcaagagcagcaacttcagcaccgccaccagcgtgtacagcaaccagaccaccagcacacggctgggaagcaccgccggcagcggaaccagcgtgagcagcctgcccagcaccgcacggacccggagctgggtggaggaccagagccggaacctgagccccttcatgcggggcgcccacaacatcagcttcgtgacccctcccagcagccggagcagcagccagggcaccgtgagcaccgtgcccaaggaggtgctggacagctggaccggcgccttcaacacccggcggcagcccctgttcgcccacatccggaagcggggcgagagccgggtgtgataataggctggagcctcggtggcctagcttcttgccccttgggcctccccccagcccctcctccccttcctgcacccgtacccccgtggtctttgaataaagtctgagtgggcggc

SEQ ID NO: 160 - RNA, 797 nt, Synthetic construct
  source 1..797: mol_type = other RNA
gggaaataagagagaaaagaagagtaagaagaaatataagaccccggcgccgccaccatggcccaggccatcatcggcgccatcgccgctagcaccgccggcagcgcacttggcgccggcatccaggtgggcggagaagccgccctgcagagccagcggtaccagcagaacctccagctgcaggagaacagcttcaagcacgaccgggagatgatcggctaccaggtggaggccagcaaccagctgctggccaagaacctggccacccggtactctctgctgagggccggcggtctgacctctgccgacgctgcccgcagcgtggctggagccccagtgacccggatcgtggactggaacggagtgcgggtgtccgccccagagagcagcgccaccaccctgagaagcggcggcttcatgagcgtgcccatccccttcgccagcaagcagaagcaggtgcagagcagcggcatcagcaaccccaactacagccccagcagcatcagccggaccaccagctgggtggagagccagaacagcagccggttcggcaacctgagcccctaccacgccgaggccctgaacaccgtgtggctgacacctcccggcagcaccgccagcagcaccctgagcagcgtgccccggggctacttcaacaccgaccggctgcccctgttctgataataggctggagcctcggtggcctagcttcttgccccttgggcctccccccagcccctcctccccttcctgcacccgtacccccgtggtctttgaataaagtctgagtgggcggc

SEQ ID NO: 161 - RNA, 938 nt, Synthetic construct
  source 1..938: mol_type = other RNA
gggaaataagagagaaaagaagagtaagaagaaatataagaccccggcgccgccaccatggccggagccttcttcgccggactggccagcgacgtgttgggctctggcctgggcagcctgattaacgccggggccggcgccattaaccagaaggtggagttcgagaacaaccggaaactgcagcaagccagctttcagttcagcagcaacctgcagcaggccagcttccagcacgacaaggagatgctgcaggcccagatcgaggccaccaagaagctgcagcaggagatgatgaaggtgaagcaggccatgctgctggagggcggcttcagcgagaccgacgccgccagaggcgctatcaacgctcccatgaccaaggccctggactggagcggcacccggtactgggccccagacgcccggacaaccacctacaacgccggccggttcagcacccctcaacccagcggcgctctgccaggaagagccaatctgcgtgacgccgtgccagcccacggttccagcagcaagagcagcaacttcagcaccgccaccagcgtgtacagcaaccagaccaccagcacacggctgggaagcaccgccggcagcggaaccagcgtgagcagcctgcccagcaccgcacggacccggagctgggtggaggaccagagccggaacctgagccccttcatgcggggcgcccacaacatcagcttcgtgacccctcccagcagccggagcagcagccagggcaccgtgagcaccgtgcccaaggaggtgctggacagctggaccggcgccttcaacacccggcggcagtgataataggctggagcctcggtggcctagcttcttgccccttgggcctccccccagcccctcctccccttcctgcacccgtacccccgtggtctttgaataaagtctgagtgggcggc

SEQ ID NO: 162 - RNA, 722 nt, Synthetic construct
  source 1..722: mol_type = other RNA
gggaaataagagagaaaagaagagtaagaagaaatataagaccccggcgccgccaccatggccccacccaccctgtggagccgggtgaccaagttcggcagcggctggggcttctgggtgagccccaccgtgttcatcaccaccacccacgtggtgcccaccggcgtgaaggagttcttcggcgagcccctgagcagcatcgccatccaccaggccggcgagttcacccagttccggttcagcaagaagatgcggcccgacctgaccggcatggtgctggaggagggctgtcccgagggcaccgtgtgcagcgtgctgatcaagcgggacagcggcgagctgctgcccctggccgtgcggatgggcgccatcgccagcatgcggatccagggcagactggtgcacggccagagcggcatgctgctgaccggcgccaacgccaagggcatggacctgggcaccatccccggagactgcggcgctccctacgtgcacaagcggggcaacgactgggtggtgtgtggagtccacgccgccgccaccaagagcggcaacaccgtggtgtgcgccgtgcaggccggagagggcgagaccgccctggagtgataataggctggagcctcggtggcctagcttcttgccccttgggcctccccccagcccctcctccccttcctgcacccgtacccccgtggtctttgaataaagtctgagtgggcggc

SEQ ID NO: 163 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 164 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcctgcccatcctgaccctgagcgagctgaccaacagccggttccccgtgcccatcgacagcctgttcaccgcccagaacaacgtgctgcaggtgcagtgccagaacggccggtgcaccctggacggcgagctgcagggcaccacccagctgctgcccagcggcatctgtgcctttcggggccgagtgaccgcccagatcaaccagcgggaccggtggcacatgcagctgcagaacctgaacggcaccacctacgaccccaccgacgacgtgcccgcacccctgggaacccctgacttcaagggcgtggtgttcggcatggtgagccagcggaacgtgggcaacgacgctcccggcagcaccagagcccagcaggcctgggtgagcacctacagcccacagttcgtgcccaagctgggcagcatcaacctgcggatcagcgacaacgacgacttccagttccagcccaccaagttcactcccgtgggcgtgaacgacgacgacgacggccaccccttccggcagtgggagctgcccaactacagcggcgagctgaccctgaacatgaacctggcacctcctgtggcccctaacttccccggcgagcagctgctgttcttccggagcttcgtgccctgcagcggcggctacaaccagggcatcatcgactgcctgataccccaggagtggatccagcacttctaccaggagagcgcccctagccagagcgacgtggccctgatccggtacgtgaaccccgacacaggccggaccctgttcgaggccaagctgcaccggagcggctacatcaccgtggcccacagcggcgactatcccctggtggtgcccgccaacggccacttccggttcgacagctgggtgaaccagttctacagcctggccccaatggggacaggcaacggccggagacgggcccagtgataataggctggagcctcggtggcctagcttcttgccccttgggcctccccccagcccctcctccccttcctgcacccgtacccccgtggtctttgaataaagtctgagtgggcggc

SEQ ID NO: 165 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcctgcccatcctgaccctgagcgagctgaccaacagccggttccccgtgcccatcgacagcctgttcaccgcccagaacaacgtgctgcaggtgcagtgccagaacggccggtgcaccctggacggcgagctgcagggcaccacccagctgctgcccagcggcatctgtgcctttcggggccgagtgaccgcccagatcaaccagcgggaccggtggcacatgcagctgcagaacctgaacggcaccacctacgaccccaccgacgacgtgcccgcacccctgggaacccctgacttcaagggcgtggtgttcggcatggtgagccagcggaacgtgggcaacgacgctcccggcagcaccagagcccagcaggcctgggtgagcacctacagcccacagttcgtgcccaagctgggcagcatcaacctgcggatcagcgacaacgacgacttccagttccagcccaccaagttcactcccgtgggcgtgaacgacgacgacgacggccaccccttccggcagtgggagctgcccaactacagcggcgagctgaccctgaacatgaacctggcacctcctgtggcccctaacttccccggcgagcagctgctgttcttccggagcttcgtgccctgcagcggcggctacaaccagggcatcatcgactgcctgataccccaggagtggatccagcacttctaccaggagagcgcccctagccagagcgacgtggccctgatccggtacgtgaaccccgacacaggccggaccctgttcgaggccaagctgcaccggagcggctacatcaccgtggcccacagcggcgactatcccctggtggtgcccgccaacggccacttccggttcgacagctgggtgaaccagttctacagcctggccccaatggggacaggcaacggccggagacgggcccagtgataataggctggagcctcggtggcctagcttcttgccccttgggcctccccccagcccctcctccccttcctgcacccgtacccccgtggtctttgaataaagtctgagtgggcggc

SEQ ID NO: 166 - RNA, 1136 nt, Synthetic construct
  source 1..1136: mol_type = other RNA
gggaaataagagagaaaagaagagtaagaagaaatataagaccccggcgccgccaccatgcctggcagcaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 167 - RNA, 1238 nt, Synthetic construct
  source 1..1238: mol_type = other RNA
gggaaataagagagaaaagaagagtaagaagaaatataagaccccggcgccgccaccatgaacccaaaccagaagatcatcaccatcggcagtatctgcatgaccatcggcaccgccaacctgatcctgcagatcggcaacatcatcagcatctgggtgagccctggcagcaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccgggactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcgacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 168 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 169 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaacttttgctgcgcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagcccctgccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 170 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaacttttgctgcgcccctggcggcgagttcaccgtgtcccctgccaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagcccctgccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacgactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgtgcacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 171 - RNA, 1796 nt, Synthetic construct; identical to SEQ ID NO: 170
  source 1..1796: mol_type = other RNA

SEQ ID NO: 172 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccacaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccaaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcgccggacgtagacgggccctgtgataataggctggagcctcggtggcctagcttcttgccccttgggcctccccccagcccctcctccccttcctgcacccgtacccccgtggtctttgaataaagtctgagtgggcggc

SEQ ID NO: 173 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 174 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgtgcgcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagcccctgccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 175 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgtgcgcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgtgcatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 176 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggctgcgccttcacctgcggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 177 - RNA, 1820 nt, Synthetic construct
  source 1..1820: mol_type = other RNA
gggaaataagagagaaaagaagagtaagaagaaatataagaccccggcgccgccaccatgaagatggccagcaacgacgccgccccaagcaacgacggtgctgctgggctggtgcccgagatcaacaacgaggccatggcactcgaaccagtagcaggggctgctattgctgctccactgaccggccagcagaacatcatcgacccctggatcatgaacaacttcgtgcaggcaccaggcggcgagttcaccgtgtctccccggaacagccccggcgaagtccttctgaacctggagctgggccccgagatcaacccctacctggcccacctggcccggatgtacaacgggtacgcaggaggattcgaggtgcaagtcgtgctcgccggcaacgccttcaccgccggcaagatcatcttcgccgccattcctcctaactttccaatcgacaacctgagcgccgcccagatcaccatgtgtcctcacgtcatcgtggacgtcaggcagctggagcccgtgaacctgcccatgccagacgtaaggaacaacttcttccactacaaccagggcagcgacagccggctgcggctcatcgctatgctgtacactccactgcgggccaacaacagcggcgacgacgtgttcaccgtgagctgtagggttctaacccggcccagccccgacttcagcttcaacttcctggtgcctcctaccgtggagagcaagaccaagcccttcagcctgcccatcctgaccatcagcgagatgagcaacagccggttccccgtgcccatcgacagcctgcacaccagccccaccgagaacatcgtggtgcagtgccagaacgggcgcgtgaccctagacggggagctgatgggcaccacccagctgctgcccagccagatctgcgccttccggggcgtgctgatccggagcaccagtagggcaagcgatcaagcggacaccgccactcctcggctgttcaactactactggcacatccagctggacaacctgaacggcactccttacgaccccgccgaggacatccccgggcctctgggcactccagacttccggggcaaggtgttcggcgtggccagccagcggaaccctgacacaacaactcgggcacacgaagccaagatcgacaccacctcaggccggttcactccaaagctgggcagcctggagatcagcaccgagagcgacgacttcgaccagaacaagcctacccggttcacacctgtgggcatcggcgtggaccacgaggccgacttccagcagtggaccctgcccgactacgccggccagttcacccacaacatgaacctggctccagccgtggctcctaacttccccggcgagcagctgctgttcttccggagccaattaccatcctctggaggtcgcagcaacggcatcctggactgcctggtgccccaggagtgggtgcagcacttctaccaggagagcgctccaagccagagccaggtggccctggtgcggtacatcaaccccgacaccggacgagttctgttcgaggccaagctgcacaagctgggcttcatcaccatcgccaagaacggcgacagccccatcaccgtgccccctaacggctacttccggttcgagagctgggtgaaccccttctacaccctggcgccaatgggcaccggcaacggacgtcgccggatccagtgataataggctggagcctcggtggcctagcttcttgccccttgggcctccccccagcccctcctccccttcctgcacccgtacccccgtggtctttgaataaagtctgagtgggcggc

SEQ ID NO: 178 - RNA, 1781 nt, Synthetic construct
  source 1..1781: mol_type = other RNA
gggaaataagagagaaaagaagagtaagaagaaatataagaccccggcgccgccaccatgaagatggccagcaacgacgccgccccaagcaacgacggtgctgctgggctggtgcccgaggtgaacaacgagaccatggcactcgaaccagtagcaggggcttctattgctgctccactgaccggccagaacaacgtgatcgacccctggatccggctgaacttcgtgcaggcaccaaacggcgagttcaccgtgtctccccggaacagccccggcgaagtccttctgaacctggagctgggccccgagctgaacccctacctggcccacctgagccggatgtacaacgggtacgcaggaggagtcgaggtgcaagtcttgctcgccggcaacgccttcaccgccggcaagctggtgttcgccgccgtgcctcctcactttccactggagaacatcagccccggccagatcaccatgttccctcacgtcatcattgacgtcaggaccctggagcccgtgctgctgcccctgccagacgtaaggaacagcttcttccactacaaccagcagaacgagccccggatgcggctcgtcgctatgctgtacactccactgcggagcaacggcagcggcgacgacgtgttcaccgtgagctgtagggttctaacccggcccagccccgacttcgacttcaactacctggtgcctcctaccgtggagagcaagaccaagcccttcaccctgcccgtgctgaccatcggcgagctgaccaacagccggttccccgtgcccatcgacgagctgtacaccagccccaacgagagcctggtggtgcagccccagaacgggcgctgtgcactagacggggagctgcagggcaccacccagctgctgcccaccgccatctgcagcttccggggccggatcaaccagaaggtgagcggcgagaaccacgtttggaacatgcaggtgaccaacatcaacggcactcctttcgaccccaccgaggacgtgcccgctcctctgggcactccagacttcagcggcaagctgttcggcgtgctgagccagcgggaccacgacaacgcctgccggagccacgacgccgtgatcgccaccgactcaacgaagttcactccaaagctgggcgccgtgcagatcggcacctgggagcaggacgacgtgcacatcaaccagcctaccaagttcacacctgtgggcctgttcgagaacgagggcttcaaccagtggaccctgcccaactacagcggcgccctgaccctgaacatgggcctggctccacccgtggctcctaccttccccggcgagcagatcctgttcttccggagccacattccactgaaaggaggtgtagctgatccagtgatcgactgcctgctgccccaggagtggatccagcacctgtaccaggagagcgctccaagccagaccgacgtggccctgatccggttcaccaaccccgacaccggacgagttctgttcgaggccaagctgcaccggagcggctacatcaccgtggccaacaccggcagccggcccatcgtggtgcccgccaacggctacttccggttcgacagctgggtgaaccagttctacagcctggcgccaatgggcaccggcaacggacgtcgccggatgcagtgataataggctggagcctcggtggcctagcttcttgccccttgggcctccccccagcccctcctccccttcctgcacccgtacccccgtggtctttgaataaagtctgagtgggcggc

SEQ ID NO: 179 - RNA, 1811 nt, Synthetic construct
  source 1..1811: mol_type = other RNA
gggaaataagagagaaaagaagagtaagaagaaatataagaccccggcgccgccaccatgatgatggccagcaaggacgcccctaccaacatggacggcactagcggcgcaggccagctggtgcccgaggccaacaccgccgagcccatcagcatggaacccgtgtgcggcgctgcaaccgcagcctgtaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggcaacgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 180 - RNA, 1811 nt, Synthetic construct
  source 1..1811: mol_type = other RNA
gggaaataagagagaaaagaagagtaagaagaaatataagaccccggcgccgccaccatgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgtgcgcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggcaacgccttcaccgccggcaagatcatcatcagctgcatcccacccggcttcgtggcccagaacgtgagcatctgccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 181 - RNA, 1811 nt, Synthetic construct
  source 1..1811: mol_type = other RNA
gggaaataagagagaaaagaagagtaagaagaaatataagaccccggcgccgccaccatgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggctgcgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgaggtgcagcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 182 - RNA, 1811 nt, Synthetic construct
  source 1..1811: mol_type = other RNA
gggaaataagagagaaaagaagagtaagaagaaatataagaccccggcgccgccaccatgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggctgcgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggcctgcggcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 183 - RNA, 1811 nt, Synthetic construct
  source 1..1811: mol_type = other RNA
gggaaataagagagaaaagaagagtaagaagaaatataagaccccggcgccgccaccatgatgatggccagcaaggacgcccctaccaacatggacggcactagcggcgcaggccagctggtgcccgaggccaacaccgccgagcccatcagcatggaacccgtggcaggcgctgcaaccgcagccgcaaccgcagggcaggtgaacatgatcgacccctggatcatgaacaactacgtgcaggcccctcagggcgagttcaccatcagccccaacaacacgcccggcgacatcctgttcgacctgcagctggggccccacctgaaccccttcctgagccacctggcccagatgtacaacggctgggtgggcaacatgaaggtgaaggtgctgctggccggctgcgccttcaccgccggcaagatcatcatcagctgcatcccacccggcttcgtggcccagaacgtgagcatcgcccaggccaccatgttcccacacgtgattgccgacgtgcgggtgctggagcccatcgaggtgcccctggaggacgtgcggaacgtgctgttccacaacaacgacaacaccccaaccatgcggctggtgtgcatgctgtacacccctctgagggccagctgcagcagcagcggcaccgaccccttcgtgatcgccggccgggtgctgacctgccccagccccgacttcagcttcctgttcctggtgccacctaacgtggagcagaagaccaagcccttcagcgtgcccaacctgcccctgaacaccctgagcaacagccgggtgcccagcctgatcaagagcatgatggtgagccgggaccacggccagatggtgcagttccagaacggccgggtgaccctggacggccagctgcaaggcaccacccctaccagcgccagccagctgtgcaagatccggggcagcgtgttccacgccaacggcggcaacggctacaacctgaccgagctggacggcagcccctaccacgccttcgagagccccgccccaatcggcttccccgacctgggcgagtgcgactggcacatggaggccagccccaccacccagttcaacaccggcgacgtgatcaagcagatcaacgtgaagcaggagagcgccttcgccccacacctgggcaccatccaggccgacggcctgagcgacgtgagcgtgaacaccaacatgatcgccaagctgggctgggtgagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggcccctacccggggtgaagccgccctgctgcactacctggaccccgacacccaccggaacctgggtgagttcaagctgtaccccgacggcttcatgacctgcgtgcccaatagctctggcaccggtccccagaccctgcccatcaacggcgtgttcgtgttcgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 184 - RNA, 1808 nt, Synthetic construct
  source 1..1808: mol_type = other RNA
gggaaataagagagaaaagaagagtaagaagaaatataagaccccggcgccgccaccatgatgatggccagcaaggacgccaccagcagcgtggacggcgcaagcggcgcaggccagctggtgcccgaggtgaacgccagcgaccctctggccatggatcccgtggcaggctccagcaccgcagtggcaaccgcagggcaggtgaaccccatcgacccgtggatcatcaacaacttcgtgcaagcccctcagggcgagttcactatcagccccaacaacacgcccggcgacgttctgttcgacctgagcctgggtccccacctgaaccccttccttctgcatctgagccagatgtacaacggttgggtgggcaacatgcgggtgcggatcatgctggccggctgcgccttcaccgccggcaagatcatcgtgagctgcatcccacccggctttggcagccacaacctgaccatcgcacaggccaccctgttcccacacgtgattgccgacgttcggaccctggatcccatcgaggtgcccctggaggacgtgcggaacgtgctgttccacaacaacgaccggaaccagcagaccatgcggctggtgtgcatgctgtacacccctctgaggacctgcggcggcacaggcgacagcttcgtggtggccggacgggtgatgacgtgtcccagccccgacttcaacttcctgttcctggtgccacctaacgtggagcagaagaccaagcccttcagcgtgcccaacctgcccctgaacaccctgagcaacagccgggttcccagcctgatcaagagcatgatggtgagccgggaccacggccagatggtacagttccagaacggccgggtcaccctggacggtcagctgcaaggcaccacacctaccagcgccagccagctgtgcaagatccggggcagcgtgttccacgccaacggcggcaacggctacaaccttaccgagctggacggcagcccctaccacgccttcgagagccccgccccaatcggcttccccgacctgggcgagtgcgactggcacatggaggccagccccaccacccagttcaacaccggggacgtgatcaagcagatcaacgtgaagcaggagagcgccttcgccccacacctgggcaccatccaggccgacggcctcagcgacgtgagcgtgaacaccaacatgatcgccaagctgggctgggttagccccgtgagcgacggccacaagggcgacgtggacccctgggtgatcccacggtacggcagcaccctgaccgaagctgcccagctcgcccctccaatctatcctccgggcttcggcgaggccatcgtgttcttcatgagcgacttccccatcgcccacggcaccaacggcctgagtgtgccctgcaccattccccaggagttcgtgacccacttcgtgaacgagcaggccccaacccggggtgaagccgccctgctgcactacctggaccccgacacccaccggaatctgggtgagttcaagctgtaccccgacggcttcatgacctgtgtgcccaatagctctggcaccggtccccagaccctgcccatcaacggcgtgttcgtgtttgtgagctgggtgtcacggttctaccagctgaagcccgtgggcaccgctggacccgccagacggctgggcatccggcggagctgataataggctggagcctcggtggcctagcttcttgccccttgggcctccccccagcccctcctccccttcctgcacccgtacccccgtggtctttgaataaagtctgagtgggcggc

SEQ ID NO: 185 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccaccggcctgagccccagccaggtgaccatgttccctcacatcatcgtggacgtgcgacagctggaacccgtgctgatacccctgcccgacgtgcggaacaatttctaccactacaaccagagcaacgaccccaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaacttccggggcgacgtgacccacatcaccggaagccggaactacaccatgaacctggccagccagaactggaataactacgaccctacggaggagatccccgcacccctgggcactcccgacttcgtgggcaagattcagggcatgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggtggtgtgataataggctggagcctcggtggcctagcttcttgccccttgggcctccccccagcccctcctccccttcctgcacccgtacccccgtggtctttgaataaagtctgagtgggcggc

SEQ ID NO: 186 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccaccgccacctgagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 187 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcagattcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgattggtgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 188 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcagattcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccaccaccacctgagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgattggtgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 189 - RNA, 1802 nt, Synthetic construct
  source 1..1802: mol_type = other RNA
gggaaataagagagaaaagaagagtaagaagaaatataagaccccggcgccgccaccatgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggcgagttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccacctcctcccgagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgacagctgggtgaaccagttctacagcctggccccaatgggaaccggtaacggacgccggcgggtgcagtgataataggctggagcctcggtggcctagcttcttgccccttgggcctccccccagcccctcctccccttcctgcacccgtacccccgtggtctttgaataaagtctgagtgggcggc

SEQ ID NO: 190 - RNA, 1802 nt, Synthetic construct
  source 1..1802: mol_type = other RNA
gggaaataagagagaaaagaagagtaagaagaaatataagaccccggcgccgccaccatgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggccggttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccacccaccgtggagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgactggtgggtgaaccagttctacagcctggccccaatgggaaccggtaacggacgccggcgggtgcagtgataataggctggagcctcggtggcctagcttcttgccccttgggcctccccccagcccctcctccccttcctgcacccgtacccccgtggtctttgaataaagtctgagtgggcggc

SEQ ID NO: 191 - RNA, 1802 nt, Synthetic construct
  source 1..1802: mol_type = other RNA
gggaaataagagagaaaagaagagtaagaagaaatataagaccccggcgccgccaccatgaagatggccagcaacgacgccgcacccagcaccgacggcgcagccggactggtgcccgagagcaacaacgaggtgatggccctggaacctgtggcaggcgccgctctggctgcacccgtgaccggccagaccaacatcatcgacccctggatccgggccaacttcgtgcaggctcccaacggccggttcaccgtgtctccccggaacgctcccggcgaggtgctgctgaacctggagctgggccccgagctgaacccctacctggcccacctggcccggatgtacaacggctacgccggcggcatggaggtgcaggtgatgctggccggcaacgccttcaccgccggcaagctggtgttcgccgccgtgccaccacacttccccgtggagaacctgtctccccagcagatcaccatgttccctcacgtgatcatagacgtgcggaccctggagcccgtgctgctgcccctgcccgacgtgcggaacaacttcttccactacaaccagaaggacgaccccaagatgcggatcgtggccatgctgtacactcccctgcggagcaacggcagcggcgacgacgtgttcaccgtgagctgccgggtgctgaccagacccagccccgacttcgacttcacctacctggtgccaccacctcccgagagcaagaccaagcccttcaccctgcccatcctgaccctgggcgagctgagcaacagccggttccccgtgagcatcgaccagatgtacaccagccccaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagccccttcgaccccagcgaggacatccccgcaccactgggcgtgcccgacttccagggccgggtgttcggcatcatcagccagcgggacaagcacaacagccccggccacaacgagcctgccaaccgcggacacgacgccgtggtgcccacctacaccgcccagtacaccccaaagctgggccagatccagatcggcacctggcagaccgacgacctgaccgtgaaccagcccgtgaagttcacacccgtgggcctgaacgacaccgagcacttcaaccagtgggtggtcccacggtacgccggtgccctgaacctgaacaccaacctggctcccagcgtggctcccgtgttccccggcgagcggctgctgttcttccggagctacatcccactgaagggcggctacggcaaccccgccatcgactgcctgctgccccaggagtgggtgcagcacttctaccaggaggccgccccaagcatgagcgaggtggccctggtgcggtacatcaaccccgacaccggcagagccctgttcgaggccaagctgcaccgggccggcttcatgaccgtgagcagcaacaccagcgcccctgtggtggtgcccgccaacggctacttccggttcgactggtgggtgaaccagttctacagcctggccccaatgggaaccggtaacggacgccggcgggtgcagtgataataggctggagcctcggtggcctagcttcttgccccttgggcctccccccagcccctcctccccttcctgcacccgtacccccgtggtctttgaataaagtctgagtgggcggc

SEQ ID NO: 192 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgaccatgttccctcacatcatcgtggacgtgcgacagctggaacccgtgctgatacccctgcccgacgtgcggaacaatttctaccactacaaccagagcaacgacagcaccatcaagctgatcgccatgctgtactgcccactgcgggccaacaacgccggcgacgacgtgtttaccgtgtgctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 193 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgtgcttccctaccgagggcctgagccccagccaggtgaccatgttccctcacatcatcgtggacgtgcgacagctggaacccgtgctgatacccctgcccgacgtgcggaacaatttctaccactgcaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 194 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgacctgcttccctcacatcatcgtggacgtgcgacagctggaacccgtgctgatacccctgcccgacgtgcggaacaatttctaccactacaaccagagcaacgacagcaccatcaagctgatcgccatgctgtacaccccactgcgggccaacaacgccggcgacgacgtgtttaccgtgagctgccgggttctgacccggcccagcccagacttcgacttcatctttctggtgtgccccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 195 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgtgcttccctaccgagggcctgagccccagccaggtgaccatgttccctcacatcatcgtggacgtgcgacagctggaacccgtgctgatacccctgcccgacgtgcggaacaatttctaccactgcaaccagagcaacgacagcaccatcaagctgatcgccatgctgtactgcccactgcgggccaacaacgccggcgacgacgtgtttaccgtgtgctgccgggttctgacccggcccagcccagacttcgacttcatctttctggtgccacccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

SEQ ID NO: 196 - RNA, 1796 nt, Synthetic construct
  source 1..1796: mol_type = other RNA
gggaaataagagagaaaagaagagtaagaagaaatataagaccccggcgccgccaccatgaagatggccagcagcgacgccaacccaagcgacggcagtgccgccaacctggtgcccgaggtgaacaacgaggtgatggccctggagcccgtggtaggtgccgcgatcgctgcccctgtggccggacagcagaacgtgatcgacccctggatccggaacaactttgtgcaggcccctggcggcgagttcaccgtgtcccctcggaacgctcccggcgagatcctttggagcgcgccgttaggccccgacctgaacccctacctgagccacctggcccggatgtacaacggctacgctggcggcttcgaggtgcaggtgatcctggccggcaacgccttcaccgccggcaagatcatcttcgccgccgtgccaccgaacttccctaccgagggcctgagccccagccaggtgacctgcttccctcacatcatcgtggacgtgcgacagctggaacccgtgctgatacccctgcccgacgtgcggaacaatttctaccactacaaccagagcaacgacagcaccatcaagctgatcgccatgctgtactgcccactgcgggccaacaacgccggcgacgacgtgtttaccgtgtgctgccgggttctgacccggcccagcccagacttcgacttcatctttctggtgtgccccaccgtggagagccggaccaagcccttcagcgtgccggtgctgaccgtcgaggagatgaccaacagccggttccccattcccctggagaagctgttcaccggcccctccagcgccttcgtggtgcagccccagaacggccggtgcaccaccgacggcgtgctgttgggcactacccagctcagccccgtgaacatctgcaccttccggggcgacgtgacccacatcaccggaagccggaactacaccatgaacctggccagccagaactggaataactacgaccctacggaggagatccccgcacccctgggcactcccgacttcgtgggcaagattcagggcgtgctgacccagaccacccggaccgacgggagcacacggggccacaaggccacagtgtacaccggcagcgcggacttcgcgcccaaactgggccgcgtgcagttcgagaccgacaccgaccacgacttcgaggccaaccagaacaccaagttcacacccgtgggcgtgatccaggacggcagcacaacccaccggaacgaaccccagcagtgggtcctgcccagctacagcggccggaacacccacaacgtgcacctggcacccgccgtggcacccaccttccccggcgaacagctgctgttcttccggagcaccatgcccggctgcagcggctaccccaacatggacctggactgcctgctgccccaggagtgggtgcagtacttctaccaggaggcagctcccgcccagagcgacgtggccctgctgcggttcgtgaaccccgataccggccgggtgctgttcgagtgcaagctgcacaagtcgggctacgtgaccgtggcccacaccggccagcacgacctggtgatccctcctaacgggtacttccggttcgatagctgggtgaaccagttctacaccctggcgcctatgggcaacggcaccggacgtagacgggccgtgtgataataggctggagcctcggtggcctagcttcttgccccttgggcctccccccagcccctcctccccttcctgcacccgtacccccgtggtctttgaataaagtctgagtgggcggc

(19) United States
(12) Patent Application Publication
(10) Pub. No.: US 2026/0263582 A1
(43) Pub. Date: Sep. 10, 2026

(54) NOROVIRUS MRNA VACCINES

(71) Applicant: ModernaTX, Inc., Cambridge, MA (US)

(72) Inventors: Brooke Bollman, Brookline, MA (US); Patricia Jorquera, Cambridge, MA (US); Guha Asthagiri Arunkumar, Cambridge, MA (US)

(73) Assignee: ModernaTX, Inc., Cambridge, MA (US)

(21) Appl. No.: 18/881,241

(22) PCT Filed: Jul. 13, 2023
(86) PCT No.: PCT/US2023/070100

§ 371 Date: Jan. 3, 2025

Related U.S. Application Data
(60) Provisional application No. 63/388,982, filed on Jul. 13, 2022.
(60) Provisional application No. 63/503,704, filed on May 22, 2023.

(51) Int. Cl. A61K 39/125; A61K 9/00; A61K 31/7105; A61K 39/00; A61P 31/14

(52) U.S. Cl. CPC A61K 39/125; A61K 9/0019; A61K 31/7105; A61P 31/14; A61K 2039/53; A61K 2039/54; A61K 2039/55555; A61K 2039/57; C12N 2770/16023; C12N 2770/16034; C12N 2770/16052; C12N 2770/16071

(57) ABSTRACT

Aspects of the disclosure relate to compositions of messenger RNA vaccines and methods of administration thereof. Compositions provided herein include one or more RNA polynucleotides having an open reading frame encoding a norovirus capsid protein (VP1).

### RELATED APPLICATIONS

This application claims the benefit under 35 U.S.C. § 119(e) of U.S. provisional application No. 63/388,982, filed Jul. 13, 2022, and U.S. provisional application No. 63/503,704, filed May 22, 2023, each of which is incorporated by reference herein in its entirety.

### REFERENCE TO AN ELECTRONIC SEQUENCE LISTING

The contents of the electronic sequence listing (M137870192WO00-SEQ-EAS.xml; Size: 360,865 bytes; and Date of Creation: Jul. 13, 2023) is herein incorporated by reference in its entirety.

### BACKGROUND

Noroviruses belong to the family Caliciviridae and are a group of non-enveloped, single-stranded RNA viruses that are the primary cause of epidemic and sporadic outbreaks of acute gastroenteritis (AGE) worldwide. Disease is typically resolved in two to three days, but severe cases can lead to hospitalizations and deaths. Young children, elderly, and immunocompromised patients are considered high-risk groups for norovirus infections. It has been estimated that around 19 to 21 million norovirus infections occur annually in the United States alone, including approximately 465,000 emergency department visits, 109,000 hospitalizations, and 900 deaths.

Vaccination is an effective way to provide prophylactic protection against infectious diseases, including, but not limited to, viral, bacterial, and/or parasitic diseases, such as influenza, HIV, hepatitis virus infection, cholera, malaria and tuberculosis, and many other diseases. However, developing vaccines targeting noroviruses has proven difficult, at least in part, because human norovirus cannot be grown in cell culture for experimentation purposes and due to the high genetic and antigenic diversity among norovirus genotypes.

Due to recent advances in recombinant DNA techniques, the use of virus-like particles (VLPs) has become of increasing interest for use in vaccine development. VLPs, which are formed by self-assembling viral structural proteins that mimic the morphology of a pathogen, have been shown to be both non-infective and highly immunogenic. However, previous attempts to generate norovirus VLP vaccines have been met with limited success due, at least in part, to the ineffectiveness of norovirus VLPs inducing an effective B and T cell responses. For example, bivalent norovirus VLPs expressed in insect cells were not found to induce any IgA mucosal antibody response, which would be expected for effective protection against AGE. Additional difficulties are associated with achieving proper expression and/or folding of the structural proteins required to form a VLP that mimics the native norovirus pathogen.

### SUMMARY

Described herein are compositions and methods of nucleic acid vaccines (e.g., mRNA vaccines). In particular, the present disclosure further describes how mRNA can be used to encode and deliver a structural protein encoding the norovirus major capsid protein (VP1) and subsequently allows for a higher order structure in the form of a VLP to serve as an immunogen in vivo. It is therefore of great interest to develop norovirus VLPs from mRNAs for use as norovirus vaccines as a new approach to combatting infectious disease and infectious agents.

In some aspects the invention is a composition comprising (i) at least one, but not more than ten, messenger ribonucleic acid (mRNA) comprising an open reading frame (ORF) encoding a norovirus major capsid protein (VP1); and (ii) a lipid nanoparticle (LNP). In some embodiments, the composition comprises at least two, at least three, at least four, at least five, at least six, or at least seven, messenger ribonucleic acid (mRNA) comprising an open reading frame (ORF) encoding a norovirus major capsid protein (VP1), wherein the at least two, at least three, at least four, at least five, at least six, or at least seven mRNA encode for at least two, at least three, at least four, at least five, at least six, or at least seven norovirus VP1 are each of different genotypes. In some embodiments, the genotypes are selected from genogroup GI and/or genogroup GII.

In some aspects the invention is a composition comprising (i) a first messenger ribonucleic acid (mRNA) comprising an open reading frame (ORF) encoding a first norovirus VP1; (ii) a second messenger ribonucleic acid (mRNA) comprising an open reading frame (ORF) encoding a second norovirus VP1; (iii) a third messenger ribonucleic acid (mRNA) comprising an open reading frame (ORF) encoding a third norovirus VP1; and (vi) a lipid nanoparticle (LNP), wherein the first norovirus VP1 comprises a different genogroup relative to at least one of the second, and/or third norovirus VP1s, and wherein the first, second, and third norovirus VP1 all comprise different genotypes.

In some aspects the invention is a composition comprising (i) a first messenger ribonucleic acid (mRNA) comprising an open reading frame (ORF) encoding a first norovirus VP1; (ii) a second messenger ribonucleic acid (mRNA) comprising an open reading frame (ORF) encoding a second norovirus VP1; (iii) a third messenger ribonucleic acid (mRNA) comprising an open reading frame (ORF) encoding a third norovirus VP1; (iv) a fourth messenger ribonucleic acid (mRNA) comprising an open reading frame (ORF) encoding a fourth norovirus VP1; (v) a fifth messenger ribonucleic acid (mRNA) comprising an open reading frame (ORF) encoding a fifth norovirus VP1; and (vi) a lipid nanoparticle (LNP), wherein the first norovirus VP1 comprises a different genogroup relative to at least one of the second, third, fourth, and/or fifth norovirus VP1s, and wherein the first, second, third, fourth, and/or fifth norovirus VP1 all comprise different genotypes.

In some aspects the invention is a composition comprising (i) a first messenger ribonucleic acid (mRNA) comprising an open reading frame (ORF) encoding a first norovirus VP1; (ii) a second messenger ribonucleic acid (mRNA) comprising an open reading frame (ORF) encoding a second norovirus VP1; (iii) a third messenger ribonucleic acid (mRNA) comprising an open reading frame (ORF) encoding a third norovirus VP1; (iv) a fourth messenger ribonucleic acid (mRNA) comprising an open reading frame (ORF) encoding a fourth norovirus VP1; (v) a fifth messenger ribonucleic acid (mRNA) comprising an open reading frame (ORF) encoding a fifth norovirus VP1; (vi) a sixth messenger ribonucleic acid (mRNA) comprising an open reading frame (ORF) encoding a six norovirus VP1; (vii) a seventh messenger ribonucleic acid (mRNA) comprising an open reading frame (ORF) encoding a seventh norovirus VP1, and (viii) a lipid nanoparticle (LNP), wherein the first norovirus VP1 comprises a different genogroup relative to at least one of the second, third, fourth, fifth, sixth, and/or seventh norovirus VP1s, and wherein first, second, third, fourth, fifth, sixth, and/or seventh norovirus VP1s all comprise different genotypes.

In some embodiments, the first, second, third, fourth, fifth, sixth, and/or seventh norovirus VP1s each comprise a shell domain (S-domain) and a protruding domain (P-domain), wherein the S-domain and P-domain are linked by a hinge domain (H-domain).

In some embodiments, two norovirus VP1s form a homodimer.

In some embodiments, the first, second, third, fourth, fifth, sixth, and/or seventh norovirus VP1s are from a norovirus classified as genogroup GI. In some embodiments, the first, second, third, fourth, fifth, sixth, and/or seventh norovirus VP1s are selected from a norovirus classified as genotype GI.1, GI.2, GI.3, GI.4, GI.5, GI.6, GI.7, GI.8, or GI.9. In some embodiments, the first, second, third, fourth, fifth, sixth, and/or seventh norovirus VP1s are from a norovirus classified as genotype GI.3. In some embodiments, the first, second, third, fourth, fifth, sixth, and/or seventh norovirus VP1s comprise an amino acid sequence having at least 90% identity to the amino acid sequence of SEQ ID NO: 24.

In some embodiments, the first, second, third, fourth, fifth, sixth, and/or seventh norovirus VP1s are from a norovirus classified as genogroup GII. In some embodiments, the first, second, third, fourth, fifth, sixth, and/or seventh norovirus VP1s are selected from a norovirus classified as genotype GII.1, GII.2, GII.3, GII.4, GII.5, GII.6, GII.7, GII.8, GII.9, GII.10, GII.11, GII.12, GII.13, GII.14, GII.15, GII.16, GII.17, GII.18, GII.19 or GII.20. In some embodiments, the first, second, third, fourth, fifth, sixth, and/or seventh norovirus VP1s are from a norovirus classified as genotype GII.4. In some embodiments, the first, second, third, fourth, fifth, sixth, and/or seventh norovirus VP1s comprise an amino acid sequence having at least 90% identity to the amino acid sequence of SEQ ID NO: 3. In some embodiments, the first, second, third, fourth, fifth, sixth, and/or seventh norovirus VP1s are from a norovirus classified as genotype GII.17. In some embodiments, the first, second, third, fourth, fifth, sixth, and/or seventh norovirus VP1s comprise an amino acid sequence having at least 90% identity to the amino acid sequence of SEQ ID NO: 20. In some embodiments, the first, second, third, fourth, fifth, sixth, and/or seventh norovirus VP1s are from a norovirus classified as genotype GII.2. In some embodiments, the first, second, third, fourth, fifth, sixth, and/or seventh norovirus VP1s comprise an amino acid sequence having at least 90% identity to the amino acid sequence of SEQ ID NO: 18. In some embodiments, the first, second, third, fourth, fifth, sixth, and/or seventh norovirus VP1s are from a norovirus classified as genotype GII.3. In some embodiments, the first, second, third, fourth, fifth, sixth, and/or seventh norovirus VP1s comprise an amino acid sequence having at least 90% identity to the amino acid sequence of SEQ ID NO: 64. In some embodiments, the first, second, third, fourth, fifth, sixth, and/or seventh norovirus VP1s are from a norovirus classified as genotype GII.6. In some embodiments, the first, second, third, fourth, fifth, sixth, and/or seventh norovirus VP1s comprise an amino acid sequence having at least 90% identity to the amino acid sequence of SEQ ID NO: 16.

In some embodiments, the different genotypes are GI.3, GII.3, and GII.4.

In some embodiments, the different genotypes are GI.3, GII.2, GII.3, GII.4, and GII.6.

In some embodiments, at least one LNP comprises the mRNA comprising the ORF encoding the first norovirus VP1, the mRNA comprising the ORF encoding the second norovirus VP1, and the mRNA comprising the ORF encoding the third norovirus VP1. In some embodiments, at least one LNP comprises the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, and the mRNA comprising the ORF encoding the fifth norovirus VP1. In some embodiments, at least one LNP comprises the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, the mRNA comprising the ORF encoding the fifth norovirus VP1, the mRNA comprising the ORF encoding the sixth norovirus VP1, and/or the mRNA comprising the ORF encoding the seventh norovirus VP1.

In some embodiments, a first LNP comprises the mRNA comprising the ORF encoding the first norovirus VP1, a second LNP comprises the mRNA comprising the ORF encoding the second norovirus VP1, and a third LNP comprises the mRNA comprising the ORF encoding the third norovirus VP1. In some embodiments, a first LNP comprises the mRNA comprising the ORF encoding the first norovirus VP1, a second LNP comprises the mRNA comprising the ORF encoding the second norovirus VP1, a third LNP comprises the mRNA comprising the ORF encoding the third norovirus VP1, a fourth LNP comprises the mRNA comprising the ORF encoding the fourth norovirus VP1, and a fifth LNP comprises the mRNA comprising the ORF encoding the fifth norovirus VP1 comprise separate LNPs. In some embodiments, a first LNP comprises the mRNA comprising the ORF encoding the first norovirus VP1, a second LNP comprises the mRNA comprising the ORF encoding the second norovirus VP1, a third LNP comprises the mRNA comprising the ORF encoding the third norovirus VP1, a fourth LNP comprises the mRNA comprising the ORF encoding the fourth norovirus VP1, a fifth LNP comprises the mRNA comprising the ORF encoding the fifth norovirus VP1, a sixth LNP comprises the mRNA comprising the ORF encoding the sixth norovirus VP1, and/or a seventh LNP comprises the mRNA comprising the ORF encoding the seventh norovirus VP1.

In some embodiments, the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, the mRNA comprising the ORF encoding the fifth norovirus VP1, the mRNA comprising the ORF encoding the sixth norovirus VP1, and/or the mRNA comprising the ORF encoding the seventh norovirus VP1 are present in one of the following ratios: (i) 1:1, 1:2, 1:3, 2:3; (ii) 1:1:1, 1:3:1, 2:3:1; (iii) 1:1:1:1, 1:3:1:1, 2:3:1:1; (iv) 1:1:1:1:1, 1:3:1:1:1, 2:3:1:1:1; (v) 1:1:1:1:1:1, 1:3:1:1:1:1, 2:3:1:1:1:1; or (vi) 1:1:1:1:1:1:1; 1:3:1:1:1:1:1, 2:3:1:1:1:1:1. In some embodiments, the mRNA comprising the ORF encoding the first norovirus VP1, the mRNA comprising the ORF encoding the second norovirus VP1, and the mRNA comprising the ORF encoding the third norovirus VP1 are present in the following ratio: 1:1:3. In some embodiments, the first norovirus VP1 is GI.3, the second norovirus VP1 is GII.3, and the third norovirus VP1 is GII.4.

In some embodiments, the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, and the mRNA comprising the ORF encoding the fifth norovirus VP1 are present in the following ratio: 1:1:3:1:1. In some embodiments, the first norovirus VP1 is GI.3, the second norovirus VP1 is GII.3, the third norovirus VP1 is GII.4, the fourth norovirus VP1 is GII.2, and the fifth norovirus VP1 is GII.6.

In some embodiments, the LNP comprises an ionizable amino lipid, a sterol, neutral lipid, and a PEG-modified lipid. In some embodiments, the LNP comprises 40-50 mol % ionizable amino lipid, 35-45 mol % sterol, 10-15 mol % neutral lipid, and 2-4 mol % PEG-modified lipid. In some embodiments, the LNP comprises 45 mol %, 46 mol %, 47 mol %, 48 mol %, 49 mol %, or 50 mol % ionizable amino lipid. In some embodiments, the ionizable amino lipid has the structure of Compound 1:

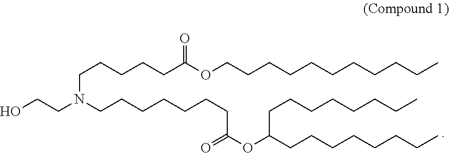 [SMILES: CCCCCCCCCCCOC(=O)CCCCCN(CCO)CCCCCCCC(=O)OC(CCCCCCCC)CCCCCCCC]

In some embodiments, the sterol is cholesterol or a variant thereof. In some embodiments, the neutral lipid is 1,2 distearoyl-sn-glycero-3-phosphocholine (DSPC).

In some aspects the invention is a method comprising administering to a subject an immunogenic composition comprising: (i) a messenger ribonucleic acid (mRNA) comprising an open reading frame encoding a first norovirus VP1; (ii) a messenger ribonucleic acid (mRNA) comprising an open reading frame encoding a second norovirus VP1; (iii) a messenger ribonucleic acid (mRNA) comprising an open reading frame encoding a third norovirus VP1; in an amount effective to induce in the subject an immune response against a viral infection from a member of a Norovirus genus.

In some aspects the invention is a method comprising administering to a subject an immunogenic composition comprising: (i) a messenger ribonucleic acid (mRNA) comprising an open reading frame encoding a first norovirus VP1; (ii) a messenger ribonucleic acid (mRNA) comprising an open reading frame encoding a second norovirus VP1; (iii) a messenger ribonucleic acid (mRNA) comprising an open reading frame encoding a third norovirus VP1; (iv) a messenger ribonucleic acid (mRNA) comprising an open reading frame encoding a fourth norovirus VP1; and/or (v) a messenger ribonucleic acid (mRNA) comprising an open reading frame encoding a fifth norovirus VP1; in an amount effective to induce in the subject an immune response against a viral infection from a member of a Norovirus genus.

In some aspects the invention is a method comprising administering to a subject an immunogenic composition comprising: (i) a messenger ribonucleic acid (mRNA) comprising an open reading frame encoding a first norovirus VP1; (ii) a messenger ribonucleic acid (mRNA) comprising an open reading frame encoding a second norovirus VP1; (iii) a messenger ribonucleic acid (mRNA) comprising an open reading frame encoding a third norovirus VP1; (iv) a messenger ribonucleic acid (mRNA) comprising an open reading frame encoding a fourth norovirus VP1; (v) a messenger ribonucleic acid (mRNA) comprising an open reading frame encoding a fifth norovirus VP1; (vi) a messenger ribonucleic acid (mRNA) comprising an open reading frame encoding a six norovirus VP1; and/or (vii) a messenger ribonucleic acid (mRNA) comprising an open reading frame encoding a seventh norovirus VP1, in an amount effective to induce in the subject an immune response against a viral infection from a member of a Norovirus genus.

In some embodiments, the immune response includes a binding antibody titer to a human norovirus of the genogroup GII. In some embodiments, the human norovirus is genotype GII.4. In some embodiments, the human norovirus is genotype GII.6. In some embodiments, the human norovirus is genotype GII.2. In some embodiments, the human norovirus is genotype GII.3.

In some embodiments, the immune response includes a binding antibody titer to a human norovirus of the genogroup GI. In some embodiments, the human norovirus is genotype GI.3. In some embodiments, the immune response includes a T cell response to a human norovirus.

In some embodiments, the immunogenic composition further comprises at least one lipid nanoparticle. In some embodiments, the mRNA of (i), (ii), and/or (iii) are administered to the subject at the same time. In some embodiments, the immunogenic composition further comprises at least one lipid nanoparticle. In some embodiments, the mRNA of (i), (ii), (iii), (iv), and/or (v) are administered to the subject at the same time. In some embodiments, the immunogenic composition further comprises at least one lipid nanoparticle. In some embodiments, the mRNA of (i), (ii), (iii), (iv), (v), (vi), and/or (vii) are administered to the subject at the same time.

In some embodiments, the immunogenic composition further comprises at least two, but no more than three lipid nanoparticles. In some embodiments, the mRNA of (i), (ii), and/or (iii) are administered to the subject at the same time. In some embodiments, the immunogenic composition further comprises at least two, at least three, at least four, but no more than five lipid nanoparticles. In some embodiments, the mRNA of (i), (ii), (iii), (iv), and/or (v) are administered to the subject at the same time. In some embodiments, the immunogenic composition further comprises at least two, at least three, at least four, at least five, at least six, but no more than seven lipid nanoparticles. In some embodiments, the mRNAs of (i), (ii), (iii), (iv), (v), (vi), and/or (vii) are administered to the subject at the same time.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic depicting phylogenetic trees for norovirus genogroups (left) and genotypes (right). Norovirus genogroups and genotypes are grouped based on the major capsid protein (VP1) amino acid sequence. Viruses of genogroups GI, GII, and GIV infect humans.

FIG. 2 is a schematic depicting the shell and protruding domains of a norovirus major capsid protein (VP1). The S-domain refers to “shell domain”, the P1-subdomain and P2-subdomain refer to “protruding-subdomain 1” and “protruding-subdomain 2”, respectively. The N domain refers to the N-terminal domain. The “hinge” region is shown between the S-domain and the first P1-subdomain. The VLP comprising VP1 dimers is also depicted.

FIG. 3 is a western blot image depicting VP1 expression in cells. GI.1 is ~58.8 kDa; GII.4 is ~59.2 kDa and MNV1 is ~58.7 kDa.

FIGS. 4A and 4B are western blot images depicting VP1 expression in supernatant of Expi293 cells transfected with VP1 mRNAs. FIG. 4A depicts GI.1 VP1 mRNA and GII.4 VP1 mRNA collected on each of days 1-7. FIG. 4B depicts a mock transfection and MNV1 VP1 mRNA collected on each of days 1-7.

FIGS. 5A-5B are series of Electron Microscopy images depicting VLP formation in purified Expi293 cell lysate following transfection with GI.1 VP1 mRNA (FIG. 5A) and GI.3 VP1 mRNA (FIG. 5B).

FIG. 6 is a series of Electron Microscopy images of GI.1, GII.4 and GII.17 norovirus VLPs produced in insect cells.

FIGS. 7A-7B are graphs depicting titers of VP1-specific IgG antibodies elicited by GI.1 VP1 and GII.4 VP1 mRNA. FIG. 7A depicts GI.1 VLP serum IgG ELISA data from a primary endpoint on day 21 “PD1” and a secondary endpoint on day 36 “PD2” in Balb/c mice that received one of the following materials: PBS; 1 μg or 10 μg of GI.1 mRNA VP1 vaccine; 1 μg or 10 μg of GII.4 mRNA VP1 vaccine; 2 μg total or 20 μg total (1 μg of GI.1 mRNA VP1 vaccine and 1 μg of GII.4 mRNA VP1 vaccine or 10 μg of GI.1 mRNA VP1 vaccine and 10 μg of GII.4 mRNA VP1 vaccine); and 10 μg of GI.1 VP1 recombinant protein. Data is shown as reciprocal binding titers on a GI.I VLP ELISA. Recombinant proteins were co-administrated with an adjuvant (polyI:C). FIG. 7B is a graph depicting GII.4 VLP serum IgG ELISA data from a primary endpoint on day 21 “PD1” and a secondary endpoint on day 36 “PD2” in Balb/c mice that received one of the following materials: PBS; 1 μg or 10 μg of GI.1 mRNA VP1 vaccine; 1 μg or 10 μg of GII.4 mRNA VP1 vaccine; 2 μg or 20 μg total (1 μg of GI.1 mRNA VP1 vaccine and 1 μg of GII.4 mRNA VP1 vaccine or 10 μg of GI.1 mRNA VP1 vaccine and 10 μg of GII.4 mRNA VP1 vaccine); and 10 μg of GII.4 VP1 recombinant protein. Data is shown as reciprocal binding titers on a GI.I VLP ELISA. Recombinant proteins were co-administrated with an adjuvant (polyI:C).

FIGS. 8A-8B are graphs depicting titers of VP1-specific IgA detected in mice vaccinated with GI.1 VP1 and GII.4 VP1 mRNA. FIG. 8A depicts GI.1 VLP serum IgA ELISA data from fecal samples in Balb/c mice that received one of the following materials: PBS; 1 μg or 10 μg of GI.1 mRNA VP1 vaccine; 1 μg or 10 μg of GII.4 mRNA VP1 vaccine; 2 μg total or 20 μg total (1 μg of GI.1 mRNA VP1 vaccine and 1 μg of GII.4 mRNA VP1 vaccine or 10 μg of GI.1 mRNA VP1 vaccine and 10 μg of GII.4 mRNA VP1 vaccine); and 10 μg of GI.1 VP1 recombinant protein. Data is shown as reciprocal binding titers on a GI.I VLP ELISA. Recombinant proteins were co-administrated with an adjuvant (polyI:C). FIG. 8B depicts GII.4 VLP serum IgA ELISA data from a fecal samples in Balb/c mice that received one of the following materials: PBS; 1 μg or 10 μg of GI.1 mRNA VP1 vaccine; 1 μg or 10 μg of GII.4 mRNA VP1 vaccine; 2 μg total or 20 μg total (1 μg of GI.1 mRNA VP1 vaccine and 1 μg of GII.4 mRNA VP1 vaccine or 10 μg of GI.1 mRNA VP1 vaccine and 10 μg of GII.4 mRNA VP1 vaccine); and 10 μg of GII.4 VP1 recombinant protein. Data is shown as reciprocal binding titers on a GII.4 VLP ELISA. Recombinant proteins were co-administrated with an adjuvant (polyI:C).

FIGS. 9A-9B are graphs depicting VLP blockade antibodies in mice vaccinated with GI.1 VP1 and GII.4 VP1 mRNA. FIG. 9A depicts VLP blockade data in serum collected on day 36 post vaccination from Balb/c mice that received one of the following materials: PBS; 1 μg or 10 μg of GI.1 mRNA VP1 vaccine; 1 μg or 10 μg of GII.4 mRNA VP1 vaccine; 2 μg total or 20 μg total (1 μg of GI.1 mRNA VP1 vaccine and 1 μg of GII.4 mRNA VP1 vaccine or 10 μg of GI.1 mRNA VP1 vaccine and 10 μg of GII.4 mRNA VP1 vaccine); and 10 μg of GI.1 VP1 recombinant protein. Data is shown as the reciprocal titer that blocks 50% of binding to PGM III (BT50). Recombinant proteins were co-administrated with an adjuvant (polyI:C). FIG. 9B depicts VLP blockade data in serum collected on day 36 post vaccination from Balb/c mice that received one of the following materials: PBS; 1 μg or 10 μg of GI.1 mRNA VP1 vaccine; 1 μg or 10 μg of GII.4 mRNA VP1 vaccine; 2 μg or 20 μg total (1 μg of GI.1 mRNA VP1 vaccine and 1 μg of GII.4 mRNA VP1 vaccine or 10 μg of GI.1 mRNA VP1 vaccine and 10 μg of GII.4 mRNA VP1 vaccine); and 10 μg of GII.4 VP1 recombinant protein. Data is shown as the reciprocal titer that blocks 50% of binding to PGM III (BT50). Recombinant proteins were co-administrated with an adjuvant (polyI:C).

FIG. 10 is a graph depicting the binding of human sera from 28 subjects to a Norovirus VLP ELISA. Circle data points depict GII.4 binding, square data points depict GI.1 data points.

FIGS. 11A-11E detail a mouse study evaluating Norovirus VP1 mRNA with HRV 3CD protease. FIG. 11A depicts the study design for nine different test groups: Group 1 (n=6 mice) PBS; Group 2 (n=6 mice) GI.1 DS1 (“DS1” refers to disulfide stabilization); Group 3 (n=6 mice) GI.1 DS1+HRV 3CD protease; Group 4 (n=6 mice) GII.4 2018A; Group 5 (n=6 mice) GII.4 2018A+HRV 3CD protease; Group 6 (n=6 mice) GI.1 DS1+GII.4 2018A; Group 7 (n=8 mice) GI.1 DS1+GII.4 2018A+HRV 3CD (ratio of 1:1:1); Group 8 (n=8 mice) GI.1 DS1+GII.4 2018A+HRV 3CD (ratio of 1:1:0.2); and Group 9 (n=6 mice) HRV 3CD. FIGS. 11B-11C are graphs depicting GI.1 VLP serum IgG ELISA data (FIG. 11B) and GII.4 VLP serum IgG ELISA data (FIG. 11C) from a primary endpoint on day 21 and day 36 of Balb/c mice that received vaccines with various norovirus VP1 mRNAs and HRV3CD protease. Data is shown as reciprocal binding titers on a GI.1 VLP ELISA and GII.4 VLP ELISA. FIGS. 11D-11E are graphs depicting blockade titers following the secondary endpoint for GI.1 DS1 BT50 (FIG. 11D) and GII.4 2012 BT50 (FIG. 11E).

FIGS. 12A-12C detail a mouse study evaluating novel Norovirus genotypes and multivalent GI and GII combination mRNA vaccines. FIG. 12A depicts the study design for fourteen different test groups: Group 1 (n=6 mice) PBS; Group 2 (n=8 mice) GI.3; Group 3 (n=8 mice) GII.2; Group 4 (n=8 mice) GII.6; Group 5 (n=8 mice) GII.10; Group 6 (n=8 mice) GII.17; Group 7 (n=8 mice) GI.1 DS1+GI.3; Group 8 (n=8 mice) GII.2+GII.4 2018A+GII.6+GII.10+GII.17; Group 9 (n=8 mice) GI.1 DS1+GI.3+GII.2+GII.4 2018A+GII.6+GII.10+GII.17; Group 10 (n=8 mice) GI.1 DS1+GI.3 (LD); Group 11 (n=8 mice) GII.2+GII.4 2018A+GII.6+GII.10+GII.17 (LD); Group 12 (n=8 mice) GI.1 DS1+GI.3+GII.2+GII.4 2018A+GII.6+GII.10+GII.17 (LD); Group 13 (n=6 mice) GI.1 DS1+GII.4 2018A (ratio 1:3); and Group 14 (n=6 mice) GI.1 DS1+GI.3+GII.4 2018A+GII.17 (ratio 1:1:1:1). FIG. 12B is a graph depicting GI.1 VLP, GII.4 VLP, and GII.17 VLP IgG ELISAs conducted after the primary and secondary endpoints. Data is shown as reciprocal binding titers on a GI.1 VLP ELISA, GII.4 VLP ELISA, and GII.17 VLP ELISA. FIG. 12C is a graph depicting GI.1 VLP, GII.4, and GII.17 VLP blockade assays conducted after the secondary endpoint.

FIGS. 13A-13H detailing a mouse study evaluating different ratios for multivalent Norovirus GI and GII combination mRNA vaccines. FIG. 13A depicts the study design for thirteen different test groups: Group 1 (n=6 mice) GI.1 DS1; Group 2 (n=6 mice) GII.4 2012; Group 3 (n=6 mice) GII.17; Group 4 (n=6 mice) GI.1 DS1+GII.4 2012 (ratio 1:3); Group 5 (n=6 mice) GI.1 DS1+GII.4 2012+GII.17 (ratio 1:1:1) (HD); Group 6 (n=6 mice) GI.1 DS1+GII.4 2012+GII.17 (ratio 1:3:1) (HD); Group 7 (n=6 mice) GI.1 DS1+GII.4 2012+GII.17 (ratio 2:3:1) (HD); Group 8 (n=6 mice) GI.1 DS1+GII.4 2012+GII.17 (ratio 1:1:1) (LD); Group 9 (n=6 mice) GI.1 DS1+GII.4 2012+GII.17 (ratio 1:3:1) (LD); Group 10 (n=6 mice) GI.1 DS1+GII.4 2012+GII.17 (ratio 2:3:1) (LD); Group 11 (n=6 mice) GI.1 DS1+GII.4 2012+GII.17+GI.3; Group 12 (n=6 mice) GI.1 DS1+GII.4 2012+GII.17+GII.2; Group 13 (n=6 mice) GI.1 DS1+GII.4 2012+GII.17+GII.6. FIG. 13B includes graphs depicting GI.1 VLP, GII.4 VLP, and GII.17 VLP IgG ELISAs conducted after the primary endpoint. FIG. 13C includes graphs depicting cross-genotype IgG binding ELISAs (genotypes GI.3, GII.2, and GII.6). FIG. 13D includes graphs depicting reciprocal binding titers on a GII.4 specific Fecal IgA and IgG assay. FIG. 13E includes graphs depicting blockade titers in GI.1 DS1 and GI.3 in sera from mice vaccinated with the respective mRNAs. FIG. 13F includes graphs depicting blockade titers against GII.2 and GII.4 in sera from mice vaccinated with the respective mRNAs. FIG. 13G is a graph depicting GII.2 blockade in sera from mice vaccinated with the respective mRNAs. FIG. 13H includes graphs depicting blockade titers of against GII.6 and GII.17 in sera from mice vaccinated with the respective mRNAs.

FIG. 14 details a mouse study evaluating the composition and ratio of a Norovirus VP1 based mRNA vaccine with 21 different test groups: Group 1 (n=4 mice) PBS; Group 2 (n=5 mice) GI.3; Group 3 (n=5 mice) GII.2; Group 4 (n=5 mice) GII.3; Group 5 (n=5 mice) GII.4 (HD); Group 6 (n=5 mice) GII.4 (LD); Group 7 (n=5 mice) GII.6; Group 8 (n=5 mice) GII.12; Group 9 (n=6 mice) GII.17; Group 10 (n=6 mice) GII.3+GII.4 (HD); Group 11 (n=6 mice) GII.2+GII.3+GII.4 (HD); Group 12 (n=6 mice) GII.2+GII.3+GII.4+GII.6 (HD); Group 13 (n=6 mice) GII.2+GII.3+GII.4+GII.6+GII.17 (HD); Group 14 (n=6 mice) GI.3+GII.2+GII.3+GII.4+GII.6 (HD); Group 15 (n=6 mice) GI.3+GII.2+GII.3+GII.4+GII.6+GII.17 (HD); Group 16 (n=6 mice) GII.3+GII.4 (LD); Group 17 (n=6 mice) GII.2+GII.3+GII.4 (LD); Group 18 (n=6 mice) GII.2+GII.3+GII.4+GII.6 (LD); Group 19 (n=6 mice) GII.2+GII.3+GII.4+GII.6+GII.17 (LD); Group 20 (n=6 mice) GI.3+GII.2+GII.3+GII.4+GII.6 (LD); and Group 21 (n=6 mice) GI.3+GII.2+GII.3+GII.4+GII.6+GII.17 (LD).

FIGS. 15A-15K detail a mouse study evaluating the immunogenicity of Norovirus VP1 based mRNA vaccines. FIG. 15A depicts the study design with 8 different test groups: Group 1 (n=12 mice) PBS; Group 2 (n=12 mice) GI.3; Group 3 (n=12 mice) GII.2; Group 4 (n=12 mice) GII.3; Group 5 (n=12 mice) GII.4; Group 6 (n=12 mice) GII.6; Group 7 also referred to as “mRNA-1403” (n=12 mice) GI.3, GII.3, and GII.4; and Group 8 also referred to as “mRNA-1405” (n=12 mice) GI.3, GII.2, GII.3, GII.4, and GII.6. Matched and cross-genotype IgG binding ELISAs were conducted after the primary (Day 21, left graph bars) and secondary (Day 36, right graph bars) endpoints in the mouse study for the following genotypes: GI.3 (FIG. 15B), GII.3 (FIG. 15C), GII.4 (FIG. 15D), GII.2 (FIG. 15E), and GII.6 (FIG. 15F). Serum blockade titers were conducted after the secondary endpoint in the mouse study for the following genotypes: GI.3 (FIG. 15G, GII.3 (FIG. 15H), GII.4 (FIG. 15I), GII.2 (FIG. 15J), and GII.6 (FIG. 15K).

FIGS. 16A-16S detail a mouse study evaluating the immunogenicity of multivalent Norovirus VP1-based mRNA vaccines. FIG. 16A depicts the study design with 7 different test groups: Group 1 (n=12 mice) PBS; Group 2 (n=12 mice) mRNA-1403 (GI.3, GII.3, and GII.4) high dose; Group 3 (n=12 mice) mRNA-1403 (GI.3, GII.3, and GII.4) intermediate dose; Group 4 (n=12 mice) mRNA-1403 (GI.3, GII.3, and GII.4) low dose; Group 5 (n=12 mice) mRNA-1405 (GI.3, GII.2, GII.3, GII.4, and GII.6) high dose; Group 6 (n=12 mice) mRNA-1405 (GI.3, GII.2, GII.3, GII.4, and GII.6) intermediate dose; and Group 7 (n=12 mice) mRNA-1405 (GI.3, GII.2, GII.3, GII.4, and GII.6) low dose. Matched and cross-genotype IgG binding ELISAs were conducted after the primary (Day 21) and secondary (Day 36) endpoints in the mouse study for the following genotypes: GI.3 (FIG. 16B), GII.3 (FIG. 16C), GII.4 (FIG. 16D), GII.2 (FIG. 16E), and GII.6 (FIG. 16F). FIG. 16G is a table detailing the concentration (μg) of each mRNA in mRNA-1403 and mRNA-1405 in the high dose, intermediate dose, and low dose groups. Serum blockade titers were conducted after the secondary endpoint in the mouse study for the following genotypes: GI.3 (FIG. 16H), GII.3 (FIG. 16I), GII.4 (FIG. 16J), GII.2 (FIG. 16K), and GII.6 (FIG. 16L). FIG. 16M is a table detailing the concentration (μg) of each mRNA in mRNA-1403 and mRNA-1405 in the high dose, intermediate dose, and low dose groups. Serum IgA ELISA data from fecal samples of study mice were conducted for the following genotypes: GI.3 (FIG. 16N), GII.3 (FIG. 16O), GII.4 (FIG. 16P), GII.2 (FIG. 16Q), and GII.6 (FIG. 16R). FIG. 16S is a table detailing the concentration (μg) of each mRNA in mRNA-1403 and mRNA-1405 in the high dose, intermediate dose, and low dose groups.

FIGS. 17A-17K detail a rat study evaluating the immunogenicity of multivalent Norovirus VP1-based mRNA vaccines. FIG. 17A depicts the study design with 7 different test groups: Group 1 (n=10 rats, 5 female and 5 male) PBS; Group 2 (n=10 rats, 5 female and 5 male) mRNA-1403 (GI.3, GII.3, and GII.4) low dose; Group 3 (n=10 rats, 5 female and 5 male) mRNA-1403 (GI.3, GII.3, and GII.4) intermediate dose; Group 4 (n=10 rats, 5 female and 5 male) mRNA-1403 (GI.3, GII.3, and GII.4) high dose; Group 5 (n=10 rats, 5 female and 5 male) mRNA-1405 (GI.3, GII.2, GII.3, GII.4, and GII.6) low dose; Group 6 (n=10 rats, 5 female and 5 male) mRNA-1405 (GI.3, GII.2, GII.3, GII.4, and GII.6) intermediate dose; and Group 7 (n=10 rats, 5 female and 5 male) mRNA-1405 (GI.3, GII.2, GII.3, GII.4, and GII.6) high dose. Matched and cross-genotype IgG binding ELISAs were conducted after the primary (Day 21, left graph bars) and secondary (Day 36, right graph bars) endpoints in the rat study for the following genotypes: GI.3 (FIG. 17B), GII.4 (FIG. 17C), GII.3 (FIG. 17D), GII.2 (FIG. 17E), GII.6 (FIG. 17F). Matched and cross-genotype IgG binding ELISAs comparing male and female rats for the following genotypes: GI.3 (FIG. 17G), GII.3 (FIG. 17H), GII.4 (FIG. 17I), GII.2 (FIG. 17J), and GII.6 (FIG. 17K).

### DETAILED DESCRIPTION

Virus-like particles (VLPs) are spherical particles that closely resemble live viruses in structural characteristics and antigenicity. However, VLPs are distinguished from live viruses in that VLPs do not comprise any viral genetic material and are therefore non-infective. Due to their antigenic, yet non-infective nature, there is an increased interest in exploring the application of VLPs in vaccinations.

Currently, the majority of VLPs are generated using recombinant or cloning strategies. A VLP may be a self-assembled particle. Non-limiting examples of self-assembled VLPs and methods of making the self-assembled VLPs are described in International Patent Publication No. WO2013122262, the contents of which are herein incorporated by reference in its entirety.

VLPs are formed from the assembly of structural viral proteins (e.g., envelope and/or capsid proteins). The size and morphology of a VLP depends, at least in part, on the particular structural viral proteins that are incorporated into the particle upon assembly. A VLP assembled from the structural viral proteins of an enveloped virus may comprise, for example, one or more envelope proteins and one or more capsid proteins. A VLP assembled from the structural viral proteins of a non-enveloped virus may comprise, for example, one or more capsid proteins.

Norovirus is a nonenveloped virus comprising one major capsid protein (VP1) and one minor capsid protein (VP2), with VP1 expression alone being responsible for VLP formation. The minor capsid protein VP2 may aid in stabilization of the viral particle, but it is not required for VLP formation. Although attempts have been made to produce VLPs that mimic viruses from the Norovirus genus, including expressing recombinant VLPs in insect cells against two different norovirus genotypes, GI.1 and GII.4c, the resulting recombinant norovirus VLPs had limited efficacy with respect to inducing an effective T cell response. In addition, the production and purification process of recombinant VLPs is cumbersome, resulting in a combination of intact VLPs, broken particles and VP1 dimers. Vaccination with a heterogeneous population of VP1 antigen would induce suboptimal neutralizing antibody responses. Limitations in previous attempts to produce norovirus VLPs may be the result of an inability of recombinant VLPs to effectively mimic the morphology of a native norovirus VLP.

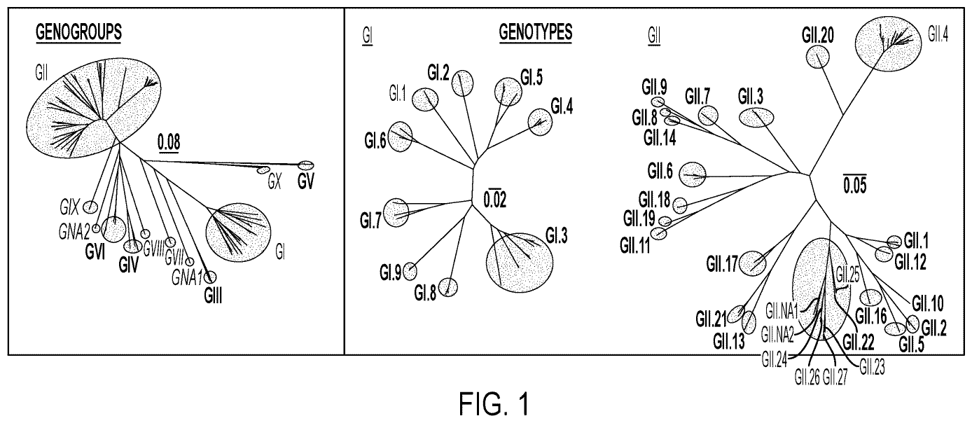

Quite surprisingly, the inventors have discovered, according to aspects of the invention, that RNA encoding a norovirus major capsid protein (VP1) can be delivered such that the VP1 is capable of first forming a VP1-VP1 homodimer and subsequently capable of forming a multimer VLP. For instance, in some cases a complex structure such as a VLP may be assembled properly from one or more VP1 proteins, which are expressed in a cell from messenger ribonucleic acid (mRNA) that is delivered in lipid nanoparticles (LNPs). For instance, the inventors identified that compositions comprising a mRNA comprising an open reading frame (ORF) encoding a norovirus major capsid protein (VP1) is sufficient for the formation of a virus-like particle. In some aspects, the inventors identified that compositions comprising a first mRNA comprising an ORF encoding a norovirus VP1 and further comprising a second mRNA comprising an ORF encoding a second norovirus VP1 is sufficient for the formation of one or more virus-like-particles. In some aspects, the first norovirus VP1 and second norovirus VP1 are from the same norovirus genogroup (FIG. 1). In some aspects, the first norovirus VP1 and second norovirus VP1 are from different norovirus genogroups. In some aspects, the first norovirus VP1 and second norovirus VP1 are from the same norovirus genotypes (FIG. 1). In some aspects, the first norovirus VP1 and second norovirus VP1 are from different norovirus genotypes. In some aspects, the compositions further comprise one or more lipid nanoparticles.

The inventors have also discovered, according to aspects of the invention, that compositions comprising multivalent mRNA comprising open reading frames encoding at least one but not more than ten norovirus VP1 are sufficient for the formation of one or more virus-like-particles. In some aspects, a composition comprises at least three different mRNA comprising three different ORFs encoding three different norovirus VP1 proteins. In some aspects, a composition comprises at least four, at least five, at least six, and/or at least seven different mRNA comprising at least four, at least five, at least six, and/or at least seven different ORFs encoding at least four, at least five, at least six, and/or at least seven different norovirus VP1 proteins. In some aspects, the first, second, third, fourth, fifth, sixth, and/or seventh norovirus VP1 are from the same norovirus genogroup. In some aspects, the first, second, third, fourth, fifth, sixth, and/or seventh norovirus VP1 are from the different norovirus genotypes. In some aspects, the compositions further comprise one or more lipid nanoparticles.

The inventors have also discovered, according to aspects of the invention methods comprising administering an immunogenic composition comprising a mRNA comprising an ORF encoding a norovirus VP1 in an amount effective to induce in the subject an immune response against a viral infection from a member of the Norovirus genus.

Described herein are compositions comprising one or more polynucleotides encoding a norovirus major capsid protein (VP1). As such the present invention is directed, in part, to polynucleotides, specifically messenger ribonucleic acid (mRNA) comprising an open reading frame encoding one or more norovirus VP1 proteins and/or components thereof.

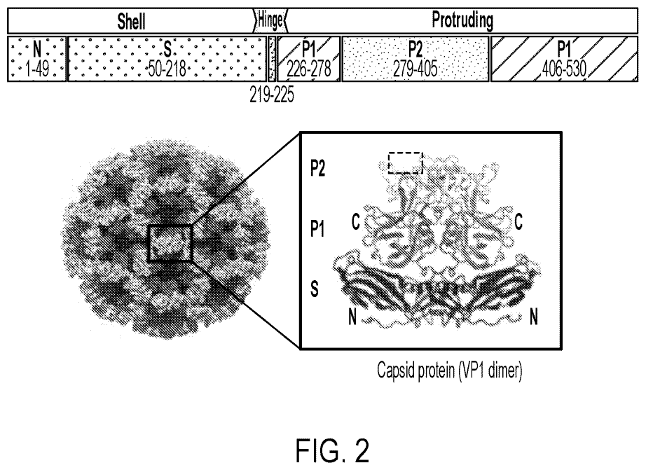

In some embodiments of the present invention, a norovirus major capsid protein (VP1) may be encoded by a single ribonucleic acid (RNA) molecule comprising a single open reading frame. In some aspects, the VP1 protein encoded by an RNA molecule comprises multiple domains. For example, a VP1 protein comprises a shell or S-domain and a protruding or P-domain (FIG. 2). The shell domain is a highly conserved region that makes up the core of the viral-particle and/or virus-like particle. In some aspects, the shell domain further comprises two domains, an N-domain and an S-domain. In some aspects, an RNA molecule may comprise a sequence coding for a shell domain that comprises both an N-domain and an S-domain. In some aspects, an RNA molecule may include a sequence coding for a shell domain that comprises an S-domain but does not comprise an N-domain. The protruding domain is a highly variable region that makes up the external region of the viral-particle and/or virus-like particle. In some aspects, the protruding domain further comprises two subdomains, a P1 subdomain and a P2 subdomain. In some aspects, an RNA molecule may comprise a sequence coding for a protruding domain that comprises a first P1 subdomain, a P2 subdomain, and a second P1 subdomain.

In some aspects, an RNA molecule may comprise a sequence coding for a shell domain and a protruding domain, wherein the shell domain is N-terminal to the protruding domain and wherein the shell domain is linked to the protruding domain by a hinge domain.

In some aspects, an RNA molecule may comprise one or more mutations in a sequence coding for a shell domain and a protruding domain, wherein the one or more mutations reduces the ability for the RNA molecule to be cleaved by a protease enzyme. In some aspects, the protease enzyme is 3CD (e.g., HRV 3CD). In some aspects, an RNA molecule comprising one or more mutations in a sequence coding for a shell domain and a protruding domain exhibits increased stability of the resulting viral-particle and/or virus-like particle.

Noroviruses are classified based on the VP1 amino acid sequence into ten different genogroups: GI, GII, GIII, GIV, GV, GVI, GVII, GVIII, GIX, and GX (FIG. 1). Noroviruses are further subclassified into approximately 49 different genotypes (FIG. 1). Examples of viruses which may be immunized against using the compositions or constructs of the present invention include, but are not limited to, members of the norovirus genogroups: GI, GII, GIII, GIV, GV, GVI, GVII, GVIII, GIX, and GX. Examples of viruses which may be immunized against using the compositions or constructs of the present invention include, but are not limited to, members of the 49 confirmed capsid (VP1) genotypes and the 60 confirmed P-types (RdRp regions) genotypes as described by Chhabra et al. J. Gen. Virol. 100.10 (2019): 1393, which is incorporated in its entirety by reference herein.

Noroviruses belonging to genogroups GI, GII, and GIV infect humans. Noroviruses belonging to genogroups GI and GII are the most predominant in humans and are of particular interest for purposes of vaccine development. For example, noroviruses belonging to genotype GII.4 are responsible for the majority of human infections. Some genotypes within genogroup GII infect other non-human mammals. For example, genotypes GII.11, GII.18 and GII.19 are detected in pigs and GIV.2 has been detected in cats and dogs.

In some embodiments, mRNAs of the present disclosure encode a norovirus major capsid protein (VP1) that is specific to norovirus genogroup GI. In some embodiments, the mRNA encoding a norovirus major capsid protein (VP1) is specific to one of the following genotypes: GI.1, GI.2, GI.3, GI.4, GI.5, GI.6, GI.7, GI.8, or GI.9.

In some embodiments, the mRNA encoding a norovirus major capsid protein (VP1) is specific to genotype GI.1. In some embodiments, the mRNA encoding a norovirus major capsid protein (VP1) is GI.1 (Norwalk) VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 5. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 6.

In some embodiments, the mRNA encoding a norovirus major capsid protein (VP1) is GI.3 VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 23. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 24.

In some embodiments, mRNAs of the present disclosure encode a norovirus major capsid protein (VP1) that is specific to norovirus genogroup GII. In some embodiments, the mRNA encoding a norovirus major capsid protein (VP1) is specific to one of the following genotypes: GII.1, GII.2, GII.3, GII.4, GII.5, GII.6, GII.7, GII.8, GII.9, GII.10, GII.11, GII.12, GII.13, GII.14, GII.15, GII.16, GII.17, GII.18, GII.19 or GII.20.

In some embodiments, the mRNA encoding a norovirus major capsid protein (VP1) is specific to genotype GII.4. In some embodiments, the mRNA encoding a norovirus major capsid protein (VP1) is GII.4 (Sydney) VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 2. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 3.

In some embodiments, the mRNA encoding a norovirus major capsid protein (VP1) is specific to genotype GII.2. In some embodiments, the mRNA encoding a norovirus major capsid protein (VP1) is GII.2 VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 17. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 18.

In some embodiments, the mRNA encoding a norovirus major capsid protein (VP1) is specific to genotype GII.3. In some embodiments, the mRNA encoding a norovirus major capsid protein (VP1) is GII.3 VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 63. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 64.

In some embodiments, the mRNA encoding a norovirus major capsid protein (VP1) is specific to genotype GII.6. In some embodiments, the mRNA encoding a norovirus major capsid protein (VP1) is GII.6 VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 15. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 16.

In some embodiments, the mRNA encoding a norovirus major capsid protein (VP1) is specific to genotype GII.17. In some embodiments, the mRNA encoding a norovirus major capsid protein (VP1) is GII.17 (Guangzhou) VP1. In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 19.

In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 20.

In some embodiments, the mRNA encoding a norovirus major capsid protein (VP1) comprises a nucleotide sequence sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 2, 5, 7, 9, 11, 13, 15, 17, 19, 21, 23, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, or 101. In some embodiments, the nucleotide sequence encodes an amino acid sequence of a norovirus major capsid protein (VP1) sharing at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 3, 6, 8, 10, 12, 14, 16, 18, 20, 22, 24, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, or 102.

In some embodiments, a norovirus major capsid protein (VP1) encoded by a polynucleotide forms a homodimer with another norovirus major capsid protein (VP1). In some embodiments, the homodimers form a VLP. For a non-limiting example, a norovirus major capsid protein (VP1) encoded by a polynucleotide specific to genotype GI.1 may homodimerize with another norovirus major capsid protein (VP1) encoded by a polynucleotide specific to genotype GI.1. In some embodiments, the GI.1-GI.1 homodimer may assemble with other GI.1-GI.1 homodimers and form a VLP.

For another non-limiting example, a norovirus major capsid protein (VP1) encoded by a polynucleotide specific to genotype GII.4 may homodimerize with another norovirus major capsid protein (VP1) encoded by a polynucleotide specific to genotype GII.4. In some embodiments, the GII.4-GII.4 homodimer may assemble with other GII.4-GII.4 homodimers and form a VLP.

For another non-limiting example, a norovirus major capsid protein (VP1) encoded by a polynucleotide specific to genotype GII.17 may homodimerize with another norovirus major capsid protein (VP1) encoded by a polynucleotide specific to genotype GII.17. In some embodiments, the GII.17-GII.17 homodimer may assemble with other GII.17-GII.17 homodimers and form a VLP.

Other non-limiting examples include a GI.2-GI.2 homodimer, a GI.3-GI.3 homodimer, a GI.4-GI.4 homodimer, a GI.5-GI.5 homodimer, a GI.6-GI.6 homodimer, a GI.7-GI.7 homodimer, a GI.8-GI.8 homodimer, a GI.9-GI.9 homodimer, a GII.1-GII.1 homodimer, a GII.2-GII.2 homodimer, a GII.3-GII.3 homodimer, a GII.5-GII.5 homodimer, a GII.6-GII.6 homodimer, a GII.7-GII.7 homodimer, a GII.8-GII.8 homodimer, a GII.9-GII.9 homodimer, a GII.10-GII.10 homodimer, a GII.11-GII.11 homodimer, a GII.12-GII.12 homodimer, a GII.13-GII.13 homodimer, a GII.14-GII.14 homodimer, a GII.15-GII.15 homodimer, a GII.16-GII.16 homodimer, a GII.18-GII.18 homodimer, a GII.19-GII.19 homodimer, and a GII.20-GII.20 homodimer.

Blocking antibodies can be produced against surface-exposed regions of viral particles. For example, in human norovirus, the viral protein VP1 is the most surface-exposed of the viral proteins and is therefore the most immunogenic viral protein. The VP1 protruding domain (P-domain) further comprises a histo-blood group antigen (HBGA) binding pocket. Therefore, HBGA-blocking antibodies are directed against the P-domain.

In some embodiments, compositions described herein comprise at least one lipid nanoparticle (e.g., the mRNAs and at least one lipid nanoparticle). In some embodiments, the compositions described herein comprise two lipid nanoparticles (e.g., the mRNAs and at least two lipid nanoparticles). In some embodiments, the compositions described herein comprise three lipid nanoparticles (e.g., the mRNAs and at least three lipid nanoparticles). In some embodiments, the compositions described herein comprise four lipid nanoparticles (e.g., the mRNAs and at least four lipid nanoparticles). In some embodiments, the compositions described herein comprise five lipid nanoparticles (e.g., the mRNAs and at least five lipid nanoparticles). In some embodiments, the compositions described herein comprise six lipid nanoparticles (e.g., the mRNAs and at least six lipid nanoparticles). In some embodiments, the compositions described herein comprise seven lipid nanoparticles (e.g., the mRNAs and at least seven lipid nanoparticles).

The polynucleotides and/or compositions of the present invention are useful in assembling VLPs that mimic virus or a viral particle and trigger an immunogenic response when administered to a subject.

The compositions of the present disclosure may be designed as a single prophylactic therapeutic that immunizes a subject against a variety of pathogenic strains of norovirus.

In some aspects, a method of the present disclosure comprises administering to a subject an immunogenic composition described herein. As used herein, an “immunogenic composition” refers to a composition comprising an mRNA comprising an open reading frame encoding a first norovirus VP1 and an mRNA comprising an open reading frame encoding a second norovirus VP1 in an amount effective to induce in the subject an immune response against a viral infection from a member of the Norovirus genus.

In some embodiments, an immunogenic composition comprises an mRNA comprising an open reading frame encoding a first norovirus VP1, an mRNA comprising an open reading frame encoding a second norovirus VP1, and an mRNA comprising an open reading frame encoding a third norovirus VP1 in an amount effective to induce in the subject an immune response against a viral infection from a member of the Norovirus genus.

In some embodiments, an immunogenic composition comprises an mRNA comprising an open reading frame encoding a first norovirus VP1, an mRNA comprising an open reading frame encoding a second norovirus VP1, an mRNA comprising an open reading frame encoding a third norovirus VP1, an mRNA comprising an open reading frame encoding a fourth norovirus VP1, and an mRNA comprising an open reading frame encoding a fifth norovirus VP1 in an amount effective to induce in the subject an immune response against a viral infection from a member of the Norovirus genus.

In some embodiments, an immunogenic composition comprises an mRNA comprising an open reading frame encoding a first norovirus VP1, an mRNA comprising an open reading frame encoding a second norovirus VP1, an mRNA comprising an open reading frame encoding a third norovirus VP1, an mRNA comprising an open reading frame encoding a fourth norovirus VP1, an mRNA comprising an open reading frame encoding a fifth norovirus VP1, an mRNA comprising an open reading frame encoding a sixth norovirus VP1, and an mRNA comprising an open reading frame encoding a seventh norovirus VP1 in an amount effective to induce in the subject an immune response against a viral infection from a member of the Norovirus genus.

In some aspects, immunogenic compositions of the present disclosure further comprise at least one lipid nanoparticles. In some embodiments, the immunogenic compositions of the present disclosure comprise at least two, at least three, at least four, at least five, at least six, or at least seven lipid nanoparticles.

In some aspects, immunogenic compositions may induce an immune response (e.g., may produce prophylactically- and/or therapeutically-efficacious levels, concentrations and/or titers of antigen-specific antibodies in the blood or serum of a vaccinated subject).

In some embodiments, an immune response is determined by measuring binding antibody titer to a species of virus. In some embodiments, an immune response is determined by measuring a binding antibody titer to a human norovirus of specific genogroup (e.g., GI or GII). In some embodiments, an immune response is determined by measuring a binding antibody titer to a human norovirus of specific genotype (e.g., GI.1, GII.4, GII.17, GI.2, GI.3, GI.4, GI.5, GI.6, GI.7, GI.8, or GI.9, GII.1, GII.2, GII.3, GII.5, GII.6, GII.7, GII.8, GII.9, GII.10, GII.11, GII.12, GII.13, GII.14, GII.15, GII.16, GII.18, GII.19 or GII.20).

The term binding antibody titer refers to the amount of antigen-specific antibody produced in a subject. In some embodiments, the subject is a human subject. In exemplary embodiments, binding antibody titer is expressed as the inverse of the greatest dilution (in a serial dilution) that still gives a positive result. In exemplary embodiments, binding antibody titer is determined or measured by enzyme-linked immunosorbent assay (ELISA). In exemplary embodiments, binding antibody titer is determined or measured by neutralization assay, e.g., by microneutralization assay.

In exemplary aspects of the invention, antigen-specific antibodies are measured in units of μg/ml or are measured in units of IU/L (International Units per liter) or mIU/ml (milli International Units per ml).

In some embodiments, an immune response is determined by measuring a blocking antibody titer to a species of virus. In some embodiments, an immune response is determined by measuring a blocking antibody titer to a human norovirus of specific genogroup (e.g., GI or GII). In some embodiments, an immune response is determined by measuring a blocking antibody titer to a human norovirus of specific genotype (e.g., GI.1, GII.4, GII.17, GI.2, GI.3, GI.4, GI.5, GI.6, GI.7, GI.8, or GI.9, GII.1, GII.2, GII.3, GII.5, GII.6, GII.7, GII.8, GII.9, GII.10, GII.11, GII.12, GII.13, GII.14, GII.15, GII.16, GII.18, GII.19 or GII.20). In exemplary aspects of the invention, blocking antibody titer is determined or measured by blockade assay and/or neutralization assay.

In some embodiments, an immune response is determined by measuring a T cell response to a species of virus. In some embodiments, an immune response is determined by measuring a T cell response to a human norovirus of specific genogroup (e.g., GI or GII). In some embodiments, an immune response is determined by measuring a T cell response to a human norovirus of specific genotype (e.g., GI.1, GII.4 GII.17, GI.2, GI.3, GI.4, GI.5, GI.6, GI.7, GI.8, or GI.9, GII.1, GII.2, GII.3, GII.5, GII.6, GII.7, GII.8, GII.9, GII.10, GII.11, GII.12, GII.13, GII.14, GII.15, GII.16, GII.18, GII.19 or GII.20). In exemplary aspects of the invention, a T cell response is determined or measured using art recognized techniques.

According to the present invention, polynucleotides or constructs and their associated compositions may be designed to produce a commercially available vaccine, a variant or a portion thereof in vivo.

The polynucleotides of the invention may be used to protect an infant against infection or disease, including but not limited to diseases caused by noroviruses. Examples of diseases caused by noroviruses include, but are not limited to, epidemic and sporadic outbreaks of acute gastroenteritis.

In some embodiments, the polynucleotides of the invention may encode at least one norovirus major capsid protein (VP1) that forms a VLP when administered to a subject and immunizes the subsection for the prevention, management, or treatment of norovirus infections.

### Multivalent Ratios

In some embodiments, the present disclosure provides multivalent mRNA compositions. In some embodiments, the mRNAs encoding one or more norovirus major capsid protein (VP1) are present in the composition in an equal amount (e.g., a 1:1 ratio), for example, a 1:1 ratio of mRNAs encoding distinct norovirus VP1. A non-limiting example includes a 1:1 ratio of a GI.1 norovirus VP1 to GII.4 norovirus VP1.

In an exemplary vaccine comprising mRNAs encoding three different norovirus VP1, mRNAs at a “1:1 ratio” would include the mRNAs in a ratio of 1:1:1 of the first, second, and third mRNA. In an exemplary vaccine comprising mRNAs encoding four different norovirus VP1, mRNAs at a “1:1 ratio” would include the mRNAs in a ratio of 1:1:1:1 of the first, second, third and fourth mRNA. In an exemplary vaccine comprising mRNAs encoding five different norovirus VP1, mRNAs at a “1:1 ratio” would include the mRNAs in a ratio of 1:1:1:1:1 of the first, second, third, fourth, and fifth mRNA. In an exemplary vaccine comprising mRNAs encoding six different norovirus VP1, mRNAs at a “1:1 ratio” would include the mRNAs in a ratio of 1:1:1:1:1:1 of the first, second, third, fourth, fifth, and sixth mRNA. In an exemplary vaccine comprising mRNAs encoding seven different norovirus VP1, mRNAs at a “1:1 ratio” would include the mRNAs in a ratio of 1:1:1:1:1:1:1 of the first, second, third, fourth, fifth, sixth and seventh mRNA.

In some embodiments, the mRNAs encoding one or more norovirus major capsid protein (VP1) are present in the composition in different amounts (e.g., a 1:3 ratio). A non-limiting example includes a 1:3 ratio of a GI.1 norovirus VP1 to GII.4 norovirus VP1. A second non-limiting example includes a 1:3 ratio of GII.4 norovirus VP1 to GI.1 norovirus VP1.

In an exemplary vaccine comprising mRNAs encoding three different norovirus VP1, mRNAs at a “1:3 ratio” would include the mRNAs in a ratio of 1:3:1 of the first, second, and third mRNA. In some embodiments, a vaccine comprises mRNAs encoding GI.3, GII.3 and GII.4 in a ratio of about 1:1:3, respectively. In an exemplary vaccine comprising mRNAs encoding four different norovirus VP1, mRNAs at a “1:3 ratio” would include the mRNAs in a ratio of 1:3:1:1 of the first, second, third and fourth mRNA. In an exemplary vaccine comprising mRNAs encoding five different norovirus VP1, mRNAs at a “1:3 ratio” would include the mRNAs in a ratio of 1:3:1:1:1 of the first, second, third, fourth, and fifth mRNA. In some embodiments, a vaccine comprises mRNAs encoding GI.3, GII.3, GII.4, GII.2, and GII.6 in a ratio of about 1:1:3:1:1, respectively. In an exemplary vaccine comprising mRNAs encoding six different norovirus VP1, mRNAs at a “1:3 ratio” would include the mRNAs in a ratio of 1:3:1:1:1:1 of the first, second, third, fourth, fifth, and sixth mRNA. In an exemplary vaccine comprising mRNAs encoding seven different norovirus VP1, mRNAs at a “1:3 ratio” would include the mRNAs in a ratio of 1:3:1:1:1:1:1 of the first, second, third, fourth, fifth, sixth and seventh mRNA.

In some embodiments, the mRNAs encoding one or more norovirus major capsid protein (VP1) are present in the composition in different amounts (e.g., a 2:3 ratio). A non-limiting example includes a 2:3 ratio of a GI.1 norovirus VP1 to GII.4 norovirus VP1. A second non-limiting example includes a 2:3 ratio of GII.4 norovirus VP1 to GI.1 norovirus VP1.

In an exemplary vaccine comprising mRNAs encoding three different norovirus VP1, mRNAs at a “2:3 ratio” would include the mRNAs in a ratio of 2:3:1 of the first, second, and third mRNA. In an exemplary vaccine comprising mRNAs encoding four different norovirus VP1, mRNAs at a “2:3 ratio” would include the mRNAs in a ratio of 2:3:1:1 of the first, second, third and fourth mRNA. In an exemplary vaccine comprising mRNAs encoding five different norovirus VP1, mRNAs at a “2:3 ratio” would include the mRNAs in a ratio of 2:3:1:1:1 of the first, second, third, fourth, and fifth mRNA. In an exemplary vaccine comprising mRNAs encoding six different norovirus VP1, mRNAs at a “2:3 ratio” would include the mRNAs in a ratio of 2:3:1:1:1:1 of the first, second, third, fourth, fifth, and sixth mRNA. In an exemplary vaccine comprising mRNAs encoding seven different norovirus VP1, mRNAs at a “2:3 ratio” would include the mRNAs in a ratio of 2:3:1:1:1:1:1 of the first, second, third, fourth, fifth, sixth and seventh mRNA.

In some embodiments, the norovirus VP1 from genotype GI norovirus VP1 from genogroup GII ratio is 1:1, 1:2, 1:3, 1:4, 2:1, 2:3, 3:1, 4:1, 5:1, 6:1, or 7:1.

In some embodiments, the first, second, third, fourth, fifth, six, and/or seventh mRNA polynucleotides are present in the combination vaccine in a ratio of 1:1:1:1:1:1:1. In some embodiments, the combination vaccine comprises a ratio of mRNA polynucleotides encoding norovirus VP1 of 1:3:1 from the first genogroup (e.g., GI) to the second norovirus VP1 from the second genogroup (e.g., GII) to the third norovirus VP1 of the first or second genogroup (e.g., GI or GII).

In some embodiments, the combination vaccine comprises a ratio of mRNA polynucleotides encoding norovirus VP1 of 2:3:1 from the first genogroup (e.g., GI) to the second norovirus VP1 from the second genogroup (e.g., GII) to the third norovirus VP1 of the first or second genogroup (e.g., GI or GII).

In some embodiments, each of the mRNA polynucleotides in the combination vaccine is complementary with and does not interfere with each other mRNA polynucleotide in the combination vaccine. That is, VP1 of different genogroups and/or genotypes produced from administration of the combination vaccine do not significantly interfere with the immune response to any other of the VP1 produced in response to the vaccine in such a way that would diminish the ability of the VP1 to provoke a protective immune response in a subject. In some embodiments, the combination vaccine is additive with respect to neutralizing antibodies relative to each individual VP1 of different genogroups and/or genotypes in a vaccine. In some embodiments, the combination vaccine is synergistic with respect to neutralizing antibodies relative to each individual VP1 of different genogroups and/or genotypes in a vaccine.

In one embodiment, the polynucleotides of the present invention are or function as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo.

The basic components of an mRNA molecule typically include at least one coding region, a 5′ untranslated region (UTR), a 3′ UTR, a 5′ cap, and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA), and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 90% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 85% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 80% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares less than 75% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof). In some embodiments, a codon optimized sequence shares between 65% and 75 or about 80% sequence identity to a naturally-occurring or wild-type sequence (e.g., a naturally-occurring or wild-type mRNA sequence encoding a polypeptide or protein of interest, such as a picornavirus capsid polyprotein and/or picornavirus 3C protease thereof).

In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain or multichain polypeptides such as antibodies and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

In some embodiments “variant mimics” are provided. As used herein, the term “variant mimic” is one which contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic, for example, phenylalanine may act as an inactivating substitution for tyrosine; or alanine may act as an inactivating substitution for serine.

“Orthologs” refers to genes in different species that evolved from a common ancestral gene by speciation. Normally, orthologs retain the same function in the course of evolution. Identification of orthologs is critical for reliable prediction of gene function in newly sequenced genomes.

“Analogs” is meant to include polypeptide variants which differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini, or any combination thereof.

As used herein, when referring to polypeptides, the term “domain” refers to a motif of a polypeptide having one or more identifiable structural or functional characteristics or properties (e.g., binding capacity, serving as a site for protein-protein interactions).

As used herein, when referring to polypeptides, the terms “site” as it pertains to amino acid based embodiments is used synonymously with “amino acid residue” and “amino acid side chain.” As used herein, when referring to polynucleotides, the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules.

As used herein, the terms “termini” or “terminus” when referring to polypeptides or polynucleotides refers to an extremity of a polypeptide or polynucleotide, respectively. Such extremity is not limited only to the first or final site of the polypeptide or polynucleotide, but may include additional amino acids or nucleotides in the terminal regions. Polypeptide-based molecules may be characterized as having both an N-terminus (terminated by an amino acid with a free amino group (NH₂)) and a C-terminus (terminated by an amino acid with a free carboxyl group (COOH)). Proteins are in some cases made up of multiple polypeptide chains brought together by disulfide bonds or by non-covalent forces (e.g., multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% or 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g., DNA molecules and/or RNA molecules) and/or between polypeptide molecules. Polymeric molecules (e.g., nucleic acid molecules (e.g., DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

RNA (e.g., mRNA) treatments of the present disclosure may comprise at least one ribonucleic acid (RNA) polynucleotide having an open reading frame encoding a norovirus major capsid protein (VP1) that comprises at least one chemical modification. RNA (e.g., mRNA) treatments of the present disclosure may comprise at least one ribonucleic acid (RNA) polynucleotide having an open reading frame encoding a norovirus major capsid protein (VP1) that comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” if they contain amino acid substitutions, insertions, or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring, or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside comprising one or more phosphate groups. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine, or uracil. Any combination of base/sugar or linker may be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of polynucleotides (e.g., RNA polynucleotides, such as mRNA polynucleotides) that are useful in the compositions, methods and synthetic processes of the present disclosure of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; a-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′-Fluoro-N6-Bz-deoxyadenosine TP; 2′-OMe-2-Amino-ATP; 2′-O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; a-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; a-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine, N1-ethylpseudouridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; a-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy]propionic acid; Pseudouridine TP 1-[3-12-(2-[2-ethoxy]-ethoxy)-ethoxy]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine;1-(aza)-2-(thio)-3-(aza)-phenoxazin-1-yl: 1,3-(diaza)-2-(oxo)-phenthiazin-1-yl;1,3-(diaza)-2-(oxo)-phenoxazin-1-yl;1,3,5-(triaza)-2,6-(dioxa)-naphthalene;2 (amino)purine;2,4,5-(trimethyl)phenyl;2′ methyl, 2′amino, 2′azido, 2′fluro-cytidine;2′ methyl, 2′amino, 2′azido, 2′fluro-adenine;2′methyl, 2′amino, 2′azido, 2′fluro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m1ψ), 5-methoxy-uridine (mo5U), 5-methyl-cytidine (m5C), pseudouridine (ψ), α-thio-guanosine, and α-thio-adenosine. In some embodiments, polynucleotides include a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m5C). In some embodiments, polynucleotides (e.g., RNA polynucleotides, such as mRNA polynucleotides) comprise 1-methyl-pseudouridine (m1ψ). In some embodiments, polynucleotides (e.g., RNA polynucleotides, such as mRNA polynucleotides) comprise 1-methyl-pseudouridine (m1ψ) and 5-methyl-cytidine (m5C). In some embodiments, polynucleotides (e.g., RNA polynucleotides, such as mRNA polynucleotides) comprise 2-thiouridine (s2U). In some embodiments, polynucleotides (e.g., RNA polynucleotides, such as mRNA polynucleotides) comprise 2-thiouridine and 5-methyl-cytidine (m5C). In some embodiments, polynucleotides (e.g., RNA polynucleotides, such as mRNA polynucleotides) comprise methoxy-uridine (mo5U). In some embodiments, polynucleotides (e.g., RNA polynucleotides, such as mRNA polynucleotides) comprise 5-methoxy-uridine (mo5U) and 5-methyl-cytidine (m5C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m5C). In some embodiments, polynucleotides (e.g., RNA polynucleotides, such as mRNA polynucleotides) comprise N6-methyl-adenosine (m6A). In some embodiments, polynucleotides (e.g., RNA polynucleotides, such as mRNA polynucleotides) comprise N6-methyl-adenosine (m6A) and 5-methyl-cytidine (m5C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C, or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90%, or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90%, or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA treatments comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (rm5U), 1-taurinomethyl-pseudouridine, 5-taurinomethyl-2-thio-uridine(rm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, N1-ethylpseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

Antibodies and antigen binding fragments thereof of the present disclosure comprise at least one RNA polynucleotide, such as an mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

In other embodiments, the compositions of the present disclosure comprise, in some embodiments, an RNA wherein the nucleic acid comprises nucleotides and/or nucleosides that can be standard (unmodified) or modified as is known in the art, including, for example, any of the modifications disclosed herein. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a picornavirus capsid polyprotein and/or picornavirus 3C protease). In some embodiments, a codon optimized sequence shares less than 90% sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares less than 85% sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares less than 80% sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares less than 75% sequence identity to a naturally-occurring or wild-type sequence (e.g., a naturally-occurring or wild-type mRNA sequence encoding a picornavirus capsid polyprotein and/or picornavirus 3C protease).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a picornavirus capsid polyprotein and/or picornavirus 3C protease). In some embodiments, a codon optimized sequence shares between 65% and 75% or about 80% sequence identity to a naturally-occurring or wild-type sequence (e.g., a naturally-occurring or wild-type mRNA sequence encoding a picornavirus capsid polyprotein and/or picornavirus 3C protease).

In some embodiments, a codon-optimized sequence encodes a picornavirus capsid polyprotein and/or picornavirus 3C protease that is as immunogenic as, or more immunogenic than (e.g., at least 10%, at least 20%, at least 30%, at least 40%, at least 50%, at least 100%, or at least 200% more), than a picornavirus capsid polyprotein and/or picornavirus 3C protease encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 103), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 104) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-0) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises SEQ ID NO: 1.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

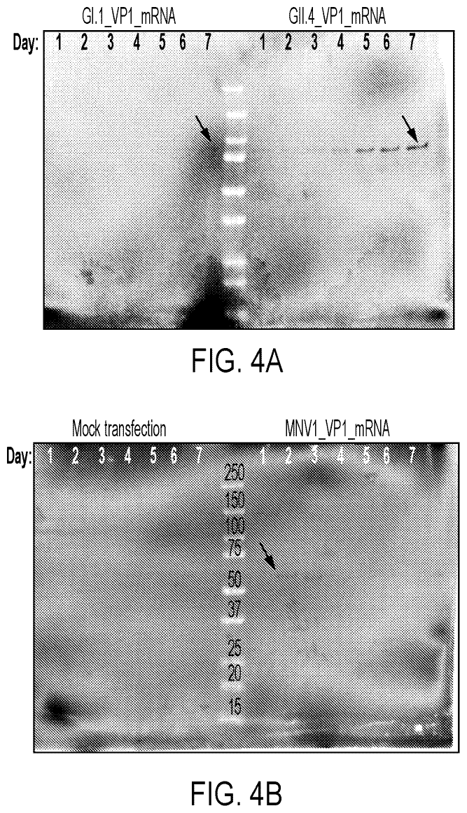

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit R globin and hepatitis B virus (HBV), a-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′UTR (WO2015101414), FIG. 4 (WO2015101415), and human albumin 7 (WO2015101415).

In some embodiments, a 3′ UTR of the present disclosure comprises SEQ ID NO: 4.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

Multivalent mRNA constructs are typically produced by transcribing one mRNA product at a time, purifying each mRNA product, and then mixing the purified mRNA products together prior to formulation. This type of process incurs significant time and monetary investment especially at the Good Manufacturing Practice (GMP) scale.

Aspects of the disclosure relate to methods for producing compositions comprising multivalent different RNAs (e.g., 2 or more different RNAs). In some aspects, methods of multivalent transcription disclosed herein involve selecting amounts of input DNA for IVT reactions that result in multivalent RNA compositions having higher purity than RNA compositions produced using previous methods. It was observed that certain characteristics or properties of DNA molecules being co-transcribed (e.g., transcribed simultaneously in vitro), such as differences in length between DNA molecules, polyA-tailing efficiency of DNA molecules, etc., and/or other reagents present in the co-IVT reaction mixture (e.g., RNA polymerase, nucleotide triphosphates (NTPs), etc.) may introduce compositional bias into the resulting multivalent RNA compositions. Surprisingly, methods were discovered that reduce such compositional bias. In some embodiments, modifying input DNA amounts results in production of multivalent RNA compositions having increased purity (e.g., as measured by percentage of RNAs comprising polyA tails) relative to RNA compositions produced by previous methods. It was also surprisingly discovered that co-IVT methods described herein result in high purity multivalent RNA compositions even when there is a large difference (e.g., >100 nucleotides) in the lengths of the input DNAs used in the IVT reaction.

Accordingly, in some aspects, the disclosure provides a method for producing a multivalent RNA composition, the method comprising simultaneously in vitro transcribing at least two DNA molecules in a reaction mixture comprising: a first population of DNA molecules encoding a first RNA; a second population of DNA molecules encoding a second RNA that is different than the first RNA; and obtaining a multivalent RNA composition having a pre-defined ratio of the first RNA to the second RNA produced by the IVT.

As used herein, the term “multivalent RNA composition” refers to a composition comprising more than two different mRNAs. A multivalent RNA composition may comprise 2 or more different RNAs, for example 2, 3, 4, 5, 6, 7, 8, 9, 10, or more different RNAs. In some embodiments, a multivalent RNA composition comprises more than 10 different RNAs. The term “different RNAs” refers to any RNA that is not the same as another RNA in a multivalent RNA composition. For example two RNAs are different if they have i) different lengths (whether or not the RNAs are identical over the entirety of the shorter of the two lengths), ii) different nucleotide sequences, iii) different chemical modification patterns, or iv) any combination of the foregoing.

In some embodiments, each input DNA (e.g., population of input DNA molecules) in a co-IVT reaction is obtained from a different source (e.g., synthesized separately, for example in different cells or populations of cells). In some embodiments, each input DNA (e.g., population of input DNA) is obtained from a different bacterial cell or population of bacterial cells. For example, in a co-IVT reaction having three populations of input DNAs, the first input DNA is produced in bacterial cell population A, the second input DNA is produced in bacterial cell population B, and the third input DNA is produced in bacterial population C, where each of A, B, and C are not the same bacterial culture (e.g., co-cultured in the same container or plate). Methods of obtaining populations of input DNAs (e.g., plasmid DNAs) are known, for example as described by Sambrook, Joseph. Molecular Cloning: a Laboratory Manual. Cold Spring Harbor, N.Y.: Cold Spring Harbor Laboratory Press, 2001.

Some aspects comprise normalizing the amount of DNA used in the multivalent co-IVT reaction. In some embodiments, the normalization is based on the molar mass of the input DNAs. In some embodiments, the normalization is based on the degradation rate of the input DNAs. In some embodiments, the normalization is based on the degradation rate of the resultant mRNAs (e.g., measured based upon polyA variants present in the reaction mixture, or T7 polymerase abortive transcripts or truncated transcripts). In some embodiments, the normalization is based on the nucleotide content (e.g., amount of A, G, C, U, or any combination thereof) of the input DNAs. In some embodiments, the normalization is based on the purity of the input DNAs. In some embodiments the normalization is based on the polyA-tailing efficiency of the input DNAs. In some embodiments, the normalization is based on the lengths of the input DNAs.

In some embodiments, mRNA is at a pre-defined mRNA ratio, which may comprise a ratio between 2, 3, 4, 5, 6, 7, 8, 9, 10, or more different RNAs (e.g., depending on the number of different RNAs in a composition). In some embodiments, a pre-defined ratio comprises a ratio between more than 10 RNAs. As used herein, a “pre-defined mRNA ratio” refers to the desired final ratio of RNA molecules in a multivalent RNA composition. The desired final ratio of an RNA composition will depend upon the final peptide(s) or polypeptide product(s) encoded by the RNAs. For example, a multivalent RNA mixture may comprise two RNAs (e.g., a RNA encoding a first antigen and a second antigen); in this instance the desired final ratio of RNA molecules may be 1 first antigen RNA:1 second antigen RNA. In another example, a multivalent RNA composition may comprise several (e.g., 3, 4, 5, 6, 7, 8, or more) RNAs encoding different antigenic peptides (e.g., for use as a vaccine); in that instance the desired ratio may comprise between 3 and 10 RNAs (e.g., a:b:c, a:b:c:d, a:b:c:d:e, a:b:c:d:e:f, a:b:c:d:e:f:g, a:b:c:d:e:f:g:h, a:b:c:d:e:f:g:h:i, a:b:c:d:e:f:g:h:i:j, etc., where each of a-j is a number between 1 and 10).

In some embodiments, the normalization is based on the lowest level present in the input DNAs (e.g., lowest molar mass, degradation rate (e.g., of the input DNA and/or output RNA), nucleotide content, purity, and/or polyA-tailing efficiency). In some embodiments, the normalization is based on the highest level present in the input DNAs (e.g., highest molar mass, degradation rate (e.g., of the input DNA and/or output RNA), nucleotide context, purity, and/or polyA-tailing efficiency). In some embodiments, the normalization is based on the rate of RNA production of the input DNAs (e.g., the highest rate of RNA production of an input DNA or the lowest rate of RNA production of an input DNA in a reaction mixture).

In some aspects, the disclosure relates to IVT methods in which the amount of input DNA (e.g., a first DNA or second DNA) is adjusted or normalized in order to improve production of multivalent RNA compositions having a pre-defined mRNA ratio of components.

As described herein, certain factors affecting multivalent RNA composition purity, such as large differences in size between input DNAs (e.g., a difference of more than 100, 200, 500, 1000, or more nucleotides in length) and/or polyA-tailing efficiency of a given DNA during IVT, may be addressed prior to the IVT by normalizing the amount of input DNA based upon one or more of those factors.

The number of input DNAs (e.g., populations of input DNA molecules) used in an IVT reaction may vary, depending upon the number of different RNA molecules desired to be included in the multivalent RNA composition. In some embodiments, an IVT reaction mixture comprises 2 or more different input DNAs, for example 2, 3, 4, 5, 6, 7, 8, 9, 10, 11, 12, 13, 14, 15, or more different input DNAs. In some embodiments, the IVT reaction comprises more than 15 different input DNAs. The term “different input DNAs” encompasses input DNAs that encode different RNAs, e.g., that have i) different lengths (whether or not the RNAs are identical over the entirety of the shorter of the two lengths), ii) different nucleotide sequences, iii) different chemical modification patterns, or iv) any combination of the foregoing.

In some embodiments, two or more of the input DNA molecules used in an IVT reaction encode mRNA molecules that have a different length (e.g., comprises a different number of nucleotides). In some embodiments, the difference in length between two or more of the mRNA molecules encoded by different input DNA molecules in an IVT reaction mixture is greater than 70 nucleotides, 80 nucleotides, 90 nucleotides, or 100 nucleotides (e.g., two input DNAs in a composition encode mRNA molecules that are not are within 70, 80, 90, or 100 nucleotides in length of one another). In some embodiments, the difference in length between two or more of the mRNA molecules encoded by different input DNA molecules is more than 100 nucleotides, for example 500 nucleotides, 1000 nucleotides, 1500 nucleotides, 2000 nucleotides, 3000 nucleotides, 4000 nucleotides, or more.

In specific embodiments, the combination vaccine (e.g., multivalent RNA composition) is produced by combining a linearized first DNA molecule encoding the first mRNA polynucleotide, a linearized second DNA molecule encoding the second mRNA polynucleotide, and a linearized third DNA molecule encoding the third mRNA polynucleotide into a single reaction vessel, wherein the first DNA molecule, the second DNA molecule, and the third DNA molecule are obtained from different sources. In some embodiments, the different sources are a first, second, and third bacterial cell culture and wherein the first, second and third bacterial cell culture are not co-cultured. In some embodiments, the different sources are a first, second, and third bacterial cell culture and wherein the first, second and third bacterial cell culture are co-cultured. In some embodiments, the amounts of the first, second and third DNA molecules present in the reaction mixture prior to the start of the in vitro transcription have been normalized.

In some embodiments, the linearized first DNA molecule, the linearized second DNA molecule and the linearized third DNA molecule are simultaneously in vitro transcribed to obtain the multivalent RNA composition.

### Non-Coding Sequences

Aspects of the disclosure relate to multivalent RNA compositions which comprise mRNAs, e.g., 2-15 mRNA polynucleotides each comprising a distinct open reading frame (ORF) encoding a norovirus major capsid protein (VP1), wherein each mRNA polynucleotide comprises one or more non-coding sequences in an untranslated region (UTR) having unique identifier sequences. As used herein, “non-coding sequence” refers to a sequence of a biological molecule (e.g., nucleic acid, protein, etc.) that when combined with the sequence another biological molecule serves to identify the other biological molecule. Typically, a non-coding sequence is a heterologous sequence that is incorporated within or appended to a sequence of a target biological molecule and utilized as a reference in order to identify a target molecule of interest. In some embodiments, a non-coding sequence is a sequence of a nucleic acid (e.g., a heterologous or synthetic nucleic acid) that is incorporated within or appended to a target nucleic acid and utilized as a reference in order to identify the target nucleic acid. In some embodiments, a non-coding sequence is of the formula (N)n. In some embodiments, n is an integer in the range of 5 to 20, 5 to 10, 10 to 20, 7 to 20, or 7 to 30. In some embodiments, n is 2, 3,4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, N are nucleotides that are each independently selected from A, G, T, U, and C, or analogues thereof. Thus, some embodiments comprise nucleic acids (e.g., mRNAs) that (i) have a target sequence of interest (e.g., a coding sequence (e.g., that encodes therapeutic peptide or therapeutic protein)); and (ii) comprises a unique non-coding sequence.

In some embodiments, one or more in vitro transcribed mRNAs comprise one or more non-coding sequences in an untranslated region (UTR), such as a 5′ UTR or 3′ UTR. Inclusion of a non-coding sequence in the UTR of an mRNA prevents the non-coding sequence from being translated into a peptide. In some embodiments, a non-coding sequence is positioned in a 3′ UTR of an mRNA. In some embodiments, the non-coding sequence is positioned upstream of the polyA tail of the mRNA. In some embodiments, the non-coding sequence is positioned downstream of (e.g., after) the polyA tail of the mRNA. In some embodiments, the non-coding sequence is positioned between the last codon of the ORF of the mRNA and the first “A” of the polyA tail of the mRNA. In some embodiments, a polynucleotide non-coding positioned in a UTR comprises between 1 and 10 nucleotides (e.g., 1, 2, 3, 4, 5, 6, 7, 8, 9, or 10 nucleotides). In some embodiments, UTR comprising a polynucleotide non-coding sequence further comprises one or more (e.g., 1, 2, 3, or more) RNAse cleavage sites, such as RNase H cleavage sites. In some embodiments, each different RNA of a multivalent RNA composition comprises a different (e.g., unique) non-coding sequence. In some embodiments, RNAs of a multivalent RNA composition are detected and/or purified according to the polynucleotide non-coding sequences of the RNAs. In some embodiments, the mRNA non-coding sequences are used to identify the presence of mRNA or determine a relative ratio of different mRNAs in a sample (e.g., a reaction product or a drug product). In some embodiments, the mRNA non-coding sequences are detected using one or more of deep sequencing, PCR, and Sanger sequencing. Exemplary non-coding sequences include: AACGUGAU; AAACAUCG; ATGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; CAAUGGAA; CACUUCGA; CAGCGUUA; CAUACCAA; CCAGUUCA; CCGAAGUA; ACAGUG; CGAUGU; UUAGGC; AUCACG; and UGACCA.

In some embodiments the multivalent RNA composition is produced by a method comprising:
(a) combining a linearized first DNA molecule encoding the first mRNA polynucleotide, a linearized second DNA molecule encoding the second mRNA polynucleotide, and a linearized third, fourth, fifth, sixth, seventh, eighth, ninth or tenth DNA molecule encoding the third, fourth, fifth, sixth, seventh, eighth, ninth or tenth mRNA polynucleotide into a single reaction vessel, wherein the first DNA molecule, the second DNA molecule, and the third, fourth, fifth, sixth, seventh, eighth, ninth or tenth DNA molecule are obtained from different sources; and
(b) simultaneously in vitro transcribing the linearized first DNA molecule, the linearized second DNA molecule and the linearized third, fourth, fifth, sixth, seventh, eighth, ninth or tenth DNA molecule to obtain a multivalent RNA composition. The different sources may be bacterial cell cultures which may not be co-cultured. In some embodiments the amounts of the first, second and third, fourth, fifth, sixth, seventh, eighth, ninth or tenth DNA molecules present in the reaction mixture prior to the start of the IVT have been normalized.

### Multivalent Vaccines

The compositions, as provided herein, may include RNA or multiple RNAs encoding two or more antigens of the same or different species (e.g., different norovirus genogroups or genotypes); that is, the compositions may be multivalent compositions (e.g., vaccines). In some embodiments, the composition includes an RNA or multiple RNAs encoding two or more norovirus major capsid protein (VP1). In some embodiments, the RNA may encode 1, 2, 3, 4, 5, 6, 7, 8, 9, 10, 11, 12, or more norovirus major capsid protein (VP1).

In some embodiments, a lipid nanoparticle may comprise two or more different mRNA encoding norovirus major capsid protein (VP1) (e.g., a single lipid nanoparticle comprises 2, 3, 4, 5, 6, 7, 8, 9, and/or 10 norovirus major capsid proteins (VP1)). In other embodiments, two or more lipid nanoparticles may comprise two or more different RNA encoding norovirus major capsid proteins (VP1) (e.g., each lipid nanoparticle comprises a single RNA). The two or more lipid nanoparticles may then be combined and administered as a single vaccine composition (e.g., comprising multiple RNA encoding multiple antigens) or may be administered separately.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

### Formula (AI)

In some embodiments, the ionizable amino lipid of the present disclosure is a compound of Formula (AI):

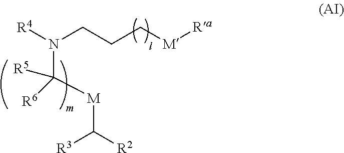

or its N-oxide, or a salt or isomer thereof,
wherein R′^{a} is R′^{branched}; wherein
R′^{branched} is:

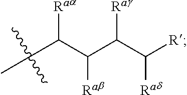

wherein

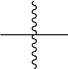

denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

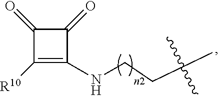

wherein

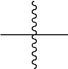

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

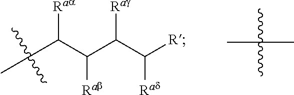

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

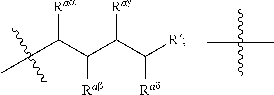

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

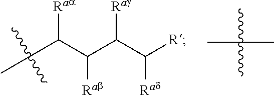

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

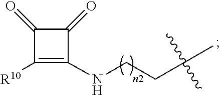

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

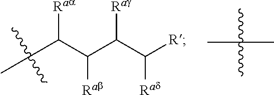

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the compound of Formula (AI) is selected from:

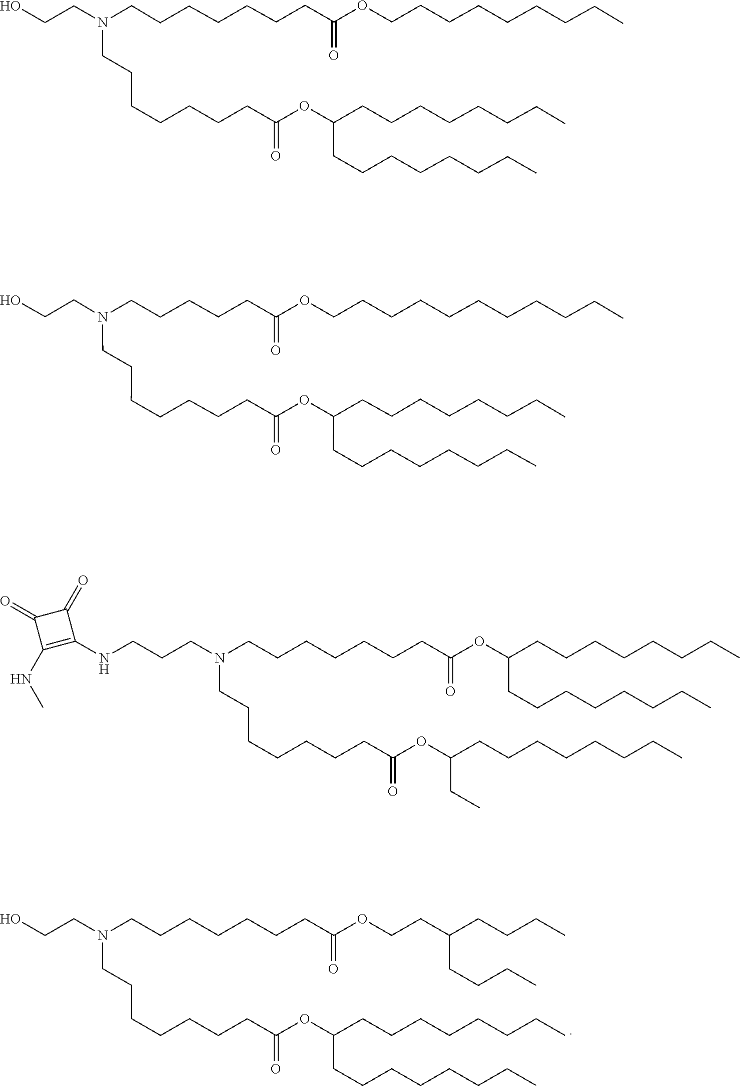 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

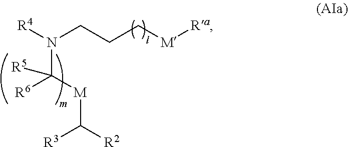

or its N-oxide, or a salt or isomer thereof,
wherein R′^{a} is R′^{branched}; wherein
R′^{branched} is:

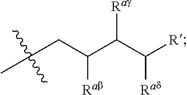

wherein

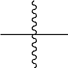

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

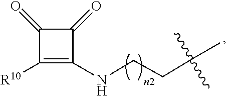

wherein

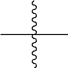

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (AIb):

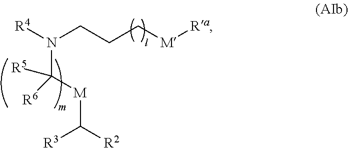

or its N-oxide, or a salt or isomer thereof,
wherein R′^{a} is R′^{branched}; wherein
R′^{branched} is:

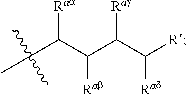

wherein

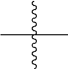

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5; each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

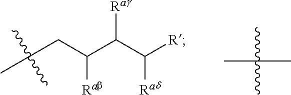

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

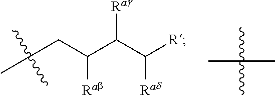

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}; R′^{branched} is

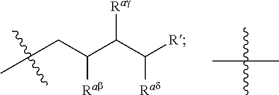

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid of Formula (AI) is a compound of Formula (AIc):

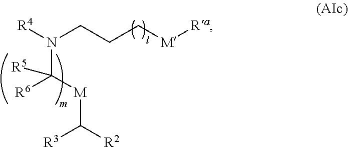

or its N-oxide, or a salt or isomer thereof,
wherein R′^{a} is R′^{branched}; wherein
R′^{branched} is:

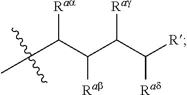

wherein

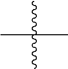

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

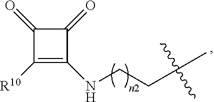

wherein

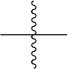

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and m is selected from the group consisting of 5, 6, 7, 8, 9, 10, 11, 12, and 13.

In some embodiments, R′^{a} is R′^{branched}; R′^{branched} is

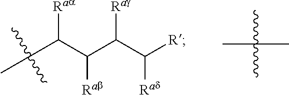

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

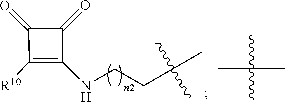

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

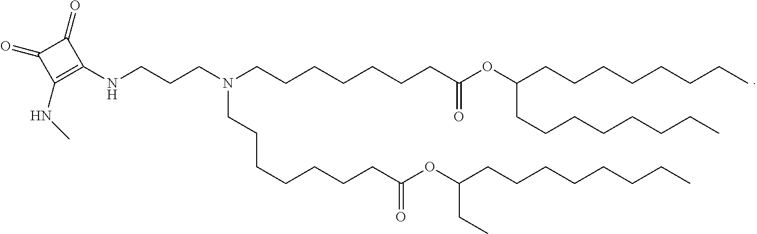 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### Formula (AII)

In some embodiments, the ionizable amino lipid is a compound of Formula (AII):

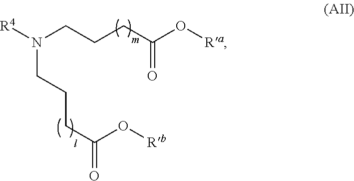

or its N-oxide, or a salt or isomer thereof,
wherein R′^{a} is R′^{branched} or R′^{cyclic}; wherein
R′^{branched} is

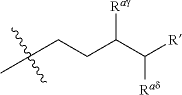

and R′^{cyclic} is:

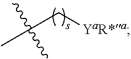

and
R′^{b} is:

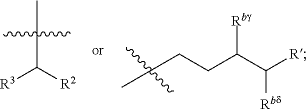

wherein

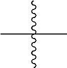

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bα} are each independently selected from the group consisting of H, C₁₋₁₂ alkyl, and C₂₋₁₂ alkenyl, wherein at least one of R^{bγ} and R^{bα} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

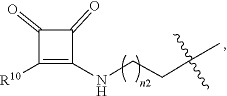

wherein

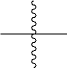

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

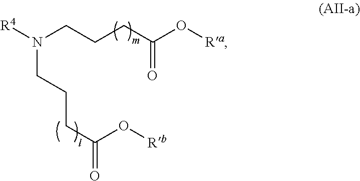

or its N-oxide, or a salt or isomer thereof, wherein R′^{a} is R′^{branched} or R′^{cyclic}; wherein
R′^{branched} is:

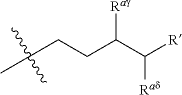

and R′^{b} is:

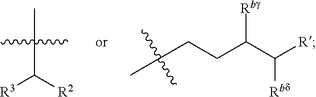

wherein

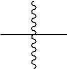

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

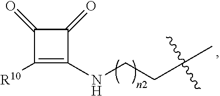

wherein

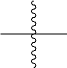

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

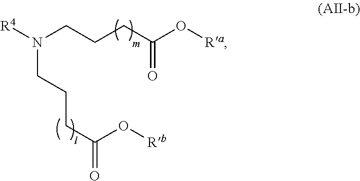

or its N-oxide, or a salt or isomer thereof,
wherein R′^{a} is R′^{branched} or R′^{cyclic}; wherein
R′^{branched} is:

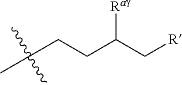

and R′^{b} is:

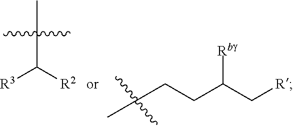

wherein

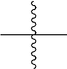

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

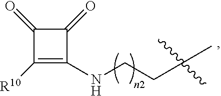

wherein

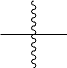

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

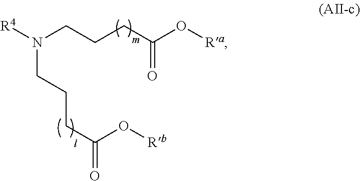

or its N-oxide, or a salt or isomer thereof,
wherein R′^{a} is R′^{branched} or R′^{cyclic}; wherein
R′^{branched} is:

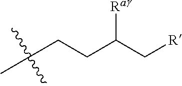

and R′^{b} is:

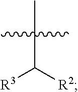

wherein

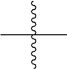

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

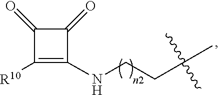

wherein

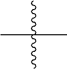

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

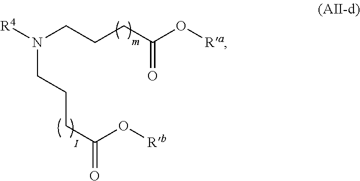

or its N-oxide, or a salt or isomer thereof,
wherein R′^{a} is R′^{branched} or R′^{cyclic}; wherein
R′^{branched} is:

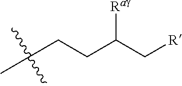

and R′^{b} is:

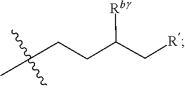

wherein

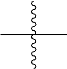

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

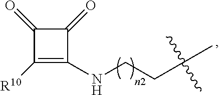

wherein

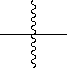

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

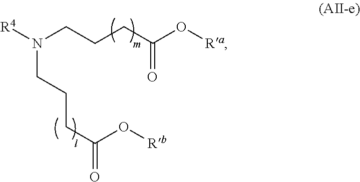

or its N-oxide, or a salt or isomer thereof,
wherein R′^{a} is R′^{branched} or R′^{cyclic}; wherein
R′^{branched} is:

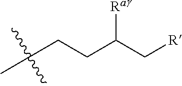

and R′^{b} is:

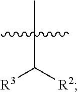

wherein

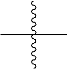

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

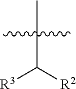

and R² and R³ are each independently a C₁₋₁₄ alkyl. In some embodiments of the compound of Formula (AII), (AII-a), (AII-b), (AII-c), (AII-d), or (AII-e), R′^{b} is:

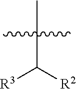

and R² and R³ are each independently a C₆₋₁₀ alkyl. In some embodiments of the compound of Formula (AII), (AII-a), (AII-b), (AII-c), (AII-d), or (AII-e), R′^{b} is:

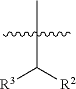

and R² and R³ are each a C₈ alkyl.

In some embodiments of the compound of Formula (AII), (AII-a), (AII-b), (AII-c), (AII-d), or (AII-e), R′^{branched} is:

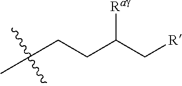

### and R′^{b} is:

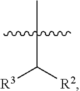

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

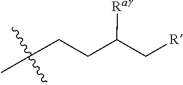

### and R′^{b} is:

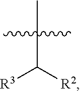

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

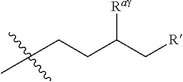

### and R′^{b} is:

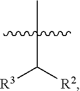

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

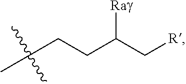

### R′^{b} is:

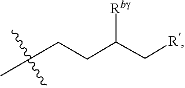

R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

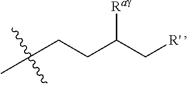

### R′^{b} is:

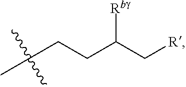

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

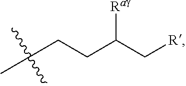

### R′^{b} is:

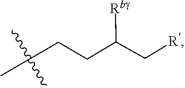

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

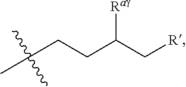

### R′^{b} is:

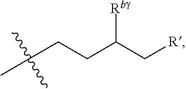

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

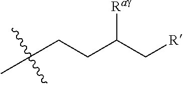

### and R′^{b} is:

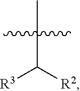

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

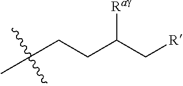

### and R′^{b} is:

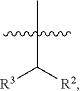

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

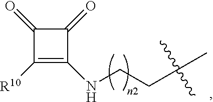

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

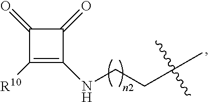

wherein R¹⁰ is NH(CH₃) and n2 is 2.

In some embodiments of the compound of Formula (AII), (AII-a), (AII-b), (AII-c), (AII-d), or (AII-e), R′^{branched} is:

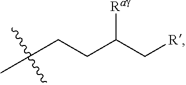

### R′^{b} is:

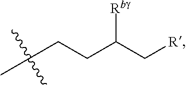

m and l are each independently selected from 4, 5, and 6, each R′ independently is a C₁₋₁₂ alkyl, R^{aγ} and R^{bγ} are each a C₁₋₁₂ alkyl, and R⁴ is

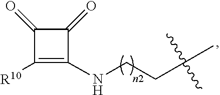

wherein R¹⁰ is NH(C₁₋₆ alkyl), and n2 is 2. In some embodiments of the compound of Formula (AII), (AII-a), (AII-b), (AII-c), (AII-d), or (AII-e). R′^{branched} is:

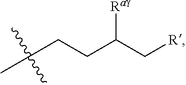

### R′^{b} is:

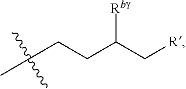

m and l are each 5, each R′ independently is a C₂₋₅ alkyl, R^{aγ} and R^{bγ} are each a C₂₋₆ alkyl, and R⁴ is

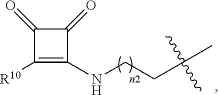

wherein R¹⁰ is NH(CH₃) and n2 is 2.

In some embodiments of the compound of Formula (AII), (AII-a), (AII-b), (AII-c), (AII-d), or (AII-e), R′^{branched} is:

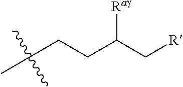

### and R′^{b} is:

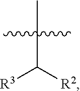

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

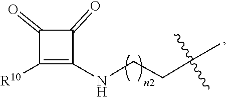

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

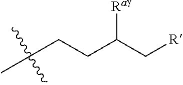

### and R′^{b} is:

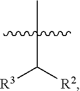

m and l are each 5, R′ is a C₂₋₅ alkyl, R^{aγ} is a C₂₋₆ alkyl, R² and R³ are each a C₈ alkyl, and R⁴ is

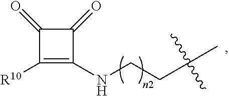

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

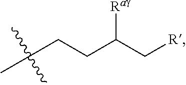

### R′^{b} is:

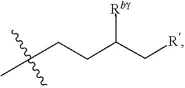

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

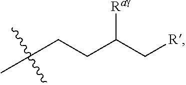

### R′^{b} is:

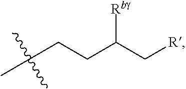

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

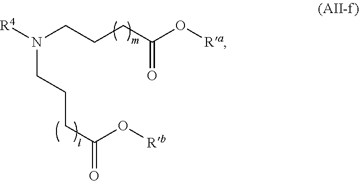

or its N-oxide, or a salt or isomer thereof,
wherein R′^{a} is R′^{branched} or R′^{cyclic}; wherein
R′branched is:

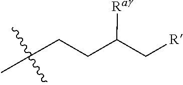

and R′b is:

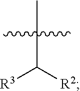

wherein

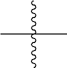

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

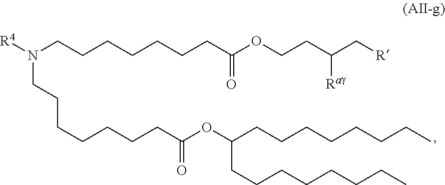

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

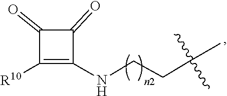

wherein

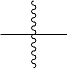

denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

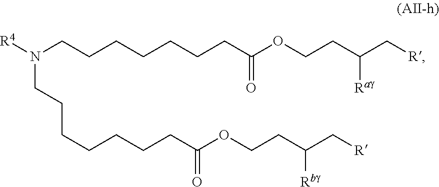

or its N-oxide, or a salt or isomer thereof; wherein
R^{aγ} and R^{bγ} are each independently a C₂₋₆ alkyl;
each R′ independently is a C₂-s alkyl; and
R⁴ is selected from the group consisting of —(CH₂)ₙOH wherein n is selected from the group consisting of 3, 4, and 5, and

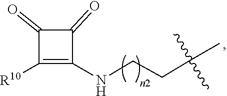

wherein

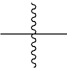

denotes a point of attachment, R¹⁰ is NH(C₁₋₆ alkyl), and n2 is selected from the group consisting of 1, 2, and 3.

In some embodiments of the compound of Formula (AI-g) or (AII-h), R⁴ is

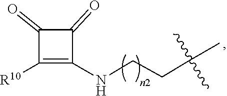

wherein
R¹⁰ is NH(CH₃) and n2 is 2.

In some embodiments of the compound of Formula (AII-g) or (AII-h), R⁴ is —(CH₂)₂OH.

### Formula (AII)

In some embodiments, the ionizable amino lipids of the present disclosure may be one or more of compounds of Formula (AIII):

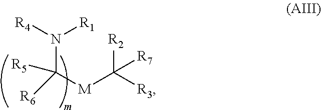

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)·CHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)·CHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (AIII) includes those of Formula (AIII-A):

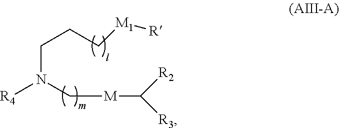

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-B):

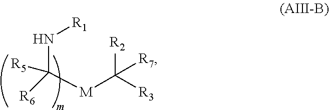

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-C):

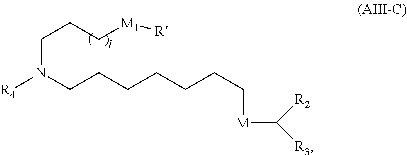

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (AIII) are of Formula (AIII-D),

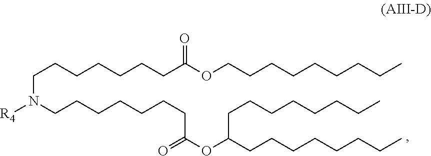

or their N-oxides, or salts or isomers thereof, wherein R₄ is as described herein.

In another embodiment, the compounds of Formula (AIII) are of Formula (AIII-E),

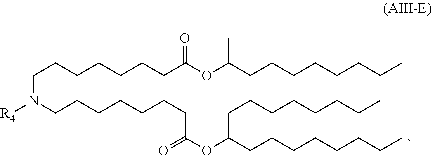

or their N-oxides, or salts or isomers thereof, wherein R₄ is as described herein.

In another embodiment, the compounds of Formula (AIII) are of Formula (AIII-F) or (AIII-G):

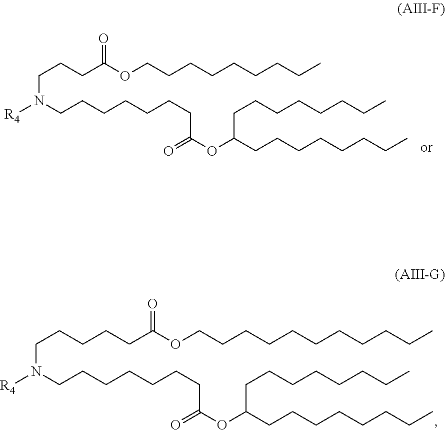

or their N-oxides, or salts or isomers thereof, wherein R₄ is as described herein.

In another embodiment, the compounds of Formula (AIII) are of Formula (AIII-H):

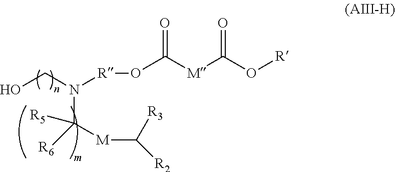

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (AIII) are of Formula (AIII-I):

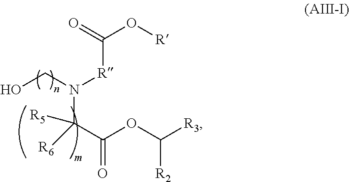

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

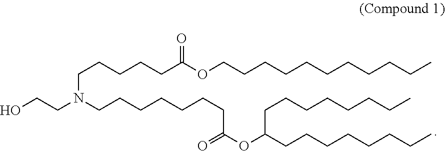 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

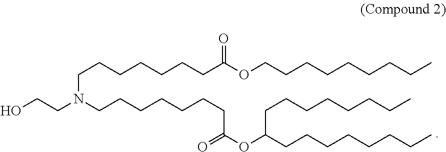 [SMILES: CCCCCCCCCOC(=O)CCCCCCCN(CCO)CCCCCCCC(=O)OC(CCCCCCCC)CCCCCCCC]

In a further embodiment, the compounds of Formula (AIII) are of Formula (AIII-J),

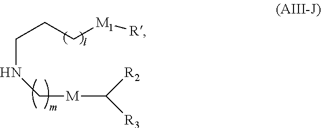

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are of Formula (AIII), or salts or isomers thereof, wherein:
R₁ is —R″M′R′;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently —OC(O)—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, an ionizable amino lipid of the disclosure comprises a compound having structure:

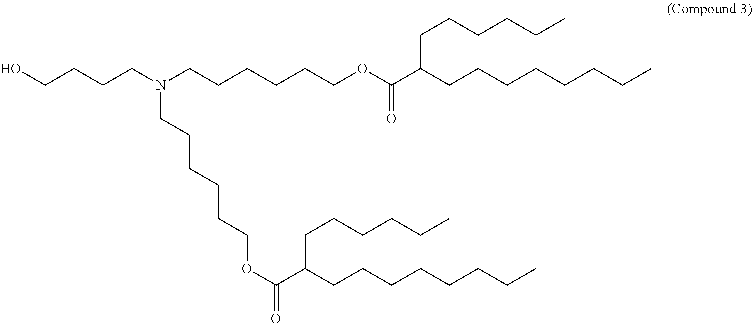 [SMILES: CCCCCCCCC(CCCCCC)C(=O)OCCCCCCN(CCCCO)CCCCCCOC(=O)C(CCCCCC)CCCCCCCC]

In some embodiments, the ionizable amino lipids are of Formula (AIII), or salts or isomers thereof, wherein:
R₁ is C₅₋₂₀ alkenyl;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently C(O)O—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, an ionizable amino lipid of the disclosure comprises a compound having structure:

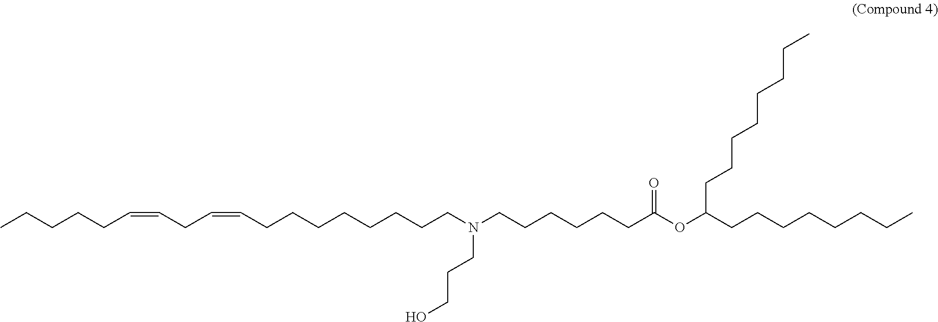 [SMILES: CCCCC/C=C\C/C=C\CCCCCCCCN(CCCO)CCCCCCC(=O)OC(CCCCCCCC)CCCCCCCC]

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (AIII), (AIII-A), (AIII-B), (AIII-C), (AIII-D), (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids of the present disclosure may be one or more of compounds of formula (AIV),

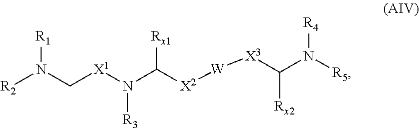

or salts or isomers thereof, wherein
W is

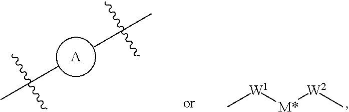

ring A is

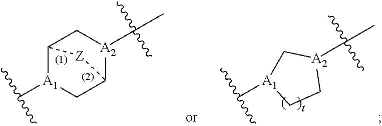

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

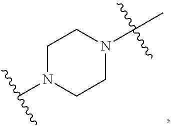 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (AIVa)-(AIVh):

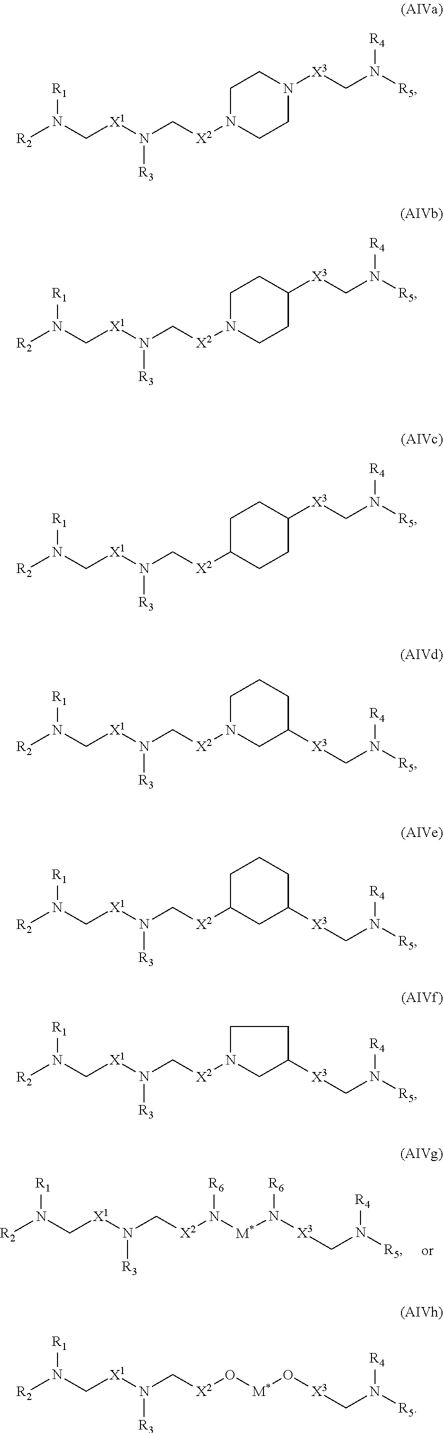

In some embodiments, the ionizable amino lipid is

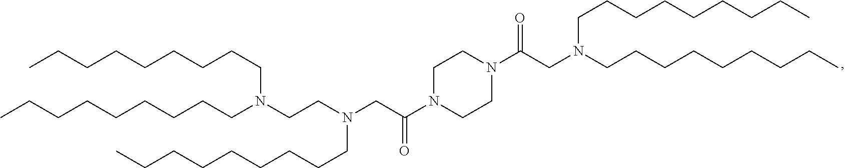 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH. Thus, a lipid may have a positive or partial positive charge at physiological pH.

### Formula (AV)

In some embodiments, the lipid nanoparticle comprises a lipid having the structure:

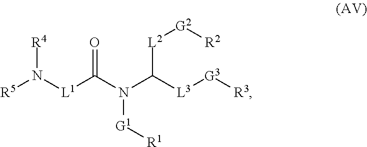

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

### Formula (AVI)

In some embodiments, the lipid nanoparticle comprises a lipid having the structure:

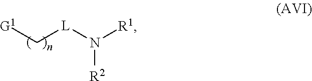

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and n is an integer from 1 to 12.

### Formula (AVII)

In some embodiments, the lipid nanoparticle comprises a lipid having the structure:

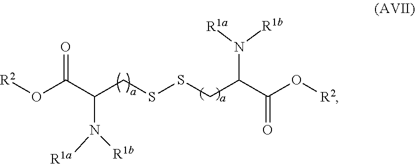

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each L₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

### Formula (AVIII)

In some embodiments, the lipid nanoparticle comprises a lipid having the structure:

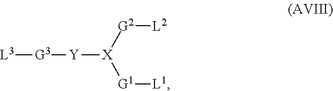

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR′, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f};
   —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{e} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

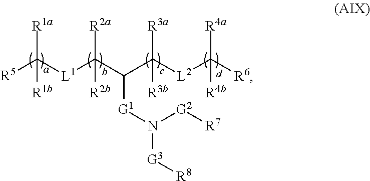

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(C═O)NR⁹R¹⁰, —NR⁹R¹⁰, —(C═O)OR″¹ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

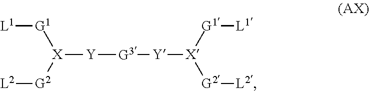

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
   a) Y is absent when X is N;
   b) Y′ is absent when X′ is N;
   c) Y is —O(C═O)—, —(C═O)O— or NR when X is CR; and
   d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{e} and R^{f} are, at each occurrence, independently C₁-C₁₂ alkyl or C₂-C₁₂ alkenyl; R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

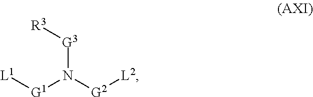

or a pharmaceutically acceptable salt, prodrug or stereoisomer thereof, wherein:
L¹ is —O(C═O)R¹, —(C═O)OR¹, —C(═O)R¹, —OR′, —S(O)ₓR¹, —S—SR¹,—C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR²,—C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f};
   —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

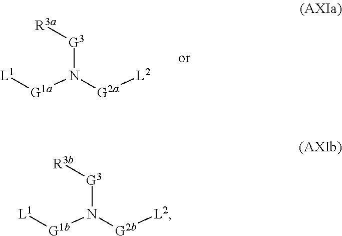

or a pharmaceutically acceptable salt, prodrug or stereoisomer thereof, wherein:

L¹ is —O(C═O)R¹, —(C═O)OR¹, —C(═O)R¹, —OR′, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f};—NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

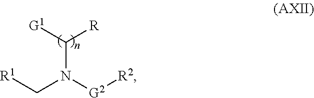

or a pharmaceutically acceptable salt, prodrug or stereoisomer thereof, wherein:
G¹ is —OH, —R³R⁴, —(C═O) R⁵ or —R³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

### Formula (AXIII)

In some embodiments, the lipid nanoparticle comprises a lipid having the structure:

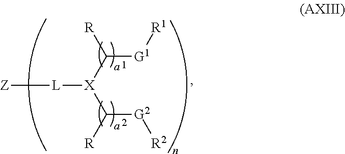

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, —O(C═O)—, wherein ~ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

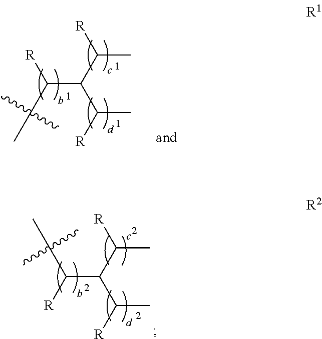

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

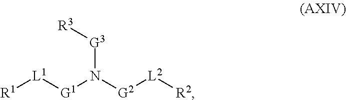

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O) R^{a}—, R^{a}C(═O) R^{a}—, —OC(═O) R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O) R^{a}—, R^{a}C(═O) R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

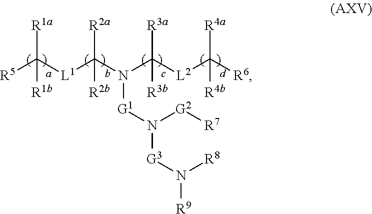

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

### Formula (AXVI)

In some embodiments, the lipid nanoparticle comprises a lipid having the structure:

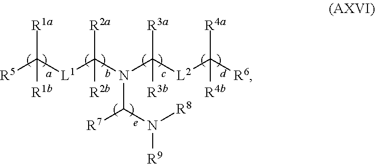

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

### Formula (AXVII)

In some embodiments, the lipid nanoparticle comprises a lipid having the structure:

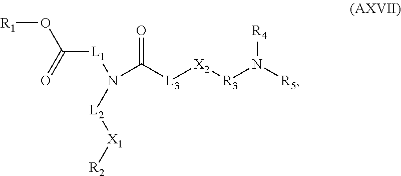

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

### Compounds (AI)-(AII)

In some embodiments, the lipid nanoparticle comprises an ionizable lipid having the structure:

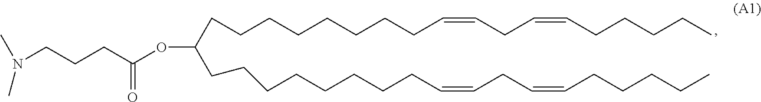 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

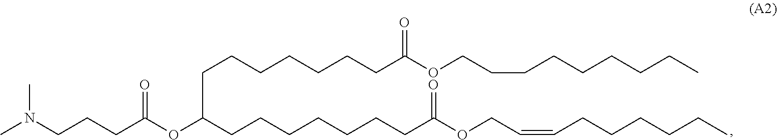 [SMILES: CCCCCC/C=C\COC(=O)CCCCCCCC(CCCCCCCC(=O)OCCCCCCCCC)OC(=O)CCCN(C)C]

or a pharmaceutically acceptable salt thereof.

In some embodiments, the lipid nanoparticle comprises a lipid having the structure:

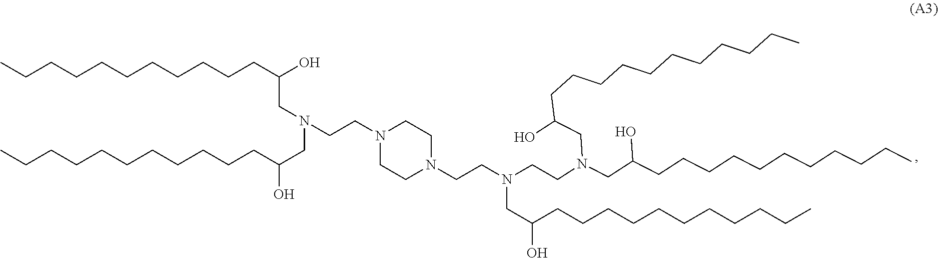 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

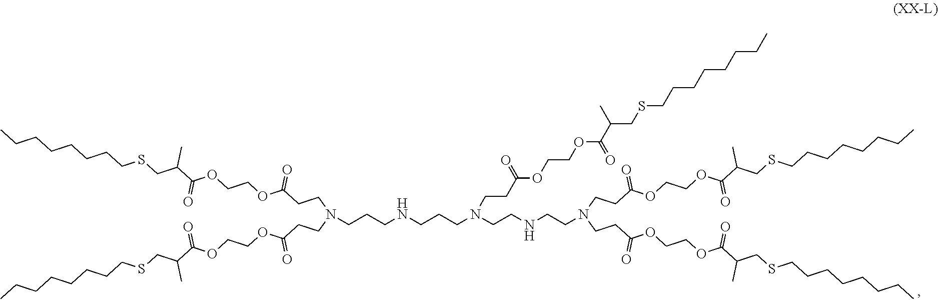 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, the lipid nanoparticle comprises a lipid having the structure:

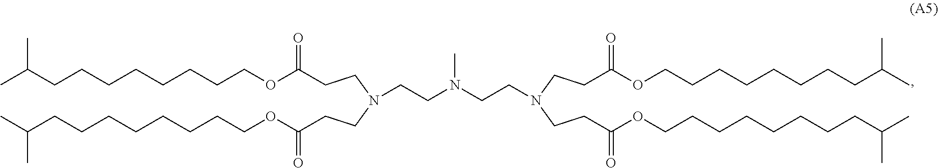 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

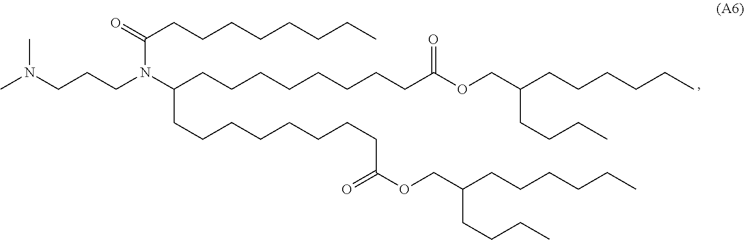 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

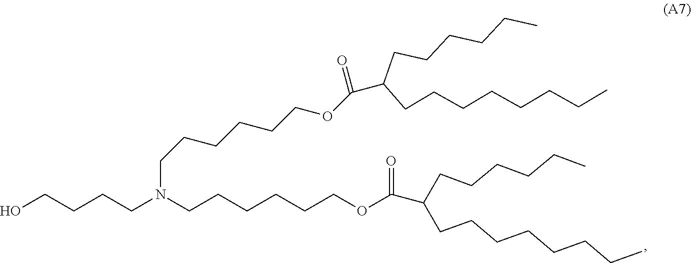 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

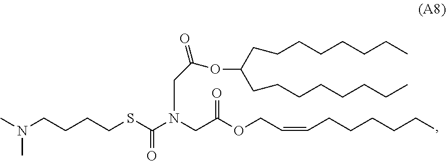

or a pharmaceutically acceptable salt thereof.

In some embodiments, the lipid nanoparticle comprises a lipid having the structure:

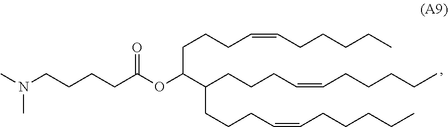 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid namoparticle comprises a lipid having the structure:

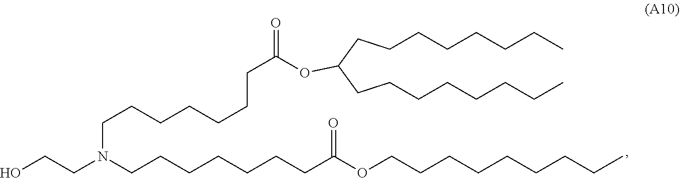 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

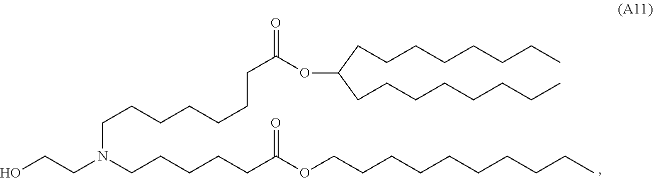 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI):

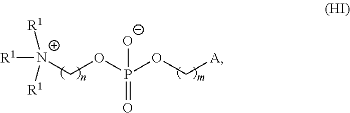

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
nis 1,2, 3, 4, 5, 6, 7, 8, 9, or 10;
m is 0, 1, 2, 3, 4, 5, 6, 7, 8, 9, or 10;
A is of the formula:

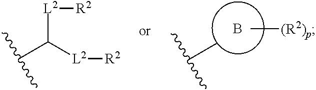

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), —N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

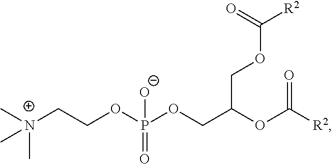

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 34-35 mol %, 35-36 mol %, 36-37 mol %, 37-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example a mPEG-NH₂, has a size of about 1000, 2000, 5000, 10,000, 15,000 or 20,000 daltons. In some embodiments, the PEG-lipid is PEG₂k-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

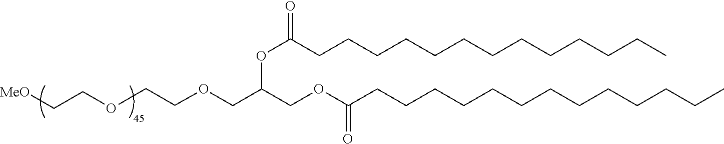

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG lipid is a compound of Formula (PI):

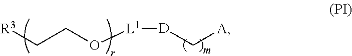

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, —OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

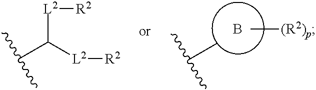

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), —N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PI) is of Formula (PI-OH):

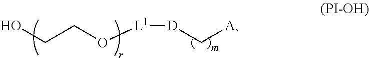

or a salt thereof.

### Formula (PII)

In certain embodiments, a PEG lipid is a PEGylated fatty acid. In certain embodiments, a PEG lipid is a compound of Formula (PII). In some embodiments, compounds of Formula (PII) have the following formula:

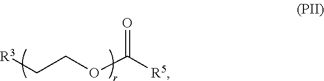

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (PII) is of Formula (PII-OH):

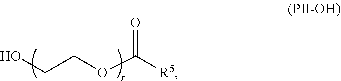

or a salt thereof. In some embodiments, r is 40-50.

In yet other embodiments the compound of Formula (PII) is:

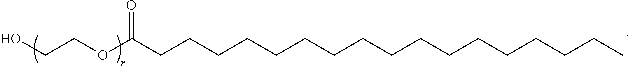

or a salt thereof.

In some embodiments, the compound of Formula (PII) is

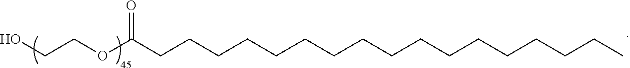

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). In embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP of the disclosure comprises an ionizable amino lipid of Compound 2, wherein the non-cationic lipid is DSPC, the structural lipid that is cholesterol, and the PEG lipid is DMG-PEG.

In some embodiments, a LNP comprises an ionizable amino lipid of any of Formula (AIII), (AIV), or (AV), a phospholipid comprising DSPC, a structural lipid, and a PEG lipid comprising PEG-DMG.

In some embodiments, a LNP comprises an ionizable amino lipid of any of Formula (AIII), (AIV), or (AV), a phospholipid comprising DSPC, a structural lipid, and a PEG lipid comprising a compound having Formula (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG lipid comprising a compound having Formula (PII).

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprise an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

Stabilizing Compounds Some embodiments of the compositions described herein are stabilized pharmaceutical compositions. Various non-viral delivery systems, including nanoparticle formulations, present attractive opportunities to overcome many challenges associated with mRNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

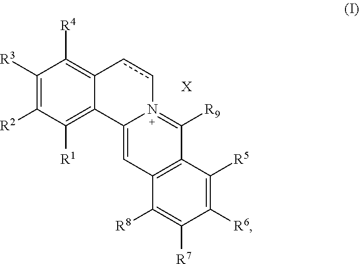

or a tautomer or solvate thereof, wherein:
[symbol] is a single bond or a double bond;
R¹ is H; R² is OCH₃, or together with R³ is OCH₂O; R³ is OCH₃, or together with R² is OCH₂O; R⁴ is H; R⁵ is H or OCH₃; R⁶ is OCH₃; R⁷ is H or OCH₃; R⁸ is H; R⁹ is H or CH₃; and X is a pharmaceutically acceptable anion, e.g., a halide such as chloride.

In some embodiments, the compound of Formula (I) has the structure of:

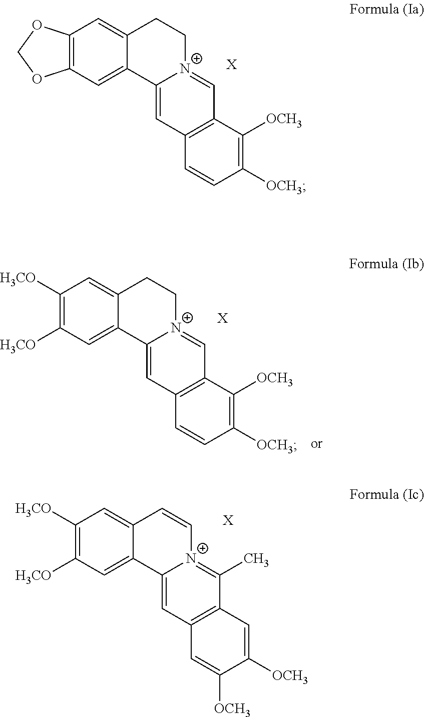

or a tautomer or solvate thereof.

In some embodiments, the stabilized pharmaceutical composition comprises a nucleic acid formulation comprising a nucleic acid and a lipid, and a compound of Formula (II):

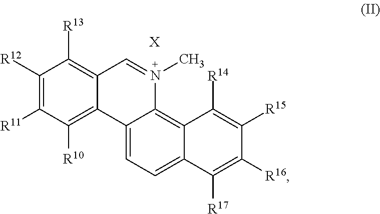

or a tautomer or solvate thereof, wherein:
R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

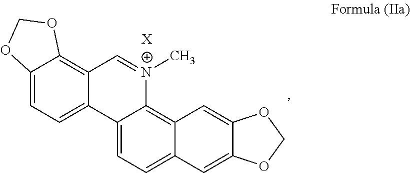

or a tautomer or solvate thereof.

Stabilizing compounds of Formulas (I), (Ia), (Ib), (Ic), (II), and (IIa) are described in International Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 10⁻³ M (e.g., less than 10⁻⁴ M, less than 10⁻⁵ M, less than 10⁻⁵ M, less than 10⁻⁷ M, less than 10⁻⁸ M, or less than 10⁻⁹ M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM.

### Insertions and Substitutions

The present disclosure also includes a polynucleotide of the present disclosure that further comprises insertions and/or substitutions.

In some embodiments, the 5′UTR of the polynucleotide can be replaced by the insertion of at least one region and/or string of nucleosides of the same base. The region and/or string of nucleotides can include, but is not limited to, at least 3, at least 4, at least 5, at least 6, at least 7 or at least 8 nucleotides and the nucleotides can be natural and/or unnatural. As a non-limiting example, the group of nucleotides can include 5-8 adenine, cytosine, thymine, a string of any of the other nucleotides disclosed herein and/or combinations thereof.

In some embodiments, the 5′UTR of the polynucleotide can be replaced by the insertion of at least two regions and/or strings of nucleotides of two different bases such as, but not limited to, adenine, cytosine, thymine, any of the other nucleotides disclosed herein and/or combinations thereof. For example, the 5′UTR can be replaced by inserting 5-8 adenine bases followed by the insertion of 5-8 cytosine bases. In another example, the 5′UTR can be replaced by inserting 5-8 cytosine bases followed by the insertion of 5-8 adenine bases.

In some embodiments, the polynucleotide can include at least one substitution and/or insertion downstream of the transcription start site that can be recognized by an RNA polymerase. As a non-limiting example, at least one substitution and/or insertion can occur downstream of the transcription start site by substituting at least one nucleic acid in the region just downstream of the transcription start site (such as, but not limited to, +1 to +6). Changes to region of nucleotides just downstream of the transcription start site can affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety). The modification, substitution and/or insertion of at least one nucleoside can cause a silent mutation of the sequence or can cause a mutation in the amino acid sequence.

In some embodiments, the polynucleotide can include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In some embodiments, the polynucleotide can include the substitution of at least 1, at least 2, at least 3, at least 4, at least 5 or at least 6 guanine bases in the region just downstream of the transcription start site. As a non-limiting example, if the nucleotides in the region are GGGAGA, the guanine bases can be substituted by at least 1, at least 2, at least 3 or at least 4 adenine nucleotides. In another non-limiting example, if the nucleotides in the region are GGGAGA the guanine bases can be substituted by at least 1, at least 2, at least 3 or at least 4 cytosine bases. In another non-limiting example, if the nucleotides in the region are GGGAGA the guanine bases can be substituted by at least 1, at least 2, at least 3 or at least 4 thymine, and/or any of the nucleotides described herein.

In some embodiments, the polynucleotide can include at least one substitution and/or insertion upstream of the start codon. For the purpose of clarity, one of skill in the art would appreciate that the start codon is the first codon of the protein coding region whereas the transcription start site is the site where transcription begins. The polynucleotide can include, but is not limited to, at least 1, at least 2, at least 3, at least 4, at least 5, at least 6, at least 7 or at least 8 substitutions and/or insertions of nucleotide bases. The nucleotide bases can be inserted or substituted at 1, at least 1, at least 2, at least 3, at least 4 or at least 5 locations upstream of the start codon. The nucleotides inserted and/or substituted can be the same base (e.g., all A or all C or all T or all G), two different bases (e.g., A and C, A and T, or C and T), three different bases (e.g., A, C and T or A, C and T) or at least four different bases.

As a non-limiting example, the guanine base upstream of the coding region in the polynucleotide can be substituted with adenine, cytosine, thymine, or any of the nucleotides described herein. In another non-limiting example the substitution of guanine bases in the polynucleotide can be designed so as to leave one guanine base in the region downstream of the transcription start site and before the start codon (see Esvelt et al. Nature (2011) 472(7344):499-503; the contents of which is herein incorporated by reference in its entirety). As a non-limiting example, at least 5 nucleotides can be inserted at 1 location downstream of the transcription start site but upstream of the start codon and the at least 5 nucleotides can be the same base type.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

Provided herein are compositions (e.g., pharmaceutical compositions), methods, kits and reagents for prevention and/or treatment of disease in humans and other mammals. The compositions can be used as therapeutic or prophylactic agents. For example, when the composition comprises a norovirus major capsid protein (VP1) the RNA encoding such a norovirus major capsid protein (VP1) is used to provide prophylactic or therapeutic protection from a Norovirus infection. Prophylactic protection from Norovirus infection can be achieved following administration of a composition (e.g., a composition comprising one or more polynucleotides encoding one or more norovirus major capsid protein (VP1)) of the present disclosure. In some embodiments, the Norovirus is a member of the Genogroup GI. In some embodiments, the Norovirus is a member of the Genogroup GII. In some embodiments, the Norovirus is a member of the Genogroup GIV. Compositions can be administered once, twice, three times, four times or more. In some aspects, the compositions can be administered to an infected individual to achieve a therapeutic response. Dosing may need to be adjusted accordingly.

It is envisioned that there may be situations where persons are at risk for infection with more than one strain of type of infectious agent. RNA (mRNA) therapeutic treatments are particularly amenable to combination vaccination approaches due to a number of factors including, but not limited to, speed of manufacture, ability to rapidly tailor treatments to accommodate perceived geographical threat, and the like. To protect against more than one strain of Norovirus, a combination treatment can be administered that includes RNA encoding at least one polypeptide (or portion thereof) of a norovirus major capsid protein (VP1) from one genogroup and further includes RNA encoding at least one additional norovirus major capsid protein (VP1) from a second genogroup. RNAs (mRNAs) can be co-formulated, for example, in a single lipid nanoparticle (LNP) or can be formulated in separate LNPs destined for co-administration. In some embodiments, a single lipid nanoparticle comprises RNAs (mRNAs). In some embodiments, at least two lipid nanoparticles comprise RNAs (mRNAs) (e.g., each lipid nanoparticle comprises a single RNA (mRNA)).

A prophylactically effective dose is a therapeutically effective dose that prevents infection with the virus at a clinically acceptable level. In some embodiments, the therapeutically effective dose is a dose listed in a package insert for the treatment. A prophylactic therapy as used herein refers to a therapy that prevents, to some extent, the infection from increasing. The infection may be prevented completely or partially.

The methods of the invention involve, in some aspects, passively immunizing a mammalian subject against an norovirus virus infection. The method involves administering to the subject a composition comprising at least one RNA polynucleotide having an open reading frame encoding at least one norovirus major capsid protein (VP1). In some aspects, methods of the present disclosure provide prophylactic treatments against an Norovirus infection. In some embodiments, the Norovirus is a member of the Genogroup GI. In some embodiments, the Norovirus is a member of the Genogroup GII. In some embodiments, the Norovirus is a member of the Genogroup GIV.

Therapeutic methods of treatment are also included within the invention. Methods of treating a Norovirus infection in a subject are provided in aspects of the disclosure. The method involves administering to the subject having a Norovirus infection a composition comprising at least one RNA polynucleotide having an open reading frame encoding at least one norovirus major capsid protein (VP1). In some embodiments, the Norovirus is a member of the Genogroup GI. In some embodiments, the Norovirus is a member of the Genogroup GII. In some embodiments, the Norovirus is a member of the Genogroup GIV

As used herein, the terms treat, treated, or treating when used with respect to a disorder such as a viral infection, refers to a treatment which increases the resistance of a subject to development of the disease or, in other words, decreases the likelihood that the subject will develop the disease in response to infection with the virus as well as a treatment after the subject has developed the disease in order to fight the infection or prevent the infection from becoming worse.

An “effective amount” of an RNA treatment of the present disclosure is provided based, at least in part, on the target tissue, target cell type, means of administration, physical characteristics of the polynucleotide (e.g., size, and extent of modified nucleosides), and other components of the RNA treatment, and other determinants. Increased antibody production may be demonstrated by increased cell transfection (the percentage of cells transfected with the RNA treatment), increased protein translation from the polynucleotide, decreased nucleic acid degradation (as demonstrated, for example, by increased duration of protein translation from a modified polynucleotide), or altered response of the host cell.

In some embodiments, RNA treatments (including polynucleotides and their encoded polypeptides) in accordance with the present disclosure may be used for treatment of the disease.

RNA treatments may be administered prophylactically or therapeutically as part of an active immunization scheme to healthy individuals or early in infection during the incubation phase or during active infection after onset of symptoms. In some embodiments, the amount of RNA treatments of the present disclosure provided to a cell, a tissue or a subject may be an amount effective for immune prophylaxis.

RNA treatments may be administered with other prophylactic or therapeutic compounds. As a non-limiting example, a prophylactic or therapeutic compound may be a vaccine containing a virus treatment with or without an adjuvant or a booster. As used herein, when referring to a prophylactic composition, such as a treatment or vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years, or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months, or 1 year.

In some embodiments, RNA treatments may be administered subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs.

Provided herein are pharmaceutical compositions including RNA treatments and RNA compositions and/or complexes optionally in combination with one or more pharmaceutically acceptable excipients.

RNA treatments may be formulated or administered in combination with one or more pharmaceutically-acceptable excipients. In some embodiments, compositions comprise at least one additional active substances, such as, for example, a therapeutically-active substance, a prophylactically-active substance, or a combination of both. Treatment compositions may be sterile, pyrogen-free or both sterile and pyrogen-free. General considerations in the formulation and/or manufacture of pharmaceutical agents, such as treatment compositions, may be found, for example, in Remington: The Science and Practice of Pharmacy 21st ed., Lippincott Williams & Wilkins, 2005 (incorporated herein by reference in its entirety).

In some embodiments, RNA treatments are administered to humans, human patients, or subjects. For the purposes of the present disclosure, the phrase “active ingredient” generally refers to the RNA treatments or the polynucleotides contained therein, for example, RNA polynucleotides (e.g., mRNA polynucleotides) encoding picornavirus capsid polyprotein and/or picornavirus 3C protease.

Formulations of the compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

RNA treatments can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (e.g., HCAb) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with RNA treatments (e.g., for transplantation into a subject), hyaluronidase, nanoparticle mimics, and combinations thereof.

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments, the RNA treatment may include one or more stabilizing elements. Stabilizing elements may include, for instance, a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA treatments include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the polynucleotides described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, or less than 975 μm.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

RNA treatments may be administered by any route which results in a therapeutically effective outcome. These include, but are not limited, to intradermal, intramuscular, and/or subcutaneous administration. The present disclosure provides methods comprising administering RNA treatments to a subject in need thereof. The exact amount required will vary from subject to subject, depending on the species, age, and general condition of the subject, the severity of the disease, the particular composition, its mode of administration, its mode of activity, and the like. RNA treatments compositions are typically formulated in dosage unit form for ease of administration and uniformity of dosage. It will be understood, however, that the total daily usage of RNA treatments compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, RNA treatments compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, RNA treatments compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, RNA treatment compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, RNA treatment compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, an RNA treatment composition may be administered three or four times.

In some embodiments, RNA treatment compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the RNA for use in a method of treating a subject is administered to the subject in a single dosage of between 10 μg/kg and 400 μg/kg of the nucleic acid treatment in an effective amount to treat the subject. In some embodiments, the RNA treatment for use in a method of treating a subject is administered to the subject in a single dosage of between 10 μg and 400 μg of the nucleic acid treatment in an effective amount to treat the subject.

An RNA pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Seronegative/Seropositive

Also provided herein are methods of administering the vaccines, methods of producing the vaccines, compositions comprising the vaccines, and nucleic acids encoding the vaccines. As described herein, the vaccines described herein may be used to induce a balanced immune response, comprising both cellular and humoral immunity, without many of the risks associated with DNA vaccination. Such a vaccine, optionally referred to herein as a multivalent vaccine or combination vaccine, can be administered to seropositive or seronegative subjects. For example, a subject may be naïve and not have antibodies that react with at least one of the respiratory virus antigenic polypeptides of the vaccine, or may have preexisting antibodies to at least one of norovirus major capsid protein (VP1) of the vaccine because they have previously had an infection with the norovirus or may have previously been administered a dose of a vaccine (e.g., an mRNA vaccine) that induces antibodies against the norovirus. In some embodiments, a subject may have preexisting antibodies to all of norovirus major capsid proteins (VP1) of the vaccine.

mRNA-Lipid Adducts

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP-IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP-IP HPLC. Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP-IP HPLC.

In some aspects, an amount of lipid aldehydes in the composition is less than about 50 ppm, including less than 50 ppm. Additionally or alternatively, in some aspects an amount of N-oxide compounds in the composition is less than about 50 ppm, including less than 50 ppm. Additionally or alternatively, in some aspects an amount of transition metals, such as Fe, in the composition is less than about 50 ppm, including less than 50 ppm. Additionally or alternatively, in some aspects an amount of alkyl halide compounds in the composition is less than about 50 ppm, including less than 50 ppm. Additionally or alternatively, in some aspects an amount of anhydride compounds in the composition is less than about 50 ppm, including less than 50 ppm. Additionally or alternatively, in some aspects an amount of ketone compounds in the composition is less than about 50 ppm, including less than 50 ppm. Additionally or alternatively, in some aspects an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition; and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (0-(2,3,4,5,6-Pentafluorobenzyl)hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof. A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl)phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9.

In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### Identification and Ratio Determination (IDR) Sequences

An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1: VP1 is Expressed in Expi293 Cells Transfected with Norovirus mRNAs

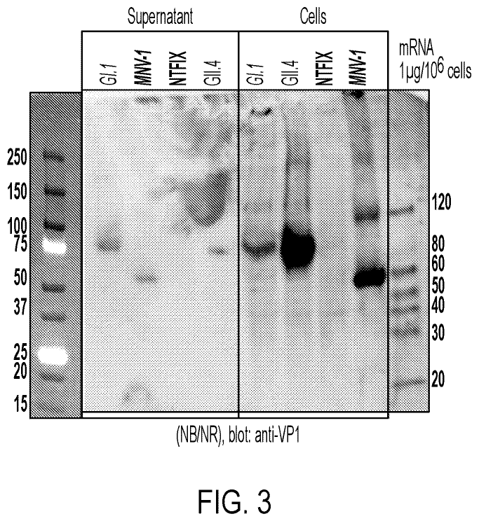

To investigate whether VP1 could be expressed in mammalian cells, Expi293 cells were transfected with Norovirus mRNAs encoding VP1. Specifically, Expi293 cells were transfected with 1 ug mRNA per 10⁶ cells of either GI.1, GII.4, or MNV1 VP1 mRNAs. Cells were grown for five days following transfection and centrifuged at 1500 rpm for 20 minutes. Both cell pellet and supernatant were analyzed by Western Blot using Anti-VP1 (P1 domain) GI/GII Abcam ab272687. VP1 from GI.1, GII.4, and MNV1 was detectable in cells (FIG. 3).

To investigate whether VP1 could be detected in the supernatant of cells transfected with Norovirus mRNAs encoding VP1, Expi293 cells were transfected with 1 ug mRNA per 10⁶ cells of either GI.1, GII.4, or MNV1. Cells were grown for 7 days following transfection and centrifuged at 1500 rpm for 20 minutes. Supernatant was analyzed by Western Blot using Anti-VP1 antibody to P1 domain of GI/GII known as Abcam ab272687. VP1 from genogroup GI.1 was detectable in supernatants collected on day 7 (FIG. 4A). VP1 from genogroup GII.4 was detectable in supernatant collected on day 1, with increasing levels detectable on days 2-7 (FIG. 4A). VP1 from MNV1 was detectable in supernatant collected on days 2 and 3 (FIG. 4B).

Transfection of Norovirus mRNAs-VP1 GI.1, GII.4 and MNV1 resulted in expression of VP1 capsid proteins that could be detected in cell lysates and cell supernatants. GII.4 VP1 mRNA seemed to express better than GI.1 and MNV1 in Expi293 cells.

### Example 2: VP1 VLPs were Imaged by Electron Microscopy (EM) Following mRNA Transfection in Expi293 Cells and Purification by Anion-Exchange Chromatography (AEX)

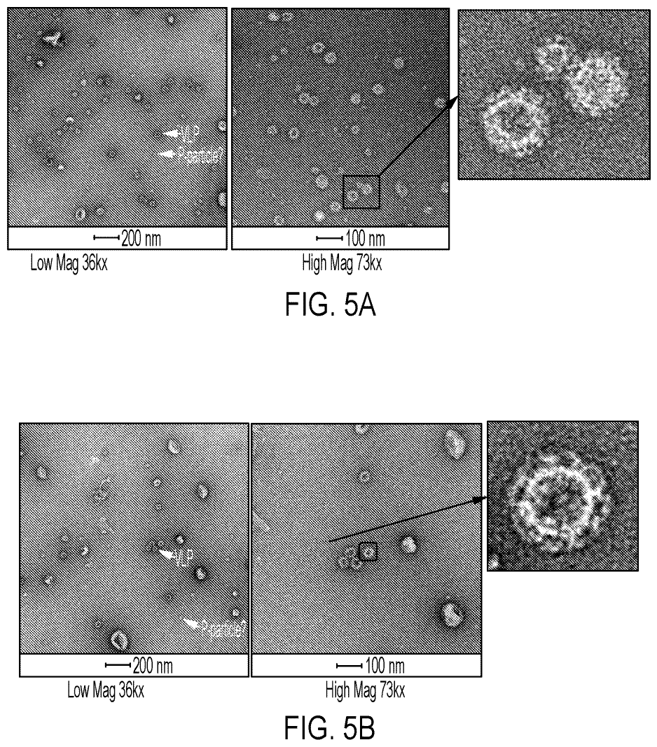

To investigate whether cells transfected with norovirus mRNAs expressing VP1 could form VLPs, VP1 was purified from Expi293 cells transfected with 1 ug GI.1 or GI.3 VP1 norovirus mRNA per 10⁶ cells. Three days following transfection, cells were centrifuged, cell pellets were sonicated, clarified lysate was precipitated with PEG600. Following precipitation, samples were run on 1) western blot to confirm VP1 presence in fractions; or 2) subject to anion exchange chromatography (AEX) purification followed by imaging by negative stain electron microscopy (EM). Expression of both GI.1 VP1 mRNA and GI.3 VP1 mRNAs resulted in VLP formation that could be detected at both high and low magnification using electron microscopy (FIGS. 5A and 5B).

### Example 3: Expression and Production of VLPs in Insect Cells

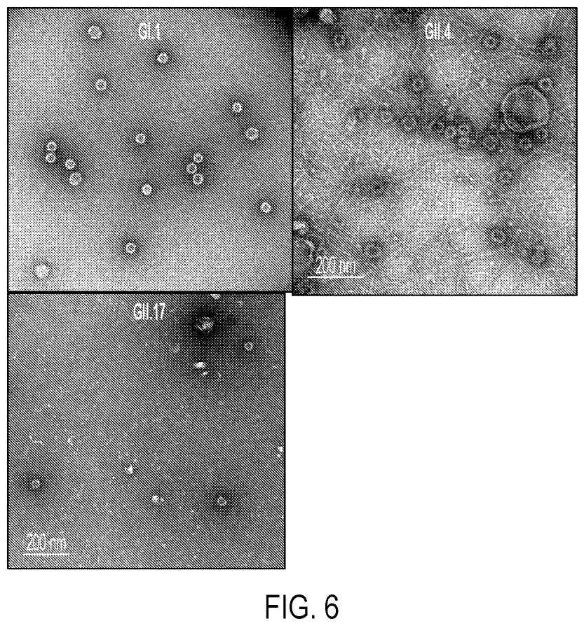

To investigate whether the VP1 mRNA sequences representing GI.1, GII.4 and GII.17 could produce VLPs in insect cells, the corresponding VP1 genes were cloned in pFastBac1. Baculoviruses were subsequently generated using the Bac-to-Bac system (Invitrogen). ExpiSf9 cells were infected with baculoviruses expressing VP1s and cell culture supernatants were collected at day 5 post infection. VLPs were precipitated overnight with PEG and purified by Anion Exchange Chromatography (FIG. 6). GI.1, GII.4 and GII.17 mRNAs resulted in VLP formation that could be detected using electron microscopy (FIG. 6).

### Example 4: Evaluation of Immunogenicity of 1st Generation Norovirus VP1 mRNA Vaccine in Mice

To investigate the immunogenicity of 1^{st} generation norovirus VP1 mRNA vaccines in vivo, a study using the BALB/c mouse model was designed. Mice were grouped by the “material” or type of VP1 mRNA vaccine received and by the dose ug/mouse received. Material groups included GI.1 Norwalk VP1 mRNA; GII.4 RockvilleD1 VP1 mRNA; and GI.1+GII.4 mRNA (1:1). Three additional material groups of PBS (blank), GI.1 VLP (protein), and GII.4 VLP (protein) were also tested. Dosage groups were as follows: 1 μg or 10 μg of GI.1 mRNA VP1 vaccine; 1 μg or 10 μg of GII.4 mRNA VP1 vaccine; 2 μg total or 20 μg total (1 μg of GI.1 mRNA VP1 vaccine and 1 μg of GII.4 mRNA VP1 vaccine or 10 μg of GI.1 mRNA VP1 vaccine and 10 μg of GI.4 mRNA VP1 vaccine); 10 μg of GI.1 VP1 recombinant protein, and 10 μg of GII.4 VP1 recombinant protein. The norovirus VP1 mRNA vaccines were administered intramuscularly to all groups on days 1 and boosted on day 22. Mice were bled on day 21 (D21) for a primary endpoint post-dose 1 “PD1” and bled on day 36 (D36) for a secondary endpoint post-dose 2 “PD2” on day 36.

PD1 and PD2 blood draws were evaluated by GI.1 VLP IgG ELISA and GII.4 VLP IgG ELISA. For ELISA, wells were coated with 100 uL/well VLP at a concentration of 1 ug/mL overnight at 4° C. Commercial VLPs obtained from The Native Antigen Company were used as ELISA antigens: GI.1 VLP comprised Hu/GI.1/CHA6A007/2010/USA VP1 and were produced in insect cells and GII.4 VLP comprises Hu/GII.4/CHDC2094/1974/US VP1 and was produced in 293T cells. Mouse sera dilution series started at 1:25. Anti-mouse IgG HRP was used for detection.

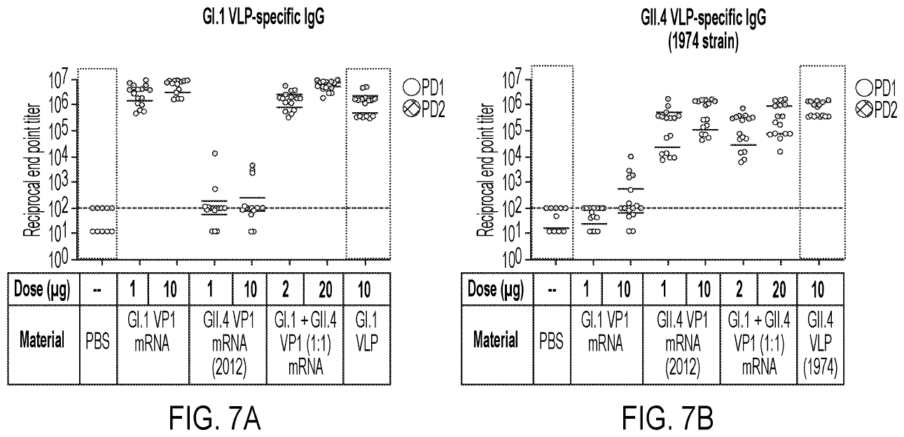

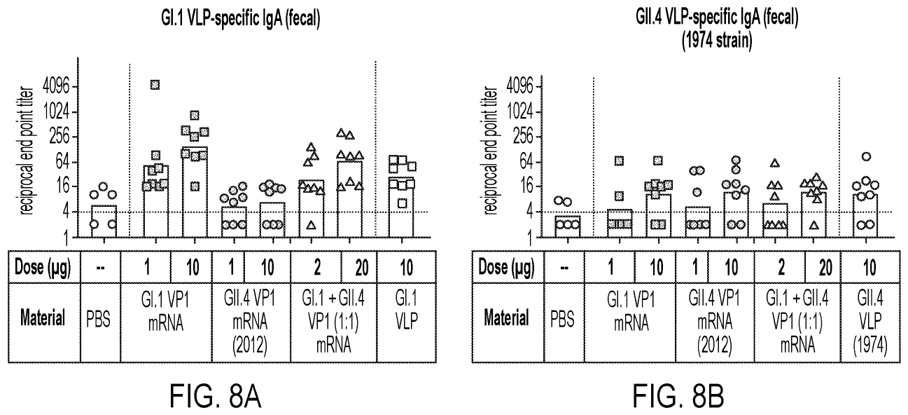

Vaccination with norovirus mRNA-encoding GI.1 and GII.4 both induced high levels of VP1-specific IgG (FIGS. 7A-7B). Similar antibody titers were achieved by 1 μg mRNA vs 10 μg mRNA. No antigen interference or synergy was observed when GI.1 and GII.4 mRNAs were co-administered (ratio 1:1). Furthermore, vaccination with norovirus mRNA-encoding GI.1 and GII.4 both induced VP1-specific IgA that could be detected in fecal extracts (FIGS. 8A-8B).

Additional primary endpoints (PD1) were evaluated by GI.1 VLP blockade assay and GII.4 VLP blockade assay. Blockade assays, which were developed as a surrogate neutralization assay, were conducted as follows. Plates were first coated with HBGA Pig gut mucin III (PGM). The PGM component comprises carbohydrates that norovirus VLPs can specifically bind. VLP+serum was transferred to PGM coated plates and incubated with decreasing amount of serum in a separate plate. Antibodies bound to norovirus VLP were able to block the binding of VLPs to the coated carbohydrates of the PGM. After the incubation period, unbound VLPs were washed away and ligand-bound VLPs were detected using an anti-VLP antibody with a biotinylated mouse-anti-VP1 secondary antibody. Bound VLP was detected with streptavidin-HRP.

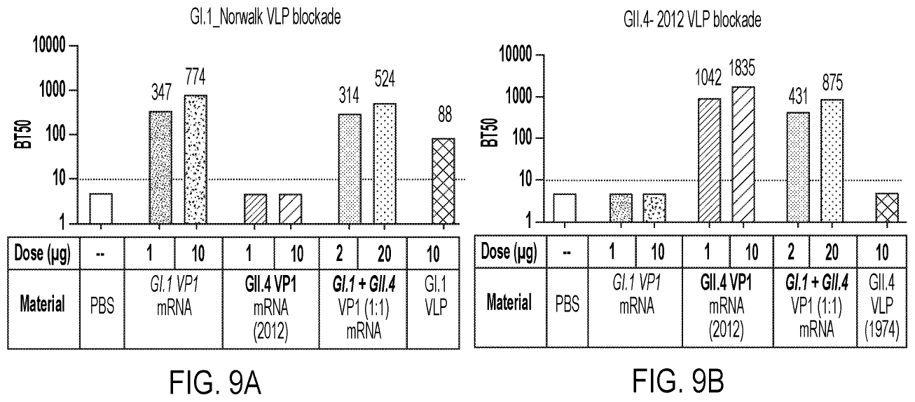

Vaccination with norovirus mRNA encoding GI.1 and GII.4 elicited blockade antibodies that were genotype- and strain-specific (FIGS. 9A-9B). No interference was detected when GI.1 and GII.4 VP1 mRNAs were co-administered (1:1).

### Example 5: Screening of Human Sera Against GI.1 VLP and GII.4 VLP

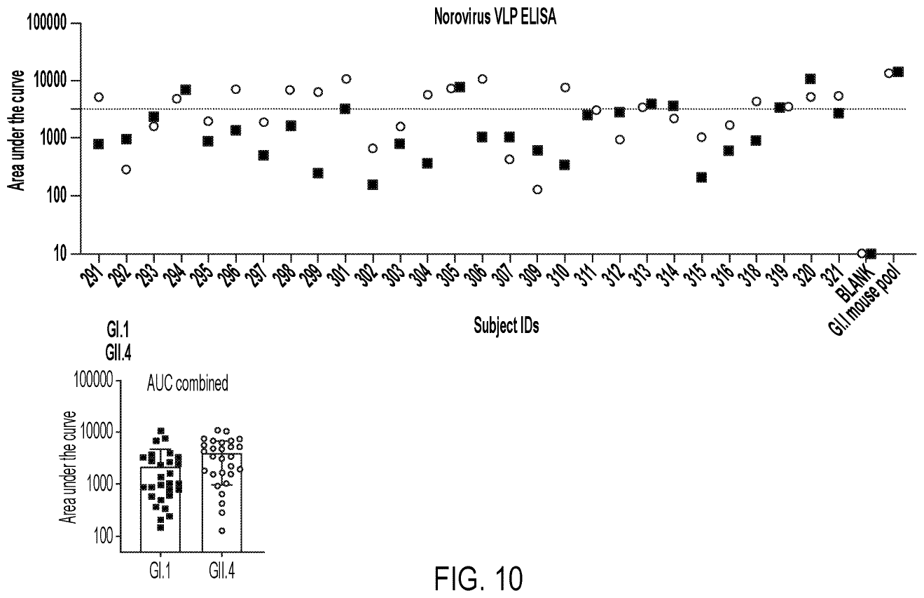

To investigate the seropositivity of human sera against the genotypes GI.1 and GII.4, human sera from 28 different subjects were evaluated against a Norovirus VLP in an ELISA as described above in Example 4 (FIG. 10). Most of the sera showed seropositivity against both genotypes, with binding antibody titers being overall higher against GII.4 than GI.1.

### Example 6: Evaluation of Norovirus VP1 mRNA with HRV 3CD Protease in Mice

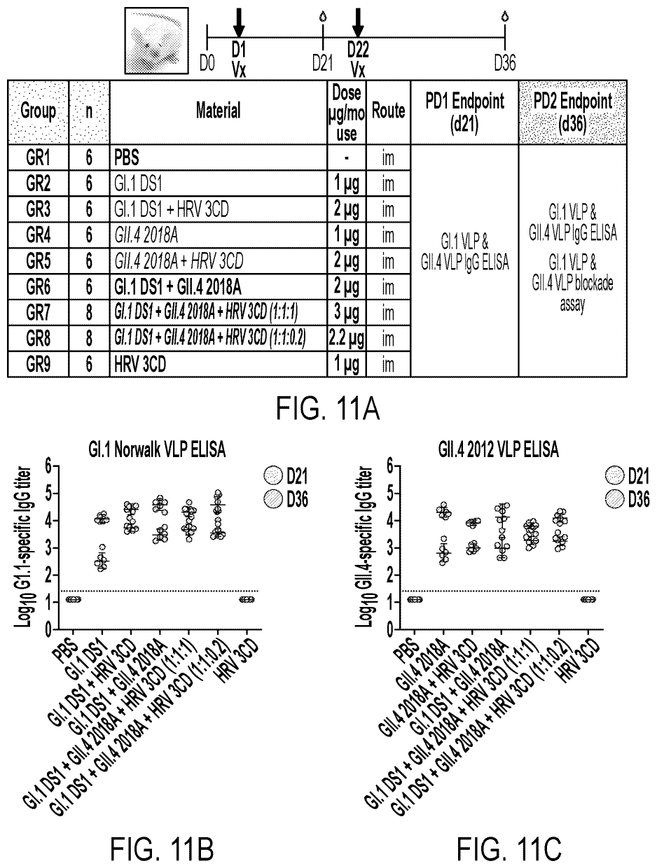

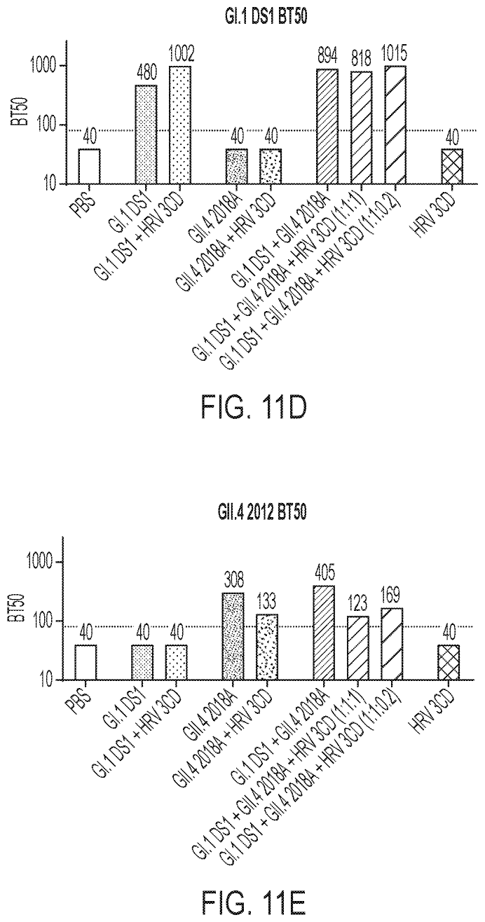

To investigate the stability of Norovirus GI.1 disulfide stabilized (DS1) and Norovirus GII.4 2018 mRNA vaccines in the presence of 3CD HRV protease, a mouse immunization study was conducted as follows. BALB/c mice (n=58) were immunized with on Day 1 and Day 22 with material from one of nine groups as shown in FIG. 11A. The primary (PD1) endpoint bleed was taken following the first dose on Day 21 and a secondary (PD2) endpoint bleed was taken following the second dose on Day 36. GI.1 VLP and GII.4 VLP IgG ELISA were conducted after the primary and secondary endpoints (FIGS. 11B-11C). GI.1 VLP and GII.4 VLP blockade assays were conducted after the secondary endpoint (FIGS. 11D-11E).

Immunization with HRV 3CD protease and VP1 mRNAs induced an increase in anti-GI.1 VP1 antibody titers (FIG. 11B) but not anti-GII.4 VP1 antibody titers (FIG. 11C). Immunization with HRV 3CD protease and VP1 mRNAs induced an increase in anti-GI.1 blockade titers (FIG. 11D) but is detrimental to anti-GII.4 blockade titers (FIG. 11E).

### Example 7: Evaluation of Novel Norovirus Genotypes and Multivalent GI and GII Combination mRNA Vaccines in Mice

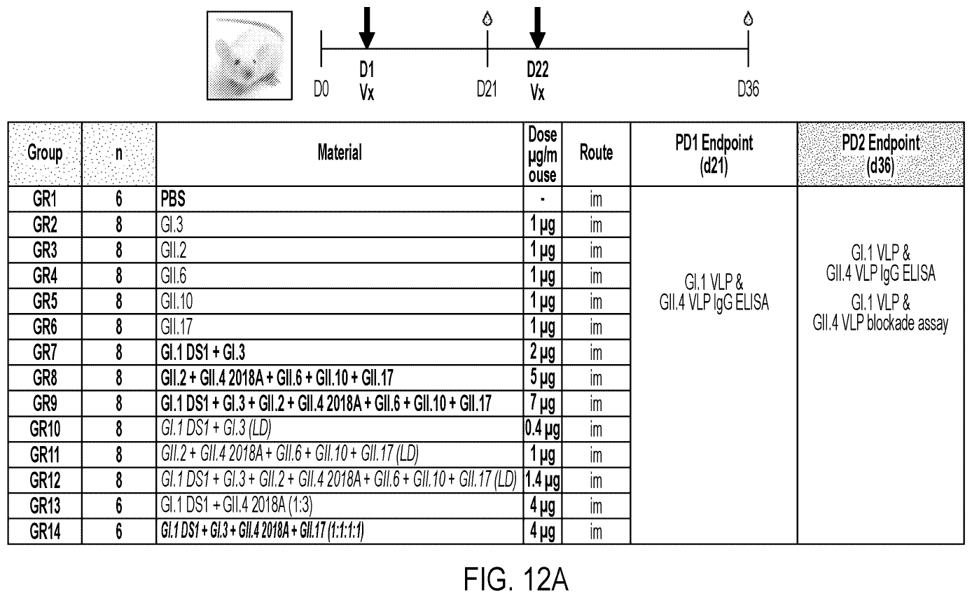

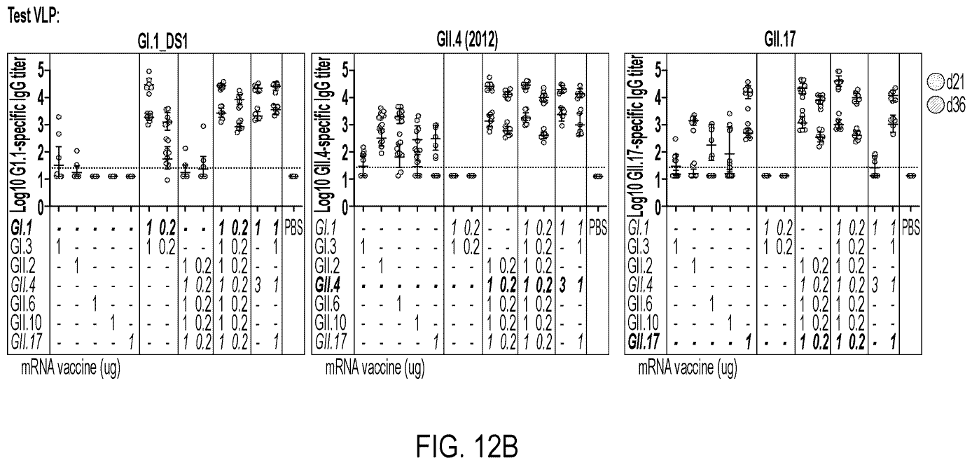

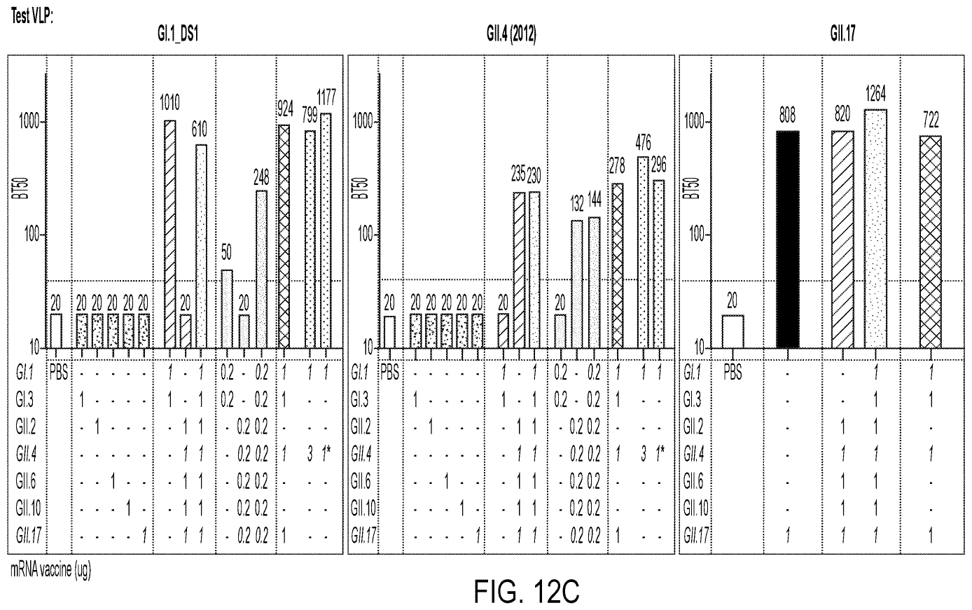

To investigate the immunogenicity of GII.6, GII.2, GII.17, GII.10, GI.3 mRNA vaccines, a mouse immunization study was conducted as follows. BALB/c mice (n=106) were immunized with on Day 1 and Day 22 with individual VLPs and combinations of VLP material from one of nine groups shown in FIG. 12A. The primary (PD1) endpoint bleed was taken following the first dose on Day 21 and a secondary (PD2) endpoint bleed was taken following the second dose on Day 36. GI.1 VLP, GII.4 VLP, and GII.17 VLP IgG ELISAs were conducted after the primary and secondary endpoints (FIG. 12B). GI.1 VLP, GII.4, and GII.17 VLP blockade assays were conducted after the secondary endpoint (FIG. 12C).

Cross-reactive anti-VP1 antibodies were induced within the same norovirus genogroup (GI or GII), and multivalent VP1 mRNA vaccines induced robust antibody titers (FIG. 12B). No significant interference was detected.

Multivalent VP1 mRNA vaccination resulted in strong blockade titers and GII.4 VP1-related interference could be recovered using a higher dose of GII.4 VP1 mRNA (FIG. 12C). No cross-reactivity was detected among genotypes and no significant interference was detected.

### Example 8: Evaluation of Different Ratios for Multivalent GI and GII Combination mRNA Vaccines in Mice

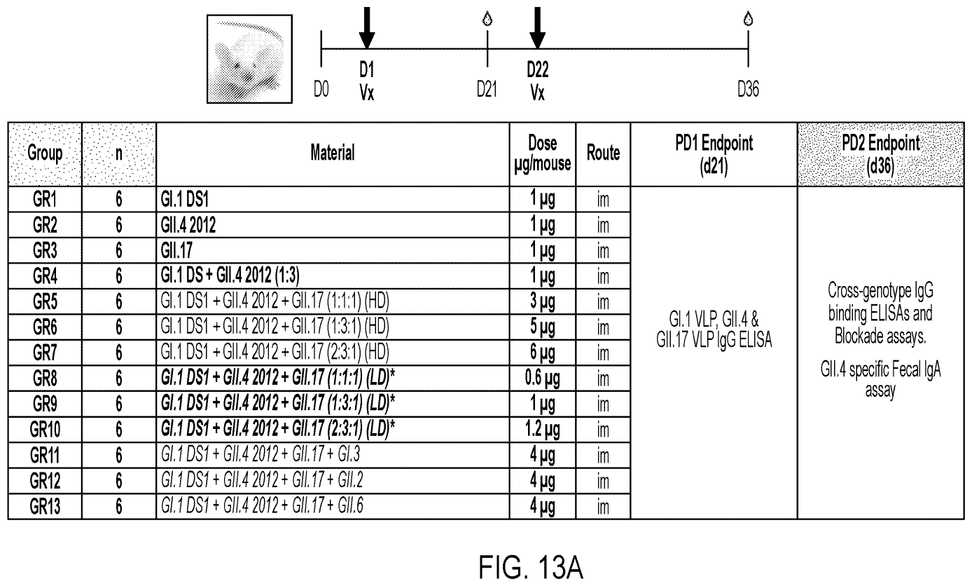

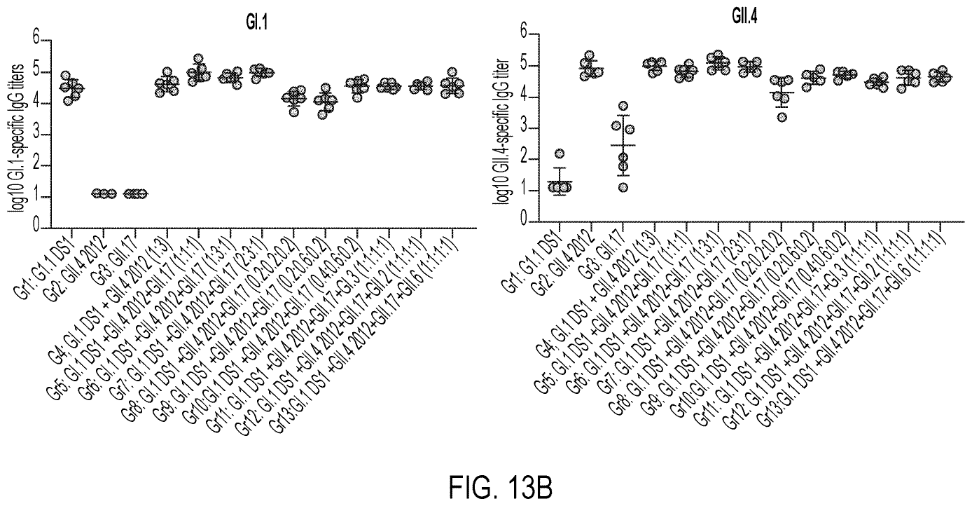

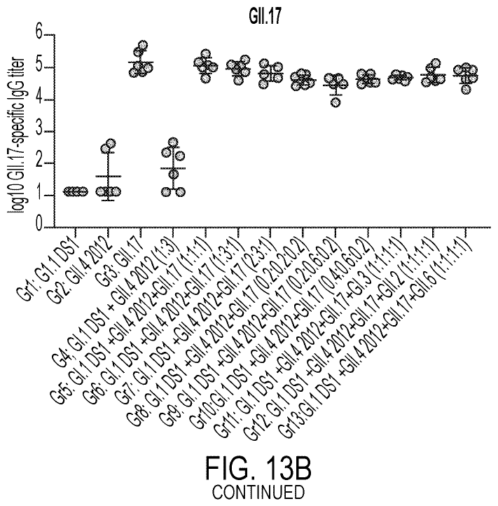

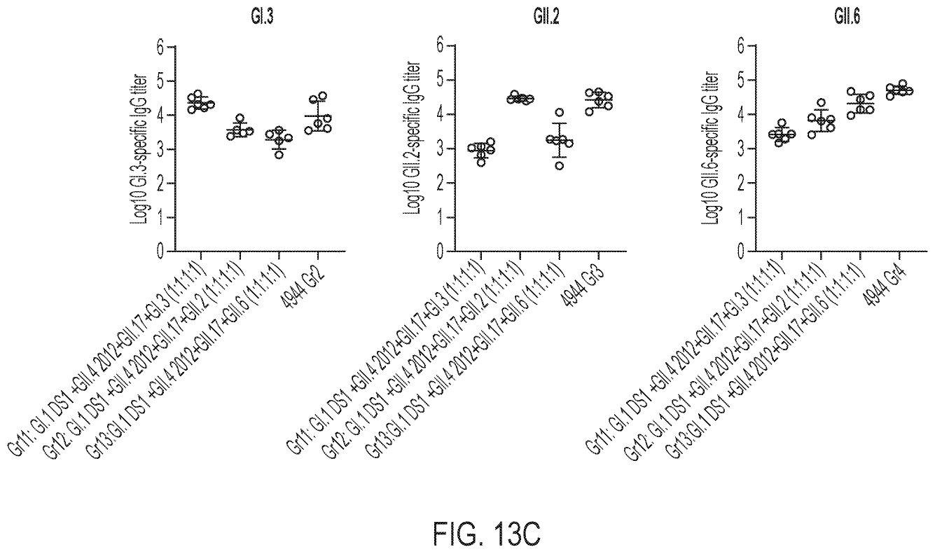

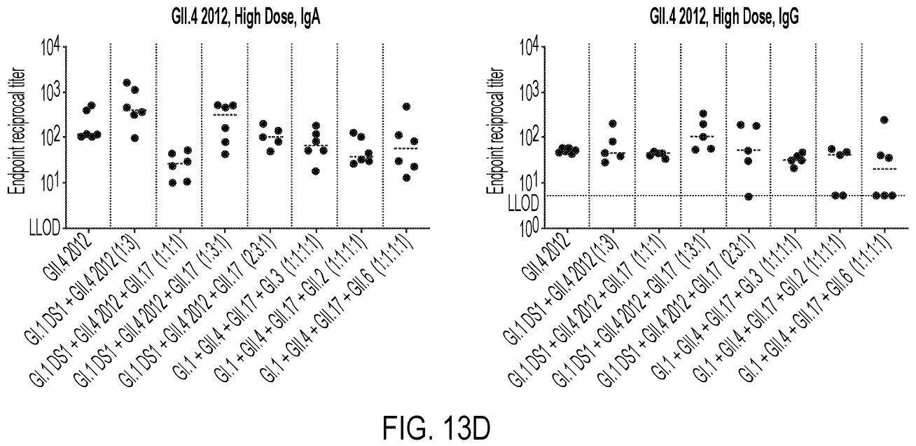

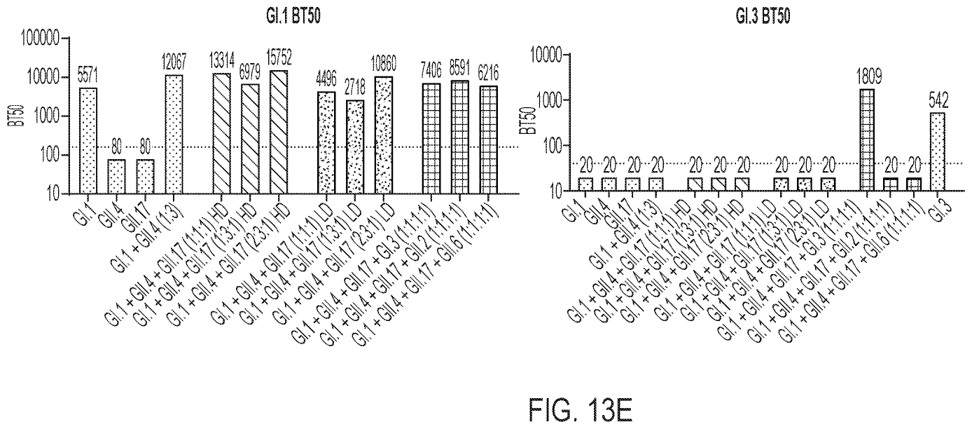

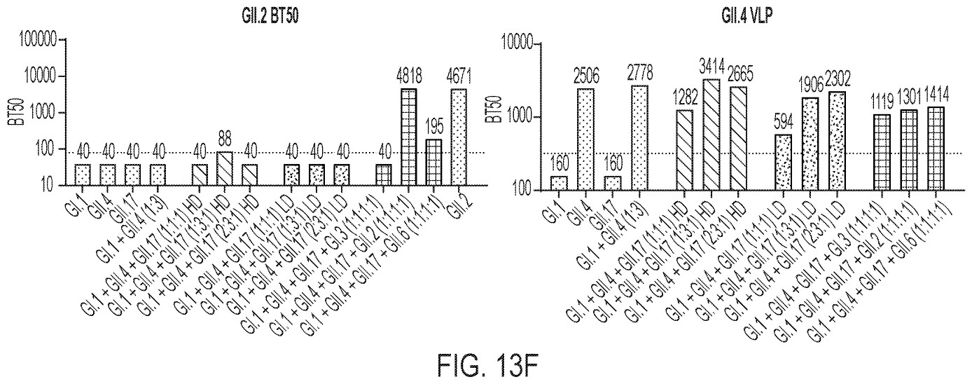

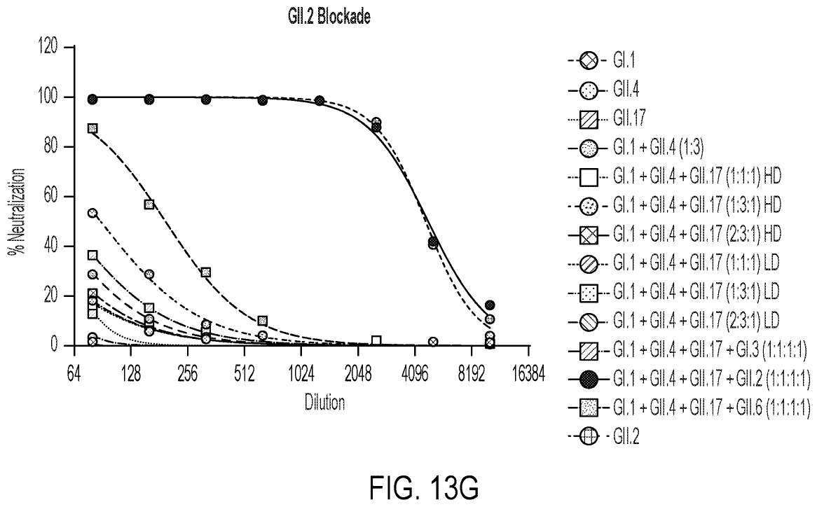

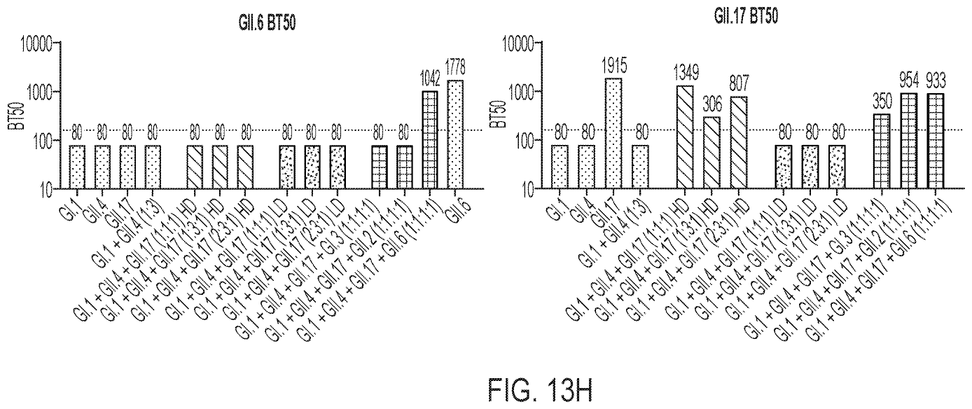

To investigate the immunogenicity of different ratios of multivalent GI and GII combination mRNA vaccines, a mouse immunization study was conducted as follows. BALB/c mice (n=78) were immunized with on Day 1 and Day 22 with material from one of thirteen groups as shown in FIG. 13A. The primary (PD1) endpoint bleed was taken following the first dose on Day 21 and a secondary (PD2) endpoint bleed was taken following the second dose on Day 36. GI.1 VLP, GII.4 VLP, and GII.17 VLP IgG ELISAs were conducted after the primary endpoint (FIG. 13B). Cross-genotype IgG binding ELISAs (FIG. 13C); GII.4 specific Fecal IgA assay (FIG. 13D) and Blockade assays (FIGS. 13E-13J) were conducted after the secondary endpoint.

Cross-reactive anti-VP1 antibodies were induced within the same genogroup and multivalent VP1 mRNA vaccines induced robust antibody titers (FIGS. 13B-13C). IgA and IgG GII.4 specific antibodies were found in murine fecal samples (FIG. 13D). Strong blockade titers were detected against GI.1 DS1 and GI.3 in sera from mice vaccinated with the respective mRNAs (FIG. 13E). GII.2 induced robust blockade titers against GII.2 and GII.4 VP1 mRNA induced low blockade titers against GII.2 (FIG. 13F). An increase of 3-fold higher GII.4 was capable of rescuing the interference (FIGS. 13F-13G). Finally, strong blockade titers were detected against GII.6 and GII.17 in sera from mice vaccinated with the respective mRNAs (FIG. 13H).

### Example 9: Determination of the Composition and Ratio of a Norovirus VP1 Based mRNA Vaccine

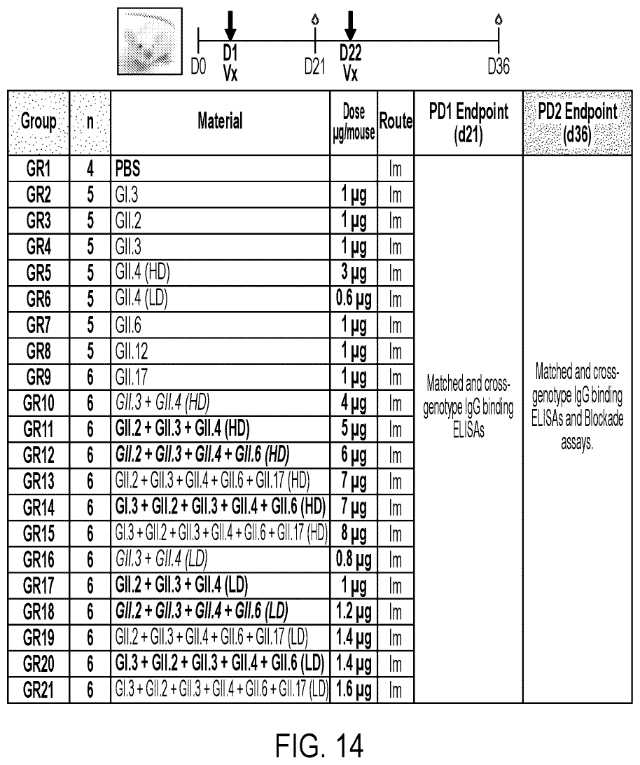

To investigate the composition and ratio of norovirus VP1 based mRNA vaccines in multiple different valencies, a mouse immunization study is conducted as follows. BALB/c mice (n=116) are immunized on Day 1 and Day 22 with material from one of twenty-one groups as shown in FIG. 14. The primary (PD1) endpoint bleed is taken following the first dose on Day 21 and a secondary (PD2) endpoint bleed is taken following the second dose on Day 36.

Matched and cross-genotype IgG binding ELISAs are conducted following the PD1 endpoint. Matched and cross-genotype IgG binding ELISAs and blockage assays are conducted following the PD2 endpoint.

### Example 10: Evaluation of Single and Multivalent mRNA Vaccines in Mice

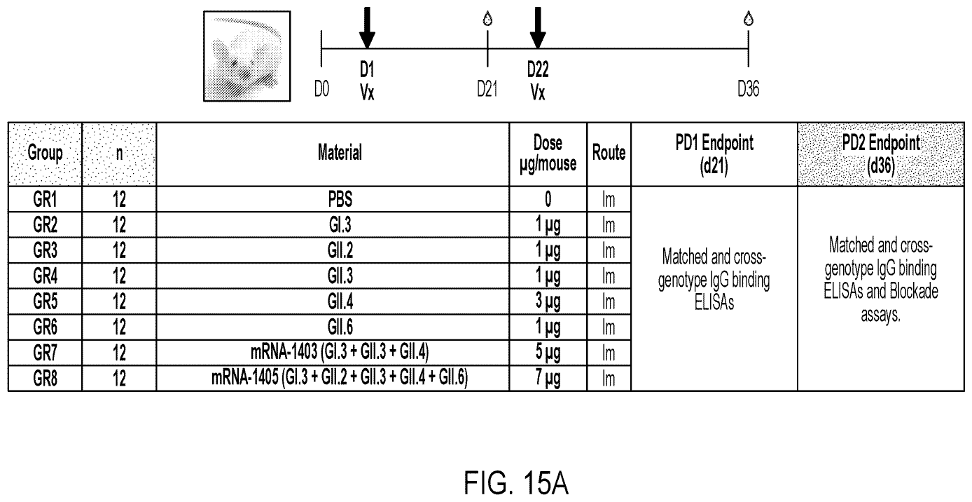

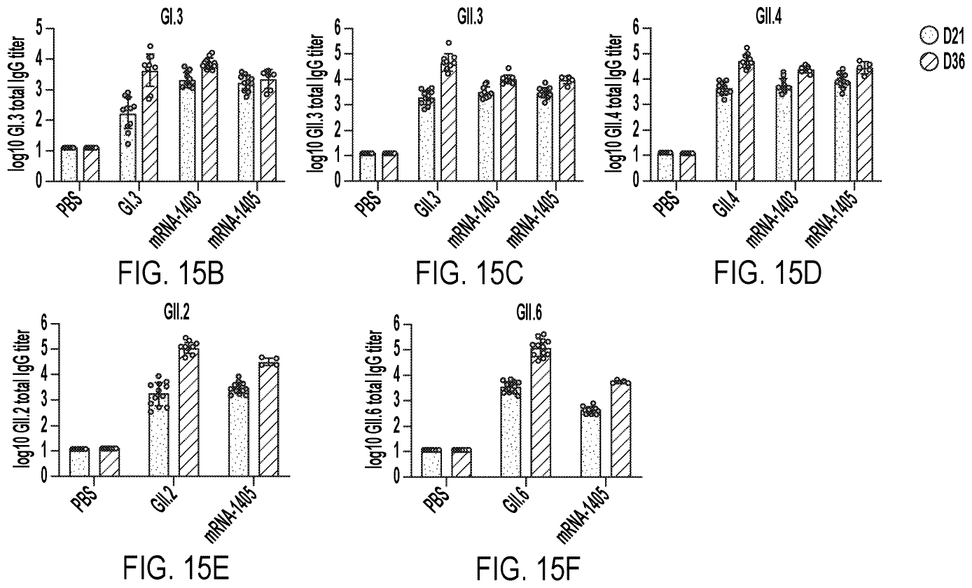

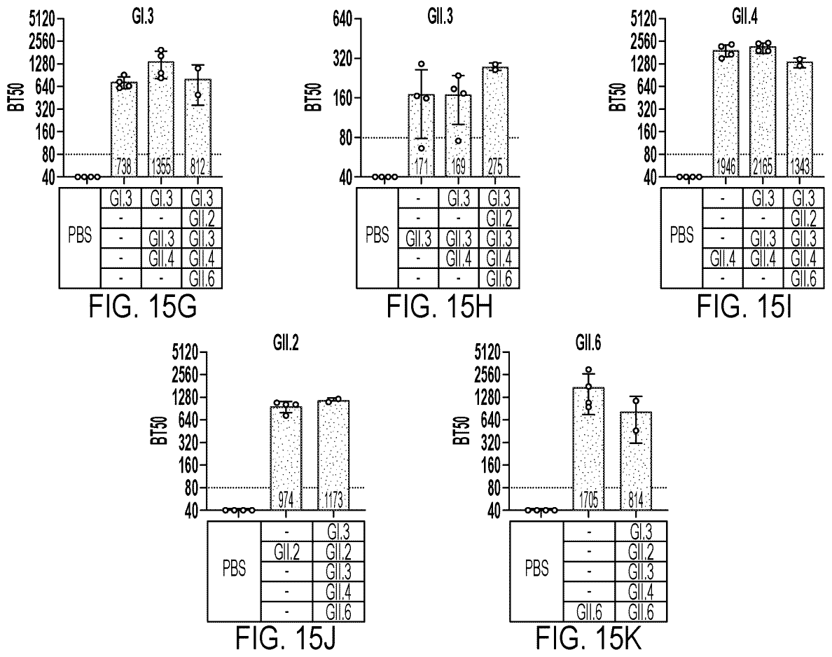

To investigate the immunogenicity of single and multivalent GI and GII mRNA vaccines, a mouse immunization study was conducted as follows. BALB/c mice (n=96) were immunized on Day 1 and Day 22 with material from one of eight groups as shown in FIG. 15A. The primary (PD1) endpoint bleed was taken following the first dose on Day 21 (left graph bars) and a secondary (PD2) endpoint bleed was taken following the second dose on Day 36 (right graph bars). Matched and cross-genotype IgG binding ELISAs were conducted after the primary endpoint and the secondary endpoint (FIGS. 15B-15F). Serum blockade titers were analyzed following the secondary endpoint (FIGS. 15G-15K).

Cross-reactive anti-VP1 antibodies were induced within the same genogroup and multivalent VP1 mRNA vaccines induced robust antibody titers, indicating that each norovirus VP1 mRNA-LNP component is immunogenic in mice (FIGS. 15B-15K).

### Example 11: Evaluation of mRNA-1403 and mRNA-1405 Multivalent Norovirus VP1-Based mRNA Vaccines in Dose-Ranging Study in Mice

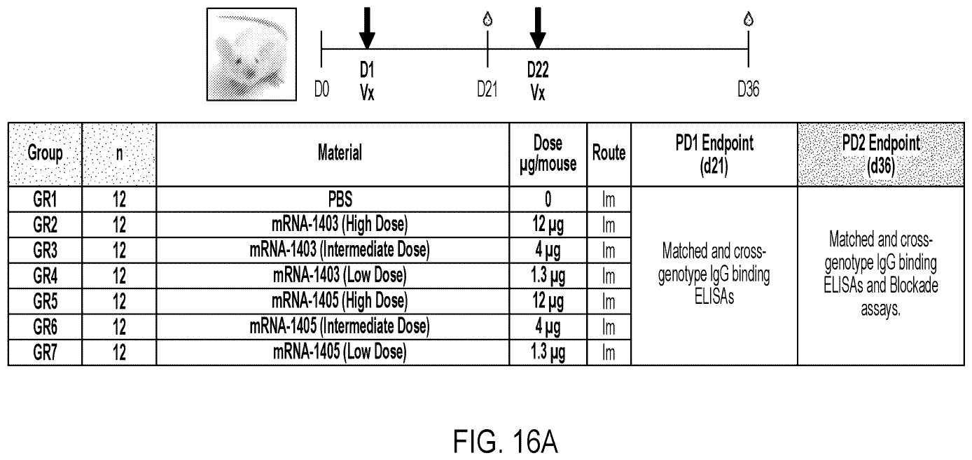

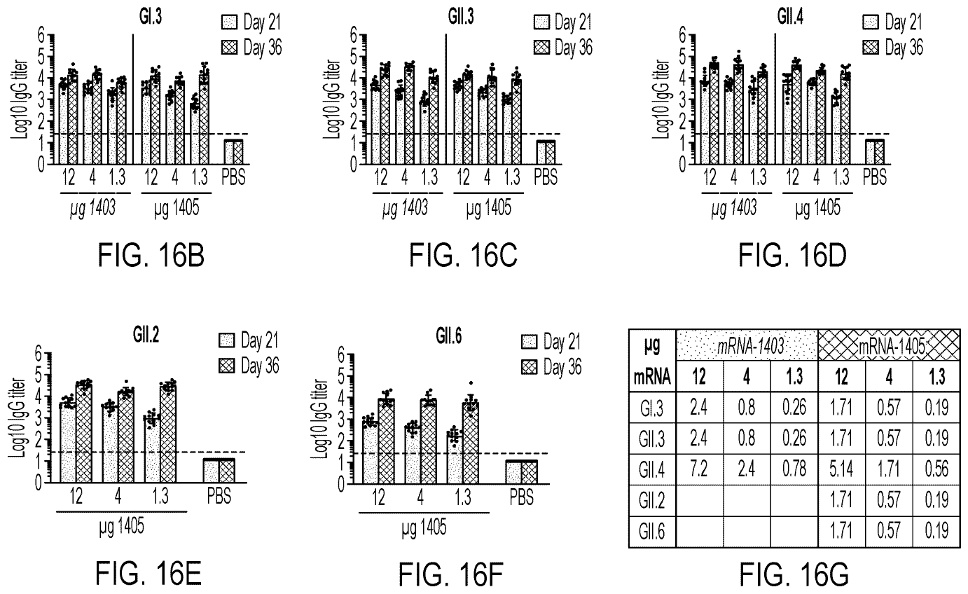

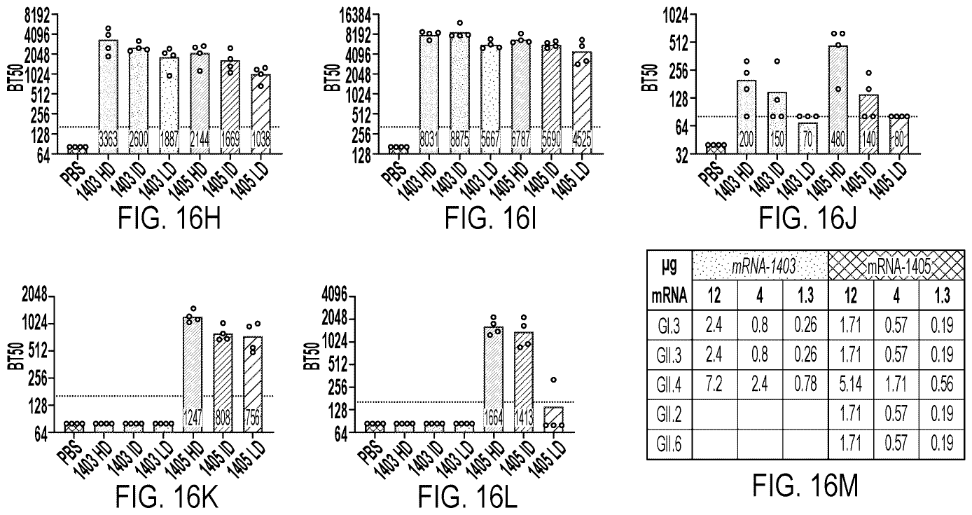

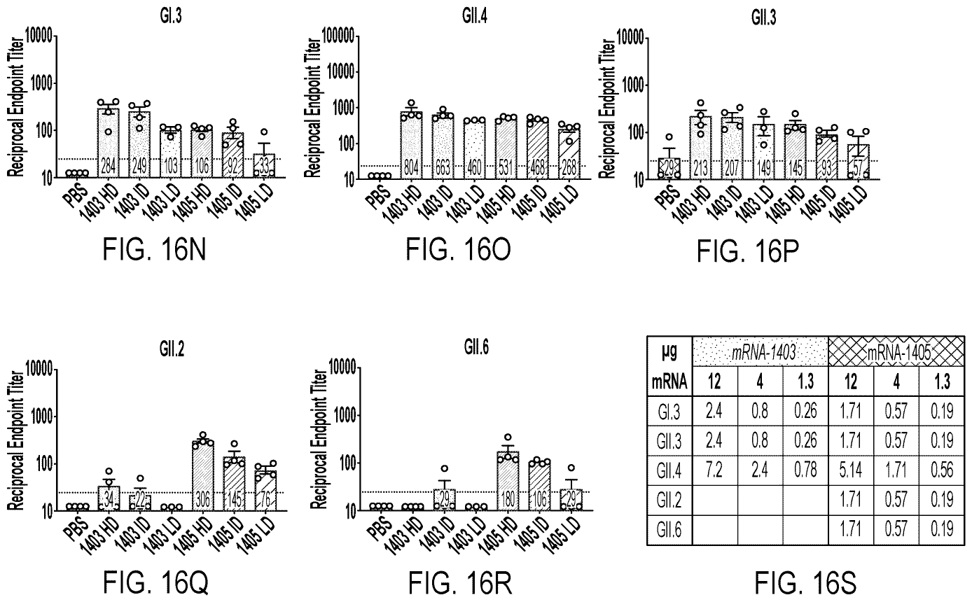

To investigate the immunogenicity of mRNA-1403 (GI.3+GII.3+GII.4) and mRNA-1405 (GI.3+GII.2+GII.3+GII.4+GII.6) multivalent Norovirus VP1-based mRNA vaccines, a dose-ranging mouse immunization study was conducted as follows. BALB/c mice (n=84) were immunized on Day 1 and Day 22 with material from one of seven groups as shown in FIG. 16A. The primary (PD1) endpoint bleed was taken following the first dose on Day 21 and a secondary (PD2) endpoint bleed was taken following the second dose on Day 36. Matched and cross-genotype IgG binding ELISAs were conducted after the primary endpoint and the secondary endpoint (FIGS. 16B-16G). Serum blockade titers were analyzed following the secondary endpoint (FIGS. 16H-16M). Fecal IgA responses were analyzed following the secondary endpoint (FIGS. 16N-16S).

Both mRNA-1403 and mRNA-1405 were immunogenic at all three dose levels (high=12 g, intermediate=4 μg and low=1.3 μg) when tested for serum IgG in binding titer ELISA (FIGS. 16B-16G). A serum IgG response was generated for each genotype in the multivalent composition. A dose-dependent response was observed for all genotypes on Day 21 (FIGS. 16B-16G). On Day 36, a weak dose response was observed for GII.3 and GII.4, while GI.3, GII.2 and GII.6 titers appeared to be saturated (FIGS. 16B-16G). Multivalent norovirus vaccine lots elicit dose dependent blockade responses in mice (FIGS. 16H-16M) and similarly elicit dose dependent fecal IgA responses in mice (FIGS. 16N-16S). Together, these data indicate that multivalent norovirus mRNA vaccines are immunogenic in mice.

### Example 12: Evaluation of mRNA-1403 and mRNA-1405 Multivalent Norovirus VP1-Based mRNA Vaccines in Rats

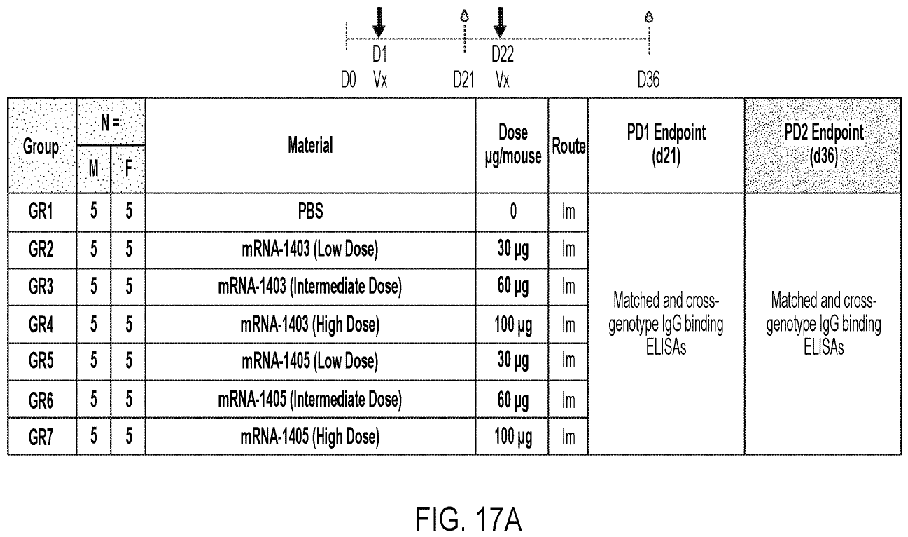

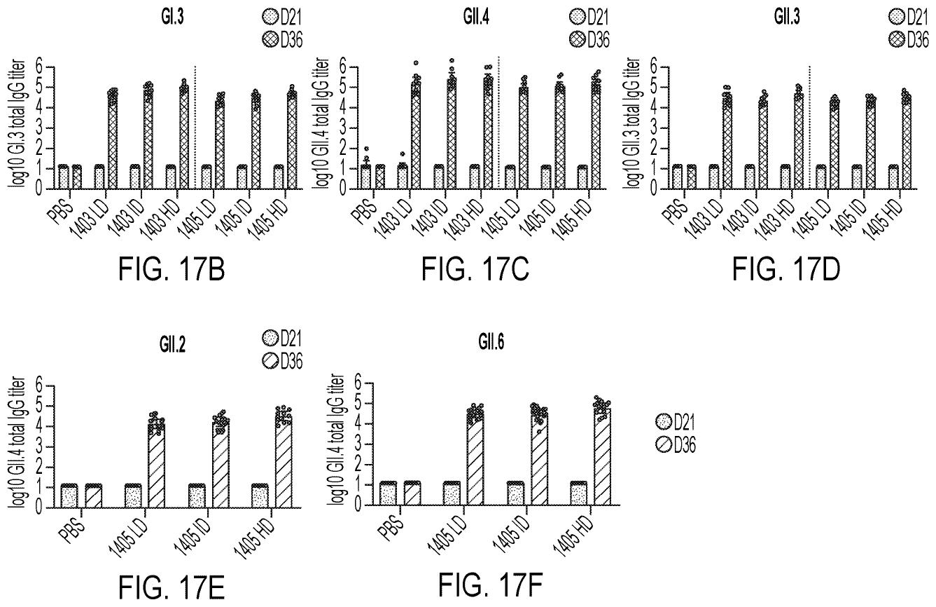

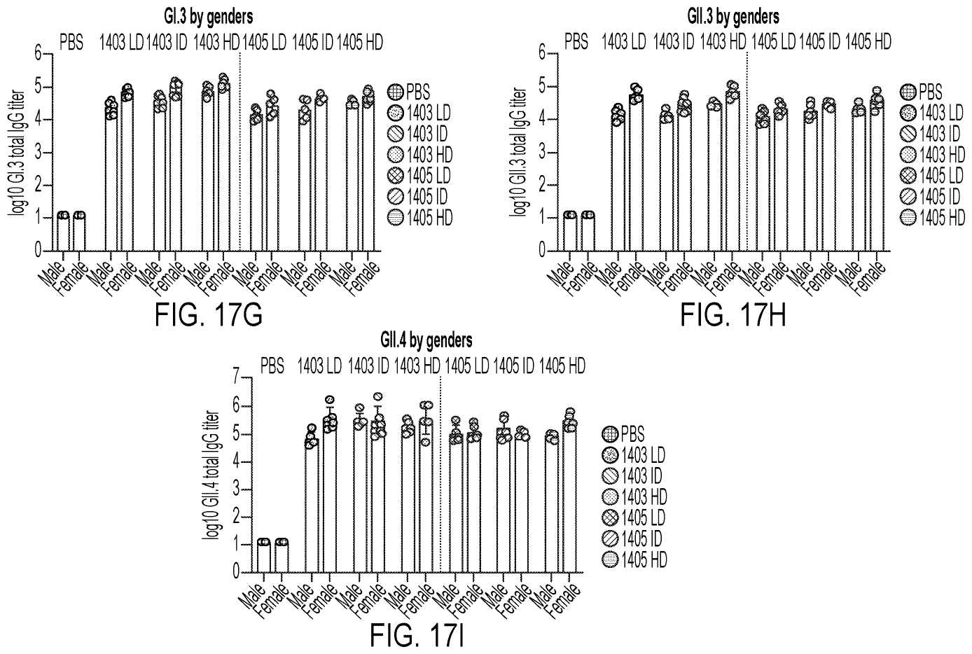

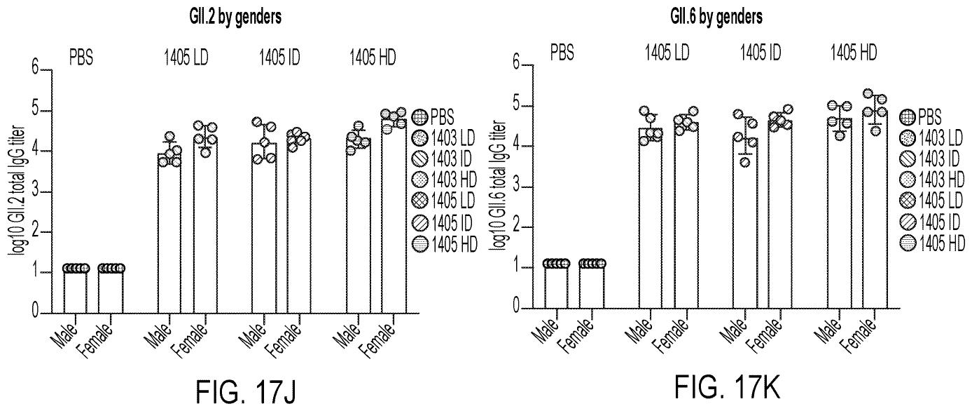

To investigate the immunogenicity of mRNA-1403 (GI.3+GII.3+GII.4) and mRNA-1405 (GI.3+GII.2+GII.3+GII.4+GII.6) multivalent Norovirus VP1-based mRNA vaccines, a dose-ranging rat immunization study was conducted as follows. Rats (n=70) were immunized on Day 1 and Day 22 with material from one of seven groups as shown in FIG. 17A. The primary (PD1) endpoint bleed was taken following the first dose on Day 21 (left graph bars) and a secondary (PD2) endpoint bleed was taken following the second dose on Day 36 (right graph bars). Matched and cross-genotype IgG binding ELISAs were conducted after the primary endpoint and the secondary endpoint (FIGS. 17B-17K).

Both mRNA-1403 and mRNA-1405 were immunogenic at all three dose levels (low=10 μg, intermediate=30 μg, and high=100 μg) when tested for serum IgG in binding titer ELISA (FIGS. 171B-17K). A serum IgG response was generated for each genotype in the multivalent composition and a dose-dependent response was observed for all genotypes on Day 36 (FIGS. 171B-17F). The serum IgG response differed slightly when rats were segregated by gender. Female rats were found to be slightly more immunogenic male rats across genotypes (FIGS. 17G-17K). Together, these data indicate that multivalent norovirus mRNA vaccines are immunogenic in rats.

TABLE 1
Sequences of the present disclosure
NV_GII.4_VP1

| SEQ ID NO: 147 consists of from 5′ end to 3′ end: 5′ UTR SEQ ID NO: 1, | | 147 |
|---|---|---|
| mRNA ORF SEQ ID NO: 2, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 2 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACCCCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCAACUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 3 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICNFRGDVTHITGSRNYTMNLASQNWNNYDP | |
| | TEEIPAPLGTPDFVGKIQGMLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

GI.1_Norwalk_1IHM_VP1

| SEQ ID NO: 148 consists of from 5′ end to 3′ end: 5′ UTR SEQ ID NO: 1, mRNA | | 148 |
|---|---|---|
| ORF SEQ ID NO: 5, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCACCAGCAGCGUGGACGG | 5 |
| Sequence | CGCUUCUGGGGCCGGACAGCUGGUGCCCGAGGUGAACGCCA | |
| (nucleotide) | GCGACCCACUGGCCAUGGACCCCGUGGCCGGAAGCAGCACC | |
| | GCCGUGGCCACCGCAGGACAGGUGAACCCCAUCGACCCUUG | |
| | GAUCAUCAACAACUUCGUGCAGGCACCCCAGGGCGAGUUCA | |
| | CCAUCAGCCCCAACAACACUCCCGGCGACGUGCUGUUCGAC | |
| | CUGUCCCUGGGCCCACACCUGAACCCCUUUCUGCUGCACCU | |
| | GAGCCAGAUGUACAACGGCUGGGUGGGCAACAUGCGGGUGC | |
| | GGAUCAUGCUGGCCGGCAACGCCUUUACCGCCGGCAAGAUC | |
| | AUCGUGAGCUGCAUCCCACCCGGCUUUGGCAGCCACAACCU | |
| | GACCAUCGCCCAGGCCACCCUGUUCCCACACGUGAUCGCCG | |
| | ACGUCCGGACCCUGGAUCCCAUCGAGGUGCCCCUGGAGGAC | |
| | GUGCGGAACGUGCUUUUCCACAACAACGACCGGAACCAGCA | |
| | GACCAUGCGCCUGGUGUGCAUGCUGUACACUCCACUUCGGA | |
| | CAGGCGGCGGAACCGGCGACAGCUUCGUGGUGGCCGGCCGG | |
| | GUGAUGACCUGCCCUUCCCCGGACUUCAACUUCCUGUUCCU | |
| | GGUGCCACCCACCGUGGAGCAGAAGACCCGGCCCUUCACCC | |
| | UGCCCAACCUGCCCCUGAGCAGCCUGAGCAACAGCCGGGCA | |
| | CCCCUGCCUAUCAGCAGCAUGGGCAUCUCACCCGACAACGU | |
| | GCAGAGCGUGCAGUUCCAGAACGGCAGGUGCACCCUCGACG | |
| | GCCGGCUGGUGGGCACCACGCCCGUGAGCCUGUCUCACGUG | |
| | GCCAAGAUCCGCGGCACCAGUAACGGCACCGUGAUUAAUCU | |
| | GACCGAGCUGGACGGCACUCCCUUCCACCCUUUCGAGGGCC | |
| | CCGCGCCAAUCGGAUUCCCCGACUUAGGUGGCUGCGACUGG | |
| | CACAUCAACAUGACCCAGUUCGGCCACAGCAGCCAGACCCA | |
| | GUACGACGUGGACACCACCCCUGACACCUUCGUGCCCCACC | |
| | UGGGCAGCAUCCAGGCCAACGGCAUCGGCAGCGGCAACUAC | |
| | GUGGGCGUGCUGAGCUGGAUCAGCCCUCCCAGCCACCCCAG | |
| | CGGCAGCCAAGUGGACCUGUGGAAGAUCCCCAACUACGGGA | |
| | GCAGCAUCACCGAAGCUACCCACCUCGCUCCCAGCGUGUAC | |
| | CCUCCUGGCUUCGGCGAGGUGCUGGUGUUCUUCAUGAGCAA | |
| | GAUGCCUGGCCCUGGCGCCUACAAUCUGCCCUGCCUGCUGC | |
| | CCCAGGAGUACAUCAGUCACCUGGCCAGCGAGCAGGCUCCA | |
| | ACAGUGGGCGAGGCCGCCUUACUGCACUACGUCGACCCCGA | |
| | CACCGGCCGGAACCUGGGAGAGUUCAAGGCCUACCCCGACG | |
| | GCUUCCUGACCUGCGUGCCCAACGGCGCAAGCAGCGGCCCU | |
| | CAGCAGCUGCCCAUCAACGGCGUGUUCGUGUUUGUGAGCUG | |
| | GGUGAGCCGGUUCUACCAGCUGAAGCCCGUAGGCACCGCCA | |
| | GCUCCGCUCGGGGAAGACUGGGCCUGCGGCGG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDATSSVDGASGAGQLVPEVNASDPLAMDPVAGSSTA | 6 |
| Amino Acid | VATAGQVNPIDPWIINNFVQAPQGEFTISPNNTPGDVLFDLSLGP | |
| Sequence | HLNPFLLHLSQMYNGWVGNMRVRIMLAGNAFTAGKIIVSCIPPG | |
| | FGSHNLTIAQATLFPHVIADVRTLDPIEVPLEDVRNVLFHNNDRN | |
| | QQTMRLVCMLYTPLRTGGGTGDSFVVAGRVMTCPSPDFNFLFL | |
| | VPPTVEQKTRPFTLPNLPLSSLSNSRAPLPISSMGISPDNVQSVQFQ | |
| | NGRCTLDGRLVGTTPVSLSHVAKIRGTSNGTVINLTELDGTPFHP | |
| | FEGPAPIGFPDLGGCDWHINMTQFGHSSQTQYDVDTTPDTFVPH | |
| | LGSIQANGIGSGNYVGVLSWISPPSHPSGSQVDLWKIPNYGSSITE | |
| | ATHLAPSVYPPGFGEVLVFFMSKMPGPGAYNLPCLLPQEYISHLA | |
| | SEQAPTVGEAALLHYVDPDTGRNLGEFKAYPDGFLTCVPNGASS | |
| | GPQQLPINGVFVFVSWVSRFYQLKPVGTASSARGRLGLRR | |
| PolyA tail | 100 nt | |

Mouse_Norovirus_6IUK_VP1

| SEQ ID NO: 149 consists of from 5′ end to 3′ end: 5′ UTR SEQ ID NO: 1, mRNA | | 149 |
|---|---|---|
| ORF SEQ ID NO: 7, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGCGGAUGAGCGACGGCGCCGCCCCUAAGGCCAACGGCAG | 7 |
| Sequence | UGAGGCCAGCGGCCAGGACUUGGUGCCCACCGCCGUGGAGC | |
| (nucleotide) | AGGCAGUGCCAAUCCAACCCGUGGCUGGCGCCGCUUUAGCC | |
| | GCCCCAGCAGCUGGCCAGAUCAACCAGAUAGACCCCUGGAU | |
| | CUUCCAGAACUUUGUGCAGUGUCCCCUGGGCGAGUUCAGCA | |
| | UCAGCCCUCGGAACACGCCCGGCGAGAUCCUGUUCGAUCUG | |
| | GCGCUGGGACCCGGCCUGAACCCCUACCUGGCCCACCUGAG | |
| | CGCCAUGUACACCGGCUGGGUGGGCAACAUGGAAGUGCAGC | |
| | UGGUGCUGGCCGGUAACGCCUUUACCGCCGGCAAGGUGGUG | |
| | GUGGCCCUGGUGCCACCCUACUUCCCCAAGGGCAGCCUGAC | |
| | CACCGCCCAGAUCACCUGCUUCCCUCACGUGAUGUGCGACG | |
| | UGAGAACCCUGGAGCCCAUCCAGCUGCCCCUGCUGGACGUG | |
| | CGGCGGGUGCUGUGGCACGCCACCCAGGACCAGGAGGAGAG | |
| | CAUGCGGCUGGUGUGCAUGCUGUACACCCCACUGCGGACCA | |
| | ACAGCCCCGGCGACGAGAGCUUCGUGGUGAGCGGCCGGCUG | |
| | CUCAGCAAGCCCGCCGCCGACUUCAACUUCGUGUACCUGAC | |
| | CCCUCCAAUCGAGCGGACCAUCUACCGGAUGGUGGACCUGC | |
| | CCGUGCUGCAACCCCGGCUGUGCACCCACGCCAGGUGGCCU | |
| | GCGCCAAUCUACGGCCUGCUGGUGGAUCCCAGCCUGCCCAG | |
| | CAACCCACAGUGGCAGAACGGCCGGGUGCACGUGGACGGCA | |
| | CCCUGCUUGGCACCACCCCUGUGAGCGGGAGCUGGGUGAGC | |
| | UGCUUCGCCGCAGAGGCCGCCUACGAGUUCCAGAGCGGCAU | |
| | CGGCGAGGUCGCCACCUUCACCCUGAUCGAGCAGGACGGCA | |
| | GCGCCUACGUGCCUGGAGACAGGGCCGCGCCUCUGGGCUAC | |
| | CCCGACUUUAGCGGACAGCUGGAGAUCGAGGUGCAGACCGA | |
| | GACCACCAAGAAGGGCGAGAAGCUGAAGGUGACCACCUUCG | |
| | AGAUGAUCCUGGGCCCCACGACCAACGUGGACCAGGUGCCC | |
| | UACCAGGGACGGGUGUACGCAAGCCUGACUGCCGCCGCCAG | |
| | CCUGGACCUGGUGGACGGACGAGUAAGAGCCGUGCCCCGGA | |
| | GCGUGUACGGCUUCCAGGACGUGGUGCCCGAGUACAACGAC | |
| | GGCCUGUUAGUGCCCCUGGCCCCACCCAUCGGCCCCUUCUU | |
| | ACCAGGCGAAGUCCUGCUGCGGUUCCGGACCUACAUGCGGC | |
| | AGAUCGACAGCACCGACGCUGCUGCCGAGGCCAUCGACUGC | |
| | GCCCUGCCCCAGGAGUUCGUGAGCUGGUUCGCCAGCAACGC | |
| | CUUCACCGUGCAGAGCGAGGCCCUGCUGCUGAGAUACCGGA | |
| | ACACCCUUACCGGUCAACUGCUGUUCGAGUGCAAGCUGUAU | |
| | AGCGAGGGCUACAUCGCCCUGAGCUACAGCGGCAGCGGCCC | |
| | UCUGACCUUCCCCACCGACGGCUUCUUCGAGGUGGUGUCUU | |
| | GGGUGCCACGGCUGUACCAGUUGGCCAGCGUGGGCUCUCUG | |
| | GCCACCGGCCGGACCCUGAAGCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MRMSDGAAPKANGSEASGQDLVPTAVEQAVPIQPVAGAALAAP | 8 |
| Amino Acid | AAGQINQIDPWIFQNFVQCPLGEFSISPRNTPGEILFDLALGPGLNP | |
| Sequence | YLAHLSAMYTGWVGNMEVQLVLAGNAFTAGKVVVALVPPYFP | |
| | KGSLTTAQITCFPHVMCDVRTLEPIQLPLLDVRRVLWHATQDQE | |
| | ESMRLVCMLYTPLRTNSPGDESFVVSGRLLSKPAADFNFVYLTPP | |
| | IERTIYRMVDLPVLQPRLCTHARWPAPIYGLLVDPSLPSNPQWQN | |
| | GRVHVDGTLLGTTPVSGSWVSCFAAEAAYEFQSGIGEVATFTLIE | |
| | QDGSAYVPGDRAAPLGYPDFSGQLEIEVQTETTKKGEKLKVTTF | |
| | EMILGPTTNVDQVPYQGRVYASLTAAASLDLVDGRVRAVPRSV | |
| | YGFQDVVPEYNDGLLVPLAPPIGPFLPGEVLLRFRTYMRQIDSTD | |
| | AAAEAIDCALPQEFVSWFASNAFTVQSEALLLRYRNTLTGQLLF | |
| | ECKLYSEGYIALSYSGSGPLTFPTDGFFEVVSWVPRLYQLASVGS | |
| | LATGRTLKQ | |
| PolyA tail | 100 nt | |

GII.4_2018A_B403

| SEQ ID NO: 150 consists of from 5′ end to 3′ end: 5′ UTR SEQ ID NO: 1, mRNA | | 150 |
|---|---|---|
| ORF SEQ ID NO: 9, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 9 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCACGACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | UGCACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MRMSDGAAPKANGSEASGQDLVPTAVEQAVPIQPVAGAALAAP | 10 |
| Amino Acid | AAGQINQIDPWIFQNFVQCPLGEFSISPRNTPGEILFDLALGPGLNP | |
| Sequence | YLAHLSAMYTGWVGNMEVQLVLAGNAFTAGKVVVALVPPYFP | |
| | KGSLTTAQITCFPHVMCDVRTLEPIQLPLLDVRRVLWHATQDQE | |
| | ESMRLVCMLYTPLRTNSPGDESFVVSGRLLSKPAADFNFVYLTPP | |
| | IERTIYRMVDLPVLQPRLCTHARWPAPIYGLLVDPSLPSNPQWQN | |
| | GRVHVDGTLLGTTPVSGSWVSCFAAEAAYEFQSGIGEVATFTLIE | |
| | QDGSAYVPGDRAAPLGYPDFSGQLEIEVQTETTKKGEKLKVTTF | |
| | EMILGPTTNVDQVPYQGRVYASLTAAASLDLVDGRVRAVPRSV | |
| | YGFQDVVPEYNDGLLVPLAPPIGPFLPGEVLLRFRTYMRQIDSTD | |
| | AAAEAIDCALPQEFVSWFASNAFTVQSEALLLRYRNTLTGQLLF | |
| | ECKLYSEGYIALSYSGSGPLTFPTDGFFEVVSWVPRLYQLASVGS | |
| | LATGRTLKQ | |
| PolyA tail | 100 nt | |

GII.4_2004A

| SEQ ID NO: 151 consists of from 5′ end to 3′ end: 5′ UTR SEQ ID NO: 1, mRNA | | 151 |
|---|---|---|
| ORF SEQ ID NO: 11, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 11 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACCCCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCAACUUCCGG | |
| | GGCGACGUGACCCACAUCGCCGACACCCACAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGCUACC | |
| | GACACCGCUGCUGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 12 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICNFRGDVTHIADTHNYTMNLASQNWNNYD | |
| | PTEEIPAPLGTPDFVGKIQGMLTQTTRTDGSTRGHKATVYTGSAD | |
| | FAPKLGRVQFATDTAADFEANQNTKFTPVGVIQDGSTTHRNEPQ | |
| | QWVLPSYSGRNTHNVHLAPAVAPTFPGEQLLFFRSTMPGCSGYP | |
| | NMDLDCLLPQEWVQYFYQEAAPAQSDVALLRFVNPDTGRVLFE | |
| | CKLHKSGYVTVAHTGQHDLVIPPNGYFRFDSWVNQFYTLAPMG | |
| | NGTGRRRVV | |
| PolyA tail | 100 nt | |

GI.1_VP1_DS1

| SEQ ID NO: 152 consists of from 5′ end to 3′ end: 5′ UTR SEQ ID NO: 1, mRNA | | 152 |
|---|---|---|
| ORF SEQ ID NO: 13, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCACCAGCAGCGUGGACGG | 13 |
| Sequence | CGCUUCUGGGGCCGGACAGCUGGUGCCCGAGGUGAACGCCA | |
| (nucleotide) | GCGACCCACUGGCCAUGGACCCCGUGGCCGGAAGCAGCACC | |
| | GCCGUGGCCACCGCAGGACAGGUGAACCCCAUCGACCCUUG | |
| | GAUCAUCAACAACUUCGUGCAGGCACCCCAGGGCGAGUUCA | |
| | CCAUCAGCCCCAACAACACUCCCGGCGACGUGCUGUUCGAC | |
| | CUGUCCCUGGGCCCACACCUGAACCCCUUUCUGCUGCACCU | |
| | GAGCCAGAUGUACAACGGCUGGGUGGGCAACAUGCGGGUGC | |
| | GGAUCAUGCUGGCCGGCUGCGCCUUUACCGCCGGCAAGAUC | |
| | AUCGUGAGCUGCAUCCCACCCGGCUUUGGCAGCCACAACCU | |
| | GACCAUCGCCCAGGCCACCCUGUUCCCACACGUGAUCGCCG | |
| | ACGUCCGGACCCUGGAUCCCAUCGAGGUGCCCCUGGAGGAC | |
| | GUGCGGAACGUGCUUUUCCACAACAACGACCGGAACCAGCA | |
| | GACCAUGCGCCUGGUGUGCAUGCUGUACACUCCACUUCGGA | |
| | CAUGCGGCGGAACCGGCGACAGCUUCGUGGUGGCCGGCCGG | |
| | GUGAUGACCUGCCCUUCCCCGGACUUCAACUUCCUGUUCCU | |
| | GGUGCCACCCACCGUGGAGCAGAAGACCCGGCCCUUCACCC | |
| | UGCCCAACCUGCCCCUGAGCAGCCUGAGCAACAGCCGGGCA | |
| | CCCCUGCCUAUCAGCAGCAUGGGCAUCUCACCCGACAACGU | |
| | GCAGAGCGUGCAGUUCCAGAACGGCAGGUGCACCCUCGACG | |
| | GCCGGCUGGUGGGCACCACGCCCGUGAGCCUGUCUCACGUG | |
| | GCCAAGAUCCGCGGCACCAGUAACGGCACCGUGAUUAAUCU | |
| | GACCGAGCUGGACGGCACUCCCUUCCACCCUUUCGAGGGCC | |
| | CCGCGCCAAUCGGAUUCCCCGACUUAGGUGGCUGCGACUGG | |
| | CACAUCAACAUGACCCAGUUCGGCCACAGCAGCCAGACCCA | |
| | GUACGACGUGGACACCACCCCUGACACCUUCGUGCCCCACC | |
| | UGGGCAGCAUCCAGGCCAACGGCAUCGGCAGCGGCAACUAC | |
| | GUGGGCGUGCUGAGCUGGAUCAGCCCUCCCAGCCACCCCAG | |
| | CGGCAGCCAAGUGGACCUGUGGAAGAUCCCCAACUACGGGA | |
| | GCAGCAUCACCGAAGCUACCCACCUCGCUCCCAGCGUGUAC | |
| | CCUCCUGGCUUCGGCGAGGUGCUGGUGUUCUUCAUGAGCAA | |
| | GAUGCCUGGCCCUGGCGCCUACAAUCUGCCCUGCCUGCUGC | |
| | CCCAGGAGUACAUCAGUCACCUGGCCAGCGAGCAGGCUCCA | |
| | ACAGUGGGCGAGGCCGCCUUACUGCACUACGUCGACCCCGA | |
| | CACCGGCCGGAACCUGGGAGAGUUCAAGGCCUACCCCGACG | |
| | GCUUCCUGACCUGCGUGCCCAACGGCGCAAGCAGCGGCCCU | |
| | CAGCAGCUGCCCAUCAACGGCGUGUUCGUGUUUGUGAGCUG | |
| | GGUGAGCCGGUUCUACCAGCUGAAGCCCGUAGGCACCGCCA | |
| | GCUCCGCUCGGGGAAGACUGGGCCUGCGGCGG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDATSSVDGASGAGQLVPEVNASDPLAMDPVAGSSTA | 14 |
| Amino Acid | VATAGQVNPIDPWIINNFVQAPQGEFTISPNNTPGDVLFDLSLGP | |
| Sequence | HLNPFLLHLSQMYNGWVGNMRVRIMLAGCAFTAGKIIVSCIPPG | |
| | FGSHNLTIAQATLFPHVIADVRTLDPIEVPLEDVRNVLFHNNDRN | |
| | QQTMRLVCMLYTPLRTCGGTGDSFVVAGRVMTCPSPDFNFLFL | |
| | VPPTVEQKTRPFTLPNLPLSSLSNSRAPLPISSMGISPDNVQSVQFQ | |
| | NGRCTLDGRLVGTTPVSLSHVAKIRGTSNGTVINLTELDGTPFHP | |
| | FEGPAPIGFPDLGGCDWHINMTQFGHSSQTQYDVDTTPDTFVPH | |
| | LGSIQANGIGSGNYVGVLSWISPPSHPSGSQVDLWKIPNYGSSITE | |
| | ATHLAPSVYPPGFGEVLVFFMSKMPGPGAYNLPCLLPQEYISHLA | |
| | SEQAPTVGEAALLHYVDPDTGRNLGEFKAYPDGFLTCVPNGASS | |
| | GPQQLPINGVFVFVSWVSRFYQLKPVGTASSARGRLGLRR | |
| PolyA tail | 100 nt | |

GII.6_VP1

| SEQ ID NO: 153 consists of from 5′ end to 3′ end: 5′ UTR SEQ ID NO: 1, mRNA | | 153 |
|---|---|---|
| ORF SEQ ID NO: 15, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCCCCAAGCAACGACGG | 15 |
| Sequence | CGCCGCCAACCUGGUGCCCGAGGCCACCAACGAGGUGAUGG | |
| (nucleotide) | CCCUGGAGCCAGUGGUGGGCGCCUCCAUCGCUGCCCCUGUG | |
| | GUGGGCCAGCAGAACAUCAUCGACCCCUGGAUCCGGGAGAA | |
| | CUUCGUGCAGGCCCCUCAGGGCGAGUUCACCGUGUCUCCCC | |
| | GGAACAGCCCCGGCGAGAUGCUGCUGAACCUGGAGCUGGGC | |
| | CCCGAGCUGAACCCCUACCUGAGCCACCUGAGCCGGAUGUA | |
| | CAACGGCUACGCCGGCGGCAUGCAGGUGCAGGUGGUGCUGG | |
| | CCGGCAACGCCUUCACCGCCGGCAAGAUCAUCUUCGCCGCC | |
| | GUGCCACCCCACUUCCCCGUGGAGAACAUCAGCGCCGCCCA | |
| | GAUCACCAUGUGUCCCCACGUGAUCGUGGACGUGCGGCAGC | |
| | UGGAGCCCGUGCUGCUGCCCCUGCCCGACAUCCGGAACCGG | |
| | UUCUUCCACUACAACCAGGAGAACACACCCCGGAUGCGGCU | |
| | GGUGGCCAUGCUGUAUACUCCCCUGCGGGCCAAUAGCGGCG | |
| | AGGACGUGUUCACCGUGAGCUGUCGGGUGCUGACCAGGCCC | |
| | GCACCCGACUUCGAGUUCACCUUCCUGGUGCCGCCCACCGU | |
| | GGAGAGCAAGACCAAGCCCUUCACCCUGCCCAUCCUGACCC | |
| | UGGGCGAGCUGAGCAACAGCCGGUUCCCCGCCCCUAUCGAC | |
| | AUGCUGUACACCGACCCCAACGAGGCCAUCGUGGUGCAGCC | |
| | CCAGAACGGCCGGUGCACCUUGGACGGCACCCUGCAGGGCA | |
| | CCACCCAGCUGGUGCCCACCCAGAUCUGCAGCUUCCGGGGC | |
| | ACCCUGAUCAGCCAGACCAGCCGGAGCGCCGACAGCACCGA | |
| | CAGCGCACCCCGGGUGCGGAACCAUCCCCUCCACGUGCAGC | |
| | UGAAGAACCUGGACGGCACUCCCUACGACCCCACCGACGAA | |
| | GUGCCCGCAGUGCUGGGCGCCAUCGACUUCAAGGGCACCGU | |
| | GUUCGGCGUGGCCAGCCAGCGGAACACCACCGGCAACUCUA | |
| | UAGGCGCCACCAGAGCCCACGAGGUGCACAUCGACACCACC | |
| | AAUCCCCGGUACACCCCUAAGCUGGGCAGCGUGCUGAUGUA | |
| | CAGCGAGAGCACCGACUUCGACGACGGCCAGCCCACCCGGU | |
| | UCACUCCCAUCGGCAUGGGCGCCGACGACUGGCACCAGUGG | |
| | GAGCUGCCCGAGUACAGCGGCCACCUGACCCUGAACAUGAA | |
| | CUUAGCCCCAGCCGUGGCUCCAGCAUUCCCCGGCGAGCGGA | |
| | UCCUGUUCUUCCGGAGCGUGGUGCCCUCAGCCGGCGGUUAC | |
| | GGCAGCGGCCACAUCGACUGCCUGAUUCCCCAGGAGUGGGU | |
| | GCAGCACUUCUACCAGGAGGCCGCCCCUAGCCAGAGCGCCG | |
| | UGGCCCUGAUCCGGUACGUGAACCCCGACACCGGCCGGAAC | |
| | AUCUUCGAGGCCAAGCUGCACCGGGAGGGCUUCAUCACCGU | |
| | CGCCAACAGCGGCAACAACCCCAUCGUGGUUCCUCCGAACG | |
| | GCUACUUCCGGUUCGAGGCCUGGGUGAACCAGUUCUACACC | |
| | CUGACCCCAAUGGGCACAGGCCAGGGCCGACGGAGGGUGCA | |
| | G | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSNDGAANLVPEATNEVMALEPVVGASIAAPVV | 16 |
| Amino Acid | GQQNIIDPWIRENFVQAPQGEFTVSPRNSPGEMLLNLELGPELNP | |
| Sequence | YLSHLSRMYNGYAGGMQVQVVLAGNAFTAGKIIFAAVPPHFPV | |
| | ENISAAQITMCPHVIVDVRQLEPVLLPLPDIRNRFFHYNQENTPR | |
| | MRLVAMLYTPLRANSGEDVFTVSCRVLTRPAPDFEFTFLVPPTV | |
| | ESKTKPFTLPILTLGELSNSRFPAPIDMLYTDPNEAIVVQPQNGRC | |
| | TLDGTLQGTTQLVPTQICSFRGTLISQTSRSADSTDSAPRVRNHPL | |
| | HVQLKNLDGTPYDPTDEVPAVLGAIDFKGTVFGVASQRNTTGNS | |
| | IGATRAHEVHIDTTNPRYTPKLGSVLMYSESTDFDDGQPTRFTPI | |
| | GMGADDWHQWELPEYSGHLTLNMNLAPAVAPAFPGERILFFRS | |
| | VVPSAGGYGSGHIDCLIPQEWVQHFYQEAAPSQSAVALIRYVNP | |
| | DTGRNIFEAKLHREGFITVANSGNNPIVVPPNGYFRFEAWVNQFY | |
| | TLTPMGTGQGRRRVQ | |
| PolyA tail | 100 nt | |

GII.2_VP1

| SEQ ID NO: 154 consists of from 5′ end to 3′ end: 5′ UTR SEQ ID NO: 1, mRNA | | 154 |
|---|---|---|
| ORF SEQ ID NO: 17, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCACCCAGCACCGACGG | 17 |
| Sequence | CGCAGCCGGACUGGUGCCCGAGAGCAACAACGAGGUGAUGG | |
| (nucleotide) | CCCUGGAACCUGUGGCAGGCGCCGCUCUGGCUGCACCCGUG | |
| | ACCGGCCAGACCAACAUCAUCGACCCCUGGAUCCGGGCCAA | |
| | CUUCGUGCAGGCUCCCAACGGCGAGUUCACCGUGUCUCCCC | |
| | GGAACGCUCCCGGCGAGGUGCUGCUGAACCUGGAGCUGGGC | |
| | CCCGAGCUGAACCCCUACCUGGCCCACCUGGCCCGGAUGUA | |
| | CAACGGCUACGCCGGCGGCAUGGAGGUGCAGGUGAUGCUGG | |
| | CCGGCAACGCCUUCACCGCCGGCAAGCUGGUGUUCGCCGCC | |
| | GUGCCACCACACUUCCCCGUGGAGAACCUGUCUCCCCAGCA | |
| | GAUCACCAUGUUCCCUCACGUGAUCAUAGACGUGCGGACCC | |
| | UGGAGCCCGUGCUGCUGCCCCUGCCCGACGUGCGGAACAAC | |
| | UUCUUCCACUACAACCAGAAGGACGACCCCAAGAUGCGGAU | |
| | CGUGGCCAUGCUGUACACUCCCCUGCGGAGCAACGGCAGCG | |
| | GCGACGACGUGUUCACCGUGAGCUGCCGGGUGCUGACCAGA | |
| | CCCAGCCCCGACUUCGACUUCACCUACCUGGUGCCACCCACC | |
| | GUGGAGAGCAAGACCAAGCCCUUCACCCUGCCCAUCCUGAC | |
| | CCUGGGCGAGCUGAGCAACAGCCGGUUCCCCGUGAGCAUCG | |
| | ACCAGAUGUACACCAGCCCCAACGAGAUCAUCAGCGUGCAG | |
| | UGCCAGAACGGCCGGUGCACCCUGGACGGCGAGCUGCAGGG | |
| | CACCACCCAGCUGCAGGUGAGCGGCAUCUGCGCCUUCAAGG | |
| | GCGAGGUGACCGCCCACCUGCACGACAACGACCACCUGUAC | |
| | AACGUGACCAUCACCAACCUGAACGGCAGCCCCUUCGACCC | |
| | CAGCGAGGACAUCCCCGCACCACUGGGCGUGCCCGACUUCC | |
| | AGGGCCGGGUGUUCGGCAUCAUCAGCCAGCGGGACAAGCAC | |
| | AACAGCCCCGGCCACAACGAGCCUGCCAACCGCGGACACGA | |
| | CGCCGUGGUGCCCACCUACACCGCCCAGUACACCCCAAAGCU | |
| | GGGCCAGAUCCAGAUCGGCACCUGGCAGACCGACGACCUGA | |
| | CCGUGAACCAGCCCGUGAAGUUCACACCCGUGGGCCUGAAC | |
| | GACACCGAGCACUUCAACCAGUGGGUGGUCCCACGGUACGC | |
| | CGGUGCCCUGAACCUGAACACCAACCUGGCUCCCAGCGUGG | |
| | CUCCCGUGUUCCCCGGCGAGCGGCUGCUGUUCUUCCGGAGC | |
| | UACAUCCCACUGAAGGGCGGCUACGGCAACCCCGCCAUCGA | |
| | CUGCCUGCUGCCCCAGGAGUGGGUGCAGCACUUCUACCAGG | |
| | AGGCCGCCCCAAGCAUGAGCGAGGUGGCCCUGGUGCGGUAC | |
| | AUCAACCCCGACACCGGCAGAGCCCUGUUCGAGGCCAAGCU | |
| | GCACCGGGCCGGCUUCAUGACCGUGAGCAGCAACACCAGCG | |
| | CCCCUGUGGUGGUGCCCGCCAACGGCUACUUCCGGUUCGAC | |
| | AGCUGGGUGAACCAGUUCUACAGCCUGGCCCCAAUGGGCAC | |
| | CGGUAACGGACGCCGGCGGGUGCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSTDGAAGLVPESNNEVMALEPVAGAALAAPVT | 18 |
| Amino Acid | GQTNIIDPWIRANFVQAPNGEFTVSPRNAPGEVLLNLELGPELNP | |
| Sequence | YLAHLARMYNGYAGGMEVQVMLAGNAFTAGKLVFAAVPPHFP | |
| | VENLSPQQITMFPHVIIDVRTLEPVLLPLPDVRNNFFHYNQKDDP | |
| | KMRIVAMLYTPLRSNGSGDDVFTVSCRVLTRPSPDFDFTYLVPPT | |
| | VESKTKPFTLPILTLGELSNSRFPVSIDQMYTSPNEIISVQCQNGRC | |
| | TLDGELQGTTQLQVSGICAFKGEVTAHLHDNDHLYNVTITNLNG | |
| | SPFDPSEDIPAPLGVPDFQGRVFGIISQRDKHNSPGHNEPANRGHD | |
| | AVVPTYTAQYTPKLGQIQIGTWQTDDLTVNQPVKFTPVGLNDTE | |
| | HFNQWVVPRYAGALNLNTNLAPSVAPVFPGERLLFFRSYIPLKG | |
| | GYGNPAIDCLLPQEWVQHFYQEAAPSMSEVALVRYINPDTGRAL | |
| | FEAKLHRAGFMTVSSNTSAPVVVPANGYFRFDSWVNQFYSLAP | |
| | MGTGNGRRRVQ | |
| PolyA tail | 100 nt | |

GII.17_VP1

| SEQ ID NO: 155 consists of from 5′ end to 3′ end: 5′ UTR SEQ ID NO: 1, mRNA | | 155 |
|---|---|---|
| ORF SEQ ID NO: 19, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCCCCAAGCAACGACGG | 19 |
| Sequence | AGCCGCUGGCCUGGUGCCCGAGGGCAACAACGAGACCCUGC | |
| (nucleotide) | CCCUGGAACCCGUGGCCGGCGCAGCCAUUGCCGCCCCAGUG | |
| | ACCGGCCAGAACAACAUCAUCGACCCCUGGAUCCGGACCAA | |
| | CUUCGUGCAGGCUCCCAACGGCGAGUUCACCGUGUCUCCCC | |
| | GGAACAGCCCCGGCGAGAUCCUGCUGAACCUGGAGCUGGGC | |
| | CCCGACCUGAACCCCUACCUGGCCCACCUGAGCCGGAUGUA | |
| | CAACGGCUACGCCGGCGGCGUUGAGGUGCAGGUGCUGCUGG | |
| | CCGGCAACGCCUUCACCGCCGGCAAGAUCCUGUUCGCCGCC | |
| | GUGCCACCCAACUUCCCCGUGGAGUUCCUGAGCCCCGCCCA | |
| | GAUCACCAUGCUGCCCCACCUGAUCGUGGACGUGCGGACCC | |
| | UGGAGCCCAUCAUGAUUCCCCUGCCCGACGUGCGGAACACC | |
| | UUCUUCCACUACAGCAACCAGCCCAACAGCCGGAUGCGGCU | |
| | GGUGGCCAUGCUGUACACUCCCCUGCGGAGCAACGGCAGCG | |
| | GCGACGACGUGUUCACCGUGAGCUGCCGGGUGCUGACACGG | |
| | CCCACUCCCGACUUCGAGUUCACCUACCUGGUGCCUCCCAGC | |
| | GUGGAGAGCAAGACCAAGCCCUUCAGCCUGCCCAUCCUGAC | |
| | CCUGAGCGAGCUGACCAACAGCCGGUUCCCCGUGCCCAUCG | |
| | ACAGCCUGUUCACCGCCCAGAACAACGUGCUGCAGGUGCAG | |
| | UGCCAGAACGGCCGGUGCACCCUGGACGGCGAGCUGCAGGG | |
| | CACCACCCAGCUGCUGCCCAGCGGCAUCUGUGCCUUUCGGG | |
| | GCCGAGUGACCGCCCAGAUCAACCAGCGGGACCGGUGGCAC | |
| | AUGCAGCUGCAGAACCUGAACGGCACCACCUACGACCCCAC | |
| | CGACGACGUGCCCGCACCCCUGGGAACCCCUGACUUCAAGG | |
| | GCGUGGUGUUCGGCAUGGUGAGCCAGCGGAACGUGGGCAAC | |
| | GACGCUCCCGGCAGCACCAGAGCCCAGCAGGCCUGGGUGAG | |
| | CACCUACAGCCCACAGUUCGUGCCCAAGCUGGGCAGCGUGA | |
| | ACCUGCGGAUCAGCGACAACGACGACUUCCAGUUCCAGCCC | |
| | ACCAAGUUCACUCCCGUGGGCGUGAACGACGACGACGACGG | |
| | CCACCCCUUCCGGCAGUGGGAGCUGCCCAACUACAGCGGCG | |
| | AGCUGACCCUGAACAUGAACCUGGCACCUCCUGUGGCCCCU | |
| | AACUUCCCCGGCGAGCAGCUGCUGUUCUUCCGGAGCUUCGU | |
| | GCCCUGCAGCGGCGGCUACAACCAGGGCAUCAUCGACUGCC | |
| | UGAUACCCCAGGAGUGGAUCCAGCACUUCUACCAGGAGAGC | |
| | GCCCCUAGCCAGAGCGACGUGGCCCUGAUCCGGUACGUGAA | |
| | CCCCGACACAGGCCGGACCCUGUUCGAGGCCAAGCUGCACC | |
| | GGAGCGGCUACAUCACCGUGGCCCACAGCGGCGACUAUCCC | |
| | CUGGUGGUGCCCGCCAACGGCCACUUCCGGUUCGACAGCUG | |
| | GGUGAACCAGUUCUACAGCCUGGCCCCAAUGGGGACAGGCA | |
| | ACGGCCGGAGACGGGCCCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSNDGAAGLVPEGNNETLPLEPVAGAAIAAPVTG | 20 |
| Amino Acid | QNNIIDPWIRTNFVQAPNGEFTVSPRNSPGEILLNLELGPDLNPYL | |
| Sequence | AHLSRMYNGYAGGVEVQVLLAGNAFTAGKILFAAVPPNFPVEF | |
| | LSPAQITMLPHLIVDVRTLEPIMIPLPDVRNTFFHYSNQPNSRMRL | |
| | VAMLYTPLRSNGSGDDVFTVSCRVLTRPTPDFEFTYLVPPSVESK | |
| | TKPFSLPILTLSELTNSRFPVPIDSLFTAQNNVLQVQCQNGRCTLD | |
| | GELQGTTQLLPSGICAFRGRVTAQINQRDRWHMQLQNLNGTTY | |
| | DPTDDVPAPLGTPDFKGVVFGMVSQRNVGNDAPGSTRAQQAW | |
| | VSTYSPQFVPKLGSVNLRISDNDDFQFQPTKFTPVGVNDDDDGH | |
| | PFRQWELPNYSGELTLNMNLAPPVAPNFPGEQLLFFRSFVPCSGG | |
| | YNQGIIDCLIPQEWIQHFYQESAPSQSDVALIRYVNPDTGRTLFEA | |
| | KLHRSGYITVAHSGDYPLVVPANGHFRFDSWVNQFYSLAPMGT | |
| | GNGRRRAQ | |
| PolyA tail | 100 nt | |

GII.10_VP1_2000

| SEQ ID NO: 156 consists of from 5′ end to 3′ end: 5′ UTR SEQ ID NO: 1, mRNA | | 156 |
|---|---|---|
| ORF SEQ ID NO: 21, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCCCCAAGCAGCGACGG | 21 |
| Sequence | AGCUGCCGGCCUGGUGCCCGAGAGCAACAACGAGGUGAUGG | |
| (nucleotide) | CCCUUGAGCCUGUGGCUGGCGCCUCUCUUGCCGCCCCUGUG | |
| | ACCGGCCAGACCAACAUCAUCGACCCCUGGAUCCGGAUGAA | |
| | CUUCGUGCAGGCCCCUAACGGCGAGUUCACCGUGUCCCCUC | |
| | GGAACAGCCCCGGCGAGGUGCUGCUGAACCUGGAGCUGGGC | |
| | CCCGAGCUGAACCCCUACCUGGCCCACCUGAGCCGGAUGUA | |
| | CAACGGCUACGCCGGCGGCAUGGAGGUGCAGAUCAUGCUGG | |
| | CCGGCAACGCCUUCACCGCCGGCAAGCUGAUCUUCGCCGCC | |
| | GUGCCUCCCCACUUCCCCAUCGAGAACCUGUCUCCUCCUCAG | |
| | AUCACCAUGUUUCCCCACGUGAUCAUCGACGUGCGGACCCU | |
| | GGAGCCCGUGCUGCUGCCCAUGCCCGACAUCCGGAACAGCU | |
| | UCUUCCACUUCAUCCAGCGGGACGAGCCCAAGAUGCGGCUG | |
| | GUGGCCAUGCUGUACACACCCCUGCGGAGCAACGGCAGCGG | |
| | CGACGACGUGUUCACCGUGAGCUGCCGGGUGCUGACCCGGC | |
| | CAACACCCGACUUCGACUUCACCUACCUGGUGCCACCAACC | |
| | GUGGAGAGCAAGAGCAAGCCCUUCACCCUGCCCAUCCUGAC | |
| | CCUGGGCGAGCUGACCAACAGCCGGUUUCCCCUGCCCAUUG | |
| | ACGUGCUGUACACCAACCCCAACGAGAGCGCCAUCGUGCAG | |
| | UGCCAGAACGGCCGGUGCACCCUGGACGGCGAGCUGCAGGG | |
| | CACCACCCAGCUGCUGCCUACCGGCAUCUGCGCCUUCCGGG | |
| | GCAAGGUGACCCAGCAGGUGCAGGACGAGCACCGGGGCACC | |
| | CACUGGAACAUGACCGUGACCAACCUGAACGGCACCCCAUU | |
| | CGACCCCACCGAGGACGUGCCCGCCCCACUGGGCACUCCCGA | |
| | CUUCAGCGGCCAGAUCUACGGCGUGAUCAGCCAGCGGAACA | |
| | CCAACACUGUACCCGGCGAGGGAGACCUGCCCGCCAAUCGG | |
| | GCCCACGAGGCCGUGAUCGCCACCUACAGCCCCAAGUUCAC | |
| | CCCGAAGCUGGGCAACAUCCAGUUCAGCACCUGGGAGACCC | |
| | AGGACGUGAGCAGCGGCCAGCCCACCAAGUUCACUCCCGUG | |
| | GGCCUGGCCAGCGUGGACGCCAACAGCCACUUCGACCAGUG | |
| | GACCCUGCCCAGCUACAGCGGCGCCCUGACCCUGAACAUGA | |
| | ACCUGGCACCCAGCGUGGCCCCUGUGUUCCCCGGCGAGUGC | |
| | CUGCUGUUCUUCCGGAGCUUCAUACCCCUGAAGGGCGGCUA | |
| | CGGCAACCCCGCCAUCGACUGCCUGAUGCCCCAGGAGUGGG | |
| | UGCAGCACCUGUACCAGGAGAGCGCUCCCAGCCUGAGCGAC | |
| | GUGGCCCUGGUGCGGUACGUGAACCCCGAGACUGGCCGGAC | |
| | CCUGUUCGAGGCCAAGCUGCACCGGAACGGCUUCCUGACCG | |
| | UGGCCAGGAACAGCGCCGGACCAGUGGUGGCCCCAACCAAC | |
| | GGCUACUUCCGGUUCGACAGCUGGGUGAACCAGUUCUACAC | |
| | CCUGGCUCCCAUGGGCAACGGCUCUGGACGGCGGCGGAUGC | |
| | AG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSSDGAAGLVPESNNEVMALEPVAGASLAAPVT | 22 |
| Amino Acid | GQTNIIDPWIRMNFVQAPNGEFTVSPRNSPGEVLLNLELGPELNP | |
| Sequence | YLAHLSRMYNGYAGGMEVQIMLAGNAFTAGKLIFAAVPPHFPIE | |
| | NLSPPQITMFPHVIIDVRTLEPVLLPMPDIRNSFFHFIQRDEPKMRL | |
| | VAMLYTPLRSNGSGDDVFTVSCRVLTRPTPDFDFTYLVPPTVESK | |
| | SKPFTLPILTLGELTNSRFPLPIDVLYTNPNESAIVQCQNGRCTLD | |
| | GELQGTTQLLPTGICAFRGKVTQQVQDEHRGTHWNMTVTNLNG | |
| | TPFDPTEDVPAPLGTPDFSGQIYGVISQRNTNTVPGEGDLPANRA | |
| | HEAVIATYSPKFTPKLGNIQFSTWETQDVSSGQPTKFTPVGLASV | |
| | DANSHFDQWTLPSYSGALTLNMNLAPSVAPVFPGECLLFFRSFIP | |
| | LKGGYGNPAIDCLMPQEWVQHLYQESAPSLSDVALVRYVNPET | |
| | GRTLFEAKLHRNGFLTVARNSAGPVVAPTNGYFRFDSWVNQFY | |
| | TLAPMGNGSGRRRMQ | |
| PolyA tail | 100 nt | |

GI.3_VP1_2007

| SEQ ID NO: 157 consists of from 5′ end to 3′ end: 5′ UTR SEQ ID NO: 1, mRNA | | 157 |
|---|---|---|
| ORF SEQ ID NO: 23, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCCCUACCAACAUGGACGG | 23 |
| Sequence | CACUAGCGGCGCAGGCCAGCUGGUGCCCGAGGCCAACACCG | |
| (nucleotide) | CCGAGCCCAUCAGCAUGGAACCCGUGGCAGGCGCUGCAACC | |
| | GCAGCCGCAACCGCAGGGCAGGUGAACAUGAUCGACCCCUG | |
| | GAUCAUGAACAACUACGUGCAGGCCCCUCAGGGCGAGUUCA | |
| | CCAUCAGCCCCAACAACACGCCCGGCGACAUCCUGUUCGACC | |
| | UGCAGCUGGGGCCCCACCUGAACCCCUUCCUGAGCCACCUG | |
| | GCCCAGAUGUACAACGGCUGGGUGGGCAACAUGAAGGUGAA | |
| | GGUGCUGCUGGCCGGCAACGCCUUCACCGCCGGCAAGAUCA | |
| | UCAUCAGCUGCAUCCCACCCGGCUUCGUGGCCCAGAACGUG | |
| | AGCAUCGCCCAGGCCACCAUGUUCCCACACGUGAUUGCCGA | |
| | CGUGCGGGUGCUGGAGCCCAUCGAGGUGCCCCUGGAGGACG | |
| | UGCGGAACGUGCUGUUCCACAACAACGACAACACCCCAACC | |
| | AUGCGGCUGGUGUGCAUGCUGUACACCCCUCUGAGGGCCAG | |
| | CGGCAGCAGCAGCGGCACCGACCCCUUCGUGAUCGCCGGCC | |
| | GGGUGCUGACCUGCCCCAGCCCCGACUUCAGCUUCCUGUUC | |
| | CUGGUGCCACCUAACGUGGAGCAGAAGACCAAGCCCUUCAG | |
| | CGUGCCCAACCUGCCCCUGAACACCCUGAGCAACAGCCGGG | |
| | UGCCCAGCCUGAUCAAGAGCAUGAUGGUGAGCCGGGACCAC | |
| | GGCCAGAUGGUGCAGUUCCAGAACGGCCGGGUGACCCUGGA | |
| | CGGCCAGCUGCAAGGCACCACCCCUACCAGCGCCAGCCAGCU | |
| | GUGCAAGAUCCGGGGCAGCGUGUUCCACGCCAACGGCGGCA | |
| | ACGGCUACAACCUGACCGAGCUGGACGGCAGCCCCUACCAC | |
| | GCCUUCGAGAGCCCCGCCCCAAUCGGCUUCCCCGACCUGGGC | |
| | GAGUGCGACUGGCACAUGGAGGCCAGCCCCACCACCCAGUU | |
| | CAACACCGGCGACGUGAUCAAGCAGAUCAACGUGAAGCAGG | |
| | AGAGCGCCUUCGCCCCACACCUGGGCACCAUCCAGGCCGAC | |
| | GGCCUGAGCGACGUGAGCGUGAACACCAACAUGAUCGCCAA | |
| | GCUGGGCUGGGUGAGCCCCGUGAGCGACGGCCACAAGGGCG | |
| | ACGUGGACCCCUGGGUGAUCCCACGGUACGGCAGCACCCUG | |
| | ACCGAAGCUGCCCAGCUCGCCCCUCCAAUCUAUCCUCCGGGC | |
| | UUCGGCGAGGCCAUCGUGUUCUUCAUGAGCGACUUCCCCAU | |
| | CGCCCACGGCACCAACGGCCUGAGUGUGCCCUGCACCAUUC | |
| | CCCAGGAGUUCGUGACCCACUUCGUGAACGAGCAGGCCCCU | |
| | ACCCGGGGUGAAGCCGCCCUGCUGCACUACCUGGACCCCGA | |
| | CACCCACCGGAACCUGGGUGAGUUCAAGCUGUACCCCGACG | |
| | GCUUCAUGACCUGCGUGCCCAAUAGCUCUGGCACCGGUCCC | |
| | CAGACCCUGCCCAUCAACGGCGUGUUCGUGUUCGUGAGCUG | |
| | GGUGUCACGGUUCUACCAGCUGAAGCCCGUGGGCACCGCUG | |
| | GACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDAPTNMDGTSGAGQLVPEANTAEPISMEPVAGAATA | 24 |
| Amino Acid | AATAGQVNMIDPWIMNNYVQAPQGEFTISPNNTPGDILFDLQLG | |
| Sequence | PHLNPFLSHLAQMYNGWVGNMKVKVLLAGNAFTAGKIIISCIPP | |
| | GFVAQNVSIAQATMFPHVIADVRVLEPIEVPLEDVRNVLFHNND | |
| | NTPTMRLVCMLYTPLRASGSSSGTDPFVIAGRVLTCPSPDFSFLFL | |
| | VPPNVEQKTKPFSVPNLPLNTLSNSRVPSLIKSMMVSRDHGQMV | |
| | QFQNGRVTLDGQLQGTTPTSASQLCKIRGSVFHANGGNGYNLTE | |
| | LDGSPYHAFESPAPIGFPDLGECDWHMEASPTTQFNTGDVIKQIN | |
| | VKQESAFAPHLGTIQADGLSDVSVNTNMIAKLGWVSPVSDGHK | |
| | GDVDPWVIPRYGSTLTEAAQLAPPIYPPGFGEAIVFFMSDFPIAHG | |
| | TNGLSVPCTIPQEFVTHFVNEQAPTRGEAALLHYLDPDTHRNLGE | |
| | FKLYPDGFMTCVPNSSGTGPQTLPINGVFVFVSWVSRFYQLKPV | |
| | GTAGPARRLGIRRS | |
| PolyA tail | 100 nt | |

VP2_GI.1_Norwalk

| SEQ ID NO: 158 consists of from 5′ end to 3′ end: 5′ UTR SEQ ID NO: 1, mRNA | | 158 |
|---|---|---|
| ORF SEQ ID NO: 25, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGGCCCAGGCCAUCAUCGGCGCCAUCGCCGCUAGCACCGC | 25 |
| Sequence | CGGCAGCGCACUUGGCGCCGGCAUCCAGGUGGGCGGAGAAG | |
| (nucleotide) | CCGCCCUGCAGAGCCAGCGGUACCAGCAGAACCUCCAGCUG | |
| | CAGGAGAACAGCUUCAAGCACGACCGGGAGAUGAUCGGCUA | |
| | CCAGGUGGAGGCCAGCAACCAGCUGCUGGCCAAGAACCUGG | |
| | CCACCCGGUACUCUCUGCUGAGGGCCGGCGGUCUGACCUCU | |
| | GCCGACGCUGCCCGCAGCGUGGCUGGAGCCCCAGUGACCCG | |
| | GAUCGUGGACUGGAACGGAGUGCGGGUGUCCGCCCCAGAGA | |
| | GCAGCGCCACCACCCUGAGAAGCGGCGGCUUCAUGAGCGUG | |
| | CCCAUCCCCUUCGCCAGCAAGCAGAAGCAGGUGCAGAGCAG | |
| | CGGCAUCAGCAACCCCAACUACAGCCCCAGCAGCAUCAGCC | |
| | GGACCACCAGCUGGGUGGAGAGCCAGAACAGCAGCCGGUUC | |
| | GGCAACCUGAGCCCCUACCACGCCGAGGCCCUGAACACCGU | |
| | GUGGCUGACACCUCCCGGCAGCACCGCCAGCAGCACCCUGA | |
| | GCAGCGUGCCCCGGGGCUACUUCAACACCGACCGGCUGCCC | |
| | CUGUUCGCCAACAACCGGCGG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MAQAIIGAIAASTAGSALGAGIQVGGEAALQSQRYQQNLQLQEN | 26 |
| Amino Acid | SFKHDREMIGYQVEASNQLLAKNLATRYSLLRAGGLTSADAARS | |
| Sequence | VAGAPVTRIVDWNGVRVSAPESSATTLRSGGFMSVPIPFASKQK | |
| | QVQSSGISNPNYSPSSISRTTSWVESQNSSRFGNLSPYHAEALNTV | |
| | WLTPPGSTASSTLSSVPRGYFNTDRLPLFANNRR | |
| PolyA tail | 100 nt | |

VP2_GII.4_Sydney

| SEQ ID NO: 159 consists of from 5′ end to 3′ end: 5′ UTR SEQ ID NO: 1, mRNA | | 159 |
|---|---|---|
| ORF SEQ ID NO: 27, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGGCCGGAGCCUUCUUCGCCGGACUGGCCAGCGACGUGUU | 27 |
| Sequence | GGGCUCUGGCCUGGGCAGCCUGAUUAACGCCGGGGCCGGCG | |
| (nucleotide) | CCAUUAACCAGAAGGUGGAGUUCGAGAACAACCGGAAACUG | |
| | CAGCAAGCCAGCUUUCAGUUCAGCAGCAACCUGCAGCAGGC | |
| | CAGCUUCCAGCACGACAAGGAGAUGCUGCAGGCCCAGAUCG | |
| | AGGCCACCAAGAAGCUGCAGCAGGAGAUGAUGAAGGUGAAG | |
| | CAGGCCAUGCUGCUGGAGGGCGGCUUCAGCGAGACCGACGC | |
| | CGCCAGAGGCGCUAUCAACGCUCCCAUGACCAAGGCCCUGG | |
| | ACUGGAGCGGCACCCGGUACUGGGCCCCAGACGCCCGGACA | |
| | ACCACCUACAACGCCGGCCGGUUCAGCACCCCUCAACCCAGC | |
| | GGCGCUCUGCCAGGAAGAGCCAAUCUGCGUGACGCCGUGCC | |
| | AGCCCACGGUUCCAGCAGCAAGAGCAGCAACUUCAGCACCG | |
| | CCACCAGCGUGUACAGCAACCAGACCACCAGCACACGGCUG | |
| | GGAAGCACCGCCGGCAGCGGAACCAGCGUGAGCAGCCUGCC | |
| | CAGCACCGCACGGACCCGGAGCUGGGUGGAGGACCAGAGCC | |
| | GGAACCUGAGCCCCUUCAUGCGGGGCGCCCACAACAUCAGC | |
| | UUCGUGACCCCUCCCAGCAGCCGGAGCAGCAGCCAGGGCAC | |
| | CGUGAGCACCGUGCCCAAGGAGGUGCUGGACAGCUGGACCG | |
| | GCGCCUUCAACACCCGGCGGCAGCCCCUGUUCGCCCACAUCC | |
| | GGAAGCGGGGCGAGAGCCGGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MAGAFFAGLASDVLGSGLGSLINAGAGAINQKVEFENNRKLQQ | 28 |
| Amino Acid | ASFQFSSNLQQASFQHDKEMLQAQIEATKKLQQEMMKVKQAM | |
| Sequence | LLEGGFSETDAARGAINAPMTKALDWSGTRYWAPDARTTTYNA | |
| | GRFSTPQPSGALPGRANLRDAVPAHGSSSKSSNFSTATSVYSNQT | |
| | TSTRLGSTAGSGTSVSSLPSTARTRSWVEDQSRNLSPFMRGAHNI | |
| | SFVTPPSSRSSSQGTVSTVPKEVLDSWTGAFNTRRQPLFAHIRKR | |
| | GESRV | |
| PolyA tail | 100 nt | |

VP2_GI.1_deltaNLS

| SEQ ID NO: 160 consists of from 5′ end to 3′ end: 5′ UTR SEQ ID NO: 1, mRNA | | 160 |
|---|---|---|
| ORF SEQ ID NO: 29, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGGCCCAGGCCAUCAUCGGCGCCAUCGCCGCUAGCACCGC | 29 |
| Sequence | CGGCAGCGCACUUGGCGCCGGCAUCCAGGUGGGCGGAGAAG | |
| (nucleotide) | CCGCCCUGCAGAGCCAGCGGUACCAGCAGAACCUCCAGCUG | |
| | CAGGAGAACAGCUUCAAGCACGACCGGGAGAUGAUCGGCUA | |
| | CCAGGUGGAGGCCAGCAACCAGCUGCUGGCCAAGAACCUGG | |
| | CCACCCGGUACUCUCUGCUGAGGGCCGGCGGUCUGACCUCU | |
| | GCCGACGCUGCCCGCAGCGUGGCUGGAGCCCCAGUGACCCG | |
| | GAUCGUGGACUGGAACGGAGUGCGGGUGUCCGCCCCAGAGA | |
| | GCAGCGCCACCACCCUGAGAAGCGGCGGCUUCAUGAGCGUG | |
| | CCCAUCCCCUUCGCCAGCAAGCAGAAGCAGGUGCAGAGCAG | |
| | CGGCAUCAGCAACCCCAACUACAGCCCCAGCAGCAUCAGCC | |
| | GGACCACCAGCUGGGUGGAGAGCCAGAACAGCAGCCGGUUC | |
| | GGCAACCUGAGCCCCUACCACGCCGAGGCCCUGAACACCGU | |
| | GUGGCUGACACCUCCCGGCAGCACCGCCAGCAGCACCCUGA | |
| | GCAGCGUGCCCCGGGGCUACUUCAACACCGACCGGCUGCCC | |
| | CUGUUC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MAQAIIGAIAASTAGSALGAGIQVGGEAALQSQRYQQNLQLQEN | 30 |
| Amino Acid | SFKHDREMIGYQVEASNQLLAKNLATRYSLLRAGGLTSADAARS | |
| Sequence | VAGAPVTRIVDWNGVRVSAPESSATTLRSGGFMSVPIPFASKQK | |
| | QVQSSGISNPNYSPSSISRTTSWVESQNSSRFGNLSPYHAEALNTV | |
| | WLTPPGSTASSTLSSVPRGYFNTDRLPLF | |
| PolyA tail | 100 nt | |

VP2_GII.4_deltaNLS

| SEQ ID NO: 161 consists of from 5′ end to 3′ end: 5′ UTR SEQ ID NO: 1, mRNA | | 161 |
|---|---|---|
| ORF SEQ ID NO: 31, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGGCCGGAGCCUUCUUCGCCGGACUGGCCAGCGACGUGUU | 31 |
| Sequence | GGGCUCUGGCCUGGGCAGCCUGAUUAACGCCGGGGCCGGCG | |
| (nucleotide) | CCAUUAACCAGAAGGUGGAGUUCGAGAACAACCGGAAACUG | |
| | CAGCAAGCCAGCUUUCAGUUCAGCAGCAACCUGCAGCAGGC | |
| | CAGCUUCCAGCACGACAAGGAGAUGCUGCAGGCCCAGAUCG | |
| | AGGCCACCAAGAAGCUGCAGCAGGAGAUGAUGAAGGUGAAG | |
| | CAGGCCAUGCUGCUGGAGGGCGGCUUCAGCGAGACCGACGC | |
| | CGCCAGAGGCGCUAUCAACGCUCCCAUGACCAAGGCCCUGG | |
| | ACUGGAGCGGCACCCGGUACUGGGCCCCAGACGCCCGGACA | |
| | ACCACCUACAACGCCGGCCGGUUCAGCACCCCUCAACCCAGC | |
| | GGCGCUCUGCCAGGAAGAGCCAAUCUGCGUGACGCCGUGCC | |
| | AGCCCACGGUUCCAGCAGCAAGAGCAGCAACUUCAGCACCG | |
| | CCACCAGCGUGUACAGCAACCAGACCACCAGCACACGGCUG | |
| | GGAAGCACCGCCGGCAGCGGAACCAGCGUGAGCAGCCUGCC | |
| | CAGCACCGCACGGACCCGGAGCUGGGUGGAGGACCAGAGCC | |
| | GGAACCUGAGCCCCUUCAUGCGGGGCGCCCACAACAUCAGC | |
| | UUCGUGACCCCUCCCAGCAGCCGGAGCAGCAGCCAGGGCAC | |
| | CGUGAGCACCGUGCCCAAGGAGGUGCUGGACAGCUGGACCG | |
| | GCGCCUUCAACACCCGGCGGCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MAGAFFAGLASDVLGSGLGSLINAGAGAINQKVEFENNRKLQQ | 32 |
| Amino Acid | ASFQFSSNLQQASFQHDKEMLQAQIEATKKLQQEMMKVKQAM | |
| Sequence | LLEGGFSETDAARGAINAPMTKALDWSGTRYWAPDARTTTYNA | |
| | GRFSTPQPSGALPGRANLRDAVPAHGSSSKSSNFSTATSVYSNQT | |
| | TSTRLGSTAGSGTSVSSLPSTARTRSWVEDQSRNLSPFMRGAHNI | |
| | SFVTPPSSRSSSQGTVSTVPKEVLDSWTGAFNTRRQ | |
| PolyA tail | 100 nt | |

NS6_3CLP_GI.1

| SEQ ID NO: 162 consists of from 5′ end to 3′ end: 5′ UTR SEQ ID NO: 1, mRNA | | 162 |
|---|---|---|
| ORF SEQ ID NO: 33, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGGCCCCACCCACCCUGUGGAGCCGGGUGACCAAGUUCGG | 33 |
| Sequence | CAGCGGCUGGGGCUUCUGGGUGAGCCCCACCGUGUUCAUCA | |
| (nucleotide) | CCACCACCCACGUGGUGCCCACCGGCGUGAAGGAGUUCUUC | |
| | GGCGAGCCCCUGAGCAGCAUCGCCAUCCACCAGGCCGGCGA | |
| | GUUCACCCAGUUCCGGUUCAGCAAGAAGAUGCGGCCCGACC | |
| | UGACCGGCAUGGUGCUGGAGGAGGGCUGUCCCGAGGGCACC | |
| | GUGUGCAGCGUGCUGAUCAAGCGGGACAGCGGCGAGCUGCU | |
| | GCCCCUGGCCGUGCGGAUGGGCGCCAUCGCCAGCAUGCGGA | |
| | UCCAGGGCAGACUGGUGCACGGCCAGAGCGGCAUGCUGCUG | |
| | ACCGGCGCCAACGCCAAGGGCAUGGACCUGGGCACCAUCCC | |
| | CGGAGACUGCGGCGCUCCCUACGUGCACAAGCGGGGCAACG | |
| | ACUGGGUGGUGUGUGGAGUCCACGCCGCCGCCACCAAGAGC | |
| | GGCAACACCGUGGUGUGCGCCGUGCAGGCCGGAGAGGGCGA | |
| | GACCGCCCUGGAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MAPPTLWSRVTKFGSGWGFWVSPTVFITTTHVVPTGVKEFFGEP | 34 |
| Amino Acid | LSSIAIHQAGEFTQFRFSKKMRPDLTGMVLEEGCPEGTVCSVLIK | |
| Sequence | RDSGELLPLAVRMGAIASMRIQGRLVHGQSGMLLTGANAKGMD | |
| | LGTIPGDCGAPYVHKRGNDWVVCGVHAAATKSGNTVVCAVQA | |
| | GEGETALE | |
| PolyA tail | 100 nt | |

VP1_R69A_GII.4

| SEQ ID NO: 163 consists of from 5′ end to 3′ end: 5′ UTR SEQ ID NO: 1, mRNA | | 163 |
|---|---|---|
| ORF SEQ ID NO: 35, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 35 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUG | |
| | CCAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACCCCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCAACUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 36 |
| Amino Acid | GQQNVIDPWIRNNFVQAPGGEFTVSPANAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICNFRGDVTHITGSRNYTMNLASQNWNNYDP | |
| | TEEIPAPLGTPDFVGKIQGMLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

VP1_R69A_Sdom_GII.4/Pdom_GII.17

| SEQ ID NO: 164 consists of from 5′ end to 3′ end: 5′ UTR SEQ ID NO: 1, mRNA | | 164 |
|---|---|---|
| ORF SEQ ID NO: 37, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 37 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUG | |
| | CCAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACCCCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCCUGCCCAUCCUGA | |
| | CCCUGAGCGAGCUGACCAACAGCCGGUUCCCCGUGCCCAUC | |
| | GACAGCCUGUUCACCGCCCAGAACAACGUGCUGCAGGUGCA | |
| | GUGCCAGAACGGCCGGUGCACCCUGGACGGCGAGCUGCAGG | |
| | GCACCACCCAGCUGCUGCCCAGCGGCAUCUGUGCCUUUCGG | |
| | GGCCGAGUGACCGCCCAGAUCAACCAGCGGGACCGGUGGCA | |
| | CAUGCAGCUGCAGAACCUGAACGGCACCACCUACGACCCCA | |
| | CCGACGACGUGCCCGCACCCCUGGGAACCCCUGACUUCAAG | |
| | GGCGUGGUGUUCGGCAUGGUGAGCCAGCGGAACGUGGGCAA | |
| | CGACGCUCCCGGCAGCACCAGAGCCCAGCAGGCCUGGGUGA | |
| | GCACCUACAGCCCACAGUUCGUGCCCAAGCUGGGCAGCAUC | |
| | AACCUGCGGAUCAGCGACAACGACGACUUCCAGUUCCAGCC | |
| | CACCAAGUUCACUCCCGUGGGCGUGAACGACGACGACGACG | |
| | GCCACCCCUUCCGGCAGUGGGAGCUGCCCAACUACAGCGGC | |
| | GAGCUGACCCUGAACAUGAACCUGGCACCUCCUGUGGCCCC | |
| | UAACUUCCCCGGCGAGCAGCUGCUGUUCUUCCGGAGCUUCG | |
| | UGCCCUGCAGCGGCGGCUACAACCAGGGCAUCAUCGACUGC | |
| | CUGAUACCCCAGGAGUGGAUCCAGCACUUCUACCAGGAGAG | |
| | CGCCCCUAGCCAGAGCGACGUGGCCCUGAUCCGGUACGUGA | |
| | ACCCCGACACAGGCCGGACCCUGUUCGAGGCCAAGCUGCAC | |
| | CGGAGCGGCUACAUCACCGUGGCCCACAGCGGCGACUAUCC | |
| | CCUGGUGGUGCCCGCCAACGGCCACUUCCGGUUCGACAGCU | |
| | GGGUGAACCAGUUCUACAGCCUGGCCCCAAUGGGGACAGGC | |
| | AACGGCCGGAGACGGGCCCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 38 |
| Amino Acid | GQQNVIDPWIRNNFVQAPGGEFTVSPANAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSLPILTLSELTNSRFPVPIDSLFTAQNNVLQVQCQNGRCTLD | |
| | GELQGTTQLLPSGICAFRGRVTAQINQRDRWHMQLQNLNGTTY | |
| | DPTDDVPAPLGTPDFKGVVFGMVSQRNVGNDAPGSTRAQQAW | |
| | VSTYSPQFVPKLGSINLRISDNDDFQFQPTKFTPVGVNDDDDGHP | |
| | FRQWELPNYSGELTLNMNLAPPVAPNFPGEQLLFFRSFVPCSGG | |
| | YNQGIIDCLIPQEWIQHFYQESAPSQSDVALIRYVNPDTGRTLFEA | |
| | KLHRSGYITVAHSGDYPLVVPANGHFRFDSWVNQFYSLAPMGT | |
| | GNGRRRAQ | |
| PolyA tail | 100 nt | |

VP1_Sdom_GII.4/Pdom_GII.17

| SEQ ID NO: 165 consists of from 5′ end to 3′ end: 5′ UTR SEQ ID NO: 1, mRNA | | 165 |
|---|---|---|
| ORF SEQ ID NO: 39, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 39 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACCCCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCCUGCCCAUCCUGA | |
| | CCCUGAGCGAGCUGACCAACAGCCGGUUCCCCGUGCCCAUC | |
| | GACAGCCUGUUCACCGCCCAGAACAACGUGCUGCAGGUGCA | |
| | GUGCCAGAACGGCCGGUGCACCCUGGACGGCGAGCUGCAGG | |
| | GCACCACCCAGCUGCUGCCCAGCGGCAUCUGUGCCUUUCGG | |
| | GGCCGAGUGACCGCCCAGAUCAACCAGCGGGACCGGUGGCA | |
| | CAUGCAGCUGCAGAACCUGAACGGCACCACCUACGACCCCA | |
| | CCGACGACGUGCCCGCACCCCUGGGAACCCCUGACUUCAAG | |
| | GGCGUGGUGUUCGGCAUGGUGAGCCAGCGGAACGUGGGCAA | |
| | CGACGCUCCCGGCAGCACCAGAGCCCAGCAGGCCUGGGUGA | |
| | GCACCUACAGCCCACAGUUCGUGCCCAAGCUGGGCAGCAUC | |
| | AACCUGCGGAUCAGCGACAACGACGACUUCCAGUUCCAGCC | |
| | CACCAAGUUCACUCCCGUGGGCGUGAACGACGACGACGACG | |
| | GCCACCCCUUCCGGCAGUGGGAGCUGCCCAACUACAGCGGC | |
| | GAGCUGACCCUGAACAUGAACCUGGCACCUCCUGUGGCCCC | |
| | UAACUUCCCCGGCGAGCAGCUGCUGUUCUUCCGGAGCUUCG | |
| | UGCCCUGCAGCGGCGGCUACAACCAGGGCAUCAUCGACUGC | |
| | CUGAUACCCCAGGAGUGGAUCCAGCACUUCUACCAGGAGAG | |
| | CGCCCCUAGCCAGAGCGACGUGGCCCUGAUCCGGUACGUGA | |
| | ACCCCGACACAGGCCGGACCCUGUUCGAGGCCAAGCUGCAC | |
| | CGGAGCGGCUACAUCACCGUGGCCCACAGCGGCGACUAUCC | |
| | CCUGGUGGUGCCCGCCAACGGCCACUUCCGGUUCGACAGCU | |
| | GGGUGAACCAGUUCUACAGCCUGGCCCCAAUGGGGACAGGC | |
| | AACGGCCGGAGACGGGCCCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 40 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSLPILTLSELTNSRFPVPIDSLFTAQNNVLQVQCQNGRCTLD | |
| | GELQGTTQLLPSGICAFRGRVTAQINQRDRWHMQLQNLNGTTY | |
| | DPTDDVPAPLGTPDFKGVVFGMVSQRNVGNDAPGSTRAQQAW | |
| | VSTYSPQFVPKLGSINLRISDNDDFQFQPTKFTPVGVNDDDDGHP | |
| | FRQWELPNYSGELTLNMNLAPPVAPNFPGEQLLFFRSFVPCSGG | |
| | YNQGIIDCLIPQEWIQHFYQESAPSQSDVALIRYVNPDTGRTLFEA | |
| | KLHRSGYITVAHSGDYPLVVPANGHFRFDSWVNQFYSLAPMGT | |
| | GNGRRRAQ | |
| PolyA tail | 100 nt | |

GII.4_Pdomain

| SEQ ID NO: 166 consists of from 5′ end to 3′ end: 5′ UTR SEQ ID NO: 1, mRNA | | 166 |
|---|---|---|
| ORF SEQ ID NO: 41, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGCCUGGCAGCAAGCCCUUCAGCGUGCCGGUGCUGACCGU | 41 |
| Sequence | CGAGGAGAUGACCAACAGCCGGUUCCCCAUUCCCCUGGAGA | |
| (nucleotide) | AGCUGUUCACCGGCCCCUCCAGCGCCUUCGUGGUGCAGCCC | |
| | CAGAACGGCCGGUGCACCACCGACGGCGUGCUGUUGGGCAC | |
| | UACCCAGCUCAGCCCCGUGAACAUCUGCAACUUCCGGGGCG | |
| | ACGUGACCCACAUCACCGGAAGCCGGAACUACACCAUGAAC | |
| | CUGGCCAGCCAGAACUGGAAUAACUACGACCCUACGGAGGA | |
| | GAUCCCCGCACCCCUGGGCACUCCCGACUUCGUGGGCAAGA | |
| | UUCAGGGCAUGCUGACCCAGACCACCCGGACCGACGGGAGC | |
| | ACACGGGGCCACAAGGCCACAGUGUACACCGGCAGCGCGGA | |
| | CUUCGCGCCCAAACUGGGCCGCGUGCAGUUCGAGACCGACA | |
| | CCGACCACGACUUCGAGGCCAACCAGAACACCAAGUUCACA | |
| | CCCGUGGGCGUGAUCCAGGACGGCAGCACAACCCACCGGAA | |
| | CGAACCCCAGCAGUGGGUCCUGCCCAGCUACAGCGGCCGGA | |
| | ACACCCACAACGUGCACCUGGCACCCGCCGUGGCACCCACCU | |
| | UCCCCGGCGAACAGCUGCUGUUCUUCCGGAGCACCAUGCCC | |
| | GGCUGCAGCGGCUACCCCAACAUGGACCUGGACUGCCUGCU | |
| | GCCCCAGGAGUGGGUGCAGUACUUCUACCAGGAGGCAGCUC | |
| | CCGCCCAGAGCGACGUGGCCCUGCUGCGGUUCGUGAACCCC | |
| | GAUACCGGCCGGGUGCUGUUCGAGUGCAAGCUGCACAAGUC | |
| | GGGCUACGUGACCGUGGCCCACACCGGCCAGCACGACCUGG | |
| | UGAUCCCUCCUAACGGGUACUUCCGGUUCGAUAGCUGGGUG | |
| | AACCAGUUCUACACCCUGGCGCCUAUGGGCAACGGCACCGG | |
| | ACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGGGC | |
| Corresponding | MPGSKPFSVPVLTVEEMTNSRFPIPLEKLFTGPSSAFVVQPQNGR | 42 |
| Amino Acid | CTTDGVLLGTTQLSPVNICNFRGDVTHITGSRNYTMNLASQNWN | |
| Sequence | NYDPTEEIPAPLGTPDFVGKIQGMLTQTTRTDGSTRGHKATVYT | |
| | GSADFAPKLGRVQFETDTDHDFEANQNTKFTPVGVIQDGSTTHR | |
| | NEPQQWVLPSYSGRNTHNVHLAPAVAPTFPGEQLLFFRSTMPGC | |
| | SGYPNMDLDCLLPQEWVQYFYQEAAPAQSDVALLRFVNPDTGR | |
| | VLFECKLHKSGYVTVAHTGQHDLVIPPNGYFRFDSWVNQFYTL | |
| | APMGNGTGRRRVV | |
| PolyA tail | 100 nt | |

N1_TM_Pdom_delGlyc_GII.4

| SEQ ID NO: 167 consists of from 5′ end to 3′ end: 5′ UTR SEQ ID NO: 1, mRNA | | 167 |
|---|---|---|
| ORF SEQ ID NO: 43, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAACCCAAACCAGAAGAUCAUCACCAUCGGCAGUAUCUG | 43 |
| Sequence | CAUGACCAUCGGCACCGCCAACCUGAUCCUGCAGAUCGGCA | |
| (nucleotide) | ACAUCAUCAGCAUCUGGGUGAGCCCUGGCAGCAAGCCCUUC | |
| | AGCGUGCCGGUGCUGACCGUCGAGGAGAUGACCAACAGCCG | |
| | GUUCCCCAUUCCCCUGGAGAAGCUGUUCACCGGCCCCUCCA | |
| | GCGCCUUCGUGGUGCAGCCCCAGAACGGCCGGUGCACCACC | |
| | GACGGCGUGCUGUUGGGCACUACCCAGCUCAGCCCCGUGAA | |
| | CAUCUGCAACUUCCGGGGCGACGUGACCCACAUCACCGGAA | |
| | GCCGGGACUACACCAUGAACCUGGCCAGCCAGAACUGGAAU | |
| | AACUACGACCCUACGGAGGAGAUCCCCGCACCCCUGGGCAC | |
| | UCCCGACUUCGUGGGCAAGAUUCAGGGCAUGCUGACCCAGA | |
| | CCACCCGGACCGACGGGAGCACACGGGGCCACAAGGCCACA | |
| | GUGUACACCGGCAGCGCGGACUUCGCGCCCAAACUGGGCCG | |
| | CGUGCAGUUCGAGACCGACACCGACCACGACUUCGAGGCCA | |
| | ACCAGAACACCAAGUUCACACCCGUGGGCGUGAUCCAGGAC | |
| | GGCAGCACAACCCACCGGAACGAACCCCAGCAGUGGGUCCU | |
| | GCCCAGCUACAGCGGCCGGAACACCCACAACGUGCACCUGG | |
| | CACCCGCCGUGGCACCCACCUUCCCCGGCGAACAGCUGCUGU | |
| | UCUUCCGGAGCACCAUGCCCGGCUGCAGCGGCUACCCCAAC | |
| | AUGGACCUGGACUGCCUGCUGCCCCAGGAGUGGGUGCAGUA | |
| | CUUCUACCAGGAGGCAGCUCCCGCCCAGAGCGACGUGGCCC | |
| | UGCUGCGGUUCGUGAACCCCGAUACCGGCCGGGUGCUGUUC | |
| | GAGUGCAAGCUGCACAAGUCGGGCUACGUGACCGUGGCCCA | |
| | CACCGGCCAGCACGACCUGGUGAUCCCUCCUAACGGGUACU | |
| | UCCGGUUCGAUAGCUGGGUGAACCAGUUCUACACCCUGGCG | |
| | CCUAUGGGCGACGGCACCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MNPNQKIITIGSICMTIGTANLILQIGNIISIWVSPGSKPFSVPVLTV | 44 |
| Amino Acid | EEMTNSRFPIPLEKLFTGPSSAFVVQPQNGRCTTDGVLLGTTQLS | |
| Sequence | PVNICNFRGDVTHITGSRDYTMNLASQNWNNYDPTEEIPAPLGTP | |
| | DFVGKIQGMLTQTTRTDGSTRGHKATVYTGSADFAPKLGRVQF | |
| | ETDTDHDFEANQNTKFTPVGVIQDGSTTHRNEPQQWVLPSYSGR | |
| | NTHNVHLAPAVAPTFPGEQLLFFRSTMPGCSGYPNMDLDCLLPQ | |
| | EWVQYFYQEAAPAQSDVALLRFVNPDTGRVLFECKLHKSGYVT | |
| | VAHTGQHDLVIPPNGYFRFDSWVNQFYTLAPMGDGTGRRRVV | |
| PolyA tail | 100 nt | |

GII.4_2018A_R69A

| SEQ ID NO: 168 consists of from 5′ end to 3′ end: 5′ UTR SEQ ID NO: 1, mRNA | | 168 |
|---|---|---|
| ORF SEQ ID NO: 45, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 45 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUG | |
| | CCAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCACGACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | UGCACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 46 |
| Amino Acid | GQQNVIDPWIRNNFVQAPGGEFTVSPANAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSHDYTMNLASQNWNNYDP | |
| | TEEIPAPLGTPDFVGKIQGMLTQTTRTDGSTRGHKATVYTGSVHF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

GII.4_2018A_DS2

| SEQ ID NO: 169 consists of from 5′ end to 3′ end: 5′ UTR SEQ ID NO: 1, mRNA | | 169 |
|---|---|---|
| ORF SEQ ID NO: 47, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 47 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUUGCUGCGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCUGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCACGACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | UGCACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 48 |
| Amino Acid | GQQNVIDPWIRNNFCCAPGGEFTVSPRNAPGEILWSAPLGPDLNP | |
| Sequence | YLSHLARMYNGYAGGFEVQVILAGNAFTAGKIIFAAVPPNFPTE | |
| | GLSPCQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSHDYTMNLASQNWNNYDP | |
| | TEEIPAPLGTPDFVGKIQGMLTQTTRTDGSTRGHKATVYTGSVHF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

GII.4_2018A_R69A_DS2

| SEQ ID NO: 170 consists of from 5′ end to 3′ end: 5′ UTR SEQ ID NO: 1, mRNA | | 170 |
|---|---|---|
| ORF SEQ ID NO: 49, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 49 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUUGCUGCGCCCCUGGCGGCGAGUUCACCGUGUCCCCUG | |
| | CCAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCUGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCACGACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | UGCACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 50 |
| Amino Acid | GQQNVIDPWIRNNFCCAPGGEFTVSPANAPGEILWSAPLGPDLNP | |
| Sequence | YLSHLARMYNGYAGGFEVQVILAGNAFTAGKIIFAAVPPNFPTE | |
| | GLSPCQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSHDYTMNLASQNWNNYDP | |
| | TEEIPAPLGTPDFVGKIQGMLTQTTRTDGSTRGHKATVYTGSVHF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

GII.4_2012_R69A_DS2

| SEQ ID NO: 171 consists of from 5′ end to 3′ end: 5′ UTR SEQ ID NO: 1, mRNA | | 171 |
|---|---|---|
| ORF SEQ ID NO: 51, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 51 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUUGCUGCGCCCCUGGCGGCGAGUUCACCGUGUCCCCUG | |
| | CCAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCUGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCACGACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | UGCACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 52 |
| Amino Acid | GQQNVIDPWIRNNFCCAPGGEFTVSPANAPGEILWSAPLGPDLNP | |
| Sequence | YLSHLARMYNGYAGGFEVQVILAGNAFTAGKIIFAAVPPNFPTE | |
| | GLSPCQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICNFRGDVTHITGSRNYTMNLASQNWNNYDP | |
| | TEEIPAPLGTPDFVGKIQGMLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

GII.4c 2019-2021

| SEQ ID NO: 172 consists of from 5′ end to 3′ end: 5′ UTR SEQ ID NO: 1, mRNA | | 172 |
|---|---|---|
| ORF SEQ ID NO: 53, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 53 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCACAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCAUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCAACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCG | |
| | CCGGACGUAGACGGGCCCUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 54 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSHNYTMNLASQNWNNYDP | |
| | TEEIPAPLGTPDFVGKIQGMLTQTTRTDGSTRGHKATVYTGSADF | |
| | APKLGRVQFETDTNHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GAGRRRAL | |
| PolyA tail | 100 nt | |

GII.4c_USA

| SEQ ID NO: 173 consists of from 5′ end to 3′ end: 5′ UTR SEQ ID NO: 1, mRNA | | 173 |
|---|---|---|
| ORF SEQ ID NO: 55, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 55 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 56 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| PolyA tail | 100 nt | |

GII.4c_USA_Q58C_S136C

| SEQ ID NO: 174 consists of from 5′ end to 3′ end: 5′ UTR SEQ ID NO: 1, mRNA | | 174 |
|---|---|---|
| ORF SEQ ID NO: 57, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 57 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGUGCGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCUGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 58 |
| Amino Acid | GQQNVIDPWIRNNFVCAPGGEFTVSPRNAPGEILWSAPLGPDLNP | |
| Sequence | YLSHLARMYNGYAGGFEVQVILAGNAFTAGKIIFAAVPPNFPTE | |
| | GLSPCQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRAV | |
| PolyA tail | 100 nt | |

GII.4c_USA_Q58C_T139C

| SEQ ID NO: 175 consists of from 5′ end to 3′ end: 5′ UTR SEQ ID NO: 1, mRNA | | 175 |
|---|---|---|
| ORF SEQ ID NO: 59, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 59 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGUGCGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGUGCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 60 |
| Amino Acid | GQQNVIDPWIRNNFVCAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVCMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRAV | |
| PolyA tail | 100 nt | |

GII.4c_USA_N112C_A116C

| SEQ ID NO: 176 consists of from 5′ end to 3′ end: 5′ UTR SEQ ID NO: 1, mRNA | | 176 |
|---|---|---|
| ORF SEQ ID NO: 61, and 3′ UTR SEQ ID NO: 4. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 61 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCUGCGCCUUCACCUGCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 62 |
| Amino Acid | GQQNVIDPWIRNNFVQAPGGEFTVSPRNAPGEILWSAPLGPDLNP | |
| Sequence | YLSHLARMYNGYAGGFEVQVILAGCAFTCGKIIFAAVPPNFPTEG | |
| | LSPSQVTMFPHIIVDVRQLEPVLIPLPDVRNNFYHYNQSNDSTIKL | |
| | IAMLYTPLRANNAGDDVFTVSCRVLTRPSPDFDFIFLVPPTVESRT | |
| | KPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRAV | |
| PolyA tail | 100 nt | |

GII.3 Hu/GII.3/SW4/2012/TN

| SEQ ID NO: 177 consists of from 5′ end to 3′ end: 5′ UTR SEQ ID NO: 1, mRNA | | 177 |
|---|---|---|
| ORF SEQ ID NO: 63, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCCCCAAGCAACGACGG | 63 |
| Sequence | UGCUGCUGGGCUGGUGCCCGAGAUCAACAACGAGGCCAUGG | |
| (nucleotide) | CACUCGAACCAGUAGCAGGGGCUGCUAUUGCUGCUCCACUG | |
| | ACCGGCCAGCAGAACAUCAUCGACCCCUGGAUCAUGAACAA | |
| | CUUCGUGCAGGCACCAGGCGGCGAGUUCACCGUGUCUCCCC | |
| | GGAACAGCCCCGGCGAAGUCCUUCUGAACCUGGAGCUGGGC | |
| | CCCGAGAUCAACCCCUACCUGGCCCACCUGGCCCGGAUGUA | |
| | CAACGGGUACGCAGGAGGAUUCGAGGUGCAAGUCGUGCUCG | |
| | CCGGCAACGCCUUCACCGCCGGCAAGAUCAUCUUCGCCGCC | |
| | AUUCCUCCUAACUUUCCAAUCGACAACCUGAGCGCCGCCCA | |
| | GAUCACCAUGUGUCCUCACGUCAUCGUGGACGUCAGGCAGC | |
| | UGGAGCCCGUGAACCUGCCCAUGCCAGACGUAAGGAACAAC | |
| | UUCUUCCACUACAACCAGGGCAGCGACAGCCGGCUGCGGCU | |
| | CAUCGCUAUGCUGUACACUCCACUGCGGGCCAACAACAGCG | |
| | GCGACGACGUGUUCACCGUGAGCUGUAGGGUUCUAACCCGG | |
| | CCCAGCCCCGACUUCAGCUUCAACUUCCUGGUGCCUCCUACC | |
| | GUGGAGAGCAAGACCAAGCCCUUCAGCCUGCCCAUCCUGAC | |
| | CAUCAGCGAGAUGAGCAACAGCCGGUUCCCCGUGCCCAUCG | |
| | ACAGCCUGCACACCAGCCCCACCGAGAACAUCGUGGUGCAG | |
| | UGCCAGAACGGGCGCGUGACCCUAGACGGGGAGCUGAUGGG | |
| | CACCACCCAGCUGCUGCCCAGCCAGAUCUGCGCCUUCCGGG | |
| | GCGUGCUGAUCCGGAGCACCAGUAGGGCAAGCGAUCAAGCG | |
| | GACACCGCCACUCCUCGGCUGUUCAACUACUACUGGCACAU | |
| | CCAGCUGGACAACCUGAACGGCACUCCUUACGACCCCGCCG | |
| | AGGACAUCCCCGGGCCUCUGGGCACUCCAGACUUCCGGGGC | |
| | AAGGUGUUCGGCGUGGCCAGCCAGCGGAACCCUGACACAAC | |
| | AACUCGGGCACACGAAGCCAAGAUCGACACCACCUCAGGCC | |
| | GGUUCACUCCAAAGCUGGGCAGCCUGGAGAUCAGCACCGAG | |
| | AGCGACGACUUCGACCAGAACAAGCCUACCCGGUUCACACC | |
| | UGUGGGCAUCGGCGUGGACCACGAGGCCGACUUCCAGCAGU | |
| | GGACCCUGCCCGACUACGCCGGCCAGUUCACCCACAACAUG | |
| | AACCUGGCUCCAGCCGUGGCUCCUAACUUCCCCGGCGAGCA | |
| | GCUGCUGUUCUUCCGGAGCCAAUUACCAUCCUCUGGAGGUC | |
| | GCAGCAACGGCAUCCUGGACUGCCUGGUGCCCCAGGAGUGG | |
| | GUGCAGCACUUCUACCAGGAGAGCGCUCCAAGCCAGAGCCA | |
| | GGUGGCCCUGGUGCGGUACAUCAACCCCGACACCGGACGAG | |
| | UUCUGUUCGAGGCCAAGCUGCACAAGCUGGGCUUCAUCACC | |
| | AUCGCCAAGAACGGCGACAGCCCCAUCACCGUGCCCCCUAA | |
| | CGGCUACUUCCGGUUCGAGAGCUGGGUGAACCCCUUCUACA | |
| | CCCUGGCGCCAAUGGGCACCGGCAACGGACGUCGCCGGAUC | |
| | CAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSNDGAAGLVPEINNEAMALEPVAGAAIAAPLTG | 64 |
| Amino Acid | QQNIIDPWIMNNFVQAPGGEFTVSPRNSPGEVLLNLELGPEINPYL | |
| Sequence | AHLARMYNGYAGGFEVQVVLAGNAFTAGKIIFAAIPPNFPIDNLS | |
| | AAQITMCPHVIVDVRQLEPVNLPMPDVRNNFFHYNQGSDSRLRL | |
| | IAMLYTPLRANNSGDDVFTVSCRVLTRPSPDFSFNFLVPPTVESK | |
| | TKPFSLPILTISEMSNSRFPVPIDSLHTSPTENIVVQCQNGRVTLDG | |
| | ELMGTTQLLPSQICAFRGVLIRSTSRASDQADTATPRLFNYYWHI | |
| | QLDNLNGTPYDPAEDIPGPLGTPDFRGKVFGVASQRNPDTTTRA | |
| | HEAKIDTTSGRFTPKLGSLEISTESDDFDQNKPTRFTPVGIGVDHE | |
| | ADFQQWTLPDYAGQFTHNMNLAPAVAPNFPGEQLLFFRSQLPSS | |
| | GGRSNGILDCLVPQEWVQHFYQESAPSQSQVALVRYINPDTGRV | |
| | LFEAKLHKLGFITIAKNGDSPITVPPNGYFRFESWVNPFYTLAPM | |
| | GTGNGRRRIQ | |
| PolyA tail | 100 nt | |

GII.12 HU/GII.P16_GII.12/AlbertaEI464/CA/2018

| SEQ ID NO: 178 consists of from 5′ end to 3′ end: 5′ UTR SEQ ID NO: 1, mRNA | | 178 |
|---|---|---|
| ORF SEQ ID NO: 65, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCCCCAAGCAACGACGG | 65 |
| Sequence | UGCUGCUGGGCUGGUGCCCGAGGUGAACAACGAGACCAUGG | |
| (nucleotide) | CACUCGAACCAGUAGCAGGGGCUUCUAUUGCUGCUCCACUG | |
| | ACCGGCCAGAACAACGUGAUCGACCCCUGGAUCCGGCUGAA | |
| | CUUCGUGCAGGCACCAAACGGCGAGUUCACCGUGUCUCCCC | |
| | GGAACAGCCCCGGCGAAGUCCUUCUGAACCUGGAGCUGGGC | |
| | CCCGAGCUGAACCCCUACCUGGCCCACCUGAGCCGGAUGUA | |
| | CAACGGGUACGCAGGAGGAGUCGAGGUGCAAGUCUUGCUCG | |
| | CCGGCAACGCCUUCACCGCCGGCAAGCUGGUGUUCGCCGCC | |
| | GUGCCUCCUCACUUUCCACUGGAGAACAUCAGCCCCGGCCA | |
| | GAUCACCAUGUUCCCUCACGUCAUCAUUGACGUCAGGACCC | |
| | UGGAGCCCGUGCUGCUGCCCCUGCCAGACGUAAGGAACAGC | |
| | UUCUUCCACUACAACCAGCAGAACGAGCCCCGGAUGCGGCU | |
| | CGUCGCUAUGCUGUACACUCCACUGCGGAGCAACGGCAGCG | |
| | GCGACGACGUGUUCACCGUGAGCUGUAGGGUUCUAACCCGG | |
| | CCCAGCCCCGACUUCGACUUCAACUACCUGGUGCCUCCUACC | |
| | GUGGAGAGCAAGACCAAGCCCUUCACCCUGCCCGUGCUGAC | |
| | CAUCGGCGAGCUGACCAACAGCCGGUUCCCCGUGCCCAUCG | |
| | ACGAGCUGUACACCAGCCCCAACGAGAGCCUGGUGGUGCAG | |
| | CCCCAGAACGGGCGCUGUGCACUAGACGGGGAGCUGCAGGG | |
| | CACCACCCAGCUGCUGCCCACCGCCAUCUGCAGCUUCCGGGG | |
| | CCGGAUCAACCAGAAGGUGAGCGGCGAGAACCACGUUUGGA | |
| | ACAUGCAGGUGACCAACAUCAACGGCACUCCUUUCGACCCC | |
| | ACCGAGGACGUGCCCGCUCCUCUGGGCACUCCAGACUUCAG | |
| | CGGCAAGCUGUUCGGCGUGCUGAGCCAGCGGGACCACGACA | |
| | ACGCCUGCCGGAGCCACGACGCCGUGAUCGCCACCGACUCA | |
| | ACGAAGUUCACUCCAAAGCUGGGCGCCGUGCAGAUCGGCAC | |
| | CUGGGAGCAGGACGACGUGCACAUCAACCAGCCUACCAAGU | |
| | UCACACCUGUGGGCCUGUUCGAGAACGAGGGCUUCAACCAG | |
| | UGGACCCUGCCCAACUACAGCGGCGCCCUGACCCUGAACAU | |
| | GGGCCUGGCUCCACCCGUGGCUCCUACCUUCCCCGGCGAGC | |
| | AGAUCCUGUUCUUCCGGAGCCACAUUCCACUGAAAGGAGGU | |
| | GUAGCUGAUCCAGUGAUCGACUGCCUGCUGCCCCAGGAGUG | |
| | GAUCCAGCACCUGUACCAGGAGAGCGCUCCAAGCCAGACCG | |
| | ACGUGGCCCUGAUCCGGUUCACCAACCCCGACACCGGACGA | |
| | GUUCUGUUCGAGGCCAAGCUGCACCGGAGCGGCUACAUCAC | |
| | CGUGGCCAACACCGGCAGCCGGCCCAUCGUGGUGCCCGCCA | |
| | ACGGCUACUUCCGGUUCGACAGCUGGGUGAACCAGUUCUAC | |
| | AGCCUGGCGCCAAUGGGCACCGGCAACGGACGUCGCCGGAU | |
| | GCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSNDGAAGLVPEVNNETMALEPVAGASIAAPLTG | 66 |
| Amino Acid | QNNVIDPWIRLNFVQAPNGEFTVSPRNSPGEVLLNLELGPELNPY | |
| Sequence | LAHLSRMYNGYAGGVEVQVLLAGNAFTAGKLVFAAVPPHFPLE | |
| | NISPGQITMFPHVIIDVRTLEPVLLPLPDVRNSFFHYNQQNEPRMR | |
| | LVAMLYTPLRSNGSGDDVFTVSCRVLTRPSPDFDFNYLVPPTVES | |
| | KTKPFTLPVLTIGELTNSRFPVPIDELYTSPNESLVVQPQNGRCAL | |
| | DGELQGTTQLLPTAICSFRGRINQKVSGENHVWNMQVTNINGTP | |
| | FDPTEDVPAPLGTPDFSGKLFGVLSQRDHDNACRSHDAVIATDST | |
| | KFTPKLGAVQIGTWEQDDVHINQPTKFTPVGLFENEGENQWTLP | |
| | NYSGALTLNMGLAPPVAPTFPGEQILFFRSHIPLKGGVADPVIDC | |
| | LLPQEWIQHLYQESAPSQTDVALIRFTNPDTGRVLFEAKLHRSGY | |
| | ITVANTGSRPIVVPANGYFRFDSWVNQFYSLAPMGTGNGRRRM | |
| | Q | |
| PolyA tail | 100 nt | |

GI.3_A37C_A44C

| SEQ ID NO: 179 consists of from 5′ end to 3′ end: 5′ UTR SEQ ID NO: 1, mRNA | | 179 |
|---|---|---|
| ORF SEQ ID NO: 67, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCCCUACCAACAUGGACGG | 67 |
| Sequence | CACUAGCGGCGCAGGCCAGCUGGUGCCCGAGGCCAACACCG | |
| (nucleotide) | CCGAGCCCAUCAGCAUGGAACCCGUGUGCGGCGCUGCAACC | |
| | GCAGCCUGUACCGCAGGGCAGGUGAACAUGAUCGACCCCUG | |
| | GAUCAUGAACAACUACGUGCAGGCCCCUCAGGGCGAGUUCA | |
| | CCAUCAGCCCCAACAACACGCCCGGCGACAUCCUGUUCGACC | |
| | UGCAGCUGGGGCCCCACCUGAACCCCUUCCUGAGCCACCUG | |
| | GCCCAGAUGUACAACGGCUGGGUGGGCAACAUGAAGGUGAA | |
| | GGUGCUGCUGGCCGGCAACGCCUUCACCGCCGGCAAGAUCA | |
| | UCAUCAGCUGCAUCCCACCCGGCUUCGUGGCCCAGAACGUG | |
| | AGCAUCGCCCAGGCCACCAUGUUCCCACACGUGAUUGCCGA | |
| | CGUGCGGGUGCUGGAGCCCAUCGAGGUGCCCCUGGAGGACG | |
| | UGCGGAACGUGCUGUUCCACAACAACGACAACACCCCAACC | |
| | AUGCGGCUGGUGUGCAUGCUGUACACCCCUCUGAGGGCCAG | |
| | CGGCAGCAGCAGCGGCACCGACCCCUUCGUGAUCGCCGGCC | |
| | GGGUGCUGACCUGCCCCAGCCCCGACUUCAGCUUCCUGUUC | |
| | CUGGUGCCACCUAACGUGGAGCAGAAGACCAAGCCCUUCAG | |
| | CGUGCCCAACCUGCCCCUGAACACCCUGAGCAACAGCCGGG | |
| | UGCCCAGCCUGAUCAAGAGCAUGAUGGUGAGCCGGGACCAC | |
| | GGCCAGAUGGUGCAGUUCCAGAACGGCCGGGUGACCCUGGA | |
| | CGGCCAGCUGCAAGGCACCACCCCUACCAGCGCCAGCCAGCU | |
| | GUGCAAGAUCCGGGGCAGCGUGUUCCACGCCAACGGCGGCA | |
| | ACGGCUACAACCUGACCGAGCUGGACGGCAGCCCCUACCAC | |
| | GCCUUCGAGAGCCCCGCCCCAAUCGGCUUCCCCGACCUGGGC | |
| | GAGUGCGACUGGCACAUGGAGGCCAGCCCCACCACCCAGUU | |
| | CAACACCGGCGACGUGAUCAAGCAGAUCAACGUGAAGCAGG | |
| | AGAGCGCCUUCGCCCCACACCUGGGCACCAUCCAGGCCGAC | |
| | GGCCUGAGCGACGUGAGCGUGAACACCAACAUGAUCGCCAA | |
| | GCUGGGCUGGGUGAGCCCCGUGAGCGACGGCCACAAGGGCG | |
| | ACGUGGACCCCUGGGUGAUCCCACGGUACGGCAGCACCCUG | |
| | ACCGAAGCUGCCCAGCUCGCCCCUCCAAUCUAUCCUCCGGGC | |
| | UUCGGCGAGGCCAUCGUGUUCUUCAUGAGCGACUUCCCCAU | |
| | CGCCCACGGCACCAACGGCCUGAGUGUGCCCUGCACCAUUC | |
| | CCCAGGAGUUCGUGACCCACUUCGUGAACGAGCAGGCCCCU | |
| | ACCCGGGGUGAAGCCGCCCUGCUGCACUACCUGGACCCCGA | |
| | CACCCACCGGAACCUGGGUGAGUUCAAGCUGUACCCCGACG | |
| | GCUUCAUGACCUGCGUGCCCAAUAGCUCUGGCACCGGUCCC | |
| | CAGACCCUGCCCAUCAACGGCGUGUUCGUGUUCGUGAGCUG | |
| | GGUGUCACGGUUCUACCAGCUGAAGCCCGUGGGCACCGCUG | |
| | GACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDAPTNMDGTSGAGQLVPEANTAEPISMEPVCGAATA | 68 |
| Amino Acid | ACTAGQVNMIDPWIMNNYVQAPQGEFTISPNNTPGDILFDLQLG | |
| Sequence | PHLNPFLSHLAQMYNGWVGNMKVKVLLAGNAFTAGKIIISCIPP | |
| | GFVAQNVSIAQATMFPHVIADVRVLEPIEVPLEDVRNVLFHNND | |
| | NTPTMRLVCMLYTPLRASGSSSGTDPFVIAGRVLTCPSPDFSFLFL | |
| | VPPNVEQKTKPFSVPNLPLNTLSNSRVPSLIKSMMVSRDHGQMV | |
| | QFQNGRVTLDGQLQGTTPTSASQLCKIRGSVFHANGGNGYNLTE | |
| | LDGSPYHAFESPAPIGFPDLGECDWHMEASPTTQFNTGDVIKQIN | |
| | VKQESAFAPHLGTIQADGLSDVSVNTNMIAKLGWVSPVSDGHK | |
| | GDVDPWVIPRYGSTLTEAAQLAPPIYPPGFGEAIVFFMSDFPIAHG | |
| | TNGLSVPCTIPQEFVTHFVNEQAPTRGEAALLHYLDPDTHRNLGE | |
| | FKLYPDGFMTCVPNSSGTGPQTLPINGVFVFVSWVSRFYQLKPV | |
| | GTAGPARRLGIRRS | |
| PolyA tail | 100 nt | |

GI.3 Q62C A140C

| SEQ ID NO: 180 consists of from 5′ end to 3′ end: 5′ UTR SEQ ID NO: 1, mRNA | | 180 |
|---|---|---|
| ORF SEQ ID NO: 69, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCCCUACCAACAUGGACGG | 69 |
| Sequence | CACUAGCGGCGCAGGCCAGCUGGUGCCCGAGGCCAACACCG | |
| (nucleotide) | CCGAGCCCAUCAGCAUGGAACCCGUGGCAGGCGCUGCAACC | |
| | GCAGCCGCAACCGCAGGGCAGGUGAACAUGAUCGACCCCUG | |
| | GAUCAUGAACAACUACGUGUGCGCCCCUCAGGGCGAGUUCA | |
| | CCAUCAGCCCCAACAACACGCCCGGCGACAUCCUGUUCGACC | |
| | UGCAGCUGGGGCCCCACCUGAACCCCUUCCUGAGCCACCUG | |
| | GCCCAGAUGUACAACGGCUGGGUGGGCAACAUGAAGGUGAA | |
| | GGUGCUGCUGGCCGGCAACGCCUUCACCGCCGGCAAGAUCA | |
| | UCAUCAGCUGCAUCCCACCCGGCUUCGUGGCCCAGAACGUG | |
| | AGCAUCUGCCAGGCCACCAUGUUCCCACACGUGAUUGCCGA | |
| | CGUGCGGGUGCUGGAGCCCAUCGAGGUGCCCCUGGAGGACG | |
| | UGCGGAACGUGCUGUUCCACAACAACGACAACACCCCAACC | |
| | AUGCGGCUGGUGUGCAUGCUGUACACCCCUCUGAGGGCCAG | |
| | CGGCAGCAGCAGCGGCACCGACCCCUUCGUGAUCGCCGGCC | |
| | GGGUGCUGACCUGCCCCAGCCCCGACUUCAGCUUCCUGUUC | |
| | CUGGUGCCACCUAACGUGGAGCAGAAGACCAAGCCCUUCAG | |
| | CGUGCCCAACCUGCCCCUGAACACCCUGAGCAACAGCCGGG | |
| | UGCCCAGCCUGAUCAAGAGCAUGAUGGUGAGCCGGGACCAC | |
| | GGCCAGAUGGUGCAGUUCCAGAACGGCCGGGUGACCCUGGA | |
| | CGGCCAGCUGCAAGGCACCACCCCUACCAGCGCCAGCCAGCU | |
| | GUGCAAGAUCCGGGGCAGCGUGUUCCACGCCAACGGCGGCA | |
| | ACGGCUACAACCUGACCGAGCUGGACGGCAGCCCCUACCAC | |
| | GCCUUCGAGAGCCCCGCCCCAAUCGGCUUCCCCGACCUGGGC | |
| | GAGUGCGACUGGCACAUGGAGGCCAGCCCCACCACCCAGUU | |
| | CAACACCGGCGACGUGAUCAAGCAGAUCAACGUGAAGCAGG | |
| | AGAGCGCCUUCGCCCCACACCUGGGCACCAUCCAGGCCGAC | |
| | GGCCUGAGCGACGUGAGCGUGAACACCAACAUGAUCGCCAA | |
| | GCUGGGCUGGGUGAGCCCCGUGAGCGACGGCCACAAGGGCG | |
| | ACGUGGACCCCUGGGUGAUCCCACGGUACGGCAGCACCCUG | |
| | ACCGAAGCUGCCCAGCUCGCCCCUCCAAUCUAUCCUCCGGGC | |
| | UUCGGCGAGGCCAUCGUGUUCUUCAUGAGCGACUUCCCCAU | |
| | CGCCCACGGCACCAACGGCCUGAGUGUGCCCUGCACCAUUC | |
| | CCCAGGAGUUCGUGACCCACUUCGUGAACGAGCAGGCCCCU | |
| | ACCCGGGGUGAAGCCGCCCUGCUGCACUACCUGGACCCCGA | |
| | CACCCACCGGAACCUGGGUGAGUUCAAGCUGUACCCCGACG | |
| | GCUUCAUGACCUGCGUGCCCAAUAGCUCUGGCACCGGUCCC | |
| | CAGACCCUGCCCAUCAACGGCGUGUUCGUGUUCGUGAGCUG | |
| | GGUGUCACGGUUCUACCAGCUGAAGCCCGUGGGCACCGCUG | |
| | GACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDAPTNMDGTSGAGQLVPEANTAEPISMEPVAGAATA | 70 |
| Amino Acid | AATAGQVNMIDPWIMNNYVCAPQGEFTISPNNTPGDILFDLQLG | |
| Sequence | PHLNPFLSHLAQMYNGWVGNMKVKVLLAGNAFTAGKIIISCIPP | |
| | GFVAQNVSICQATMFPHVIADVRVLEPIEVPLEDVRNVLFHNND | |
| | NTPTMRLVCMLYTPLRASGSSSGTDPFVIAGRVLTCPSPDFSFLFL | |
| | VPPNVEQKTKPFSVPNLPLNTLSNSRVPSLIKSMMVSRDHGQMV | |
| | QFQNGRVTLDGQLQGTTPTSASQLCKIRGSVFHANGGNGYNLTE | |
| | LDGSPYHAFESPAPIGFPDLGECDWHMEASPTTQFNTGDVIKQIN | |
| | VKQESAFAPHLGTIQADGLSDVSVNTNMIAKLGWVSPVSDGHK | |
| | GDVDPWVIPRYGSTLTEAAQLAPPIYPPGFGEAIVFFMSDFPIAHG | |
| | TNGLSVPCTIPQEFVTHFVNEQAPTRGEAALLHYLDPDTHRNLGE | |
| | FKLYPDGFMTCVPNSSGTGPQTLPINGVFVFVSWVSRFYQLKPV | |
| | GTAGPARRLGIRRS | |
| PolyA tail | 100 nt | |

GI.3 N116C A191C

| SEQ ID NO: 181 consists of from 5′ end to 3′ end: 5′ UTR SEQ ID NO: 1, mRNA | | 181 |
|---|---|---|
| ORF SEQ ID NO: 71, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCCCUACCAACAUGGACGG | 71 |
| Sequence | CACUAGCGGCGCAGGCCAGCUGGUGCCCGAGGCCAACACCG | |
| (nucleotide) | CCGAGCCCAUCAGCAUGGAACCCGUGGCAGGCGCUGCAACC | |
| | GCAGCCGCAACCGCAGGGCAGGUGAACAUGAUCGACCCCUG | |
| | GAUCAUGAACAACUACGUGCAGGCCCCUCAGGGCGAGUUCA | |
| | CCAUCAGCCCCAACAACACGCCCGGCGACAUCCUGUUCGACC | |
| | UGCAGCUGGGGCCCCACCUGAACCCCUUCCUGAGCCACCUG | |
| | GCCCAGAUGUACAACGGCUGGGUGGGCAACAUGAAGGUGAA | |
| | GGUGCUGCUGGCCGGCUGCGCCUUCACCGCCGGCAAGAUCA | |
| | UCAUCAGCUGCAUCCCACCCGGCUUCGUGGCCCAGAACGUG | |
| | AGCAUCGCCCAGGCCACCAUGUUCCCACACGUGAUUGCCGA | |
| | CGUGCGGGUGCUGGAGCCCAUCGAGGUGCCCCUGGAGGACG | |
| | UGCGGAACGUGCUGUUCCACAACAACGACAACACCCCAACC | |
| | AUGCGGCUGGUGUGCAUGCUGUACACCCCUCUGAGGUGCAG | |
| | CGGCAGCAGCAGCGGCACCGACCCCUUCGUGAUCGCCGGCC | |
| | GGGUGCUGACCUGCCCCAGCCCCGACUUCAGCUUCCUGUUC | |
| | CUGGUGCCACCUAACGUGGAGCAGAAGACCAAGCCCUUCAG | |
| | CGUGCCCAACCUGCCCCUGAACACCCUGAGCAACAGCCGGG | |
| | UGCCCAGCCUGAUCAAGAGCAUGAUGGUGAGCCGGGACCAC | |
| | GGCCAGAUGGUGCAGUUCCAGAACGGCCGGGUGACCCUGGA | |
| | CGGCCAGCUGCAAGGCACCACCCCUACCAGCGCCAGCCAGCU | |
| | GUGCAAGAUCCGGGGCAGCGUGUUCCACGCCAACGGCGGCA | |
| | ACGGCUACAACCUGACCGAGCUGGACGGCAGCCCCUACCAC | |
| | GCCUUCGAGAGCCCCGCCCCAAUCGGCUUCCCCGACCUGGGC | |
| | GAGUGCGACUGGCACAUGGAGGCCAGCCCCACCACCCAGUU | |
| | CAACACCGGCGACGUGAUCAAGCAGAUCAACGUGAAGCAGG | |
| | AGAGCGCCUUCGCCCCACACCUGGGCACCAUCCAGGCCGAC | |
| | GGCCUGAGCGACGUGAGCGUGAACACCAACAUGAUCGCCAA | |
| | GCUGGGCUGGGUGAGCCCCGUGAGCGACGGCCACAAGGGCG | |
| | ACGUGGACCCCUGGGUGAUCCCACGGUACGGCAGCACCCUG | |
| | ACCGAAGCUGCCCAGCUCGCCCCUCCAAUCUAUCCUCCGGGC | |
| | UUCGGCGAGGCCAUCGUGUUCUUCAUGAGCGACUUCCCCAU | |
| | CGCCCACGGCACCAACGGCCUGAGUGUGCCCUGCACCAUUC | |
| | CCCAGGAGUUCGUGACCCACUUCGUGAACGAGCAGGCCCCU | |
| | ACCCGGGGUGAAGCCGCCCUGCUGCACUACCUGGACCCCGA | |
| | CACCCACCGGAACCUGGGUGAGUUCAAGCUGUACCCCGACG | |
| | GCUUCAUGACCUGCGUGCCCAAUAGCUCUGGCACCGGUCCC | |
| | CAGACCCUGCCCAUCAACGGCGUGUUCGUGUUCGUGAGCUG | |
| | GGUGUCACGGUUCUACCAGCUGAAGCCCGUGGGCACCGCUG | |
| | GACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDAPTNMDGTSGAGQLVPEANTAEPISMEPVAGAATA | 72 |
| Amino Acid | AATAGQVNMIDPWIMNNYVQAPQGEFTISPNNTPGDILFDLQLG | |
| Sequence | PHLNPFLSHLAQMYNGWVGNMKVKVLLAGCAFTAGKIIISCIPP | |
| | GFVAQNVSIAQATMFPHVIADVRVLEPIEVPLEDVRNVLFHNND | |
| | NTPTMRLVCMLYTPLRCSGSSSGTDPFVIAGRVLTCPSPDFSFLFL | |
| | VPPNVEQKTKPFSVPNLPLNTLSNSRVPSLIKSMMVSRDHGQMV | |
| | QFQNGRVTLDGQLQGTTPTSASQLCKIRGSVFHANGGNGYNLTE | |
| | LDGSPYHAFESPAPIGFPDLGECDWHMEASPTTQFNTGDVIKQIN | |
| | VKQESAFAPHLGTIQADGLSDVSVNTNMIAKLGWVSPVSDGHK | |
| | GDVDPWVIPRYGSTLTEAAQLAPPIYPPGFGEAIVFFMSDFPIAHG | |
| | TNGLSVPCTIPQEFVTHFVNEQAPTRGEAALLHYLDPDTHRNLGE | |
| | FKLYPDGFMTCVPNSSGTGPQTLPINGVFVFVSWVSRFYQLKPV | |
| | GTAGPARRLGIRRS | |
| PolyA tail | 100 nt | |

GI.3 N116C S192C

| SEQ ID NO: 182 consists of from 5′ end to 3′ end: 5′ UTR SEQ ID NO: 1, mRNA | | 182 |
|---|---|---|
| ORF SEQ ID NO: 73, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCCCUACCAACAUGGACGG | 73 |
| Sequence | CACUAGCGGCGCAGGCCAGCUGGUGCCCGAGGCCAACACCG | |
| (nucleotide) | CCGAGCCCAUCAGCAUGGAACCCGUGGCAGGCGCUGCAACC | |
| | GCAGCCGCAACCGCAGGGCAGGUGAACAUGAUCGACCCCUG | |
| | GAUCAUGAACAACUACGUGCAGGCCCCUCAGGGCGAGUUCA | |
| | CCAUCAGCCCCAACAACACGCCCGGCGACAUCCUGUUCGACC | |
| | UGCAGCUGGGGCCCCACCUGAACCCCUUCCUGAGCCACCUG | |
| | GCCCAGAUGUACAACGGCUGGGUGGGCAACAUGAAGGUGAA | |
| | GGUGCUGCUGGCCGGCUGCGCCUUCACCGCCGGCAAGAUCA | |
| | UCAUCAGCUGCAUCCCACCCGGCUUCGUGGCCCAGAACGUG | |
| | AGCAUCGCCCAGGCCACCAUGUUCCCACACGUGAUUGCCGA | |
| | CGUGCGGGUGCUGGAGCCCAUCGAGGUGCCCCUGGAGGACG | |
| | UGCGGAACGUGCUGUUCCACAACAACGACAACACCCCAACC | |
| | AUGCGGCUGGUGUGCAUGCUGUACACCCCUCUGAGGGCCUG | |
| | CGGCAGCAGCAGCGGCACCGACCCCUUCGUGAUCGCCGGCC | |
| | GGGUGCUGACCUGCCCCAGCCCCGACUUCAGCUUCCUGUUC | |
| | CUGGUGCCACCUAACGUGGAGCAGAAGACCAAGCCCUUCAG | |
| | CGUGCCCAACCUGCCCCUGAACACCCUGAGCAACAGCCGGG | |
| | UGCCCAGCCUGAUCAAGAGCAUGAUGGUGAGCCGGGACCAC | |
| | GGCCAGAUGGUGCAGUUCCAGAACGGCCGGGUGACCCUGGA | |
| | CGGCCAGCUGCAAGGCACCACCCCUACCAGCGCCAGCCAGCU | |
| | GUGCAAGAUCCGGGGCAGCGUGUUCCACGCCAACGGCGGCA | |
| | ACGGCUACAACCUGACCGAGCUGGACGGCAGCCCCUACCAC | |
| | GCCUUCGAGAGCCCCGCCCCAAUCGGCUUCCCCGACCUGGGC | |
| | GAGUGCGACUGGCACAUGGAGGCCAGCCCCACCACCCAGUU | |
| | CAACACCGGCGACGUGAUCAAGCAGAUCAACGUGAAGCAGG | |
| | AGAGCGCCUUCGCCCCACACCUGGGCACCAUCCAGGCCGAC | |
| | GGCCUGAGCGACGUGAGCGUGAACACCAACAUGAUCGCCAA | |
| | GCUGGGCUGGGUGAGCCCCGUGAGCGACGGCCACAAGGGCG | |
| | ACGUGGACCCCUGGGUGAUCCCACGGUACGGCAGCACCCUG | |
| | ACCGAAGCUGCCCAGCUCGCCCCUCCAAUCUAUCCUCCGGGC | |
| | UUCGGCGAGGCCAUCGUGUUCUUCAUGAGCGACUUCCCCAU | |
| | CGCCCACGGCACCAACGGCCUGAGUGUGCCCUGCACCAUUC | |
| | CCCAGGAGUUCGUGACCCACUUCGUGAACGAGCAGGCCCCU | |
| | ACCCGGGGUGAAGCCGCCCUGCUGCACUACCUGGACCCCGA | |
| | CACCCACCGGAACCUGGGUGAGUUCAAGCUGUACCCCGACG | |
| | GCUUCAUGACCUGCGUGCCCAAUAGCUCUGGCACCGGUCCC | |
| | CAGACCCUGCCCAUCAACGGCGUGUUCGUGUUCGUGAGCUG | |
| | GGUGUCACGGUUCUACCAGCUGAAGCCCGUGGGCACCGCUG | |
| | GACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGGGC | |
| Corresponding | MMMASKDAPTNMDGTSGAGQLVPEANTAEPISMEPVAGAATA | 74 |
| Amino Acid | AATAGQVNMIDPWIMNNYVQAPQGEFTISPNNTPGDILFDLQLG | |
| Sequence | PHLNPFLSHLAQMYNGWVGNMKVKVLLAGCAFTAGKIIISCIPP | |
| | GFVAQNVSIAQATMFPHVIADVRVLEPIEVPLEDVRNVLFHNND | |
| | NTPTMRLVCMLYTPLRACGSSSGTDPFVIAGRVLTCPSPDFSFLF | |
| | LVPPNVEQKTKPFSVPNLPLNTLSNSRVPSLIKSMMVSRDHGQM | |
| | VQFQNGRVTLDGQLQGTTPTSASQLCKIRGSVFHANGGNGYNLT | |
| | ELDGSPYHAFESPAPIGFPDLGECDWHMEASPTTQFNTGDVIKQI | |
| | NVKQESAFAPHLGTIQADGLSDVSVNTNMIAKLGWVSPVSDGH | |
| | KGDVDPWVIPRYGSTLTEAAQLAPPIYPPGFGEAIVFFMSDFPIAH | |
| | GTNGLSVPCTIPQEFVTHFVNEQAPTRGEAALLHYLDPDTHRNL | |
| | GEFKLYPDGFMTCVPNSSGTGPQTLPINGVFVFVSWVSRFYQLK | |
| | PVGTAGPARRLGIRRS | |
| PolyA tail | 100 nt | |

GI.3 N116C G193C

| SEQ ID NO: 183 consists of from 5′ end to 3′ end: 5′ UTR SEQ ID NO: 1, mRNA | | 183 |
|---|---|---|
| ORF SEQ ID NO: 75, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCCCUACCAACAUGGACGG | 75 |
| Sequence | CACUAGCGGCGCAGGCCAGCUGGUGCCCGAGGCCAACACCG | |
| (nucleotide) | CCGAGCCCAUCAGCAUGGAACCCGUGGCAGGCGCUGCAACC | |
| | GCAGCCGCAACCGCAGGGCAGGUGAACAUGAUCGACCCCUG | |
| | GAUCAUGAACAACUACGUGCAGGCCCCUCAGGGCGAGUUCA | |
| | CCAUCAGCCCCAACAACACGCCCGGCGACAUCCUGUUCGACC | |
| | UGCAGCUGGGGCCCCACCUGAACCCCUUCCUGAGCCACCUG | |
| | GCCCAGAUGUACAACGGCUGGGUGGGCAACAUGAAGGUGAA | |
| | GGUGCUGCUGGCCGGCUGCGCCUUCACCGCCGGCAAGAUCA | |
| | UCAUCAGCUGCAUCCCACCCGGCUUCGUGGCCCAGAACGUG | |
| | AGCAUCGCCCAGGCCACCAUGUUCCCACACGUGAUUGCCGA | |
| | CGUGCGGGUGCUGGAGCCCAUCGAGGUGCCCCUGGAGGACG | |
| | UGCGGAACGUGCUGUUCCACAACAACGACAACACCCCAACC | |
| | AUGCGGCUGGUGUGCAUGCUGUACACCCCUCUGAGGGCCAG | |
| | CUGCAGCAGCAGCGGCACCGACCCCUUCGUGAUCGCCGGCC | |
| | GGGUGCUGACCUGCCCCAGCCCCGACUUCAGCUUCCUGUUC | |
| | CUGGUGCCACCUAACGUGGAGCAGAAGACCAAGCCCUUCAG | |
| | CGUGCCCAACCUGCCCCUGAACACCCUGAGCAACAGCCGGG | |
| | UGCCCAGCCUGAUCAAGAGCAUGAUGGUGAGCCGGGACCAC | |
| | GGCCAGAUGGUGCAGUUCCAGAACGGCCGGGUGACCCUGGA | |
| | CGGCCAGCUGCAAGGCACCACCCCUACCAGCGCCAGCCAGCU | |
| | GUGCAAGAUCCGGGGCAGCGUGUUCCACGCCAACGGCGGCA | |
| | ACGGCUACAACCUGACCGAGCUGGACGGCAGCCCCUACCAC | |
| | GCCUUCGAGAGCCCCGCCCCAAUCGGCUUCCCCGACCUGGGC | |
| | GAGUGCGACUGGCACAUGGAGGCCAGCCCCACCACCCAGUU | |
| | CAACACCGGCGACGUGAUCAAGCAGAUCAACGUGAAGCAGG | |
| | AGAGCGCCUUCGCCCCACACCUGGGCACCAUCCAGGCCGAC | |
| | GGCCUGAGCGACGUGAGCGUGAACACCAACAUGAUCGCCAA | |
| | GCUGGGCUGGGUGAGCCCCGUGAGCGACGGCCACAAGGGCG | |
| | ACGUGGACCCCUGGGUGAUCCCACGGUACGGCAGCACCCUG | |
| | ACCGAAGCUGCCCAGCUCGCCCCUCCAAUCUAUCCUCCGGGC | |
| | UUCGGCGAGGCCAUCGUGUUCUUCAUGAGCGACUUCCCCAU | |
| | CGCCCACGGCACCAACGGCCUGAGUGUGCCCUGCACCAUUC | |
| | CCCAGGAGUUCGUGACCCACUUCGUGAACGAGCAGGCCCCU | |
| | ACCCGGGGUGAAGCCGCCCUGCUGCACUACCUGGACCCCGA | |
| | CACCCACCGGAACCUGGGUGAGUUCAAGCUGUACCCCGACG | |
| | GCUUCAUGACCUGCGUGCCCAAUAGCUCUGGCACCGGUCCC | |
| | CAGACCCUGCCCAUCAACGGCGUGUUCGUGUUCGUGAGCUG | |
| | GGUGUCACGGUUCUACCAGCUGAAGCCCGUGGGCACCGCUG | |
| | GACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDAPTNMDGTSGAGQLVPEANTAEPISMEPVAGAATA | 76 |
| Amino Acid | AATAGQVNMIDPWIMNNYVQAPQGEFTISPNNTPGDILFDLQLG | |
| Sequence | PHLNPFLSHLAQMYNGWVGNMKVKVLLAGCAFTAGKIIISCIPP | |
| | GFVAQNVSIAQATMFPHVIADVRVLEPIEVPLEDVRNVLFHNND | |
| | NTPTMRLVCMLYTPLRASCSSSGTDPFVIAGRVLTCPSPDFSFLFL | |
| | VPPNVEQKTKPFSVPNLPLNTLSNSRVPSLIKSMMVSRDHGQMV | |
| | QFQNGRVTLDGQLQGTTPTSASQLCKIRGSVFHANGGNGYNLTE | |
| | LDGSPYHAFESPAPIGFPDLGECDWHMEASPTTQFNTGDVIKQIN | |
| | VKQESAFAPHLGTIQADGLSDVSVNTNMIAKLGWVSPVSDGHK | |
| | GDVDPWVIPRYGSTLTEAAQLAPPIYPPGFGEAIVFFMSDFPIAHG | |
| | TNGLSVPCTIPQEFVTHFVNEQAPTRGEAALLHYLDPDTHRNLGE | |
| | FKLYPDGFMTCVPNSSGTGPQTLPINGVFVFVSWVSRFYQLKPV | |
| | GTAGPARRLGIRRS | |
| PolyA tail | 100 nt | |

GI.IDS1S/GI.3P

| SEQ ID NO: 184 consists of from 5′ end to 3′ end: 5′ UTR SEQ ID NO: 1, mRNA | | 184 |
|---|---|---|
| ORF SEQ ID NO: 77, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAUGAUGGCCAGCAAGGACGCCACCAGCAGCGUGGACGG | 77 |
| Sequence | CGCAAGCGGCGCAGGCCAGCUGGUGCCCGAGGUGAACGCCA | |
| (nucleotide) | GCGACCCUCUGGCCAUGGAUCCCGUGGCAGGCUCCAGCACC | |
| | GCAGUGGCAACCGCAGGGCAGGUGAACCCCAUCGACCCGUG | |
| | GAUCAUCAACAACUUCGUGCAAGCCCCUCAGGGCGAGUUCA | |
| | CUAUCAGCCCCAACAACACGCCCGGCGACGUUCUGUUCGAC | |
| | CUGAGCCUGGGUCCCCACCUGAACCCCUUCCUUCUGCAUCU | |
| | GAGCCAGAUGUACAACGGUUGGGUGGGCAACAUGCGGGUGC | |
| | GGAUCAUGCUGGCCGGCUGCGCCUUCACCGCCGGCAAGAUC | |
| | AUCGUGAGCUGCAUCCCACCCGGCUUUGGCAGCCACAACCU | |
| | GACCAUCGCACAGGCCACCCUGUUCCCACACGUGAUUGCCG | |
| | ACGUUCGGACCCUGGAUCCCAUCGAGGUGCCCCUGGAGGAC | |
| | GUGCGGAACGUGCUGUUCCACAACAACGACCGGAACCAGCA | |
| | GACCAUGCGGCUGGUGUGCAUGCUGUACACCCCUCUGAGGA | |
| | CCUGCGGCGGCACAGGCGACAGCUUCGUGGUGGCCGGACGG | |
| | GUGAUGACGUGUCCCAGCCCCGACUUCAACUUCCUGUUCCU | |
| | GGUGCCACCUAACGUGGAGCAGAAGACCAAGCCCUUCAGCG | |
| | UGCCCAACCUGCCCCUGAACACCCUGAGCAACAGCCGGGUU | |
| | CCCAGCCUGAUCAAGAGCAUGAUGGUGAGCCGGGACCACGG | |
| | CCAGAUGGUACAGUUCCAGAACGGCCGGGUCACCCUGGACG | |
| | GUCAGCUGCAAGGCACCACACCUACCAGCGCCAGCCAGCUG | |
| | UGCAAGAUCCGGGGCAGCGUGUUCCACGCCAACGGCGGCAA | |
| | CGGCUACAACCUUACCGAGCUGGACGGCAGCCCCUACCACG | |
| | CCUUCGAGAGCCCCGCCCCAAUCGGCUUCCCCGACCUGGGCG | |
| | AGUGCGACUGGCACAUGGAGGCCAGCCCCACCACCCAGUUC | |
| | AACACCGGGGACGUGAUCAAGCAGAUCAACGUGAAGCAGGA | |
| | GAGCGCCUUCGCCCCACACCUGGGCACCAUCCAGGCCGACG | |
| | GCCUCAGCGACGUGAGCGUGAACACCAACAUGAUCGCCAAG | |
| | CUGGGCUGGGUUAGCCCCGUGAGCGACGGCCACAAGGGCGA | |
| | CGUGGACCCCUGGGUGAUCCCACGGUACGGCAGCACCCUGA | |
| | CCGAAGCUGCCCAGCUCGCCCCUCCAAUCUAUCCUCCGGGCU | |
| | UCGGCGAGGCCAUCGUGUUCUUCAUGAGCGACUUCCCCAUC | |
| | GCCCACGGCACCAACGGCCUGAGUGUGCCCUGCACCAUUCC | |
| | CCAGGAGUUCGUGACCCACUUCGUGAACGAGCAGGCCCCAA | |
| | CCCGGGGUGAAGCCGCCCUGCUGCACUACCUGGACCCCGAC | |
| | ACCCACCGGAAUCUGGGUGAGUUCAAGCUGUACCCCGACGG | |
| | CUUCAUGACCUGUGUGCCCAAUAGCUCUGGCACCGGUCCCC | |
| | AGACCCUGCCCAUCAACGGCGUGUUCGUGUUUGUGAGCUGG | |
| | GUGUCACGGUUCUACCAGCUGAAGCCCGUGGGCACCGCUGG | |
| | ACCCGCCAGACGGCUGGGCAUCCGGCGGAGC | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MMMASKDATSSVDGASGAGQLVPEVNASDPLAMDPVAGSSTA | 78 |
| Amino Acid | VATAGQVNPIDPWIINNFVQAPQGEFTISPNNTPGDVLFDLSLGP | |
| Sequence | HLNPFLLHLSQMYNGWVGNMRVRIMLAGCAFTAGKIIVSCIPPG | |
| | FGSHNLTIAQATLFPHVIADVRTLDPIEVPLEDVRNVLFHNNDRN | |
| | QQTMRLVCMLYTPLRTCGGTGDSFVVAGRVMTCPSPDFNFLFL | |
| | VPPNVEQKTKPFSVPNLPLNTLSNSRVPSLIKSMMVSRDHGQMV | |
| | QFQNGRVTLDGQLQGTTPTSASQLCKIRGSVFHANGGNGYNLTE | |
| | LDGSPYHAFESPAPIGFPDLGECDWHMEASPTTQFNTGDVIKQIN | |
| | VKQESAFAPHLGTIQADGLSDVSVNTNMIAKLGWVSPVSDGHK | |
| | GDVDPWVIPRYGSTLTEAAQLAPPIYPPGFGEAIVFFMSDFPIAHG | |
| | TNGLSVPCTIPQEFVTHFVNEQAPTRGEAALLHYLDPDTHRNLGE | |
| | FKLYPDGFMTCVPNSSGTGPQTLPINGVFVFVSWVSRFYQLKPV | |
| | GTAGPARRLGIRRS | |
| PolyA tail | 100 nt | |

GII.4_Sydney_2012_E131T

| SEQ ID NO: 185 consists of from 5′ end to 3′ end: 5′ UTR SEQ ID NO: 1, mRNA | | 185 |
|---|---|---|
| ORF SEQ ID NO: 79, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 79 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCACCGGCCUGAGCCCCAGCCAG | |
| | GUGACCAUGUUCCCUCACAUCAUCGUGGACGUGCGACAGCU | |
| | GGAACCCGUGCUGAUACCCCUGCCCGACGUGCGGAACAAUU | |
| | UCUACCACUACAACCAGAGCAACGACCCCACCAUCAAGCUG | |
| | AUCGCCAUGCUGUACACCCCACUGCGGGCCAACAACGCCGG | |
| | CGACGACGUGUUUACCGUGAGCUGCCGGGUUCUGACCCGGC | |
| | CCAGCCCAGACUUCGACUUCAUCUUUCUGGUGCCACCCACC | |
| | GUGGAGAGCCGGACCAAGCCCUUCAGCGUGCCGGUGCUGAC | |
| | CGUCGAGGAGAUGACCAACAGCCGGUUCCCCAUUCCCCUGG | |
| | AGAAGCUGUUCACCGGCCCCUCCAGCGCCUUCGUGGUGCAG | |
| | CCCCAGAACGGCCGGUGCACCACCGACGGCGUGCUGUUGGG | |
| | CACUACCCAGCUCAGCCCCGUGAACAUCUGCAACUUCCGGG | |
| | GCGACGUGACCCACAUCACCGGAAGCCGGAACUACACCAUG | |
| | AACCUGGCCAGCCAGAACUGGAAUAACUACGACCCUACGGA | |
| | GGAGAUCCCCGCACCCCUGGGCACUCCCGACUUCGUGGGCA | |
| | AGAUUCAGGGCAUGCUGACCCAGACCACCCGGACCGACGGG | |
| | AGCACACGGGGCCACAAGGCCACAGUGUACACCGGCAGCGC | |
| | GGACUUCGCGCCCAAACUGGGCCGCGUGCAGUUCGAGACCG | |
| | ACACCGACCACGACUUCGAGGCCAACCAGAACACCAAGUUC | |
| | ACACCCGUGGGCGUGAUCCAGGACGGCAGCACAACCCACCG | |
| | GAACGAACCCCAGCAGUGGGUCCUGCCCAGCUACAGCGGCC | |
| | GGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGUGGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 80 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTT | |
| | GLSPSQVTMFPHIIVDVRQLEPVLIPLPDVRNNFYHYNQSNDPTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICNFRGDVTHITGSRNYTMNLASQNWNNYDP | |
| | TEEIPAPLGTPDFVGKIQGMLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRVV | |
| PolyA tail | 100 nt | |

GII.4c_USA_T219P_V220P

| SEQ ID NO: 186 consists of from 5′ end to 3′ end: 5′ UTR SEQ ID NO: 1, mRNA | | 186 |
|---|---|---|
| ORF SEQ ID NO: 81, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 81 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCGCC | |
| | ACCUGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 82 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYTPLRANNAGDDVFTVSCRVLTRPSPDFDFIFLVPPPPESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRAV | |
| PolyA tail | 100 nt | |

GII.4c_USA_E63R_S519W

| SEQ ID NO: 187 consists of from 5′ end to 3′ end: 5′ UTR SEQ ID NO: 1, mRNA | | 187 |
|---|---|---|
| ORF SEQ ID NO: 83, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 83 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCAGAUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUUGGUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 84 |
| Amino Acid | GQQNVIDPWIRNNFVQAPGGRFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDWWVNQFYTLAPMG | |
| | NGTGRRRAV | |
| PolyA tail | 100 nt | |

GII.4c_USA_E63R_S519W_T219P_V220P

| SEQ ID NO: 188 consists of from 5′ end to 3′ end: 5′ UTR SEQ ID NO: 1, mRNA | | 188 |
|---|---|---|
| ORF SEQ ID NO: 85, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 85 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCAGAUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCACC | |
| | ACCUGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUUGGUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 86 |
| Amino Acid | GQQNVIDPWIRNNFVQAPGGRFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYTPLRANNAGDDVFTVSCRVLTRPSPDFDFIFLVPPPPESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDWWVNQFYTLAPMG | |
| | NGTGRRRAV | |
| PolyA tail | 100 nt | |

GII.2_T219P_V220P

| SEQ ID NO: 189 consists of from 5′ end to 3′ end: 5′ UTR SEQ ID NO: 1, mRNA | | 189 |
|---|---|---|
| ORF SEQ ID NO: 87, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCACCCAGCACCGACGG | 87 |
| Sequence | CGCAGCCGGACUGGUGCCCGAGAGCAACAACGAGGUGAUGG | |
| (nucleotide) | CCCUGGAACCUGUGGCAGGCGCCGCUCUGGCUGCACCCGUG | |
| | ACCGGCCAGACCAACAUCAUCGACCCCUGGAUCCGGGCCAA | |
| | CUUCGUGCAGGCUCCCAACGGCGAGUUCACCGUGUCUCCCC | |
| | GGAACGCUCCCGGCGAGGUGCUGCUGAACCUGGAGCUGGGC | |
| | CCCGAGCUGAACCCCUACCUGGCCCACCUGGCCCGGAUGUA | |
| | CAACGGCUACGCCGGCGGCAUGGAGGUGCAGGUGAUGCUGG | |
| | CCGGCAACGCCUUCACCGCCGGCAAGCUGGUGUUCGCCGCC | |
| | GUGCCACCACACUUCCCCGUGGAGAACCUGUCUCCCCAGCA | |
| | GAUCACCAUGUUCCCUCACGUGAUCAUAGACGUGCGGACCC | |
| | UGGAGCCCGUGCUGCUGCCCCUGCCCGACGUGCGGAACAAC | |
| | UUCUUCCACUACAACCAGAAGGACGACCCCAAGAUGCGGAU | |
| | CGUGGCCAUGCUGUACACUCCCCUGCGGAGCAACGGCAGCG | |
| | GCGACGACGUGUUCACCGUGAGCUGCCGGGUGCUGACCAGA | |
| | CCCAGCCCCGACUUCGACUUCACCUACCUGGUGCCACCUCCU | |
| | CCCGAGAGCAAGACCAAGCCCUUCACCCUGCCCAUCCUGACC | |
| | CUGGGCGAGCUGAGCAACAGCCGGUUCCCCGUGAGCAUCGA | |
| | CCAGAUGUACACCAGCCCCAACGAGAUCAUCAGCGUGCAGU | |
| | GCCAGAACGGCCGGUGCACCCUGGACGGCGAGCUGCAGGGC | |
| | ACCACCCAGCUGCAGGUGAGCGGCAUCUGCGCCUUCAAGGG | |
| | CGAGGUGACCGCCCACCUGCACGACAACGACCACCUGUACA | |
| | ACGUGACCAUCACCAACCUGAACGGCAGCCCCUUCGACCCC | |
| | AGCGAGGACAUCCCCGCACCACUGGGCGUGCCCGACUUCCA | |
| | GGGCCGGGUGUUCGGCAUCAUCAGCCAGCGGGACAAGCACA | |
| | ACAGCCCCGGCCACAACGAGCCUGCCAACCGCGGACACGAC | |
| | GCCGUGGUGCCCACCUACACCGCCCAGUACACCCCAAAGCU | |
| | GGGCCAGAUCCAGAUCGGCACCUGGCAGACCGACGACCUGA | |
| | CCGUGAACCAGCCCGUGAAGUUCACACCCGUGGGCCUGAAC | |
| | GACACCGAGCACUUCAACCAGUGGGUGGUCCCACGGUACGC | |
| | CGGUGCCCUGAACCUGAACACCAACCUGGCUCCCAGCGUGG | |
| | CUCCCGUGUUCCCCGGCGAGCGGCUGCUGUUCUUCCGGAGC | |
| | UACAUCCCACUGAAGGGCGGCUACGGCAACCCCGCCAUCGA | |
| | CUGCCUGCUGCCCCAGGAGUGGGUGCAGCACUUCUACCAGG | |
| | AGGCCGCCCCAAGCAUGAGCGAGGUGGCCCUGGUGCGGUAC | |
| | AUCAACCCCGACACCGGCAGAGCCCUGUUCGAGGCCAAGCU | |
| | GCACCGGGCCGGCUUCAUGACCGUGAGCAGCAACACCAGCG | |
| | CCCCUGUGGUGGUGCCCGCCAACGGCUACUUCCGGUUCGAC | |
| | AGCUGGGUGAACCAGUUCUACAGCCUGGCCCCAAUGGGAAC | |
| | CGGUAACGGACGCCGGCGGGUGCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSTDGAAGLVPESNNEVMALEPVAGAALAAPVT | 88 |
| Amino Acid | GQTNIIDPWIRANFVQAPNGEFTVSPRNAPGEVLLNLELGPELNP | |
| Sequence | YLAHLARMYNGYAGGMEVQVMLAGNAFTAGKLVFAAVPPHFP | |
| | VENLSPQQITMFPHVIIDVRTLEPVLLPLPDVRNNFFHYNQKDDP | |
| | KMRIVAMLYTPLRSNGSGDDVFTVSCRVLTRPSPDFDFTYLVPPP | |
| | PESKTKPFTLPILTLGELSNSRFPVSIDQMYTSPNEIISVQCQNGRC | |
| | TLDGELQGTTQLQVSGICAFKGEVTAHLHDNDHLYNVTITNLNG | |
| | SPFDPSEDIPAPLGVPDFQGRVFGIISQRDKHNSPGHNEPANRGHD | |
| | AVVPTYTAQYTPKLGQIQIGTWQTDDLTVNQPVKFTPVGLNDTE | |
| | HFNQWVVPRYAGALNLNTNLAPSVAPVFPGERLLFFRSYIPLKG | |
| | GYGNPAIDCLLPQEWVQHFYQEAAPSMSEVALVRYINPDTGRAL | |
| | FEAKLHRAGFMTVSSNTSAPVVVPANGYFRFDSWVNQFYSLAP | |
| | MGTGNGRRRVQ | |
| PolyA tail | 100 nt | |

GII.2_E63R_S519W

| SEQ ID NO: 190 consists of from 5′ end to 3′ end: 5′ UTR SEQ ID NO: 1, mRNA | | 190 |
|---|---|---|
| ORF SEQ ID NO: 89, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCACCCAGCACCGACGG | 89 |
| Sequence | CGCAGCCGGACUGGUGCCCGAGAGCAACAACGAGGUGAUGG | |
| (nucleotide) | CCCUGGAACCUGUGGCAGGCGCCGCUCUGGCUGCACCCGUG | |
| | ACCGGCCAGACCAACAUCAUCGACCCCUGGAUCCGGGCCAA | |
| | CUUCGUGCAGGCUCCCAACGGCCGGUUCACCGUGUCUCCCC | |
| | GGAACGCUCCCGGCGAGGUGCUGCUGAACCUGGAGCUGGGC | |
| | CCCGAGCUGAACCCCUACCUGGCCCACCUGGCCCGGAUGUA | |
| | CAACGGCUACGCCGGCGGCAUGGAGGUGCAGGUGAUGCUGG | |
| | CCGGCAACGCCUUCACCGCCGGCAAGCUGGUGUUCGCCGCC | |
| | GUGCCACCACACUUCCCCGUGGAGAACCUGUCUCCCCAGCA | |
| | GAUCACCAUGUUCCCUCACGUGAUCAUAGACGUGCGGACCC | |
| | UGGAGCCCGUGCUGCUGCCCCUGCCCGACGUGCGGAACAAC | |
| | UUCUUCCACUACAACCAGAAGGACGACCCCAAGAUGCGGAU | |
| | CGUGGCCAUGCUGUACACUCCCCUGCGGAGCAACGGCAGCG | |
| | GCGACGACGUGUUCACCGUGAGCUGCCGGGUGCUGACCAGA | |
| | CCCAGCCCCGACUUCGACUUCACCUACCUGGUGCCACCCACC | |
| | GUGGAGAGCAAGACCAAGCCCUUCACCCUGCCCAUCCUGAC | |
| | CCUGGGCGAGCUGAGCAACAGCCGGUUCCCCGUGAGCAUCG | |
| | ACCAGAUGUACACCAGCCCCAACGAGAUCAUCAGCGUGCAG | |
| | UGCCAGAACGGCCGGUGCACCCUGGACGGCGAGCUGCAGGG | |
| | CACCACCCAGCUGCAGGUGAGCGGCAUCUGCGCCUUCAAGG | |
| | GCGAGGUGACCGCCCACCUGCACGACAACGACCACCUGUAC | |
| | AACGUGACCAUCACCAACCUGAACGGCAGCCCCUUCGACCC | |
| | CAGCGAGGACAUCCCCGCACCACUGGGCGUGCCCGACUUCC | |
| | AGGGCCGGGUGUUCGGCAUCAUCAGCCAGCGGGACAAGCAC | |
| | AACAGCCCCGGCCACAACGAGCCUGCCAACCGCGGACACGA | |
| | CGCCGUGGUGCCCACCUACACCGCCCAGUACACCCCAAAGCU | |
| | GGGCCAGAUCCAGAUCGGCACCUGGCAGACCGACGACCUGA | |
| | CCGUGAACCAGCCCGUGAAGUUCACACCCGUGGGCCUGAAC | |
| | GACACCGAGCACUUCAACCAGUGGGUGGUCCCACGGUACGC | |
| | CGGUGCCCUGAACCUGAACACCAACCUGGCUCCCAGCGUGG | |
| | CUCCCGUGUUCCCCGGCGAGCGGCUGCUGUUCUUCCGGAGC | |
| | UACAUCCCACUGAAGGGCGGCUACGGCAACCCCGCCAUCGA | |
| | CUGCCUGCUGCCCCAGGAGUGGGUGCAGCACUUCUACCAGG | |
| | AGGCCGCCCCAAGCAUGAGCGAGGUGGCCCUGGUGCGGUAC | |
| | AUCAACCCCGACACCGGCAGAGCCCUGUUCGAGGCCAAGCU | |
| | GCACCGGGCCGGCUUCAUGACCGUGAGCAGCAACACCAGCG | |
| | CCCCUGUGGUGGUGCCCGCCAACGGCUACUUCCGGUUCGAC | |
| | UGGUGGGUGAACCAGUUCUACAGCCUGGCCCCAAUGGGAAC | |
| | CGGUAACGGACGCCGGCGGGUGCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSTDGAAGLVPESNNEVMALEPVAGAALAAPVT | 90 |
| Amino Acid | GQTNIIDPWIRANFVQAPNGRFTVSPRNAPGEVLLNLELGPELNP | |
| Sequence | YLAHLARMYNGYAGGMEVQVMLAGNAFTAGKLVFAAVPPHFP | |
| | VENLSPQQITMFPHVIIDVRTLEPVLLPLPDVRNNFFHYNQKDDP | |
| | KMRIVAMLYTPLRSNGSGDDVFTVSCRVLTRPSPDFDFTYLVPPT | |
| | VESKTKPFTLPILTLGELSNSRFPVSIDQMYTSPNEIISVQCQNGRC | |
| | TLDGELQGTTQLQVSGICAFKGEVTAHLHDNDHLYNVTITNLNG | |
| | SPFDPSEDIPAPLGVPDFQGRVFGIISQRDKHNSPGHNEPANRGHD | |
| | AVVPTYTAQYTPKLGQIQIGTWQTDDLTVNQPVKFTPVGLNDTE | |
| | HFNQWVVPRYAGALNLNTNLAPSVAPVFPGERLLFFRSYIPLKG | |
| | GYGNPAIDCLLPQEWVQHFYQEAAPSMSEVALVRYINPDTGRAL | |
| | FEAKLHRAGFMTVSSNTSAPVVVPANGYFRFDWWVNQFYSLAP | |
| | MGTGNGRRRVQ | |
| PolyA tail | 100 nt | |

GII.2_E63R_S519W_T219P_V220P

| SEQ ID NO: 191 consists of from 5′ end to 3′ end: 5′ UTR SEQ ID NO: 1, mRNA | | 191 |
|---|---|---|
| ORF SEQ ID NO: 91, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAACGACGCCGCACCCAGCACCGACGG | 91 |
| Sequence | CGCAGCCGGACUGGUGCCCGAGAGCAACAACGAGGUGAUGG | |
| (nucleotide) | CCCUGGAACCUGUGGCAGGCGCCGCUCUGGCUGCACCCGUG | |
| | ACCGGCCAGACCAACAUCAUCGACCCCUGGAUCCGGGCCAA | |
| | CUUCGUGCAGGCUCCCAACGGCCGGUUCACCGUGUCUCCCC | |
| | GGAACGCUCCCGGCGAGGUGCUGCUGAACCUGGAGCUGGGC | |
| | CCCGAGCUGAACCCCUACCUGGCCCACCUGGCCCGGAUGUA | |
| | CAACGGCUACGCCGGCGGCAUGGAGGUGCAGGUGAUGCUGG | |
| | CCGGCAACGCCUUCACCGCCGGCAAGCUGGUGUUCGCCGCC | |
| | GUGCCACCACACUUCCCCGUGGAGAACCUGUCUCCCCAGCA | |
| | GAUCACCAUGUUCCCUCACGUGAUCAUAGACGUGCGGACCC | |
| | UGGAGCCCGUGCUGCUGCCCCUGCCCGACGUGCGGAACAAC | |
| | UUCUUCCACUACAACCAGAAGGACGACCCCAAGAUGCGGAU | |
| | CGUGGCCAUGCUGUACACUCCCCUGCGGAGCAACGGCAGCG | |
| | GCGACGACGUGUUCACCGUGAGCUGCCGGGUGCUGACCAGA | |
| | CCCAGCCCCGACUUCGACUUCACCUACCUGGUGCCACCACCU | |
| | CCCGAGAGCAAGACCAAGCCCUUCACCCUGCCCAUCCUGACC | |
| | CUGGGCGAGCUGAGCAACAGCCGGUUCCCCGUGAGCAUCGA | |
| | CCAGAUGUACACCAGCCCCAACGAGAUCAUCAGCGUGCAGU | |
| | GCCAGAACGGCCGGUGCACCCUGGACGGCGAGCUGCAGGGC | |
| | ACCACCCAGCUGCAGGUGAGCGGCAUCUGCGCCUUCAAGGG | |
| | CGAGGUGACCGCCCACCUGCACGACAACGACCACCUGUACA | |
| | ACGUGACCAUCACCAACCUGAACGGCAGCCCCUUCGACCCC | |
| | AGCGAGGACAUCCCCGCACCACUGGGCGUGCCCGACUUCCA | |
| | GGGCCGGGUGUUCGGCAUCAUCAGCCAGCGGGACAAGCACA | |
| | ACAGCCCCGGCCACAACGAGCCUGCCAACCGCGGACACGAC | |
| | GCCGUGGUGCCCACCUACACCGCCCAGUACACCCCAAAGCU | |
| | GGGCCAGAUCCAGAUCGGCACCUGGCAGACCGACGACCUGA | |
| | CCGUGAACCAGCCCGUGAAGUUCACACCCGUGGGCCUGAAC | |
| | GACACCGAGCACUUCAACCAGUGGGUGGUCCCACGGUACGC | |
| | CGGUGCCCUGAACCUGAACACCAACCUGGCUCCCAGCGUGG | |
| | CUCCCGUGUUCCCCGGCGAGCGGCUGCUGUUCUUCCGGAGC | |
| | UACAUCCCACUGAAGGGCGGCUACGGCAACCCCGCCAUCGA | |
| | CUGCCUGCUGCCCCAGGAGUGGGUGCAGCACUUCUACCAGG | |
| | AGGCCGCCCCAAGCAUGAGCGAGGUGGCCCUGGUGCGGUAC | |
| | AUCAACCCCGACACCGGCAGAGCCCUGUUCGAGGCCAAGCU | |
| | GCACCGGGCCGGCUUCAUGACCGUGAGCAGCAACACCAGCG | |
| | CCCCUGUGGUGGUGCCCGCCAACGGCUACUUCCGGUUCGAC | |
| | UGGUGGGUGAACCAGUUCUACAGCCUGGCCCCAAUGGGAAC | |
| | CGGUAACGGACGCCGGCGGGUGCAG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASNDAAPSTDGAAGLVPESNNEVMALEPVAGAALAAPVT | 92 |
| Amino Acid | GQTNIIDPWIRANFVQAPNGRFTVSPRNAPGEVLLNLELGPELNP | |
| Sequence | YLAHLARMYNGYAGGMEVQVMLAGNAFTAGKLVFAAVPPHFP | |
| | VENLSPQQITMFPHVIIDVRTLEPVLLPLPDVRNNFFHYNQKDDP | |
| | KMRIVAMLYTPLRSNGSGDDVFTVSCRVLTRPSPDFDFTYLVPPP | |
| | PESKTKPFTLPILTLGELSNSRFPVSIDQMYTSPNEIISVQCQNGRC | |
| | TLDGELQGTTQLQVSGICAFKGEVTAHLHDNDHLYNVTITNLNG | |
| | SPFDPSEDIPAPLGVPDFQGRVFGIISQRDKHNSPGHNEPANRGHD | |
| | AVVPTYTAQYTPKLGQIQIGTWQTDDLTVNQPVKFTPVGLNDTE | |
| | HFNQWVVPRYAGALNLNTNLAPSVAPVFPGERLLFFRSYIPLKG | |
| | GYGNPAIDCLLPQEWVQHFYQEAAPSMSEVALVRYINPDTGRAL | |
| | FEAKLHRAGFMTVSSNTSAPVVVPANGYFRFDWWVNQFYSLAP | |
| | MGTGNGRRRVQ | |
| PolyA tail | 100 nt | |

GII.4c_USA_T184C_S199C

| SEQ ID NO: 192 consists of from 5′ end to 3′ end: 5′ UTR SEQ ID NO: 1, mRNA | | 192 |
|---|---|---|
| ORF SEQ ID NO: 93, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 93 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACUGCCCACUGCGGGCCAACAACGCCG | |
| | GCGACGACGUGUUUACCGUGUGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 94 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTMFPHIIVDVRQLEPVLIPLPDVRNNFYHYNQSNDSTIK | |
| | LIAMLYCPLRANNAGDDVFTVCCRVLTRPSPDFDFIFLVPPTVES | |
| | RTKPFSVPVLTVEEMTNSRFPIPLEKLFTGPSSAFVVQPQNGRCTT | |
| | DGVLLGTTQLSPVNICTFRGDVTHITGSRNYTMNLASQNWNNY | |
| | DPTEEIPAPLGTPDFVGKIQGVLTQTTRTDGSTRGHKATVYTGSA | |
| | DFAPKLGRVQFETDTDHDFEANQNTKFTPVGVIQDGSTTHRNEP | |
| | QQWVLPSYSGRNTHNVHLAPAVAPTFPGEQLLFFRSTMPGCSGY | |
| | PNMDLDCLLPQEWVQYFYQEAAPAQSDVALLRFVNPDTGRVLF | |
| | ECKLHKSGYVTVAHTGQHDLVIPPNGYFRFDSWVNQFYTLAPM | |
| | GNGTGRRRAV | |
| PolyA tail | 100 nt | |

GII.4c_USA_N127C_Y168C

| SEQ ID NO: 193 consists of from 5′ end to 3′ end: 5′ UTR SEQ ID NO: 1, mRNA | | 193 |
|---|---|---|
| ORF SEQ ID NO: 95, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 95 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGUGCUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUGCAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 96 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPCFPTE | |
| | GLSPSQVTMFPHIIVDVRQLEPVLIPLPDVRNNFYHCNQSNDSTIK | |
| | LIAMLYTPLRANNAGDDVFTVSCRVLTRPSPDFDFIFLVPPTVESR | |
| | TKPFSVPVLTVEEMTNSRFPIPLEKLFTGPSSAFVVQPQNGRCTTD | |
| | GVLLGTTQLSPVNICTFRGDVTHITGSRNYTMNLASQNWNNYDP | |
| | TEEIPAPLGTPDFVGKIQGVLTQTTRTDGSTRGHKATVYTGSADF | |
| | APKLGRVQFETDTDHDFEANQNTKFTPVGVIQDGSTTHRNEPQQ | |
| | WVLPSYSGRNTHNVHLAPAVAPTFPGEQLLFFRSTMPGCSGYPN | |
| | MDLDCLLPQEWVQYFYQEAAPAQSDVALLRFVNPDTGRVLFEC | |
| | KLHKSGYVTVAHTGQHDLVIPPNGYFRFDSWVNQFYTLAPMGN | |
| | GTGRRRAV | |
| PolyA tail | 100 nt | |

GII.4c_USA_M140C_P217C

| SEQ ID NO: 194 consists of from 5′ end to 3′ end: 5′ UTR SEQ ID NO: 1, mRNA | | 194 |
|---|---|---|
| ORF SEQ ID NO: 97, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 97 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCUGCUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACACCCCACUGCGGGCCAACAACGCCG | |
| | GCGACGACGUGUUUACCGUGAGCUGCCGGGUUCUGACCCGG | |
| | CCCAGCCCAGACUUCGACUUCAUCUUUCUGGUGUGCCCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 98 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTCFPHIIVDVRQLEPVLIPLPDVRNNFYHYNQSNDSTIK | |
| | LIAMLYTPLRANNAGDDVFTVSCRVLTRPSPDFDFIFLVCPTVES | |
| | RTKPFSVPVLTVEEMTNSRFPIPLEKLFTGPSSAFVVQPQNGRCTT | |
| | DGVLLGTTQLSPVNICTFRGDVTHITGSRNYTMNLASQNWNNY | |
| | DPTEEIPAPLGTPDFVGKIQGVLTQTTRTDGSTRGHKATVYTGSA | |
| | DFAPKLGRVQFETDTDHDFEANQNTKFTPVGVIQDGSTTHRNEP | |
| | QQWVLPSYSGRNTHNVHLAPAVAPTFPGEQLLFFRSTMPGCSGY | |
| | PNMDLDCLLPQEWVQYFYQEAAPAQSDVALLRFVNPDTGRVLF | |
| | ECKLHKSGYVTVAHTGQHDLVIPPNGYFRFDSWVNQFYTLAPM | |
| | GNGTGRRRAV | |
| PolyA tail | 100 nt | |

GII.4c_USA_N127C_Y168C_T184C_S199C

| SEQ ID NO: 195 consists of from 5′ end to 3′ end: 5′ UTR SEQ ID NO: 1, mRNA | | 195 |
|---|---|---|
| ORF SEQ ID NO: 99, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 99 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGUGCUUCCCUACCGAGGGCCUGAGCCCCAGCCA | |
| | GGUGACCAUGUUCCCUCACAUCAUCGUGGACGUGCGACAGC | |
| | UGGAACCCGUGCUGAUACCCCUGCCCGACGUGCGGAACAAU | |
| | UUCUACCACUGCAACCAGAGCAACGACAGCACCAUCAAGCU | |
| | GAUCGCCAUGCUGUACUGCCCACUGCGGGCCAACAACGCCG | |
| | GCGACGACGUGUUUACCGUGUGCUGCCGGGUUCUGACCCGG | |
| | CCCAGCCCAGACUUCGACUUCAUCUUUCUGGUGCCACCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 100 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPCFPTE | |
| | GLSPSQVTMFPHIIVDVRQLEPVLIPLPDVRNNFYHCNQSNDSTIK | |
| | LIAMLYCPLRANNAGDDVFTVCCRVLTRPSPDFDFIFLVPPTVES | |
| | RTKPFSVPVLTVEEMTNSRFPIPLEKLFTGPSSAFVVQPQNGRCTT | |
| | DGVLLGTTQLSPVNICTFRGDVTHITGSRNYTMNLASQNWNNY | |
| | DPTEEIPAPLGTPDFVGKIQGVLTQTTRTDGSTRGHKATVYTGSA | |
| | DFAPKLGRVQFETDTDHDFEANQNTKFTPVGVIQDGSTTHRNEP | |
| | QQWVLPSYSGRNTHNVHLAPAVAPTFPGEQLLFFRSTMPGCSGY | |
| | PNMDLDCLLPQEWVQYFYQEAAPAQSDVALLRFVNPDTGRVLF | |
| | ECKLHKSGYVTVAHTGQHDLVIPPNGYFRFDSWVNQFYTLAPM | |
| | GNGTGRRRAV | |
| PolyA tail | 100 nt | |

GII.4c_USA_M140C_P217C_T184C_S199C

| SEQ ID NO: 196 consists of from 5′ end to 3′ end: 5′ UTR SEQ ID NO: 1, mRNA | | 196 |
|---|---|---|
| ORF SEQ ID NO: 101, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAG | 1 |
| | ACCCCGGCGCCGCCACC | |
| ORF of mRNA | AUGAAGAUGGCCAGCAGCGACGCCAACCCAAGCGACGGCAG | 101 |
| Sequence | UGCCGCCAACCUGGUGCCCGAGGUGAACAACGAGGUGAUGG | |
| (nucleotide) | CCCUGGAGCCCGUGGUAGGUGCCGCGAUCGCUGCCCCUGUG | |
| | GCCGGACAGCAGAACGUGAUCGACCCCUGGAUCCGGAACAA | |
| | CUUUGUGCAGGCCCCUGGCGGCGAGUUCACCGUGUCCCCUC | |
| | GGAACGCUCCCGGCGAGAUCCUUUGGAGCGCGCCGUUAGGC | |
| | CCCGACCUGAACCCCUACCUGAGCCACCUGGCCCGGAUGUA | |
| | CAACGGCUACGCUGGCGGCUUCGAGGUGCAGGUGAUCCUGG | |
| | CCGGCAACGCCUUCACCGCCGGCAAGAUCAUCUUCGCCGCC | |
| | GUGCCACCGAACUUCCCUACCGAGGGCCUGAGCCCCAGCCA | |
| | GGUGACCUGCUUCCCUCACAUCAUCGUGGACGUGCGACAGC | |
| | UGGAACCCGUGCUGAUACCCCUGCCCGACGUGCGGAACAAU | |
| | UUCUACCACUACAACCAGAGCAACGACAGCACCAUCAAGCU | |
| | GAUCGCCAUGCUGUACUGCCCACUGCGGGCCAACAACGCCG | |
| | GCGACGACGUGUUUACCGUGUGCUGCCGGGUUCUGACCCGG | |
| | CCCAGCCCAGACUUCGACUUCAUCUUUCUGGUGUGCCCCAC | |
| | CGUGGAGAGCCGGACCAAGCCCUUCAGCGUGCCGGUGCUGA | |
| | CCGUCGAGGAGAUGACCAACAGCCGGUUCCCCAUUCCCCUG | |
| | GAGAAGCUGUUCACCGGCCCCUCCAGCGCCUUCGUGGUGCA | |
| | GCCCCAGAACGGCCGGUGCACCACCGACGGCGUGCUGUUGG | |
| | GCACUACCCAGCUCAGCCCCGUGAACAUCUGCACCUUCCGG | |
| | GGCGACGUGACCCACAUCACCGGAAGCCGGAACUACACCAU | |
| | GAACCUGGCCAGCCAGAACUGGAAUAACUACGACCCUACGG | |
| | AGGAGAUCCCCGCACCCCUGGGCACUCCCGACUUCGUGGGC | |
| | AAGAUUCAGGGCGUGCUGACCCAGACCACCCGGACCGACGG | |
| | GAGCACACGGGGCCACAAGGCCACAGUGUACACCGGCAGCG | |
| | CGGACUUCGCGCCCAAACUGGGCCGCGUGCAGUUCGAGACC | |
| | GACACCGACCACGACUUCGAGGCCAACCAGAACACCAAGUU | |
| | CACACCCGUGGGCGUGAUCCAGGACGGCAGCACAACCCACC | |
| | GGAACGAACCCCAGCAGUGGGUCCUGCCCAGCUACAGCGGC | |
| | CGGAACACCCACAACGUGCACCUGGCACCCGCCGUGGCACCC | |
| | ACCUUCCCCGGCGAACAGCUGCUGUUCUUCCGGAGCACCAU | |
| | GCCCGGCUGCAGCGGCUACCCCAACAUGGACCUGGACUGCC | |
| | UGCUGCCCCAGGAGUGGGUGCAGUACUUCUACCAGGAGGCA | |
| | GCUCCCGCCCAGAGCGACGUGGCCCUGCUGCGGUUCGUGAA | |
| | CCCCGAUACCGGCCGGGUGCUGUUCGAGUGCAAGCUGCACA | |
| | AGUCGGGCUACGUGACCGUGGCCCACACCGGCCAGCACGAC | |
| | CUGGUGAUCCCUCCUAACGGGUACUUCCGGUUCGAUAGCUG | |
| | GGUGAACCAGUUCUACACCCUGGCGCCUAUGGGCAACGGCA | |
| | CCGGACGUAGACGGGCCGUG | |
| 3′ UTR | UGAUAAUAGGCUGGAGCCUCGGUGGCCUAGCUUCUUGCCCC | 4 |
| | UUGGGCCUCCCCCCAGCCCCUCCUCCCCUUCCUGCACCCGUA | |
| | CCCCCGUGGUCUUUGAAUAAAGUCUGAGUGGGCGGC | |
| Corresponding | MKMASSDANPSDGSAANLVPEVNNEVMALEPVVGAAIAAPVA | 102 |
| Amino Acid | GQQNVIDPWIRNNFVQAPGGEFTVSPRNAPGEILWSAPLGPDLNP | |
| Sequence | YLSHLARMYNGYAGGFEVQVILAGNAFTAGKIIFAAVPPNFPTE | |
| | GLSPSQVTCFPHIIVDVRQLEPVLIPLPDVRNNFYHYNQSNDSTIK | |
| | LIAMLYCPLRANNAGDDVFTVCCRVLTRPSPDFDFIFLVCPTVES | |
| | RTKPFSVPVLTVEEMTNSRFPIPLEKLFTGPSSAFVVQPQNGRCTT | |
| | DGVLLGTTQLSPVNICTFRGDVTHITGSRNYTMNLASQNWNNY | |
| | DPTEEIPAPLGTPDFVGKIQGVLTQTTRTDGSTRGHKATVYTGSA | |
| | DFAPKLGRVQFETDTDHDFEANQNTKFTPVGVIQDGSTTHRNEP | |
| | QQWVLPSYSGRNTHNVHLAPAVAPTFPGEQLLFFRSTMPGCSGY | |
| | PNMDLDCLLPQEWVQYFYQEAAPAQSDVALLRFVNPDTGRVLF | |
| | ECKLHKSGYVTVAHTGQHDLVIPPNGYFRFDSWVNQFYTLAPM | |
| | GNGTGRRRAV | |
| PolyA tail | 100 nt | |

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a poly(A) tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

TABLE 2
5′UTR sequences

| SEQ ID | |
|---|---|
| NO: | Sequence |
| 103 | GGGAAAUAAGAGAGAAAAGAAGAGUAAGAAGAAAUAUAAGAGCCACC |
| 104 | GGGAAAUAAGAGAGAAAAGAAGAGUAAGAAGAAAUAUAAGACCCCGGCGCC |
| | GCCACC |
| 107 | GGAAAUCGCAAAAUUUGCUCUUCGCGUUAGAUUUCUUUUAGUUUUCUCGCAA |
| | CUAGCAAGCUUUUUGUUCUCGCC |
| 108 | GGAAAUCCCCACAACCGCCUCAUAUCCAGGCUCAAGAAUAGAGCUCAGUGUU |
| | UUGUUGUUUAAUCAUUCCGACGUGUUUUGCGAUAUUCGCGCAAAGCAGCCAG |
| | UCGCGCGCUUGCUUUUAAGUAGAGUUGUUUUUCCACCCGUUUGCCAGGCAUC |
| | UUUAAUUUAACAUAUUUUUAUUUUUCAGGCUAACCUACGCCGCCACC |
| 109 | GGAAAUAAGAGAGAAAAGAAGAGUAAGAAGAAAUAUAAGAUCUCCCUGAGC |
| | UUCAGGGAGCCCCGGCGCCGCCACC |
| 110 | GGAAACCCCCCACCCCCGUAAGAGAGAAAAGAAGAGUAAGAAGAAAUAUAAG |
| | AUCUCCCUGAGCUUCAGGGAGCCCCGGCGCCGCCACC |
| 111 | GGAGAACUUCCGCUUCCGUUGGCGCAAGCGCUUUCAUUUUUUCUGCUACCGU |
| | GACUAAG |
| 112 | GGAAAUAAGAGAGAAAAGAAGAGUAAGAAGAAAUAUAAGAGCCACC |
| 113 | GGAAAUAAGAGAGAAAAGAAGAGUAAGAAGAAAUAUAAGACCCCGGCGCCG |
| | CCACC |
| 114 | GGAAAUCGCAAAAUUUGCUCUUCGCGUUAGAUUUCUUUUAGUUUUCUCGCAA |
| | CUAGCAAGCUUUUUGUUCUCGCCGCCGCC |
| 115 | GGAAAUCGCAAAAUUUUUUUUCGCGUUAGAUUUCUUUUAGUUUUCUUUCA |
| | ACUAGCAAGCUUUUUGUUCUCGCCGCCGCC |
| 116 | GGAAAUCGCAAAA(N2)X(N3)XCU(N4)X(N5)XCGCGUUAGAUU |
| | UCUUUUAGUUUUCUN6N7CAACUAGCAAGCUUUUUGUUC |
| | UCGCC(N8CC)x |
| | (N2)x is a uracil and x is an integer from 0 to 5, e.g., |
| | wherein x = 3 or 4; |
| | (N3)x is a guanine and x is an integer from 0 to 1; |
| | (N4)x is a cytosine and x is an integer from 0 to 1; |
| | (N5)x is a uracil and x is an integer from 0 to 5, e.g., |
| | wherein x = 2 or 3; |
| | N6 is a uracil or cytosine; |
| | N7 is a uracil or guanine; |
| | N8 is adenine or guanine and x is an integer from 0 to 1. |
| 117 | GGAAAAUUUUAGCCUGGAACGUUAGAUAACUGUCCUGUUGUCUUUAUAUAC |
| | UUGGUCCCCAAGUAGUUUGUCUUCCAAA |
| 118 | GGAAACUUUAUUUAGUGUUACUUUAUUUUCUGUUUAUUUGUGUUUCUUCAG |
| | UGGGUUUGUUCUAAUUUCCUUGGCCGCC |
| 119 | GGAAAAUCUGUAUUAGGUUGGCGUGUUCUUUGGUCGGUUGUUAGUAUUGUU |
| | GUUGAUUCGUUUGUGGUCGGUUGCCGCC |
| 120 | GGAAAAUUAUUAACAUCUUGGUAUUCUCGAUAACCAUUCGUUGGAUUUUAU |
| | UGUAUUCGUAGUUUGGGUUCCUGCCGCC |
| 121 | GGAAAUUAUUAUUAUUUCUAGCUACAAUUUAUCAUUGUAUUAUUUUAGCUA |
| | UUCAUCAUUAUUUACUUGGUGAUCAACA |
| 122 | GGAAAUAGGUUGUUAACCAAGUUCAAGCCUAAUAAGCUUGGAUUCUGGUGA |
| | CUUGCUUCACCGUUGGCGGGCACCGAUC |
| 123 | GGAAAUCGUAGAGAGUCGUACUUAGUACAUAUCGACUAUCGGUGGACACCAU |
| | CAAGAUUAUAAACCAGGCCAGA |
| 124 | GGAAACCCGCCCAAGCGACCCCAACAUAUCAGCAGUUGCCCAAUCCCAACUCC |
| | CAACACAAUCCCCAAGCAACGCCGCC |
| 125 | GGAAAGCGAUUGAAGGCGUCUUUUCAACUACUCGAUUAAGGUUGGGUAUCG |
| | UCGUGGGACUUGGAAAUUUGUUGUUUCC |
| 126 | GGAAACUAAUCGAAAUAAAAGAGCCCCGUACUCUUUUAUUUCUAUUAGGUUA |
| | GGAGCCUUAGCAUUUGUAUCUUAGGUA |
| 127 | GGAAAUGUGAUUUCCAGCAACUUCUUUUGAAUAUAUUGAAUUCCUAAUUCA |
| | AAGCGAACAAAUCUACAAGCCAUAUACC |
| 128 | GGAAAUCGUAGAGAGUCGUACUUACGUGGUCGCCAUUGCAUAGCGCGCGAAA |
| | GCAACAGGAACAAGAACGCGCC |
| 129 | GGAAAUCGUAGAGAGUCGUACUUAGAAUAAACAGAGUCGGGUCGACUUGUC |
| | UCUGAUACUACGACGUCACAAUC |
| 130 | GGAAAAUUUGCCUUCGGAGUUGCGUAUCCUGAACUGCCCAGCCUCCUGAUAU |
| | ACAACUGUUCCGCUUAUUCGGGCCGCC |
| 131 | GGAAAUCUGAGCAGGAAUCCUUUGUGCAUUGAAGACUUUAGAUUCCUCUCUG |
| | CGGUAGACGUGCACUUAUAAGUAUUUG |
| 132 | GGAAAGCGAUUGAAGGCGUCUUUUCAACUACUCGAUUAAGGUUGGGUAUCG |
| | UCGUGGGACUUGGAAAUUUGUUGCCACC |
| 133 | GGAAAUUUUUUUUUGAUAUUAUAAGAGUUUUUUUUUGAUAUUAAGAAAAUUU |
| | UUUUUUGAUAUUAGAAGAGUAAGAAGAAAUAUAAGACCCCGGCGCCGCCACC |
| 134 | GGAAAUAAGAGAGAAAAGAAGAGUAAGAAGAAAUAUAAGAGCCAAAAAAAA |
| | AAAACC |
| 135 | GGAAAUCUCCCUGAGCUUCAGGGAGUAAGAGAGAAAAGAAGAGUAAGAAGA |
| | AAUAUAAGACCCCGGCGCCGCCACC |
| 136 | GCCRCC, wherein R = A or G |
| 137 | GGACUCACUAUUUGUUUUCGCGCCCAGUUGCAAAAA |

TABLE 3
3′ UTR sequences (stop cassette is italicized; miR binding sites
are boldened)

| SEQ ID | |
|---|---|
| NO: | Sequence |
| 105 | UGAUAAUAGGCUGGAGCCUCGGUGGCCAUGCUUCUUGCCCCUUGGGCCUCCCCC |
| | CAGCCCCUCCUCCCCUUCCUGCACCCGUACCCCCGUGGUCUUUGAAUAAAGUCU |
| | GAGUGGGCGGC |
| 106 | UGAUAAUAGGCUGGAGCCUCGGUGGCCUAGCUUCUUGCCCCUUGGGCCUCCCCC |
| | CAGCCCCUCCUCCCCUUCCUGCACCCGUACCCCCGUGGUCUUUGAAUAAAGUCU |
| | GAGUGGGCGGC |
| 138 | UAAAGCUCCCCGGGGGCCUCGGUGGCCUAGCUUCUUGCCCCUUGGGCCUCCCCCC |
| | AGCCCCUCCUCCCCUUCCUGCACCCGUACCCCCGUGGUCUUUGAAUAAAGUCUG |
| | AGUGGGCGGC |
| 139 | UAAGUCUAAGCUGGAGCCUCCUGAGAGACCUGUGUGAACUAUUGAGAAGAUCGG |
| | AACAGCUCCUUACUCUGAGGAAGUUGGUACCCCCGUGGUCUUUGAAUAAAGUCU |
| | GAGUGGGCGGC |
| 140 | UAAAGCUCCCCGGGGCAAACACCAUUGUCACACUCCAGCCUCGGUGGCCUAGCU |
| | UCUUGCCCCUUGGGCCCAAACACCAUUGUCACACUCCAUCCCCCCAGCCCCUCC |
| | UCCCCUUCCUGCACCCGUACCCCCCAAACACCAUUGUCACACUCCAGUGGUCUU |
| | UGAAUAAAGUCUGAGUGGGGGC |
| | (miR122 binding sites boldened) |
| 141 | UAAAGCUCCCCGGGGUCCAUAAAGUAGGAAACACUACAGCUGGAGCCUCGGUGG |
| | CCUAGCUUCUUGCCCCUUGGGCCCAAACACCAUUGUCACACUCCAUCCCCCCAG |
| | CCCCUCCUCCCCUUCCUGCACCCGUACCCCCGUGGUCUUUGAAUAAAGUCUGAG |
| | UGGGCGGC |
| | (miR-142-3p and miR 122 binding sites boldened) |
| 142 | UAAAGCUCCCCGGGGGCCUCGGUGGCCUAGCUUCUUGCCCCUUGGGCCUCCCCCC |
| | AGCCCCUCCUCCCCUUCCUGCACCCGUACCCCCCAAACACCAUUGUCACACUCC |
| | AGUGGUCUUUGAAUAAAGUCUGAGUGGGGGC |
| | (miR122 binding site boldened) |
| 143 | UAAGCCCCUCCGGGGCAAACACCAUUGUCACACUCCAGCCUCGGUGGCCUAGCU |
| | UCUUGCCCCUUGGGCCCAAACACCAUUGUCACACUCCAUCCCCCCAGCCCCUCC |
| | UCCCCUUCCUGCACCCGUACCCCCCAAACACCAUUGUCACACUCCAGUGGUCUU |
| | UGAAUAAAGUCUGAGUGGGCGGC |
| | (miR 122 binding sites boldened) |
| 144 | UAAGCCCCUCCGGGGUCCAUAAAGUAGGAAACACUACAGCCUCGGUGGCCUAGC |
| | UUCUUGCCCCUUGGGCCUCCAUAAAGUAGGAAACACUACAUCCCCCCAGCCCCU |
| | CCUCCCCUUCCUGCACCCGUACCCCCCGCAUUAUUACUCACGGUACGAGUGGUC |
| | UUUGAAUAAAGUCUGAGUGGGCGGC |
| | (miR-142-3p and miR-126-3p binding sites boldened) |
| 145 | UAAGCCCCUCCGGGGUCCAUAAAGUAGGAAACACUACAGCUGGAGCCUCGGUG |
| | GCCUAGCUUCUUGCCCCUUGGGCCCAAACACCAUUGUCACACUCCAUCCCCCCA |
| | GCCCCUCCUCCCCUUCCUGCACCCGUACCCCCGUGGUCUUUGAAUAAAGUCUGA |
| | GUGGGCGGC |
| | (miR-142-3p and miR 122 binding sites boldened) |
| 146 | UAAGCCCCUCCGGGGGCCUCGGUGGCCUAGCUUCUUGCCCCUUGGGCCUCCCCCC |
| | AGCCCCUCCUCCCCUUCCUGCACCCGUACCCCCCAAACACCAUUGUCACACUCC |
| | AGUGGUCUUUGAAUAAAGUCUGAGUGGGCGGC |
| | (miR122 binding site boldened) |

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A composition comprising:
(i) at least one, but not more than ten, messenger ribonucleic acid (mRNA) comprising an open reading frame (ORF) encoding a norovirus major capsid protein (VP1); and
(ii) a lipid nanoparticle (LNP).

2. The composition of claim 1, further comprising at least two, at least three, at least four, at least five, at least six, or at least seven, messenger ribonucleic acid (mRNA) comprising an open reading frame (ORF) encoding a norovirus major capsid protein (VP1),
wherein the at least two, at least three, at least four, at least five, at least six, or at least seven mRNA encode for at least two, at least three, at least four, at least five, at least six, or at least seven norovirus VP1 are each of different genotypes.

3. The composition of claim 2, wherein the genotypes are selected from genogroup GI and/or genogroup GII.

4. A composition comprising:
(i) a first messenger ribonucleic acid (mRNA) comprising an open reading frame (ORF) encoding a first norovirus VP1;
(ii) a second messenger ribonucleic acid (mRNA) comprising an open reading frame (ORF) encoding a second norovirus VP1;
(iii) a third messenger ribonucleic acid (mRNA) comprising an open reading frame (ORF) encoding a third norovirus VP1; and
(vi) a lipid nanoparticle (LNP),
wherein the first norovirus VP1 comprises a different genogroup relative to at least one of the second, and/or third norovirus VP1s, and wherein the first, second, and third norovirus VP1 all comprise different genotypes.

5. A composition comprising:
(i) a first messenger ribonucleic acid (mRNA) comprising an open reading frame (ORF) encoding a first norovirus VP1;
(ii) a second messenger ribonucleic acid (mRNA) comprising an open reading frame (ORF) encoding a second norovirus VP1;
(iii) a third messenger ribonucleic acid (mRNA) comprising an open reading frame (ORF) encoding a third norovirus VP1;
(iv) a fourth messenger ribonucleic acid (mRNA) comprising an open reading frame (ORF) encoding a fourth norovirus VP1;
(v) a fifth messenger ribonucleic acid (mRNA) comprising an open reading frame (ORF) encoding a fifth norovirus VP1; and
(vi) a lipid nanoparticle (LNP),
wherein the first norovirus VP1 comprises a different genogroup relative to at least one of the second, third, fourth, and/or fifth norovirus VP1s, and wherein the first, second, third, fourth, and/or fifth norovirus VP1 all comprise different genotypes.

6. A composition comprising:
(i) a first messenger ribonucleic acid (mRNA) comprising an open reading frame (ORF) encoding a first norovirus VP1;
(ii) a second messenger ribonucleic acid (mRNA) comprising an open reading frame (ORF) encoding a second norovirus VP1;
(iii) a third messenger ribonucleic acid (mRNA) comprising an open reading frame (ORF) encoding a third norovirus VP1;
(iv) a fourth messenger ribonucleic acid (mRNA) comprising an open reading frame (ORF) encoding a fourth norovirus VP1;
(v) a fifth messenger ribonucleic acid (mRNA) comprising an open reading frame (ORF) encoding a fifth norovirus VP1;
(vi) a sixth messenger ribonucleic acid (mRNA) comprising an open reading frame (ORF) encoding a six norovirus VP1;
(vii) a seventh messenger ribonucleic acid (mRNA) comprising an open reading frame (ORF) encoding a seventh norovirus VP1, and
(viii) a lipid nanoparticle (LNP),
wherein the first norovirus VP1 comprises a different genogroup relative to at least one of the second, third, fourth, fifth, sixth, and/or seventh norovirus VP1s, and wherein first, second, third, fourth, fifth, sixth, and/or seventh norovirus VP1s all comprise different genotypes.

7. The composition of any one of claims 1-6, wherein the first, second, third, fourth, fifth, sixth, and/or seventh norovirus VP1s each comprise a shell domain (S-domain) and a protruding domain (P-domain), wherein the S-domain and P-domain are linked by a hinge domain (H-domain).

8. The composition of any one of claims 1-7, wherein two norovirus VP1s form a homodimer.

9. The composition of any one of claims 1-8, wherein the first, second, third, fourth, fifth, sixth, and/or seventh norovirus VP1s are from a norovirus classified as genogroup GI.

10. The composition of claim 9, wherein the first, second, third, fourth, fifth, sixth, and/or seventh norovirus VP1s are selected from a norovirus classified as genotype GI.1, GI.2, GI.3, GI.4, GI.5, GI.6, GI.7, GI.8, or GI.9.

11. The composition of claim 10, wherein the first, second, third, fourth, fifth, sixth, and/or seventh norovirus VP1s are from a norovirus classified as genotype GI.3.

12. The composition of claim 11, wherein the first, second, third, fourth, fifth, sixth, and/or seventh norovirus VP1s comprise an amino acid sequence having at least 90% identity to the amino acid sequence of SEQ ID NO: 24.

13. The composition of any one of claims 1-8, wherein the first, second, third, fourth, fifth, sixth, and/or seventh norovirus VP1s are from a norovirus classified as genogroup GII.

14. The composition of claim 13, wherein the first, second, third, fourth, fifth, sixth, and/or seventh norovirus VP1s are selected from a norovirus classified as genotype GII.1, GII.2, GII.3, GII.4, GII.5, GII.6, GII.7, GII.8, GII.9, GII.10, GII.11, GII.12, GII.13, GII.14, GII.15, GII.16, GII.17, GII.18, GII.19 or GII.20.

15. The composition of claim 14, wherein the first, second, third, fourth, fifth, sixth, and/or seventh norovirus VP1s are from a norovirus classified as genotype GII.4.

16. The composition of claim 15, wherein the first, second, third, fourth, fifth, sixth, and/or seventh norovirus VP1s comprise an amino acid sequence having at least 90% identity to the amino acid sequence of SEQ ID NO: 3.

17. The composition of claim 14, wherein the first, second, third, fourth, fifth, sixth, and/or seventh norovirus VP1s are from a norovirus classified as genotype GII.17.

18. The composition of claim 17, wherein the first, second, third, fourth, fifth, sixth, and/or seventh norovirus VP1s comprise an amino acid sequence having at least 90% identity to the amino acid sequence of SEQ ID NO: 20.

19. The composition of claim 14, wherein the first, second, third, fourth, fifth, sixth, and/or seventh norovirus VP1s are from a norovirus classified as genotype GII.2.

20. The composition of claim 19, wherein the first, second, third, fourth, fifth, sixth, and/or seventh norovirus VP1s comprise an amino acid sequence having at least 90% identity to the amino acid sequence of SEQ ID NO: 18.

21. The composition of claim 14, wherein the first, second, third, fourth, fifth, sixth, and/or seventh norovirus VP1s are from a norovirus classified as genotype GII.3.

22. The composition of claim 21 wherein the first, second, third, fourth, fifth, sixth, and/or seventh norovirus VP1s comprise an amino acid sequence having at least 90% identity to the amino acid sequence of SEQ ID NO: 64.

23. The composition of claim 14, wherein the first, second, third, fourth, fifth, sixth, and/or seventh norovirus VP1s are from a norovirus classified as genotype GII.6.

24. The composition of claim 23, wherein the first, second, third, fourth, fifth, sixth, and/or seventh norovirus VP1s comprise an amino acid sequence having at least 90% identity to the amino acid sequence of SEQ ID NO: 16.

25. The composition of claim 4, wherein the different genotypes are GI.3, GII.3, and GII.4.

26. The composition of claim 5, wherein the different genotypes are GI.3, GII.2, GII.3, GII.4, and GII.6.

27. The composition of any one of claims 4 and 7-26, wherein at least one LNP comprises the mRNA comprising the ORF encoding the first norovirus VP1, the mRNA comprising the ORF encoding the second norovirus VP1, and the mRNA comprising the ORF encoding the third norovirus VP1.

28. The composition of any one of claims 5 and 7-26, wherein at least one LNP comprises the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, and the mRNA comprising the ORF encoding the fifth norovirus VP1.

29. The composition of any one of claims 6-26, wherein at least one LNP comprises the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, the mRNA comprising the ORF encoding the fifth norovirus VP1, the mRNA comprising the ORF encoding the sixth norovirus VP1, and/or the mRNA comprising the ORF encoding the seventh norovirus VP1.

30. The composition of any one of claims 4 and 7-26, wherein a first LNP comprises the mRNA comprising the ORF encoding the first norovirus VP1, a second LNP comprises the mRNA comprising the ORF encoding the second norovirus VP1, and a third LNP comprises the mRNA comprising the ORF encoding the third norovirus VP1.

31. The composition of any one of claims 5 and 7-26, wherein a first LNP comprises the mRNA comprising the ORF encoding the first norovirus VP1, a second LNP comprises the mRNA comprising the ORF encoding the second norovirus VP1, a third LNP comprises the mRNA comprising the ORF encoding the third norovirus VP1, a fourth LNP comprises the mRNA comprising the ORF encoding the fourth norovirus VP1, and a fifth LNP comprises the mRNA comprising the ORF encoding the fifth norovirus VP1 comprise separate LNPs.

32. The composition of any one of claims 6-26, wherein a first LNP comprises the mRNA comprising the ORF encoding the first norovirus VP1, a second LNP comprises the mRNA comprising the ORF encoding the second norovirus VP1, a third LNP comprises the mRNA comprising the ORF encoding the third norovirus VP1, a fourth LNP comprises the mRNA comprising the ORF encoding the fourth norovirus VP1, a fifth LNP comprises the mRNA comprising the ORF encoding the fifth norovirus VP1, a sixth LNP comprises the mRNA comprising the ORF encoding the sixth norovirus VP1, and/or a seventh LNP comprises the mRNA comprising the ORF encoding the seventh norovirus VP1.

33. The composition of any one of claims 4-32, wherein the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, the mRNA comprising the ORF encoding the fifth norovirus VP1, the mRNA comprising the ORF encoding the sixth norovirus VP1, and/or the mRNA comprising the ORF encoding the seventh norovirus VP1 are present in one of the following ratios:
(i) 1:1, 1:2, 1:3, 2:3;
(ii) 1:1:1, 1:3:1, 2:3:1;
(iii) 1:1:1:1, 1:3:1:1, 2:3:1:1;
(iv) 1:1:1:1:1, 1:3:1:1:1, 2:3:1:1:1;
(v) 1:1:1:1:1:1, 1:3:1:1:1:1, 2:3:1:1:1:1; or
(vi) 1:1:1:1:1:1:1; 1:3:1:1:1:1:1, 2:3:1:1:1:1:1.

34. The composition of claim 33, wherein the mRNA comprising the ORF encoding the first norovirus VP1, the mRNA comprising the ORF encoding the second norovirus VP1, and the mRNA comprising the ORF encoding the third norovirus VP1 are present in the following ratio: 1:1:3.

35. The composition of claim 34, wherein the first norovirus VP1 is GI.3, the second norovirus VP1 is GII.3, and the third norovirus VP1 is GII.4.

36. The composition of claim 33, wherein the mRNA comprising the ORF encoding the first norovirus VP1, the mRNA comprising the ORF encoding the second norovirus VP1, the mRNA comprising the ORF encoding the third norovirus VP1, the mRNA comprising the ORF encoding the fourth norovirus VP1, and the mRNA comprising the ORF encoding the fifth norovirus VP1 are present in the following ratio: 1:1:3:1:1.

37. The composition of claim 36, wherein the first norovirus VP1 is GI.3, the second norovirus VP1 is GII.3, the third norovirus VP1 is GII.4, the fourth norovirus VP1 is GII.2, and the fifth norovirus VP1 is GII.6.

38. The composition of any one of claims 1-37, wherein the LNP comprises an ionizable amino lipid, a sterol, neutral lipid, and a PEG-modified lipid.

39. The composition of claim 38, wherein the LNP comprises 40-50 mol % ionizable amino lipid, 35-45 mol % sterol, 10-15 mol % neutral lipid, and 2-4 mol % PEG-modified lipid.

40. The composition of any one of the preceding claims, wherein the LNP comprises 45 mol %, 46 mol %, 47 mol %, 48 mol %, 49 mol %, or 50 mol % ionizable amino lipid.

41. The composition of any one of the preceding claims, wherein the ionizable amino lipid has the structure of Compound 1:

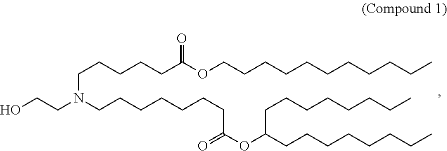 [SMILES: CCCCCCCCCCCOC(=O)CCCCCN(CCO)CCCCCCCC(=O)OC(CCCCCCCC)CCCCCCCC]

42. The composition of any one of claims 38-41, wherein the sterol is cholesterol or a variant thereof.

43. The composition of any one of claims 38-42, wherein the neutral lipid is 1,2 distearoyl-sn-glycero-3-phosphocholine (DSPC).

44. A method comprising administering to a subject an immunogenic composition comprising:
(i) a messenger ribonucleic acid (mRNA) comprising an open reading frame encoding a first norovirus VP1;
(ii) a messenger ribonucleic acid (mRNA) comprising an open reading frame encoding a second norovirus VP1;
(iii) a messenger ribonucleic acid (mRNA) comprising an open reading frame encoding a third norovirus VP1;
in an amount effective to induce in the subject an immune response against a viral infection from a member of a Norovirus genus.

45. A method comprising administering to a subject an immunogenic composition comprising:
(i) a messenger ribonucleic acid (mRNA) comprising an open reading frame encoding a first norovirus VP1;
(ii) a messenger ribonucleic acid (mRNA) comprising an open reading frame encoding a second norovirus VP1;
(iii) a messenger ribonucleic acid (mRNA) comprising an open reading frame encoding a third norovirus VP1;
(iv) a messenger ribonucleic acid (mRNA) comprising an open reading frame encoding a fourth norovirus VP1; and/or
(v) a messenger ribonucleic acid (mRNA) comprising an open reading frame encoding a fifth norovirus VP1;
in an amount effective to induce in the subject an immune response against a viral infection from a member of a Norovirus genus.

46. A method comprising administering to a subject an immunogenic composition comprising:
(i) a messenger ribonucleic acid (mRNA) comprising an open reading frame encoding a first norovirus VP1;
(ii) a messenger ribonucleic acid (mRNA) comprising an open reading frame encoding a second norovirus VP1;
(iii) a messenger ribonucleic acid (mRNA) comprising an open reading frame encoding a third norovirus VP1;
(iv) a messenger ribonucleic acid (mRNA) comprising an open reading frame encoding a fourth norovirus VP1;
(v) a messenger ribonucleic acid (mRNA) comprising an open reading frame encoding a fifth norovirus VP1;
(vi) a messenger ribonucleic acid (mRNA) comprising an open reading frame encoding a six norovirus VP1; and/or
(vii) a messenger ribonucleic acid (mRNA) comprising an open reading frame encoding a seventh norovirus VP1,
in an amount effective to induce in the subject an immune response against a viral infection from a member of a Norovirus genus.

47. The method of any one of claims 44-46, wherein the immune response includes a binding antibody titer to a human norovirus of the genogroup GI.

48. The method of claim 47, wherein the human norovirus is genotype GII.4.

49. The method of claim 47, wherein the human norovirus is genotype GII.6.

50. The method of claim 47, wherein the human norovirus is genotype GII.2.

51. The method of claim 47, wherein the human norovirus is genotype GII.3.

52. The method of any one of claims 44-46, wherein the immune response includes a binding antibody titer to a human norovirus of the genogroup GI.

53. The method of claim 52, wherein the human norovirus is genotype GI.3.

54. The method of any one of claims 44-53, wherein the immune response includes a T cell response to a human norovirus.

55. The method of claim 44, wherein the immunogenic composition further comprises at least one lipid nanoparticle.

56. The method of claim 55, wherein the mRNA of (i), (ii), and/or (iii) are administered to the subject at the same time.

57. The method of claim 45, wherein the immunogenic composition further comprises at least one lipid nanoparticle.

58. The method of claim 57, wherein the mRNA of (i), (ii), (iii), (iv), and/or (v) are administered to the subject at the same time.

59. The method of claim 46, wherein the immunogenic composition further comprises at least one lipid nanoparticle.

60. The method of claim 59, wherein the mRNA of (i), (ii), (iii), (iv), (v), (vi), and/or (vii) are administered to the subject at the same time.

61. The method of claim 44, wherein the immunogenic composition further comprises at least two, but no more than three lipid nanoparticles.

62. The method of claim 61, wherein the mRNA of (i), (ii), and/or (iii) are administered to the subject at the same time.

63. The method of claim 45, wherein the immunogenic composition further comprises at least two, at least three, at least four, but no more than five lipid nanoparticles.

64. The method of claim 63, wherein the mRNA of (i), (ii), (iii), (iv), and/or (v) are administered to the subject at the same time.

65. The method of claim 46, wherein the immunogenic composition further comprises at least two, at least three, at least four, at least five, at least six, but no more than seven lipid nanoparticles.

66. The method of claim 65, wherein the mRNAs of (i), (ii), (iii), (iv), (v), (vi), and/or (vii) are administered to the subject at the same time.
